(19) United States
(12) Patent Application Publication
(10) Pub. No.: US 2026/0008840 A1
(43) Pub. Date: Jan. 8, 2026

(54) METHODS OF DIAGNOSING AND TREATING NEURODEGENERATIVE DISORDERS

(71) Applicant: ALZPATH, INC., Carlsbad, CA (US)

(72) Inventor: Andreas Jeromin, Alachua, FL (US)

(21) Appl. No.: 19/323,142

(22) Filed: Sep. 9, 2025

Related U.S. Application Data
(63) Continuation of application No. PCT/US24/20266, filed on Mar. 15, 2024.
(60) Provisional application No. 63/490,773, filed on Mar. 16, 2023.

(51) Int. Cl. C07K 16/18; A61K 39/00; A61K 45/06; A61P 25/28; G01N 33/68

(52) U.S. Cl. CPC C07K 16/18; A61K 45/06; A61P 25/28; G01N 33/6896; A61K 2039/505; A61K 2039/545; G01N 2333/4709; G01N 2800/2821; G01N 2800/52; G01N 2800/56

(57) ABSTRACT

Provided herein are compositions and methods relating to improved assays for establishing a condition of a neurodegenerative disease and providing treatment. Further provided herein are compositions and methods comprising improved antibodies for assays including immunoassays used for diagnosing Alzheimer's disease and providing treatment.

### CROSS-REFERENCE TO RELATED APPLICATIONS

This application is a continuation application of International Patent Application No. PCT/US24/20266, filed Mar. 15, 2024, which claims the benefit of U.S. Provisional Patent Application No. 63/490,773, filed on Mar. 16, 2023 which is incorporated by reference herein in its entirety.

### BACKGROUND

The discovery of biomarkers and screening techniques of Alzheimer's disease (AD) and other tauopathies is an ongoing area of development in which these tools may be applied to screening populations to determine which non-demented individuals are at greatest risk of developing AD dementia and also to assess disease progression in patients. Proteins that are reflective of AD pathology, including amyloid beta 42 (Aβ₄₂), neurofilament light chain, and various tau isoforms have been detected by a variety of means. Abnormal or excessive phosphorylation of tau has been associated with transformation of pathologically normal tau molecules into paired-helical-filament (PHF) tau and neurofibrillary tangles (NFTs) indicative of various tauopathy pathologies.

### INCORPORATION BY REFERENCE OF SEQUENCE LISTING

The present application is being filed along with a Sequence Listing in electronic format. The Sequence Listing is provided as a file entitled 58484-707.301_SL.xml, created on Sep. 5, 2025, which is 106,416 bytes in size. The information in the electronic format of the Sequence Listing is incorporated by reference in its entirety.

### SUMMARY

In an aspect described herein are methods of treating a neurodegenerative disease in a subject, wherein the neurodegenerative disease is characterized by accumulation of amyloid-β (Aβ) peptide in a brain of the subject, the methods comprising: administering to the subject a therapeutic agent comprising (i) an inhibitor of the AB peptide or a modified form of the AB peptide or (ii) an inhibitor of a phosphorylated tau protein, for treatment of the neurodegenerative disease, wherein the subject is identified for the treatment based, at least in part, on a level of phosphorylated tau 217 (p-Tau 217) measured in a sample obtained from the subject that is above the level of the p-Tau 217 measured in the sample with a standard value of p-Tau 217 level derived from a plurality of reference samples from reference subjects that do not have the neurodegenerative disease, which is predictive that the subject has deposition of abnormal tau protein in the brain with a sensitivity that is greater than or equal to about 85%. In some embodiments, the p-Tau 217 is measured in the sample by a method comprising performing an immunoassay on the sample using an anti-tau antibody or antigen-binding fragment thereof. In some embodiments, the immunoassay is a digital immunoassay configured to measure the p-Tau 217 in a sample that is a fluid sample. In some embodiments, the digital immunoassay is a Single Molecule Array (SIMOA). In some embodiments, the immunoassay comprises an enzyme-linked immunoassay (ELISA), a radioimmunoassay (RIA), a fluoroimmunoassay (FIA), a chemiluminescent immunoassay (CLIA), or a counting immunoassay (CIA). In some embodiments, the anti-tau antibody or the antigen-binding fragment thereof comprises: a) a heavy chain comprising a variable heavy chain (VH) domain, wherein the VH domain comprises an HCDR1 sequence selected from SEQ ID NOs: 1-9, an HCDR2 sequence selected from SEQ ID NOs: 10-17, and an HCDR3 sequence selected from SEQ ID NOs: 18-23; and b) a light chain comprising a variable light chain (VL) domain, wherein the VL domain comprises an LCDR1 sequence selected from SEQ ID NOs: 24-31, an LCDR2 sequence selected from SEQ ID NOs: 32-36, and an LCDR3 sequence selected from SEQ ID NOs: 37-43. In some embodiments, the HCDR1 sequence comprises SEQ ID NO: 2, the HCDR2 sequence comprises SEQ ID NO: 11, and the HCDR3 sequence comprises SEQ ID NO: 19; and the LCDR1 sequence comprises SEQ ID NO: 25, the LCDR2 sequence comprises SEQ ID NO: 33, and the LCDR3 sequence comprises SEQ ID NO: 38. In some embodiments, the HCDR1 sequence comprises SEQ ID NO: 1, the HCDR2 sequence comprises SEQ ID NO: 10, and the HCDR3 sequence comprises SEQ ID NO: 18; and the LCDR1 sequence comprises SEQ ID NO: 24, the LCDR2 sequence comprises SEQ ID NO: 32, and the LCDR3 sequence comprises SEQ ID NO: 37. In some embodiments, the HCDR1 sequence comprises SEQ ID NO: 2, the HCDR2 sequence comprises SEQ ID NO: 11, and the HCDR3 sequence comprises SEQ ID NO: 19; and the LCDR1 sequence comprises SEQ ID NO: 26, the LCDR2 sequence comprises SEQ ID NO: 34, and the LCDR3 sequence comprises SEQ ID NO: 39. In some embodiments, the HCDR1 sequence comprises SEQ ID NO: 3, the HCDR2 sequence comprises SEQ ID NO: 12, and the HCDR3 sequence comprises SEQ ID NO: 18; and the LCDR1 sequence comprises SEQ ID NO: 27, the LCDR2 sequence comprises SEQ ID NO: 32, and the LCDR3 sequence comprises SEQ ID NO: 40. In some embodiments, the HCDR1 sequence comprises SEQ ID NO: 4, the HCDR2 sequence comprises SEQ ID NO: 11, and the HCDR3 sequence comprises SEQ ID NO: 20; and the LCDR1 sequence comprises SEQ ID NO: 28, the LCDR2 sequence comprises SEQ ID NO: 35, and the LCDR3 sequence comprises SEQ ID NO: 41. In some embodiments, the HCDR1 sequence comprises SEQ ID NO: 5, the HCDR2 sequence comprises SEQ ID NO: 13, the HCDR3 sequence comprises SEQ ID NO: 21; and the LCDR1 sequence comprises SEQ ID NO: 29, the LCDR2 sequence comprises SEQ ID NO: 33, and the LCDR3 sequence comprises SEQ ID NO: 42. In some embodiments, anti-tau antibody comprises a heavy chain (HC) sequence comprising SEQ ID NO: 57 and a light chain (LC) sequence comprising SEQ ID NO: 58. In some embodiments, the anti-tau antibody comprises a HC sequence comprising SEQ ID NO: 57 and a LC sequence comprising SEQ ID NO: 59. In some embodiments, the anti-tau antibody comprises a HC sequence comprising SEQ ID NO: 60 and a LC sequence comprising SEQ ID NO: 61. In some embodiments, the anti-tau antibody comprises a HC sequence comprising SEQ ID NO: 62 and a LC sequence comprising SEQ ID NO: 63. In some embodiments, the anti-tau antibody comprises a HC sequence comprising SEQ ID NO: 64 and a LC sequence comprising SEQ ID NO: 65. In some embodiments, the anti-tau antibody comprises a HC sequence comprising SEQ ID NO: 55 and a LC sequence comprising SEQ ID NO: 56. In some embodiments, the anti-tau antibody comprises a variable heavy (VH) domain comprising a sequence that has at least 80%, at least 85%, at least 90%, or at least 95% sequence identity to a sequence selected from SEQ ID NOs: 44-48. In some embodiments, the anti-tau antibody comprises a variable light (VL) domain comprising a sequence that has at least 80%, at least 85%, at least 90%, or at least 95% sequence identity to a sequence selected from SEQ ID NOs: 49-54. In some embodiments, the anti-tau antibody or antigen-binding fragment thereof comprises an IgG-scFv, nanobody, BiTE, diabody, DART, TandAb, scDiabody, scDiabody-CH3, triple body, mini-antibody, minibody, TriBi minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, tetravalent HCAb, scDiabody-Fc, diabody-Fc, tandem scFv-Fc, or intrabody. In some embodiments, the anti-tau antibody has an isotype selected from the group consisting of IgA1, IgA2, IgD, IgE, IgG1, IgG2, IgG3, IgG4, and IgM. In some embodiments, the anti-tau antibody or antigen-binding fragment thereof specifically binds an epitope in an N-terminal region of a tau polypeptide. In some embodiments, the anti-tau antibody or antigen-binding fragment thereof specifically binds to p-Tau 217-tau protein. In some embodiments, the anti-tau antibody or antigen-binding fragment thereof specifically binds to a cis-conformation of p-Tau 217-tau protein. In some embodiments, the anti-tau antibody or antigen-binding fragment thereof binds specifically to a tau polypeptide comprising phosphorylation at a site selected from a group consisting of pT181-tau, pT212-tau, phosphorylated-serine (pS)214-tau, pT217-tau, pT220-tau, and pT231-tau. In some embodiments, the anti-tau antibody or antigen-binding fragment thereof binds specifically to a tau polypeptide comprising phosphorylation at site pT217-tau. In some embodiments, the anti-tau antibody or antigen-binding fragment thereof binds specifically to a tau polypeptide comprising phosphorylation at site pT217-tau and pT231-tau. In some embodiments, the subject has, or is suspected of having, Alzheimer's disease (AD). In some embodiments, the AD causes, is associated with, or presents with a tauopathy in the subject. In some embodiments, the tauopathy comprises a secondary tauopathy comprising neurofibrillary tangle (NTF) pathology in the brain of the subject, wherein the tauopathy is secondary to amyloid-beta plaques in the brain of the subject. In some embodiments, method of claim 30, wherein AD comprises a variant AD selected from the group consisting of early-onset Alzheimer's disease, late-onset Alzheimer's disease, Familial Alzheimer's disease (FAD), a mixed dementia comprising Alzheimer's disease and vascular dementia, logopenic aphasia, posterior cortical atrophy, frontal variant Alzheimer's disease, and Alzheimer's disease combined with corticobasal syndrome (AD-CBS). In some embodiments, the tauopathy comprises hyperphosphorylated tau, misfolded tau, oligomeric tau, aggregated paired helical filaments (PFHs) of tau, neurofibrillary tangles (NFTs), or any combination thereof. In some embodiments, the subject is a human is age 60 or older. In some embodiments, the neurodegenerative disease comprises Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, Hallevorden-Spatz disease, mild cognitive impairment (MCI), Lewy Body's disease (LBD), Amyotrophic lateral sclerosis, spinal muscular atrophy, Friedreich's ataxia, Wernicke-Korsakoff syndrome, a prion disease, vascular dementia, Alzheimer's related dementia, Huntington's disease (HD), or a transgene-induced tauopathy. In some embodiments, the neurodegenerative disease is Alzheimer's disease. In some embodiments, a prodromal stage of the Alzheimer's disease is characterized by mild cognitive impairment (MCI). In some embodiments, a prodromal stage of Alzheimer's disease is characterized at least as. Braak stage I by a determined spatial extent of tau-PET. In some embodiments, a prodromal stage of Alzheimer's disease is characterized at least as. Braak stage II by a determined spatial extent of tau-PET. In some embodiments, the neurodegenerative disease is a tauopathy comprising hyperphosphorylated tau, misfolded tau, oligomeric tau, aggregated paired helical filaments (PFHs) of tau, neurofibrillary tangles (NFTs), or any combination thereof. In some embodiments, the inhibitor of the Aβ peptide or the modified form of the AR peptide comprises: ABvac40, ABBV-916, ACU193, AD-35, Aducanumab (Aduhelm®), APH-1105, BPN14770, Bapineuzumab, BMS-984923, Contraloid acetate, CNP520(AMG520), Crenezumab, Donanemab (LY3002813), Donepezil (Aricept), Elenbecestat (E2609), Gantenerumab, Brain Shuttle Gantenerumab (R07126209), GV-971, HT-ALZ, KHK 66401, Lanabecestat, Lecanemab (BAN2401) (Legembi®), Lu AF20513, MEDI 1814, Ponezumab, Remtemetug (LY3372993), rivastigmine, SHR-1707, Simufilam (PTI-125), Sodium oligomannate, Solanezumab, scyllo-inositol, UB-311, valiltramiprosate (ALZ-801), varoglutamstat (PQ912), verubecestat (MK-8931), VGH-AD1, an antisense RNA directed to an isoform of human amyloid beta, an siRNA directed to an isoform of human amyloid beta, an antisense oligonucleotide directed to an isoform of human amyloid beta, an LNA oligonucleotide directed to an isoform of human amyloid beta, a CRISPRn-based therapeutic targeting the human APP locus, or a CRISPRi-based therapeutic targeting the human APP locus. In some embodiments, the inhibitor of the Aβ peptide or the modified form of the Aβ peptide comprises. Donanemab. In some embodiments, the inhibitor of the AR peptide or the modified form of the Aβ peptide comprises Lecanemab. In some embodiments, the inhibitor of the Aβ peptide or the modified form of the Aβ peptide comprises Remtemetug. In some embodiments, the inhibitor of the Aβ peptide or the modified form of the Aβ peptide comprises Aducanumab. In some embodiments, the inhibitor of the AR peptide or the modified form of the Aβ peptide is selected from Table 10. In some embodiments, the inhibitor of the phosphorylated tau protein comprises an inhibitor of p-Tau 217 or a modified form of p-Tau 217, p-Tau 181, p-Tau 212, p-Tau 220, p-Tau 231, or phosphorylated-serine (pS)214-tau. In some embodiments, the inhibitor of the phosphorylated tau protein comprises an inhibitor of p-Tau 217. In some embodiments, the sensitivity is greater than or equal to about 90%. In some embodiments, the sensitivity is greater than or equal to about 92%. In some embodiments, the level of p-Tau 217 that is measured is predictive that the subject has the deposition of the abnormal tau protein in the brain with an area under the curve (AUC) that is greater than or equal to about 0.85. In some embodiments, the AUC is greater than or equal to about 0.90. In some embodiments, the level of p-Tau 217 that is measured is predictive that the subject has the deposition of the abnormal tau protein in the brain with a specificity that is greater than or equal to about 810%. In some embodiments, the specificity is greater than or equal to about 85%. In some embodiments, the level of p-Tau 217 that is measured is predictive that the subject has the deposition of the abnormal tau protein in the brain with a positive predictive value (PPV) that is greater than or equal to about 510%. In some embodiments, the PPV is greater than or equal to about 70%. In some embodiments, the level of p-Tau 217 that is measured is predictive that the subject has the deposition of the abnormal tau protein in the brain with a negative predictive value (NPV) that is greater than or equal to about 51%. In some embodiments, the NPV is greater than or equal to about 70%. In some embodiments, the level of p-Tau 217 that is measured is predictive that the subject will exhibit a positive therapeutic response to the therapeutic agent with a specificity that is greater than or equal to about 70%. In some embodiments, the level of p-Tau 217 that is measured is predictive that the subject will exhibit a positive therapeutic response to the therapeutic agent with a sensitivity that is greater than or equal to about 70%. In some embodiments, the level of p-Tau 217 that is measured is predictive that the subject will exhibit a positive therapeutic response to the therapeutic agent with a PPV that is greater than or equal to about 70%.the level of p-Tau In some embodiments, 217 that is measured is predictive that the subject will exhibit a positive therapeutic response to the therapeutic agent with a NPV that is greater than or equal to about 70%. In some embodiments, the level of p-Tau 217 that is measured is predictive that the subject will exhibit a positive therapeutic response to the therapeutic agent with an AUC that is greater than or equal to about 0.70. In some embodiments, the sample is a cerebrospinal fluid (CSF) sample, a plasma sample, a blood sample, or a serum sample. In some embodiments, the sample is a plasma sample. In some embodiments, the method comprises determining a likelihood of clinical diagnosis of the neurodegenerative disease in the subject based, at least in part, on the level of p-Tau 217 measured in the sample. In some embodiments, the method comprises establishing an early prognosis of the neurodegenerative disease in the subject based, at least in part, on the level of p-Tau 217 measured in the sample, wherein the early prognosis predates providing a diagnosis of a prodromal stage of the neurodegenerative disease in the subject. In some embodiments, the neurodegenerative disease is Alzheimer's disease, and wherein establishing the early prognosis of the Alzheimer's disease comprises establishing a likelihood of the Alzheimer's disease progressing to Alzheimer's disease dementia in the subject based, at least in part, on the level of p-Tau 217 measured in the sample. In some embodiments, the method comprises identifying an elevated risk that the neurodegenerative disease will lead to pathological memory impairment and cognitive decline in the subject, based at least in part, on the level of p-Tau 217 measured in the sample. In some embodiments, the method comprises ameliorating one or more symptoms of the neurodegenerative disease in the subject. In some embodiments, the method comprises slowing a progression of one or more symptoms of the neurodegenerative disease in the subject. In some embodiments, the therapeutic agent is administered intravenously, intramuscularly, intrathecally, intracerebrally, subcutaneously, orally, nasally, topically, buccally, or sublingually. In some embodiments, the therapeutic agent is administered directly to the CNS of the subject via intravenous delivery, intravascular delivery, intrathecal delivery, intracisternal delivery, intraspinal delivery, subpial delivery, or intracerebroventricular delivery. In some embodiments, wherein the subject is at a preclinical stage of the neurogenerative disease. In some embodiments, the subject is at a prodromal stage of the neurogenerative disease. In some embodiments, wherein the therapeutic agent is administered according to a body weight of the subject at a dosage of between about 0.1-100 mg/kg of the body weight. In some embodiments, the therapeutic agent is administered to the subject at a dosage of between about 200-1500 mg by intravenous administration. In some embodiments, the therapeutic agent is administered to the subject at a dosage of between about 700-1400 mg by intravenous administration. In some embodiments, the therapeutic agent is administered according to a body weight of the subject at a dosage of about 10 mg/kg of the body weight. In some embodiments, the therapeutic agent is administered to the subject according to a treatment regimen comprising administering the therapeutic agent about once every 2 weeks (q2 week). In some embodiments, the therapeutic agent is administered to the subject according to a treatment regimen comprising administering the therapeutic agent about once every 4 weeks (q4 week). In some embodiments, the therapeutic agent is administered to the subject is a plurality of doses, wherein a first three doses of the plurality of doses is administered to the subject at a dosage amount comprising about 700 mg per dose. In some embodiments, the doses of the plurality of doses administered to the subject after the first three doses are at a dosage amount comprising about 1400 mg per dose. In some embodiments, wherein the administering is systemic administration. In some embodiments, systemic administration comprises intravenous administration. In some embodiments, systemic administration comprises subcutaneous administration. In some embodiments, systemic administration comprises oral administration. In some embodiments, the method comprises: a)repeating the method following induction of the therapeutic agent, whereby a second level of p-Tau 217 is measured in a second sample obtained from the subject following induction of the therapeutic agent, and wherein the second level of the p-Tau 217 is lower than the level of p-Tau 217 measured prior to induction of the therapeutic agent; and b) administering another dose of the therapeutic agent to the subject.

In an aspect described herein, are kits for selecting a subject for treatment of a neurodegenerative disease, the kits comprising: a) an anti-tau antibody or the antigen-binding fragment thereof, comprising: i) a heavy chain comprising a variable heavy chain (VH) domain, wherein the VH domain comprises an HCDR1 sequence selected from SEQ ID NOs: 1-9, an HCDR2 sequence selected from SEQ ID NOs: 10-17, and an HCDR3 sequence selected from SEQ ID NOs: 18-23; and ii) a light chain comprising a variable light chain (VL) domain, wherein the VL domain comprises an LCDR1 sequence selected from SEQ ID NOs: 24-31, an LCDR2 sequence selected from SEQ ID NOs: 32-36, and an LCDR3 sequence selected from SEQ ID NOs: 37-43; ii) a heavy chain (HC) sequence comprising any one of SEQ ID NOs: 57, 60, 62, 64, or 55; and a light chain (LC) sequence comprising any one of SEQ ID NOs: 58, 59, 63, 65, or 56; or a variable heavy (VH) domain comprising a sequence that has at least 80%, at least 85%, at least 90%, at least 95% sequence identity to a sequence selected from SEQ ID NOs: 44-48; and a variable light (VL) domain comprising a sequence that has at least 80%, at least 85%, at least 90%, at least 95% sequence identity to a sequence selected from SEQ ID NOs: 49-54; b) instructions for analyzing a sample obtained from the subject using the anti-tau antibody or antigen-binding fragment thereof in an immunoassay; c) instructions for treating the neurodegenerative disease in the subject with a therapeutic agent comprising (i) an inhibitor of the Aβ peptide or a modified form of the AR peptide or (ii) an inhibitor of a phosphorylated tau protein; and d) optionally, the therapeutic agent. In some embodiments, the kit comprises the therapeutic agent. In some embodiments, the kit comprises one or more immunoassay components, wherein the immunoassay comprises Single Molecule Array (SIMOA), an enzyme-linked immunoassay (ELISA), a radioimmunoassay (RIA), a fluoroimmunoassay (FIA), a chemiluminescent immunoassay (CLIA), or a counting immunoassay (CIA). In some embodiments, the kit comprises a system for performing the immunoassay, wherein the immunoassay comprises Single Molecule Array (SIMOA), an enzyme-linked immunoassay (ELISA), a radioimmunoassay (RIA), a fluoroimmunoassay (FIA), a chemiluminescent immunoassay (CLIA), or a counting immunoassay (CIA). In some embodiments, the anti-tau antibody or the antigen-binding fragment thereof comprises a heavy chain comprising a variable heavy chain (VH) domain, wherein the VH domain comprises a HCDR1 sequence selected from SEQ ID NOs: 1-9, a HCDR2 sequence selected from SEQ ID NOs: 10-17, and a HCDR3 sequence selected from SEQ ID NOs: 18-23; and a light chain comprising a variable light chain (VL) domain, wherein the VL domain comprises a LCDR1 sequence selected from SEQ ID NOs: 24-31, a LCDR2 sequence selected from SEQ ID NOs: 32-36, and a LCDR3 sequence selected from SEQ ID NOs: 37-43. In some embodiments, the anti-tau antibody or the antigen-binding fragment thereof comprises a heavy chain (HC) sequence comprising any one of SEQ ID NOs: 57, 60, 62, 64, or 55; and a light chain (LC) sequence comprising any one of SEQ ID NOs: 58, 59, 63, 65, or 56. In some embodiments, the anti-tau antibody or the antigen-binding fragment thereof comprises a variable heavy (VH) domain comprising a sequence that has at least 80%, at least 85%, at least 90%, at least 95% sequence identity to a sequence selected from SEQ ID NOs: 44-48; and a variable light (VL) domain comprising a sequence that has at least 80%, at least 85%, at least 90%, at least 95% sequence identity to a sequence selected from SEQ ID NOs: 49-54. In some embodiments, the inhibitor of the therapeutic agent comprises: ABvac40, ABBV-916, ACU193, AD-35, Aducanumab (Aduhelm®), APH-1105, BPN14770, Bapineuzumab, BMS-984923, Contraloid acetate, CNP520(AMG520), Crenezumab, Donanemab (LY3002813), Donepezil (Aricept), Elenbecestat (E2609), Gantenerumab, Brain Shuttle Gantenerumab (R07126209), GV-971, HT-ALZ, KHK 66401, Lanabecestat, Lecanemab (BAN2401) (Legembi®), Lu AF20513, MEDI 1814, Ponezumab, Remternetug (LY3372993), rivastigmine, SHR-1707, Simufilam (PTI-125), Sodium oligomannate, Solanezumab, scyllo-inositol, UB-311, valiltramiprosate (ALZ-801), varoglutamstat (PQ912), verubecestat (MK-8931), VGH-AD1, an antisense RNA directed to an isoform of human amyloid beta, an siRNA directed to an isoform of human amyloid beta, an antisense oligonucleotide directed to an isoform of human amyloid beta, an LNA oligonucleotide directed to an isoform of human amyloid beta, a CRISPRn-based therapeutic targeting the human APP locus, or a CRISPRi-based therapeutic targeting the human APP locus. In some embodiments, the therapeutic agent comprises Donanemab. In some embodiments, the therapeutic agent comprises Lecanemab. In some embodiments, the therapeutic agent comprises Remternetug. In some embodiments, the therapeutic agent comprises Aducanumab. In some embodiments, the therapeutic agent is selected from Table 10. In some embodiments, the therapeutic agent is the inhibitor of the phosphorylated tau protein. In some embodiments, the inhibitor of the phosphorylated tau protein is an inhibitor of p-Tau 217 or a modified form of p-Tau 217, p-Tau 181, p-Tau 212, p-Tau 220, p-Tau 231, or phosphorylated-serine (pS)214-tau. In some embodiments, the inhibitor of the phosphorylated tau protein comprises an inhibitor of p-Tau 217. In some embodiments, the instructions for analyzing a sample further comprise a standard value of p-Tau 217 level derived from a plurality of reference samples from reference subjects that do not have the neurodegenerative disease.

Disclosed herein, in certain aspects, are methods of treating a neurodegenerative disease in a subject, the methods comprising: (a) detecting a level of pT217 tau in a sample from the subject and correlating the level of pT217 tau in the sample with a standard value of pT217 tau level derived from a plurality of reference samples, wherein the detecting comprises performing an immunoassay on the sample using an anti-tau antibody or antigen-binding fragment thereof comprising i) a heavy chain comprising a variable heavy chain (VH) domain and ii) a light chain comprising a variable light chain (VL) domain, wherein the VH domain comprises HCDR1 sequence selected from SEQ ID NOs: 1-9, HCDR2 sequence selected from SEQ ID NOs: 10-17, and HCDR3 sequence selected from SEQ ID NOs: 18-23, and the VL domain comprises LCDR1 sequence selected from SEQ ID NOs: 24-31, LCDR2 sequence selected from SEQ ID NOs: 32-36, and LCDR3 sequence selected from SEQ ID NOs: 37-43; (b) determining a likelihood of clinical diagnosis of the neurodegenerative disease in the subject based at least in part on the detecting; (c) establishing an early prognosis of the neurodegenerative disease in the subject based at least in part on the determining, wherein the early prognosis predates providing a diagnosis of a prodromal stage of the neurodegenerative disease in the subject; and (d) initiating a treatment comprising administration of a formulation comprising a therapeutic agent, wherein deciding the initiating is based at least in part on the establishing, wherein the therapeutic agent is selected from the group consisting of: ABvac40LY3002813, AADvac-1, ACI-35, AAV-Htert, ACU193, ASN51, ACI-242, AD-35, Aducanumab, AGB101, AL002, Allopregnanolone, APH-1105, Atuzaginstat (COR388), AVP-786, AXS-05, Baricitinib, Bepranemab, BCG vaccine, BE1Y2153, BPN14770, Bromocriptine, Bryostatin 1, BXCL-501, Blarcamesine (ANAVEX2-73), Bapineuzumab, BIIB092 (BMS-986168, IPN007, Gosuranemab), BIIB080/IONIS-MAPTRx, BIIB076, BPDO-1603, BMS-984923, BDPP (bioactive dietary polyphenol preparation), Brexpiprazole, Caffeine, CAD 1063, Canakinumab, Contraloid acetate, COR588, CORT108297, CNP520(AMG520), C2N-8E12 (ABBV-8E12), Crenezumab, CST-2032, Curcumin, CY6463, Dabigatran,DAOIB, Dapagliflozin, Daratumumab, Dasatinib, Deferiprone, Donanemab (LY3002813), Donepezil (Aricept), Dronabinol, Dutanumab, Edicotinib (JNJ-40346527), Efavirenz, Emtricitabine, Empagliflozin, Elenbecestat (E2609), E2814, Ednerpic (T-817 mA), Elayta (CT1812), ExPlas, Escitalopram, Fosgonimeton (ATH01017), Galantamine, Gantenerumab, Brain Shuttle Gantenerumab (R07126209), GB301, grapeseed extract, GV1001, Guanfacine, GV-971, Intranasal insulin, HT-ALZ, Huperzine A, Hydralazine, IGC AD1, Icosapent ethyl (IPE), JNJ-63733657, KHK 66401, Lu AF87908, Lu AF20513, Lamivudine (3TC), Lanabecestat, Lenalidomide, Levetiracetam, L-serine, Lupron, Remternetug (LY3372993), LY3372689, LMTX, LY3303560 (Zagotenemab), Lecanemab (BAN2401), Losartan, LX1001, MEDI 1814, Memantine, Metformin, MK-1942, MIB-626, Montelukast, MW150, Neflamapimod (VX-745), Nabilone, NE3107, nicotinamide, nicotine, NNI-362, Nilotinib BE, Octohydro-aminoacridine succinate, Obicetrapib, Omega-3 (DHA+EPA), perindopril, Pepinemab (VX15), Posiphen, Prazosin, Ponezumab, Protollin, PQ912, PNT001, PU-AD, Quercetin, rapamycin, REM0046127, RG7345 (R06926496), rivastigmine, RG6100, R07105705 (MTAU9937A, RG6100, Semorinemab), Salsalate, Sargramostim, Semorinemab, Senicapoc, Semaglutide, SHR-1707, Simufilam (PTI-125), Sodium oligomannate, Solanezumab, scyllo-inositol, Sovateltide (PMZ-1620), Suvorexant, T3D-959, TB006, Telmisartan, Trehalose, Tdap vaccine, THC-free CBD oil, Thiethylperazine (TEP), Troriluzole (BHV4157), TPI 2871, Tricaprilin (AC-1202), TRx0237, UB-311, UCB0107, valiltramiprosate (ALZ-801), Valacyclovir, varoglutamstat (PQ912), VGH-AD1, Vorinostat, VT301, XProl595, Xanamem, Yangxue Qingao pills, an antisense RNA directed to an isoform of human tau mRNA or an isoform of human amyloid beta, an siRNA directed to an isoform of human tau mRNA or an isoform of human amyloid beta, an antisense oligonucleotide directed to an isoform of human tau mRNA or an isoform of human amyloid beta, an LNA oligonucleotide directed to an isoform of human tau mRNA or an isoform of human amyloid beta, a CRISPRn-based therapeutic targeting the human MAPT or the human APP locus, a CRISPRi-based therapeutic targeting the human MAPT or the human APP locus, allogeneic human MSCs, SNK01 (autologous natural killer cell), allogenic adipose MSC-exosomes, CA-AC-02 (placenta derived MSCs), HUVEC-derived MSCs (NEUROSTEM), and AstroStem (autologous adipose-derived MSCs. In some embodiments, the HCDR1 sequence comprises SEQ ID NO: 2, HCDR2 sequence comprises SEQ ID NO: 11, HCDR3 sequence comprises SEQ ID NO: 19, LCDR1 sequence comprises SEQ ID NO: 25, LCDR2 sequence comprises SEQ ID NO: 33, and LCDR3 sequence comprises SEQ ID NO: 38. In some embodiments, the HCDR1 sequence comprises SEQ ID NO: 1, HCDR2 sequence comprises SEQ ID NO: 10, HCDR3 sequence comprises SEQ ID NO: 18, LCDR1 sequence comprises SEQ ID NO: 24, LCDR2 sequence comprises SEQ ID NO: 32, and LCDR3 sequence comprises SEQ ID NO: 37. In some embodiments, the HCDR1 sequence comprises SEQ ID NO: 2, HCDR2 sequence comprises SEQ ID NO: 11, HCDR3 sequence comprises SEQ ID NO: 19, LCDR1 sequence comprises SEQ ID NO: 26, LCDR2 sequence comprises SEQ ID NO: 34, and LCDR3 sequence comprises SEQ ID NO: 39. In some embodiments, the HCDR1 sequence comprises SEQ ID NO: 3, HCDR2 sequence comprises SEQ ID NO: 12, HCDR3 sequence comprises SEQ ID NO: 18, LCDR1 sequence comprises SEQ ID NO: 27, LCDR2 sequence comprises SEQ ID NO: 32, and LCDR3 sequence comprises SEQ ID NO: 40. In some embodiments, the HCDR1 sequence comprises SEQ ID NO: 4, HCDR2 sequence comprises SEQ ID NO: 11, HCDR3 sequence comprises SEQ ID NO: 20, LCDR1 sequence comprises SEQ ID NO: 28, LCDR2 sequence comprises SEQ ID NO: 35, and LCDR3 sequence comprises SEQ ID NO: 41. In some embodiments, the HCDR1 sequence comprises SEQ ID NO: 5, HCDR2 sequence comprises SEQ ID NO: 13, HCDR3 sequence comprises SEQ ID NO: 21, LCDR1 sequence comprises SEQ ID NO: 29, LCDR2 sequence comprises SEQ ID NO: 33, and LCDR3 sequence comprises SEQ ID NO: 42. In some embodiments, the neurodegenerative disease is Alzheimer's disease (AD). In some embodiments, the prodromal stage of AD is characterized by mild cognitive impairment (MCI). In some embodiments, the prodromal stage of AD is characterized at least as Braak stage I by a determined a spatial extent of tau-PET. In some embodiments, the prodromal stage of AD is characterized at least as Braak stage II by a determined a spatial extent of tau-PET. In some embodiments, the method comprises detecting the level of pT217 tau in the sample, wherein the detected level of pT217 tau is quantified as residing above the standard value of pT217 tau level derived from the plurality of reference samples, wherein the plurality of reference samples are taken from healthy individuals not suspected of having AD. In some embodiments, the subject meets inclusion criteria for initiating the treatment, and wherein the inclusion criteria comprise detecting a level of pT217 tau in a sample taken prior to d) initiating the treatment. —In some embodiments, the subject is at least 60 years of age. In some embodiments, the subject is between 60-85 years of age at the time of d) initiating the treatment. In some embodiments, the selected therapeutic agent is Donanemab. In some embodiments, the selected therapeutic agent is Lecanemab. In some embodiments, the selected therapeutic agent is Remternetug. In some embodiments, the selected therapeutic agent is Aducanumab. In some embodiments, the c) establishing the early prognosis comprises establishing a likelihood of AD progressing to AD dementia in the subject based at least in part on the a) detecting. In some embodiments, the VH domain comprises at least 80%, at least 85%, at least 90%, at least 95% sequence identity to a sequence selected from SEQ ID NOs: 44-48. In some embodiments, the VL domain comprises at least 80%, at least 85%, at least 90%, at least 95% sequence identity to a sequence selected from SEQ ID NOs: 49-54. In some embodiments, the anti-tau antibody or antigen-binding fragment thereof comprises an IgG-scFv, nanobody, BiTE, diabody, DART, TandAb, scDiabody, scDiabody-CH3, triple body, mini-antibody, minibody, TriBi minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, tetravalent HCAb, scDiabody-Fc, diabody-Fc, tandem scFv-Fc, or intrabody. In some embodiments, the anti-tau antibody has an isotype selected from the group consisting of IgA1, IgA2, IgD, IgE, IgG1, IgG2, IgG3, IgG4, and IgM. In some embodiments, the anti-tau antibody or antigen-binding fragment thereof specifically binds an epitope in an N-terminal region of a tau polypeptide. In some embodiments, the anti-tau antibody or antigen-binding fragment thereof specifically binds to pT217-tau protein. In some embodiments, the anti-tau antibody or antigen-binding fragment thereof specifically binds to a cis-conformation of pT217-tau protein. In some embodiments, the anti-tau antibody or antigen-binding fragment thereof binds specifically to a tau polypeptide comprising phosphorylation at a site selected from a group consisting of pT181-tau, pT212-tau, phosphorylated-serine (pS)214-tau, pT217-tau, pT220-tau, and pT231-tau. In some embodiments, the anti-tau antibody or antigen-binding fragment thereof binds specifically to a tau polypeptide comprising phosphorylation at site pT217-tau. In some embodiments, the anti-tau antibody or antigen-binding fragment thereof binds specifically to a tau polypeptide comprising phosphorylation at site pT217-tau and pT231-tau. In some embodiments, the neurodegenerative disease comprises Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, Hallevorden-Spatz disease, mild cognitive impairment (MCI), Lewy Body's disease (LBD), Amyotrophic lateral sclerosis, spinal muscular atrophy, Friedreich's ataxia, Wemicke-Korsakoff syndrome, a prion disease, vascular dementia, Alzheimer's related dementia, Huntington's disease (HD), or a transgene-induced tauopathy. In some embodiments, the sample is selected from a cerebrospinal fluid (CSF) sample, a plasma sample, a blood sample, or a serum sample. In some embodiments, the neurodegenerative disease comprises Alzheimer's disease. In some embodiments, one or more symptoms of the neurodegenerative disease in the subject is ameliorated. In some embodiments, a progression of one or more symptoms of the neurodegenerative disease in the subject is slowed. In some embodiments, the therapeutic agent is administered intravenously, intramuscularly, intrathecally, intracerebrally, subcutaneously, orally, nasally, topically, buccally, or sublingually. In some embodiments, the therapeutic agent is administered directly to the CNS of the subject via intravenous delivery, intravascular delivery, intrathecal delivery, intracisternal delivery, intraspinal delivery, subpial delivery, or intracerebroventricular delivery. In some embodiments, the subject is at a preclinical stage of the neurogenerative disease. In some embodiments, the subject is at a prodromal stage of the neurogenerative disease. In some embodiments, the plurality of reference samples are taken from healthy individuals not suspected of having a neurogenerative disease. In some embodiments, the plurality of samples from the reference subjects are taken from individuals at an early disease stage of a suspected neurogenerative disease. In some embodiments, the therapeutic agent is administered according to a body weight of the subject at a dosage of between about 0.1-100 mg/kg of the body weight. In some embodiments, the therapeutic agent is administered to the subject at a dosage of between about 200-1500 mg by intravenous administration. In some embodiments, the therapeutic agent is administered to the subject at a dosage of between about 700-1400 mg by intravenous administration. In some embodiments, the therapeutic agent is administered to the subject according to a treatment regimen comprising administering the therapeutic agent about once every 4 weeks (q4 week). In some embodiments, the first three doses administered to the subject comprise a dosage of about 700 mg of the therapeutic agent. In some embodiments, the doses administered to the subject after the first three doses administered comprise a dosage of about 1400 mg of the therapeutic agent. In some embodiments, systemic administration of the therapeutic agent. In some embodiments, systemic administration comprises i.v. administration of the therapeutic agent. In some embodiments, systemic administration comprises subcutaneous administration of the therapeutic agent. In some embodiments, systemic administration comprises oral administration of the therapeutic agent. In some embodiments, the anti-tau antibody comprises a HC comprising the sequence of SEQ ID NO: 55 and a LC comprising the sequence of SEQ ID NO: 56. In some embodiments, the anti-tau antibody comprises a HC comprising the sequence of SEQ ID NO: 57 and a LC comprising the sequence of SEQ ID NO: 58. In some embodiments, the anti-tau antibody comprises a HC comprising the sequence of SEQ ID NO: 57 and a LC comprising the sequence of SEQ ID NO: 59. In some embodiments, the anti-tau antibody comprises a HC comprising the sequence of SEQ ID NO: 60 and a LC comprising the sequence of SEQ ID NO: 61. In some embodiments, the anti-tau antibody comprises a HC comprising the sequence of SEQ ID NO: 62 and a LC comprising the sequence of SEQ ID NO: 63. In some embodiments, the anti-tau antibody comprises a HC comprising the sequence of SEQ ID NO: 64 and a LC comprising the sequence of SEQ ID NO: 65.

Described herein, in certain aspects, are methods of treating a neurodegenerative disease in a subject, the methods comprising: administering to the subject a therapeutically effective amount of a small molecule therapeutic agent targeting tau listed in Table 10, wherein the subject is selected based on a detection of a level of pT217 tau in a sample from the subject above a standard value of pT217 tau level derived from a plurality of reference samples and wherein the level of pT217 tau predicts a positive therapeutic response in the subject to a treatment targeting tau biological activity. In some embodiments, detection of the level of pT217 tau in the sample is performing using an anti-anti-tau antibody or the antigen-binding fragment thereof. In some embodiments, the anti-tau antibody or the antigen-binding fragment thereof comprises a variable heavy (VH) domain and a variable light (VL) domain. In some embodiments, the VH domain comprises at least 80%, at least 85%, at least 90%, at least 95% sequence identity to a sequence selected from SEQ ID NOs: 44-48. In some embodiments, the VL domain comprises at least 80%, at least 85%, at least 90%, at least 95% sequence identity to a sequence selected from SEQ ID NOs: 49-54. In some embodiments, the anti-tau antibody or antigen-binding fragment thereof comprises an IgG-scFv, nanobody, BiTE, diabody, DART, TandAb, scDiabody, scDiabody-CH3, triple body, mini-antibody, minibody, TriBi minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, tetravalent HCAb, scDiabody-Fc, diabody-Fc, tandem scFv-Fc, or intrabody. In some embodiments, the anti-tau antibody has an isotype selected from the group consisting of IgA1, IgA2, IgD, IgE, IgG1, IgG2, IgG3, IgG4, and IgM. In some embodiments, the anti-tau antibody or antigen-binding fragment thereof specifically binds an epitope in an N-terminal region of a tau polypeptide. In some embodiments, the anti-tau antibody or antigen-binding fragment thereof specifically binds to pT217-tau protein. In some embodiments, the anti-tau antibody or antigen-binding fragment thereof specifically binds to a cis-conformation of pT217-tau protein. In some embodiments, the anti-tau antibody or antigen-binding fragment thereof binds specifically to a tau polypeptide comprising phosphorylation at a site selected from a group consisting of pT181-tau, pT212-tau, phosphorylated-serine (pS)214-tau, pT217-tau, pT220-tau, and pT231-tau. In some embodiments, the anti-tau antibody or antigen-binding fragment thereof binds specifically to a tau polypeptide comprising phosphorylation at site pT217-tau. In some embodiments, the anti-tau antibody or antigen-binding fragment thereof binds specifically to a tau polypeptide comprising phosphorylation at site pT217-tau and pT231-tau. In some embodiments, the neurodegenerative disease comprises Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, Hallevorden-Spatz disease, mild cognitive impairment (MCI), Lewy Body's disease (LBD), Amyotrophic lateral sclerosis, spinal muscular atrophy, Friedreich's ataxia, Wemicke-Korsakoff syndrome, a prion disease, vascular dementia, Alzheimer's related dementia, Huntington's disease (HD), or a transgene-induced tauopathy. In some embodiments, the sample is selected from a cerebrospinal fluid (CSF) sample, a plasma sample, a blood sample, or a serum sample. In some embodiments, the neurodegenerative disease comprises Alzheimer's disease. In some embodiments, one or more symptoms of the neurodegenerative disease in the subject is ameliorated. In some embodiments, a progression of one or more symptoms of the neurodegenerative disease in the subject is slowed. In some embodiments, the therapeutic agent is administered intravenously, intramuscularly, intrathecally, intracerebrally, subcutaneously, orally, nasally, topically, buccally, or sublingually. In some embodiments, the therapeutic agent is administered directly to the CNS of the subject via intravenous delivery, intravascular delivery, intrathecal delivery, intracisternal delivery, intraspinal delivery, subpial delivery, or intracerebroventricular delivery. In some embodiments, the subject is at a preclinical stage of the neurogenerative disease. In some embodiments, the subject is at a prodromal stage of the neurogenerative disease. In some embodiments, the plurality of reference samples are taken from healthy individuals not suspected of having a neurogenerative disease. In some embodiments, the plurality of samples from the reference subjects are taken from individuals at an early disease stage of a suspected neurogenerative disease. In some embodiments, the therapeutic agent is administered according to a body weight of the subject at a dosage of between about 0.1-100 mg/kg of the body weight. In some embodiments, the therapeutic agent is administered to the subject at a dosage of between about 200-1500 mg by intravenous administration. In some embodiments, the therapeutic agent is administered to the subject at a dosage of between about 700-1400 mg by intravenous administration. In some embodiments, the therapeutic agent is administered to the subject according to a treatment regimen comprising administering the therapeutic agent about once every 4 weeks (q4 week). In some embodiments, the first three doses administered to the subject comprise a dosage of about 700 mg of the therapeutic agent. In some embodiments, the doses administered to the subject after the first three doses administered comprise a dosage of about 1400 mg of the therapeutic agent. In some embodiments, systemic administration of the therapeutic agent. In some embodiments, systemic administration comprises i.v. administration of the therapeutic agent. In some embodiments, systemic administration comprises subcutaneous administration of the therapeutic agent. In some embodiments, systemic administration comprises oral administration of the therapeutic agent. In some embodiments, the anti-tau antibody comprises a HC comprising the sequence of SEQ ID NO: 55 and a LC comprising the sequence of SEQ ID NO: 56. In some embodiments, the anti-tau antibody comprises a HC comprising the sequence of SEQ ID NO: 57 and a LC comprising the sequence of SEQ ID NO: 58. In some embodiments, the anti-tau antibody comprises a HC comprising the sequence of SEQ ID NO: 57 and a LC comprising the sequence of SEQ ID NO: 59. In some embodiments, the anti-tau antibody comprises a HC comprising the sequence of SEQ ID NO: 60 and a LC comprising the sequence of SEQ ID NO: 61. In some embodiments, the anti-tau antibody comprises a HC comprising the sequence of SEQ ID NO: 62 and a LC comprising the sequence of SEQ ID NO: 63. In some embodiments, the anti-tau antibody comprises a HC comprising the sequence of SEQ ID NO: 64 and a LC comprising the sequence of SEQ ID NO: 65.

Described herein, in certain aspects, are methods of treating a neurodegenerative disease in a subject, the methods comprising: administering to the subject a therapeutically effective amount of an antibody therapeutic agent targeting tau listed in Table 10, wherein the subject is selected based on a detection of a level of pT217 tau in a sample from the subject above a standard value of pT217 tau level derived from a plurality of reference samples and wherein the level of pT217 tau predicts a positive therapeutic response in the subject to a treatment targeting tau biological activity. In some embodiments, detection of the level of pT217 tau in the sample is performing using an anti-anti-tau antibody or the antigen-binding fragment thereof. In some embodiments, the anti-tau antibody or the antigen-binding fragment thereof comprises a variable heavy (VH) domain and a variable light (VL) domain. In some embodiments, the VH domain comprises at least 80%, at least 85%, at least 90%, at least 95% sequence identity to a sequence selected from SEQ ID NOs: 44-48. In some embodiments, the VL domain comprises at least 80%, at least 85%, at least 90%, at least 95% sequence identity to a sequence selected from SEQ ID NOs: 49-54. In some embodiments, the anti-tau antibody or antigen-binding fragment thereof comprises an IgG-scFv, nanobody, BiTE, diabody, DART, TandAb, scDiabody, scDiabody-CH3, triple body, mini-antibody, minibody, TriBi minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, tetravalent HCAb, scDiabody-Fc, diabody-Fc, tandem scFv-Fc, or intrabody. In some embodiments, the anti-tau antibody has an isotype selected from the group consisting of IgA1, IgA2, IgD, IgE, IgG1, IgG2, IgG3, IgG4, and IgM. In some embodiments, the anti-tau antibody or antigen-binding fragment thereof specifically binds an epitope in an N-terminal region of a tau polypeptide. In some embodiments, the anti-tau antibody or antigen-binding fragment thereof specifically binds to pT217-tau protein. In some embodiments, the anti-tau antibody or antigen-binding fragment thereof specifically binds to a cis-conformation of pT217-tau protein. In some embodiments, the anti-tau antibody or antigen-binding fragment thereof binds specifically to a tau polypeptide comprising phosphorylation at a site selected from a group consisting of pT181-tau, pT212-tau, phosphorylated-serine (pS)214-tau, pT217-tau, pT220-tau, and pT231-tau. In some embodiments, the anti-tau antibody or antigen-binding fragment thereof binds specifically to a tau polypeptide comprising phosphorylation at site pT217-tau. In some embodiments, the anti-tau antibody or antigen-binding fragment thereof binds specifically to a tau polypeptide comprising phosphorylation at site pT217-tau and pT231-tau. In some embodiments, the neurodegenerative disease comprises Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, Hallevorden-Spatz disease, mild cognitive impairment (MCI), Lewy Body's disease (LBD), Amyotrophic lateral sclerosis, spinal muscular atrophy, Friedreich's ataxia, Wernicke-Korsakoff syndrome, a prion disease, vascular dementia, Alzheimer's related dementia, Huntington's disease (HD), or a transgene-induced tauopathy. In some embodiments, the sample is selected from a cerebrospinal fluid (CSF) sample, a plasma sample, a blood sample, or a serum sample. In some embodiments, the neurodegenerative disease comprises Alzheimer's disease. In some embodiments, one or more symptoms of the neurodegenerative disease in the subject is ameliorated. In some embodiments, a progression of one or more symptoms of the neurodegenerative disease in the subject is slowed. In some embodiments, the therapeutic agent is administered intravenously, intramuscularly, intrathecally, intracerebrally, subcutaneously, orally, nasally, topically, buccally, or sublingually. In some embodiments, the therapeutic agent is administered directly to the CNS of the subject via intravenous delivery, intravascular delivery, intrathecal delivery, intracisternal delivery, intraspinal delivery, subpial delivery, or intracerebroventricular delivery. In some embodiments, the subject is at a preclinical stage of the neurogenerative disease. In some embodiments, the subject is at a prodromal stage of the neurogenerative disease. In some embodiments, the plurality of reference samples are taken from healthy individuals not suspected of having a neurogenerative disease. In some embodiments, the plurality of samples from the reference subjects are taken from individuals at an early disease stage of a suspected neurogenerative disease. In some embodiments, the therapeutic agent is administered according to a body weight of the subject at a dosage of between about 0.1-100 mg/kg of the body weight. In some embodiments, the therapeutic agent is administered to the subject at a dosage of between about 200-1500 mg by intravenous administration. In some embodiments, the therapeutic agent is administered to the subject at a dosage of between about 700-1400 mg by intravenous administration. In some embodiments, the therapeutic agent is administered to the subject according to a treatment regimen comprising administering the therapeutic agent about once every 4 weeks (q4 week). In some embodiments, the first three doses administered to the subject comprise a dosage of about 700 mg of the therapeutic agent. In some embodiments, the doses administered to the subject after the first three doses administered comprise a dosage of about 1400 mg of the therapeutic agent. In some embodiments, systemic administration of the therapeutic agent. In some embodiments, systemic administration comprises i.v. administration of the therapeutic agent. In some embodiments, systemic administration comprises subcutaneous administration of the therapeutic agent. In some embodiments, systemic administration comprises oral administration of the therapeutic agent. In some embodiments, the anti-tau antibody comprises a HC comprising the sequence of SEQ ID NO: 55 and a LC comprising the sequence of SEQ ID NO: 56. In some embodiments, the anti-tau antibody comprises a HC comprising the sequence of SEQ ID NO: 57 and a LC comprising the sequence of SEQ ID NO: 58. In some embodiments, the anti-tau antibody comprises a HC comprising the sequence of SEQ ID NO: 57 and a LC comprising the sequence of SEQ ID NO: 59. In some embodiments, the anti-tau antibody comprises a HC comprising the sequence of SEQ ID NO: 60 and a LC comprising the sequence of SEQ ID NO: 61. In some embodiments, the anti-tau antibody comprises a HC comprising the sequence of SEQ ID NO: 62 and a LC comprising the sequence of SEQ ID NO: 63. In some embodiments, the anti-tau antibody comprises a HC comprising the sequence of SEQ ID NO: 64 and a LC comprising the sequence of SEQ ID NO: 65.

Described herein, in certain aspects, are methods of treating a neurodegenerative disease in a subject, the methods comprising: administering to the subject a therapeutically effective amount of a small molecule therapeutic agent targeting beta amyloid listed in Table 10, wherein the subject is selected based on a detection of a level of pT217 tau in a sample from the subject above a standard value of pT217 tau level derived from a plurality of reference samples and wherein the level of pT217 tau predicts a positive therapeutic response in the subject to a treatment targeting beta amyloid biological activity. In some embodiments, detection of the level of pT217 tau in the sample is performing using an anti-anti-tau antibody or the antigen-binding fragment thereof. In some embodiments, the anti-tau antibody or the antigen-binding fragment thereof comprises a variable heavy (VH) domain and a variable light (VL) domain. In some embodiments, the VH domain comprises at least 80%, at least 85%, at least 90%, at least 95% sequence identity to a sequence selected from SEQ ID NOs: 44-48. In some embodiments, the VL domain comprises at least 80%, at least 85%, at least 90%, at least 95% sequence identity to a sequence selected from SEQ ID NOs: 49-54. In some embodiments, the anti-tau antibody or antigen-binding fragment thereof comprises an IgG-scFv, nanobody, BiTE, diabody, DART, TandAb, scDiabody, scDiabody-CH3, triple body, mini-antibody, minibody, TrⁱBi minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, tetravalent HCAb, scDiabody-Fc, diabody-Fc, tandem scFv-Fc, or intrabody. In some embodiments, the anti-tau antibody has an isotype selected from the group consisting of IgA1, IgA2, IgD, IgE, IgG1, IgG2, IgG3, IgG4, and IgM. In some embodiments, the anti-tau antibody or antigen-binding fragment thereof specifically binds an epitope in an N-terminal region of a tau polypeptide. In some embodiments, the anti-tau antibody or antigen-binding fragment thereof specifically binds to pT217-tau protein. In some embodiments, the anti-tau antibody or antigen-binding fragment thereof specifically binds to a cis-conformation of pT217-tau protein. In some embodiments, the anti-tau antibody or antigen-binding fragment thereof binds specifically to a tau polypeptide comprising phosphorylation at a site selected from a group consisting of pT181-tau, pT212-tau, phosphorylated-serine (pS)214-tau, pT217-tau, pT220-tau, and pT231-tau. In some embodiments, the anti-tau antibody or antigen-binding fragment thereof binds specifically to a tau polypeptide comprising phosphorylation at site pT217-tau. In some embodiments, the anti-tau antibody or antigen-binding fragment thereof binds specifically to a tau polypeptide comprising phosphorylation at site pT217-tau and pT231-tau. In some embodiments, the neurodegenerative disease comprises Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, Hallevorden-Spatz disease, mild cognitive impairment (MCI), Lewy Body's disease (LBD), Amyotrophic lateral sclerosis, spinal muscular atrophy, Friedreich's ataxia, Wemicke-Korsakoff syndrome, a prion disease, vascular dementia, Alzheimer's related dementia, Huntington's disease (HD), or a transgene-induced tauopathy. In some embodiments, the sample is selected from a cerebrospinal fluid (CSF) sample, a plasma sample, a blood sample, or a serum sample. In some embodiments, the neurodegenerative disease comprises Alzheimer's disease. In some embodiments, one or more symptoms of the neurodegenerative disease in the subject is ameliorated. In some embodiments, a progression of one or more symptoms of the neurodegenerative disease in the subject is slowed. In some embodiments, the therapeutic agent is administered intravenously, intramuscularly, intrathecally, intracerebrally, subcutaneously, orally, nasally, topically, buccally, or sublingually. In some embodiments, the therapeutic agent is administered directly to the CNS of the subject via intravenous delivery, intravascular delivery, intrathecal delivery, intracisternal delivery, intraspinal delivery, subpial delivery, or intracerebroventricular delivery. In some embodiments, the subject is at a preclinical stage of the neurogenerative disease. In some embodiments, the subject is at a prodromal stage of the neurogenerative disease. In some embodiments, the plurality of reference samples are taken from healthy individuals not suspected of having a neurogenerative disease. In some embodiments, the plurality of samples from the reference subjects are taken from individuals at an early disease stage of a suspected neurogenerative disease. In some embodiments, the therapeutic agent is administered according to a body weight of the subject at a dosage of between about 0.1-100 mg/kg of the body weight. In some embodiments, the therapeutic agent is administered to the subject at a dosage of between about 200-1500 mg by intravenous administration. In some embodiments, the therapeutic agent is administered to the subject at a dosage of between about 700-1400 mg by intravenous administration. In some embodiments, the therapeutic agent is administered to the subject according to a treatment regimen comprising administering the therapeutic agent about once every 4 weeks (q4 week). In some embodiments, the first three doses administered to the subject comprise a dosage of about 700 mg of the therapeutic agent. In some embodiments, the doses administered to the subject after the first three doses administered comprise a dosage of about 1400 mg of the therapeutic agent. In some embodiments, systemic administration of the therapeutic agent. In some embodiments, systemic administration comprises i.v. administration of the therapeutic agent. In some embodiments, systemic administration comprises subcutaneous administration of the therapeutic agent. In some embodiments, systemic administration comprises oral administration of the therapeutic agent. In some embodiments, the anti-tau antibody comprises a HC comprising the sequence of SEQ ID NO: 55 and a LC comprising the sequence of SEQ ID NO: 56. In some embodiments, the anti-tau antibody comprises a HC comprising the sequence of SEQ ID NO: 57 and a LC comprising the sequence of SEQ ID NO: 58. In some embodiments, the anti-tau antibody comprises a HC comprising the sequence of SEQ ID NO: 57 and a LC comprising the sequence of SEQ ID NO: 59. In some embodiments, the anti-tau antibody comprises a HC comprising the sequence of SEQ ID NO: 60 and a LC comprising the sequence of SEQ ID NO: 61. In some embodiments, the anti-tau antibody comprises a HC comprising the sequence of SEQ ID NO: 62 and a LC comprising the sequence of SEQ ID NO: 63. In some embodiments, the anti-tau antibody comprises a HC comprising the sequence of SEQ ID NO: 64 and a LC comprising the sequence of SEQ ID NO: 65.

Described herein, in certain aspects, are methods of treating a neurodegenerative disease in a subject, the methods comprising: administering to the subject a therapeutically effective amount of an antibody therapeutic agent targeting beta amyloid listed in Table 10, wherein the subject is selected based on a detection of a level of pT217 tau in a sample from the subject above a standard value of pT217 tau level derived from a plurality of reference samples and wherein the level of pT217 tau predicts a positive therapeutic response in the subject to a treatment targeting beta amyloid biological activity. In some embodiments, detection of the level of pT217 tau in the sample is performing using an anti-anti-tau antibody or the antigen-binding fragment thereof. In some embodiments, the anti-tau antibody or the antigen-binding fragment thereof comprises a variable heavy (VH) domain and a variable light (VL) domain. In some embodiments, the VH domain comprises at least 80%, at least 85%, at least 90%, at least 95% sequence identity to a sequence selected from SEQ ID NOs: 44-48. In some embodiments, the VL domain comprises at least 80%, at least 85%, at least 90%, at least 95% sequence identity to a sequence selected from SEQ ID NOs: 49-54. In some embodiments, the anti-tau antibody or antigen-binding fragment thereof comprises an IgG-scFv, nanobody, BiTE, diabody, DART, TandAb, scDiabody, scDiabody-CH3, triple body, mini-antibody, minibody, TriBi minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, tetravalent HCAb, scDiabody-Fc, diabody-Fc, tandem scFv-Fc, or intrabody. In some embodiments, the anti-tau antibody has an isotype selected from the group consisting of IgA1, IgA2, IgD, IgE, IgG1, IgG2, IgG3, IgG4, and IgM. In some embodiments, the anti-tau antibody or antigen-binding fragment thereof specifically binds an epitope in an N-terminal region of a tau polypeptide. In some embodiments, the anti-tau antibody or antigen-binding fragment thereof specifically binds to pT217-tau protein. In some embodiments, the anti-tau antibody or antigen-binding fragment thereof specifically binds to a cis-conformation of pT217-tau protein. In some embodiments, the anti-tau antibody or antigen-binding fragment thereof binds specifically to a tau polypeptide comprising phosphorylation at a site selected from a group consisting of pT181-tau, pT212-tau, phosphorylated-serine (pS)214-tau, pT217-tau, pT220-tau, and pT231-tau. In some embodiments, the anti-tau antibody or antigen-binding fragment thereof binds specifically to a tau polypeptide comprising phosphorylation at site pT217-tau. In some embodiments, the anti-tau antibody or antigen-binding fragment thereof binds specifically to a tau polypeptide comprising phosphorylation at site pT217-tau and pT231-tau. In some embodiments, the neurodegenerative disease comprises Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, Hallevorden-Spatz disease, mild cognitive impairment (MCI), Lewy Body's disease (LBD), Amyotrophic lateral sclerosis, spinal muscular atrophy, Friedreich's ataxia, Wemicke-Korsakoff syndrome, a prion disease, vascular dementia, Alzheimer's related dementia, Huntington's disease (HD), or a transgene-induced tauopathy. In some embodiments, the sample is selected from a cerebrospinal fluid (CSF) sample, a plasma sample, a blood sample, or a serum sample. In some embodiments, the neurodegenerative disease comprises Alzheimer's disease. In some embodiments, one or more symptoms of the neurodegenerative disease in the subject is ameliorated. In some embodiments, a progression of one or more symptoms of the neurodegenerative disease in the subject is slowed. In some embodiments, the therapeutic agent is administered intravenously, intramuscularly, intrathecally, intracerebrally, subcutaneously, orally, nasally, topically, buccally, or sublingually. In some embodiments, the therapeutic agent is administered directly to the CNS of the subject via intravenous delivery, intravascular delivery, intrathecal delivery, intracisternal delivery, intraspinal delivery, subpial delivery, or intracerebroventricular delivery. In some embodiments, the subject is at a preclinical stage of the neurogenerative disease. In some embodiments, the subject is at a prodromal stage of the neurogenerative disease. In some embodiments, the plurality of reference samples are taken from healthy individuals not suspected of having a neurogenerative disease. In some embodiments, the plurality of samples from the reference subjects are taken from individuals at an early disease stage of a suspected neurogenerative disease. In some embodiments, the therapeutic agent is administered according to a body weight of the subject at a dosage of between about 0.1-100 mg/kg of the body weight. In some embodiments, the therapeutic agent is administered to the subject at a dosage of between about 200-1500 mg by intravenous administration. In some embodiments, the therapeutic agent is administered to the subject at a dosage of between about 700-1400 mg by intravenous administration. In some embodiments, the therapeutic agent is administered to the subject according to a treatment regimen comprising administering the therapeutic agent about once every 4 weeks (q4 week). In some embodiments, the first three doses administered to the subject comprise a dosage of about 700 mg of the therapeutic agent. In some embodiments, the doses administered to the subject after the first three doses administered comprise a dosage of about 1400 mg of the therapeutic agent. In some embodiments, systemic administration of the therapeutic agent. In some embodiments, systemic administration comprises i.v. administration of the therapeutic agent. In some embodiments, systemic administration comprises subcutaneous administration of the therapeutic agent. In some embodiments, systemic administration comprises oral administration of the therapeutic agent. In some embodiments, the anti-tau antibody comprises a HC comprising the sequence of SEQ ID NO: 55 and a LC comprising the sequence of SEQ ID NO: 56. In some embodiments, the anti-tau antibody comprises a HC comprising the sequence of SEQ ID NO: 57 and a LC comprising the sequence of SEQ ID NO: 58. In some embodiments, the anti-tau antibody comprises a HC comprising the sequence of SEQ ID NO: 57 and a LC comprising the sequence of SEQ ID NO: 59. In some embodiments, the anti-tau antibody comprises a HC comprising the sequence of SEQ ID NO: 60 and a LC comprising the sequence of SEQ ID NO: 61. In some embodiments, the anti-tau antibody comprises a HC comprising the sequence of SEQ ID NO: 62 and a LC comprising the sequence of SEQ ID NO: 63. In some embodiments, the anti-tau antibody comprises a HC comprising the sequence of SEQ ID NO: 64 and a LC comprising the sequence of SEQ ID NO: 65.

Described herein, in certain aspects, are methods of treating a neurodegenerative disease in a subject, the methods comprising: administering to the subject a therapeutically effective amount of a cholinesterase inhibitor therapeutic agent listed in Table 10, wherein the subject is selected based on a detection of a level of pT217 tau in a sample from the subject above a standard value of pT217 tau level derived from a plurality of reference samples and wherein the level of pT217 tau predicts a positive therapeutic response in the subject to a treatment targeting inhibition of cholinesterase. In some embodiments, detection of the level of pT217 tau in the sample is performing using an anti-anti-tau antibody or the antigen-binding fragment thereof. In some embodiments, the anti-tau antibody or the antigen-binding fragment thereof comprises a variable heavy (VH) domain and a variable light (VL) domain. In some embodiments, the VH domain comprises at least 80%, at least 85%, at least 90%, at least 95% sequence identity to a sequence selected from SEQ ID NOs: 44-48. In some embodiments, the VL domain comprises at least 80%, at least 85%, at least 90%, at least 95% sequence identity to a sequence selected from SEQ ID NOs: 49-54. In some embodiments, the anti-tau antibody or antigen-binding fragment thereof comprises an IgG-scFv, nanobody, BiTE, diabody, DART, TandAb, scDiabody, scDiabody-CH3, triple body, mini-antibody, minibody, TriBi minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, tetravalent HCAb, scDiabody-Fc, diabody-Fc, tandem scFv-Fc, or intrabody. In some embodiments, the anti-tau antibody has an isotype selected from the group consisting of IgA1, IgA2, IgD, IgE, IgG1, IgG2, IgG3, IgG4, and IgM. In some embodiments, the anti-tau antibody or antigen-binding fragment thereof specifically binds an epitope in an N-terminal region of a tau polypeptide. In some embodiments, the anti-tau antibody or antigen-binding fragment thereof specifically binds to pT217-tau protein. In some embodiments, the anti-tau antibody or antigen-binding fragment thereof specifically binds to a cis-conformation of pT217-tau protein. In some embodiments, the anti-tau antibody or antigen-binding fragment thereof binds specifically to a tau polypeptide comprising phosphorylation at a site selected from a group consisting of pT181-tau, pT212-tau, phosphorylated-serine (pS)214-tau, pT217-tau, pT220-tau, and pT231-tau. In some embodiments, the anti-tau antibody or antigen-binding fragment thereof binds specifically to a tau polypeptide comprising phosphorylation at site pT217-tau. In some embodiments, the anti-tau antibody or antigen-binding fragment thereof binds specifically to a tau polypeptide comprising phosphorylation at site pT217-tau and pT231-tau. In some embodiments, the neurodegenerative disease comprises Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, Hallevorden-Spatz disease, mild cognitive impairment (MCI), Lewy Body's disease (LBD), Amyotrophic lateral sclerosis, spinal muscular atrophy, Friedreich's ataxia, Wernicke-Korsakoff syndrome, a prion disease, vascular dementia, Alzheimer's related dementia, Huntington's disease (HD), or a transgene-induced tauopathy. In some embodiments, the sample is selected from a cerebrospinal fluid (CSF) sample, a plasma sample, a blood sample, or a serum sample. In some embodiments, the neurodegenerative disease comprises Alzheimer's disease. In some embodiments, one or more symptoms of the neurodegenerative disease in the subject is ameliorated. In some embodiments, a progression of one or more symptoms of the neurodegenerative disease in the subject is slowed. In some embodiments, the therapeutic agent is administered intravenously, intramuscularly, intrathecally, intracerebrally, subcutaneously, orally, nasally, topically, buccally, or sublingually. In some embodiments, the therapeutic agent is administered directly to the CNS of the subject via intravenous delivery, intravascular delivery, intrathecal delivery, intracisternal delivery, intraspinal delivery, subpial delivery, or intracerebroventricular delivery. In some embodiments, the subject is at a preclinical stage of the neurogenerative disease. In some embodiments, the subject is at a prodromal stage of the neurogenerative disease. In some embodiments, the plurality of reference samples are taken from healthy individuals not suspected of having a neurogenerative disease. In some embodiments, the plurality of samples from the reference subjects are taken from individuals at an early disease stage of a suspected neurogenerative disease. In some embodiments, the therapeutic agent is administered according to a body weight of the subject at a dosage of between about 0.1-100 mg/kg of the body weight. In some embodiments, the therapeutic agent is administered to the subject at a dosage of between about 200-1500 mg by intravenous administration. In some embodiments, the therapeutic agent is administered to the subject at a dosage of between about 700-1400 mg by intravenous administration. In some embodiments, the therapeutic agent is administered to the subject according to a treatment regimen comprising administering the therapeutic agent about once every 4 weeks (q4 week). In some embodiments, the first three doses administered to the subject comprise a dosage of about 700 mg of the therapeutic agent. In some embodiments, the doses administered to the subject after the first three doses administered comprise a dosage of about 1400 mg of the therapeutic agent. In some embodiments, systemic administration of the therapeutic agent. In some embodiments, systemic administration comprises i.v. administration of the therapeutic agent. In some embodiments, systemic administration comprises subcutaneous administration of the therapeutic agent. In some embodiments, systemic administration comprises oral administration of the therapeutic agent. In some embodiments, the anti-tau antibody comprises a HC comprising the sequence of SEQ ID NO: 55 and a LC comprising the sequence of SEQ ID NO: 56. In some embodiments, the anti-tau antibody comprises a HC comprising the sequence of SEQ ID NO: 57 and a LC comprising the sequence of SEQ ID NO: 58. In some embodiments, the anti-tau antibody comprises a HC comprising the sequence of SEQ ID NO: 57 and a LC comprising the sequence of SEQ ID NO: 59. In some embodiments, the anti-tau antibody comprises a HC comprising the sequence of SEQ ID NO: 60 and a LC comprising the sequence of SEQ ID NO: 61. In some embodiments, the anti-tau antibody comprises a HC comprising the sequence of SEQ ID NO: 62 and a LC comprising the sequence of SEQ ID NO: 63. In some embodiments, the anti-tau antibody comprises a HC comprising the sequence of SEQ ID NO: 64 and a LC comprising the sequence of SEQ ID NO: 65.

Described herein, in certain aspects, are methods of treating a neurodegenerative disease in a subject, the methods comprising: administering to the subject a therapeutically effective amount of an NMDA receptor antagonist therapeutic agent listed in Table 10, wherein the subject is selected based on a detection of a level of pT217 tau in a sample from the subject above a standard value of pT217 tau level derived from a plurality of reference samples and wherein the level of pT217 tau predicts a positive therapeutic response in the subject to a treatment targeting inhibition of NMDA receptor function. In some embodiments, detection of the level of pT217 tau in the sample is performing using an anti-anti-tau antibody or the antigen-binding fragment thereof. In some embodiments, the anti-tau antibody or the antigen-binding fragment thereof comprises a variable heavy (VH) domain and a variable light (VL) domain. In some embodiments, the VH domain comprises at least 80%, at least 85%, at least 90%, at least 95% sequence identity to a sequence selected from SEQ ID NOs: 44-48. In some embodiments, the VL domain comprises at least 80%, at least 85%, at least 90%, at least 95% sequence identity to a sequence selected from SEQ ID NOs: 49-54. In some embodiments, the anti-tau antibody or antigen-binding fragment thereof comprises an IgG-scFv, nanobody, BiTE, diabody, DART, TandAb, scDiabody, scDiabody-CH3, triple body, mini-antibody, minibody, TrⁱBi minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, tetravalent HCAb, scDiabody-Fc, diabody-Fc, tandem scFv-Fc, or intrabody. In some embodiments, the anti-tau antibody has an isotype selected from the group consisting of IgA1, IgA2, IgD, IgE, IgG1, IgG2, IgG3, IgG4, and IgM. In some embodiments, the anti-tau antibody or antigen-binding fragment thereof specifically binds an epitope in an N-terminal region of a tau polypeptide. In some embodiments, the anti-tau antibody or antigen-binding fragment thereof specifically binds to pT217-tau protein. In some embodiments, the anti-tau antibody or antigen-binding fragment thereof specifically binds to a cis-conformation of pT217-tau protein. In some embodiments, the anti-tau antibody or antigen-binding fragment thereof binds specifically to a tau polypeptide comprising phosphorylation at a site selected from a group consisting of pT181-tau, pT212-tau, phosphorylated-serine (pS)214-tau, pT217-tau, pT220-tau, and pT231-tau. In some embodiments, the anti-tau antibody or antigen-binding fragment thereof binds specifically to a tau polypeptide comprising phosphorylation at site pT217-tau. In some embodiments, the anti-tau antibody or antigen-binding fragment thereof binds specifically to a tau polypeptide comprising phosphorylation at site pT217-tau and pT231-tau. In some embodiments, the neurodegenerative disease comprises Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, Hallevorden-Spatz disease, mild cognitive impairment (MCI), Lewy Body's disease (LBD), Amyotrophic lateral sclerosis, spinal muscular atrophy, Friedreich's ataxia, Wemicke-Korsakoff syndrome, a prion disease, vascular dementia, Alzheimer's related dementia, Huntington's disease (HD), or a transgene-induced tauopathy. In some embodiments, the sample is selected from a cerebrospinal fluid (CSF) sample, a plasma sample, a blood sample, or a serum sample. In some embodiments, the neurodegenerative disease comprises Alzheimer's disease. In some embodiments, one or more symptoms of the neurodegenerative disease in the subject is ameliorated. In some embodiments, a progression of one or more symptoms of the neurodegenerative disease in the subject is slowed. In some embodiments, the therapeutic agent is administered intravenously, intramuscularly, intrathecally, intracerebrally, subcutaneously, orally, nasally, topically, buccally, or sublingually. In some embodiments, the therapeutic agent is administered directly to the CNS of the subject via intravenous delivery, intravascular delivery, intrathecal delivery, intracisternal delivery, intraspinal delivery, subpial delivery, or intracerebroventricular delivery. In some embodiments, the subject is at a preclinical stage of the neurogenerative disease. In some embodiments, the subject is at a prodromal stage of the neurogenerative disease. In some embodiments, the plurality of reference samples are taken from healthy individuals not suspected of having a neurogenerative disease. In some embodiments, the plurality of samples from the reference subjects are taken from individuals at an early disease stage of a suspected neurogenerative disease. In some embodiments, the therapeutic agent is administered according to a body weight of the subject at a dosage of between about 0.1-100 mg/kg of the body weight. In some embodiments, the therapeutic agent is administered to the subject at a dosage of between about 200-1500 mg by intravenous administration. In some embodiments, the therapeutic agent is administered to the subject at a dosage of between about 700-1400 mg by intravenous administration. In some embodiments, the therapeutic agent is administered to the subject according to a treatment regimen comprising administering the therapeutic agent about once every 4 weeks (q4 week). In some embodiments, the first three doses administered to the subject comprise a dosage of about 700 mg of the therapeutic agent. In some embodiments, the doses administered to the subject after the first three doses administered comprise a dosage of about 1400 mg of the therapeutic agent. In some embodiments, systemic administration of the therapeutic agent. In some embodiments, systemic administration comprises i.v. administration of the therapeutic agent. In some embodiments, systemic administration comprises subcutaneous administration of the therapeutic agent. In some embodiments, systemic administration comprises oral administration of the therapeutic agent. In some embodiments, the anti-tau antibody comprises a HC comprising the sequence of SEQ ID NO: 55 and a LC comprising the sequence of SEQ ID NO: 56. In some embodiments, the anti-tau antibody comprises a HC comprising the sequence of SEQ ID NO: 57 and a LC comprising the sequence of SEQ ID NO: 58. In some embodiments, the anti-tau antibody comprises a HC comprising the sequence of SEQ ID NO: 57 and a LC comprising the sequence of SEQ ID NO: 59. In some embodiments, the anti-tau antibody comprises a HC comprising the sequence of SEQ ID NO: 60 and a LC comprising the sequence of SEQ ID NO: 61. In some embodiments, the anti-tau antibody comprises a HC comprising the sequence of SEQ ID NO: 62 and a LC comprising the sequence of SEQ ID NO: 63. In some embodiments, the anti-tau antibody comprises a HC comprising the sequence of SEQ ID NO: 64 and a LC comprising the sequence of SEQ ID NO: 65.

Described herein, in certain aspects, are methods of treating a neurodegenerative disease in a subject, the methods comprising: administering to the subject a therapeutically effective amount of an acetylcholinesterase and butyrylcholinesterase inhibitor therapeutic agent listed in Table 10, wherein the subject is selected based on a detection of a level of pT217 tau in a sample from the subject above a standard value of pT217 tau level derived from a plurality of reference samples and wherein the level of pT217 tau predicts a positive therapeutic response in the subject to a treatment targeting inhibition of acetylcholinesterase and butyrylcholinesterase function. In some embodiments, detection of the level of pT217 tau in the sample is performing using an anti-anti-tau antibody or the antigen-binding fragment thereof. In some embodiments, the anti-tau antibody or the antigen-binding fragment thereof comprises a variable heavy (VH) domain and a variable light (VL) domain. In some embodiments, the VH domain comprises at least 80%, at least 85%, at least 90%, at least 95% sequence identity to a sequence selected from SEQ ID NOs: 44-48. In some embodiments, the VL domain comprises at least 80%, at least 85%, at least 90%, at least 95% sequence identity to a sequence selected from SEQ ID NOs: 49-54. In some embodiments, the anti-tau antibody or antigen-binding fragment thereof comprises an IgG-scFv, nanobody, BiTE, diabody, DART, TandAb, scDiabody, scDiabody-CH3, triple body, mini-antibody, minibody, TriBi minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, tetravalent HCAb, scDiabody-Fc, diabody-Fc, tandem scFv-Fc, or intrabody. In some embodiments, the anti-tau antibody has an isotype selected from the group consisting of IgA1, IgA2, IgD, IgE, IgG1, IgG2, IgG3, IgG4, and IgM. In some embodiments, the anti-tau antibody or antigen-binding fragment thereof specifically binds an epitope in an N-terminal region of a tau polypeptide. In some embodiments, the anti-tau antibody or antigen-binding fragment thereof specifically binds to pT217-tau protein. In some embodiments, the anti-tau antibody or antigen-binding fragment thereof specifically binds to a cis-conformation of pT217-tau protein. In some embodiments, the anti-tau antibody or antigen-binding fragment thereof binds specifically to a tau polypeptide comprising phosphorylation at a site selected from a group consisting of pT181-tau, pT212-tau, phosphorylated-serine (pS)214-tau, pT217-tau, pT220-tau, and pT231-tau. In some embodiments, the anti-tau antibody or antigen-binding fragment thereof binds specifically to a tau polypeptide comprising phosphorylation at site pT217-tau. In some embodiments, the anti-tau antibody or antigen-binding fragment thereof binds specifically to a tau polypeptide comprising phosphorylation at site pT217-tau and pT231-tau. In some embodiments, the neurodegenerative disease comprises Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, Hallevorden-Spatz disease, mild cognitive impairment (MCI), Lewy Body's disease (LBD), Amyotrophic lateral sclerosis, spinal muscular atrophy, Friedreich's ataxia, Wemicke-Korsakoff syndrome, a prion disease, vascular dementia, Alzheimer's related dementia, Huntington's disease (HD), or a transgene-induced tauopathy. In some embodiments, the sample is selected from a cerebrospinal fluid (CSF) sample, a plasma sample, a blood sample, or a serum sample. In some embodiments, the neurodegenerative disease comprises Alzheimer's disease. In some embodiments, one or more symptoms of the neurodegenerative disease in the subject is ameliorated. In some embodiments, a progression of one or more symptoms of the neurodegenerative disease in the subject is slowed. In some embodiments, the therapeutic agent is administered intravenously, intramuscularly, intrathecally, intracerebrally, subcutaneously, orally, nasally, topically, buccally, or sublingually. In some embodiments, the therapeutic agent is administered directly to the CNS of the subject via intravenous delivery, intravascular delivery, intrathecal delivery, intracisternal delivery, intraspinal delivery, subpial delivery, or intracerebroventricular delivery. In some embodiments, the subject is at a preclinical stage of the neurogenerative disease. In some embodiments, the subject is at a prodromal stage of the neurogenerative disease. In some embodiments, the plurality of reference samples are taken from healthy individuals not suspected of having a neurogenerative disease. In some embodiments, the plurality of samples from the reference subjects are taken from individuals at an early disease stage of a suspected neurogenerative disease. In some embodiments, the therapeutic agent is administered according to a body weight of the subject at a dosage of between about 0.1-100 mg/kg of the body weight. In some embodiments, the therapeutic agent is administered to the subject at a dosage of between about 200-1500 mg by intravenous administration. In some embodiments, the therapeutic agent is administered to the subject at a dosage of between about 700-1400 mg by intravenous administration. In some embodiments, the therapeutic agent is administered to the subject according to a treatment regimen comprising administering the therapeutic agent about once every 4 weeks (q4 week). In some embodiments, the first three doses administered to the subject comprise a dosage of about 700 mg of the therapeutic agent. In some embodiments, the doses administered to the subject after the first three doses administered comprise a dosage of about 1400 mg of the therapeutic agent. In some embodiments, systemic administration of the therapeutic agent. In some embodiments, systemic administration comprises i.v. administration of the therapeutic agent. In some embodiments, systemic administration comprises subcutaneous administration of the therapeutic agent. In some embodiments, systemic administration comprises oral administration of the therapeutic agent. In some embodiments, the anti-tau antibody comprises a HC comprising the sequence of SEQ ID NO: 55 and a LC comprising the sequence of SEQ ID NO: 56. In some embodiments, the anti-tau antibody comprises a HC comprising the sequence of SEQ ID NO: 57 and a LC comprising the sequence of SEQ ID NO: 58. In some embodiments, the anti-tau antibody comprises a HC comprising the sequence of SEQ ID NO: 57 and a LC comprising the sequence of SEQ ID NO: 59. In some embodiments, the anti-tau antibody comprises a HC comprising the sequence of SEQ ID NO: 60 and a LC comprising the sequence of SEQ ID NO: 61. In some embodiments, the anti-tau antibody comprises a HC comprising the sequence of SEQ ID NO: 62 and a LC comprising the sequence of SEQ ID NO: 63. In some embodiments, the anti-tau antibody comprises a HC comprising the sequence of SEQ ID NO: 64 and a LC comprising the sequence of SEQ ID NO: 65.

Described herein, in certain aspects, are methods of treating a neurodegenerative disease in a subject, the methods comprising: administering to the subject a therapeutically effective amount of an NMDA receptor antagonist therapeutic agent listed in Table 10 and a cholinesterase inhibitor therapeutic agent listed in Table 10, wherein the subject is selected based on a detection of a level of pT217 tau in a sample from the subject above a standard value of pT217 tau level derived from a plurality of reference samples and wherein the level of pT217 tau predicts a positive therapeutic response in the subject to a treatment targeting inhibition of NMDA receptor function and inhibition of NMDA receptor function. In some embodiments, detection of the level of pT217 tau in the sample is performing using an anti-anti-tau antibody or the antigen-binding fragment thereof. In some embodiments, the anti-tau antibody or the antigen-binding fragment thereof comprises a variable heavy (VH) domain and a variable light (VL) domain. In some embodiments, the VH domain comprises at least 80%, at least 85%, at least 90%, at least 95% sequence identity to a sequence selected from SEQ ID NOs: 44-48. In some embodiments, the VL domain comprises at least 80%, at least 85%, at least 90%, at least 95% sequence identity to a sequence selected from SEQ ID NOs: 49-54. In some embodiments, the anti-tau antibody or antigen-binding fragment thereof comprises an IgG-scFv, nanobody, BiTE, diabody, DART, TandAb, scDiabody, scDiabody-CH3, triple body, mini-antibody, minibody, TriBi minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, tetravalent HCAb, scDiabody-Fc, diabody-Fc, tandem scFv-Fc, or intrabody. In some embodiments, the anti-tau antibody has an isotype selected from the group consisting of IgA1, IgA2, IgD, IgE, IgG1, IgG2, IgG3, IgG4, and IgM. In some embodiments, the anti-tau antibody or antigen-binding fragment thereof specifically binds an epitope in an N-terminal region of a tau polypeptide. In some embodiments, the anti-tau antibody or antigen-binding fragment thereof specifically binds to pT217-tau protein. In some embodiments, the anti-tau antibody or antigen-binding fragment thereof specifically binds to a cis-conformation of pT217-tau protein. In some embodiments, the anti-tau antibody or antigen-binding fragment thereof binds specifically to a tau polypeptide comprising phosphorylation at a site selected from a group consisting of pT181-tau, pT212-tau, phosphorylated-serine (pS)214-tau, pT217-tau, pT220-tau, and pT231-tau. In some embodiments, the anti-tau antibody or antigen-binding fragment thereof binds specifically to a tau polypeptide comprising phosphorylation at site pT217-tau. In some embodiments, the anti-tau antibody or antigen-binding fragment thereof binds specifically to a tau polypeptide comprising phosphorylation at site pT217-tau and pT231-tau. In some embodiments, the neurodegenerative disease comprises Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, Hallevorden-Spatz disease, mild cognitive impairment (MCI), Lewy Body's disease (LBD), Amyotrophic lateral sclerosis, spinal muscular atrophy, Friedreich's ataxia, Wernicke-Korsakoff syndrome, a prion disease, vascular dementia, Alzheimer's related dementia, Huntington's disease (HD), or a transgene-induced tauopathy. In some embodiments, the sample is selected from a cerebrospinal fluid (CSF) sample, a plasma sample, a blood sample, or a serum sample. In some embodiments, the neurodegenerative disease comprises Alzheimer's disease. In some embodiments, one or more symptoms of the neurodegenerative disease in the subject is ameliorated. In some embodiments, a progression of one or more symptoms of the neurodegenerative disease in the subject is slowed. In some embodiments, the therapeutic agent is administered intravenously, intramuscularly, intrathecally, intracerebrally, subcutaneously, orally, nasally, topically, buccally, or sublingually. In some embodiments, the therapeutic agent is administered directly to the CNS of the subject via intravenous delivery, intravascular delivery, intrathecal delivery, intracisternal delivery, intraspinal delivery, subpial delivery, or intracerebroventricular delivery. In some embodiments, the subject is at a preclinical stage of the neurogenerative disease. In some embodiments, the subject is at a prodromal stage of the neurogenerative disease. In some embodiments, the plurality of reference samples are taken from healthy individuals not suspected of having a neurogenerative disease. In some embodiments, the plurality of samples from the reference subjects are taken from individuals at an early disease stage of a suspected neurogenerative disease. In some embodiments, the therapeutic agent is administered according to a body weight of the subject at a dosage of between about 0.1-100 mg/kg of the body weight. In some embodiments, the therapeutic agent is administered to the subject at a dosage of between about 200-1500 mg by intravenous administration. In some embodiments, the therapeutic agent is administered to the subject at a dosage of between about 700-1400 mg by intravenous administration. In some embodiments, the therapeutic agent is administered to the subject according to a treatment regimen comprising administering the therapeutic agent about once every 4 weeks (q4 week). In some embodiments, the first three doses administered to the subject comprise a dosage of about 700 mg of the therapeutic agent. In some embodiments, the doses administered to the subject after the first three doses administered comprise a dosage of about 1400 mg of the therapeutic agent. In some embodiments, systemic administration of the therapeutic agent. In some embodiments, systemic administration comprises i.v. administration of the therapeutic agent. In some embodiments, systemic administration comprises subcutaneous administration of the therapeutic agent. In some embodiments, systemic administration comprises oral administration of the therapeutic agent. In some embodiments, the anti-tau antibody comprises a HC comprising the sequence of SEQ ID NO: 55 and a LC comprising the sequence of SEQ ID NO: 56. In some embodiments, the anti-tau antibody comprises a HC comprising the sequence of SEQ ID NO: 57 and a LC comprising the sequence of SEQ ID NO: 58. In some embodiments, the anti-tau antibody comprises a HC comprising the sequence of SEQ ID NO: 57 and a LC comprising the sequence of SEQ ID NO: 59. In some embodiments, the anti-tau antibody comprises a HC comprising the sequence of SEQ ID NO: 60 and a LC comprising the sequence of SEQ ID NO: 61. In some embodiments, the anti-tau antibody comprises a HC comprising the sequence of SEQ ID NO: 62 and a LC comprising the sequence of SEQ ID NO: 63. In some embodiments, the anti-tau antibody comprises a HC comprising the sequence of SEQ ID NO: 64 and a LC comprising the sequence of SEQ ID NO: 65.

Described herein, in certain aspects, are methods of treating a neurodegenerative disease in a subject, the methods comprising: administering to the subject a therapeutically effective amount of an antibody therapeutic agent targeting amyloid-beta peptide listed in Table 10, wherein the subject is selected based on a detection of a level of pT217 tau in a sample from the subject above a standard value of pT217 tau level derived from a plurality of reference samples and wherein the level of pT217 tau predicts a positive therapeutic response in the subject to a treatment targeting aggregated soluble and insoluble forms of amyloid-beta peptide. In some embodiments, detection of the level of pT217 tau in the sample is performing using an anti-anti-tau antibody or the antigen-binding fragment thereof. In some embodiments, the anti-tau antibody or the antigen-binding fragment thereof comprises a variable heavy (VH) domain and a variable light (VL) domain. In some embodiments, the VH domain comprises at least 80%, at least 85%, at least 90%, at least 95% sequence identity to a sequence selected from SEQ ID NOs: 44-48. In some embodiments, the VL domain comprises at least 80%, at least 85%, at least 90%, at least 95% sequence identity to a sequence selected from SEQ ID NOs: 49-54. In some embodiments, the anti-tau antibody or antigen-binding fragment thereof comprises an IgG-scFv, nanobody, BiTE, diabody, DART, TandAb, scDiabody, scDiabody-CH3, triple body, mini-antibody, minibody, TriBi minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, tetravalent HCAb, scDiabody-Fc, diabody-Fc, tandem scFv-Fc, or intrabody. In some embodiments, the anti-tau antibody has an isotype selected from the group consisting of IgA1, IgA2, IgD, IgE, IgG1, IgG2, IgG3, IgG4, and IgM. In some embodiments, the anti-tau antibody or antigen-binding fragment thereof specifically binds an epitope in an N-terminal region of a tau polypeptide. In some embodiments, the anti-tau antibody or antigen-binding fragment thereof specifically binds to pT217-tau protein. In some embodiments, the anti-tau antibody or antigen-binding fragment thereof specifically binds to a cis-conformation of pT217-tau protein. In some embodiments, the anti-tau antibody or antigen-binding fragment thereof binds specifically to a tau polypeptide comprising phosphorylation at a site selected from a group consisting of pT181-tau, pT212-tau, phosphorylated-serine (pS)214-tau, pT217-tau, pT220-tau, and pT231-tau. In some embodiments, the anti-tau antibody or antigen-binding fragment thereof binds specifically to a tau polypeptide comprising phosphorylation at site pT217-tau. In some embodiments, the anti-tau antibody or antigen-binding fragment thereof binds specifically to a tau polypeptide comprising phosphorylation at site pT217-tau and pT231-tau. In some embodiments, the neurodegenerative disease comprises Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, Hallevorden-Spatz disease, mild cognitive impairment (MCI), Lewy Body's disease (LBD), Amyotrophic lateral sclerosis, spinal muscular atrophy, Friedreich's ataxia, Wemicke-Korsakoff syndrome, a prion disease, vascular dementia, Alzheimer's related dementia, Huntington's disease (HD), or a transgene-induced tauopathy. In some embodiments, the sample is selected from a cerebrospinal fluid (CSF) sample, a plasma sample, a blood sample, or a serum sample. In some embodiments, the neurodegenerative disease comprises Alzheimer's disease. In some embodiments, one or more symptoms of the neurodegenerative disease in the subject is ameliorated. In some embodiments, a progression of one or more symptoms of the neurodegenerative disease in the subject is slowed. In some embodiments, the therapeutic agent is administered intravenously, intramuscularly, intrathecally, intracerebrally, subcutaneously, orally, nasally, topically, buccally, or sublingually. In some embodiments, the therapeutic agent is administered directly to the CNS of the subject via intravenous delivery, intravascular delivery, intrathecal delivery, intracisternal delivery, intraspinal delivery, subpial delivery, or intracerebroventricular delivery. In some embodiments, the subject is at a preclinical stage of the neurogenerative disease. In some embodiments, the subject is at a prodromal stage of the neurogenerative disease. In some embodiments, the plurality of reference samples are taken from healthy individuals not suspected of having a neurogenerative disease. In some embodiments, the plurality of samples from the reference subjects are taken from individuals at an early disease stage of a suspected neurogenerative disease. In some embodiments, the therapeutic agent is administered according to a body weight of the subject at a dosage of between about 0.1-100 mg/kg of the body weight. In some embodiments, the therapeutic agent is administered to the subject at a dosage of between about 200-1500 mg by intravenous administration. In some embodiments, the therapeutic agent is administered to the subject at a dosage of between about 700-1400 mg by intravenous administration. In some embodiments, the therapeutic agent is administered to the subject according to a treatment regimen comprising administering the therapeutic agent about once every 4 weeks (q4 week). In some embodiments, the first three doses administered to the subject comprise a dosage of about 700 mg of the therapeutic agent. In some embodiments, the doses administered to the subject after the first three doses administered comprise a dosage of about 1400 mg of the therapeutic agent. In some embodiments, systemic administration of the therapeutic agent. In some embodiments, systemic administration comprises i.v. administration of the therapeutic agent. In some embodiments, systemic administration comprises subcutaneous administration of the therapeutic agent. In some embodiments, systemic administration comprises oral administration of the therapeutic agent. In some embodiments, the anti-tau antibody comprises a HC comprising the sequence of SEQ ID NO: 55 and a LC comprising the sequence of SEQ ID NO: 56. In some embodiments, the anti-tau antibody comprises a HC comprising the sequence of SEQ ID NO: 57 and a LC comprising the sequence of SEQ ID NO: 58. In some embodiments, the anti-tau antibody comprises a HC comprising the sequence of SEQ ID NO: 57 and a LC comprising the sequence of SEQ ID NO: 59. In some embodiments, the anti-tau antibody comprises a HC comprising the sequence of SEQ ID NO: 60 and a LC comprising the sequence of SEQ ID NO: 61. In some embodiments, the anti-tau antibody comprises a HC comprising the sequence of SEQ ID NO: 62 and a LC comprising the sequence of SEQ ID NO: 63. In some embodiments, the anti-tau antibody comprises a HC comprising the sequence of SEQ ID NO: 64 and a LC comprising the sequence of SEQ ID NO: 65.

Described herein, in certain aspects, are methods of treating a neurodegenerative disease in a subject, the methods comprising: administering to the subject a therapeutically effective amount of an antibody therapeutic agent targeting amyloid-beta peptide listed in Table 10, wherein the subject is selected based on a detection of a level of pT217 tau in a sample from the subject above a standard value of pT217 tau level derived from a plurality of reference samples and wherein the level of pT217 tau predicts a positive therapeutic response in the subject to a treatment targeting removal of aggregated soluble and insoluble forms of amyloid-beta peptide. In some embodiments, detection of the level of pT217 tau in the sample is performing using an anti-anti-tau antibody or the antigen-binding fragment thereof. In some embodiments, the anti-tau antibody or the antigen-binding fragment thereof comprises a variable heavy (VH) domain and a variable light (VL) domain. In some embodiments, the VH domain comprises at least 80%, at least 85%, at least 90%, at least 95% sequence identity to a sequence selected from SEQ ID NOs: 44-48. In some embodiments, the VL domain comprises at least 80%, at least 85%, at least 90%, at least 95% sequence identity to a sequence selected from SEQ ID NOs: 49-54. In some embodiments, the anti-tau antibody or antigen-binding fragment thereof comprises an IgG-scFv, nanobody, BiTE, diabody, DART, TandAb, scDiabody, scDiabody-CH3, triple body, mini-antibody, minibody, TriBi minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, tetravalent HCAb, scDiabody-Fc, diabody-Fc, tandem scFv-Fc, or intrabody. In some embodiments, the anti-tau antibody has an isotype selected from the group consisting of IgA1, IgA2, IgD, IgE, IgG1, IgG2, IgG3, IgG4, and IgM. In some embodiments, the anti-tau antibody or antigen-binding fragment thereof specifically binds an epitope in an N-terminal region of a tau polypeptide. In some embodiments, the anti-tau antibody or antigen-binding fragment thereof specifically binds to pT217-tau protein. In some embodiments, the anti-tau antibody or antigen-binding fragment thereof specifically binds to a cis-conformation of pT217-tau protein. In some embodiments, the anti-tau antibody or antigen-binding fragment thereof binds specifically to a tau polypeptide comprising phosphorylation at a site selected from a group consisting of pT181-tau, pT212-tau, phosphorylated-serine (pS)214-tau, pT217-tau, pT220-tau, and pT231-tau. In some embodiments, the anti-tau antibody or antigen-binding fragment thereof binds specifically to a tau polypeptide comprising phosphorylation at site pT217-tau. In some embodiments, the anti-tau antibody or antigen-binding fragment thereof binds specifically to a tau polypeptide comprising phosphorylation at site pT217-tau and pT231-tau. In some embodiments, the neurodegenerative disease comprises Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, Hallevorden-Spatz disease, mild cognitive impairment (MCI), Lewy Body's disease (LBD), Amyotrophic lateral sclerosis, spinal muscular atrophy, Friedreich's ataxia, Wemicke-Korsakoff syndrome, a prion disease, vascular dementia, Alzheimer's related dementia, Huntington's disease (HD), or a transgene-induced tauopathy. In some embodiments, the sample is selected from a cerebrospinal fluid (CSF) sample, a plasma sample, a blood sample, or a serum sample. In some embodiments, the neurodegenerative disease comprises Alzheimer's disease. In some embodiments, one or more symptoms of the neurodegenerative disease in the subject is ameliorated. In some embodiments, a progression of one or more symptoms of the neurodegenerative disease in the subject is slowed. In some embodiments, the therapeutic agent is administered intravenously, intramuscularly, intrathecally, intracerebrally, subcutaneously, orally, nasally, topically, buccally, or sublingually. In some embodiments, the therapeutic agent is administered directly to the CNS of the subject via intravenous delivery, intravascular delivery, intrathecal delivery, intracisternal delivery, intraspinal delivery, subpial delivery, or intracerebroventricular delivery. In some embodiments, the subject is at a preclinical stage of the neurogenerative disease. In some embodiments, the subject is at a prodromal stage of the neurogenerative disease. In some embodiments, the plurality of reference samples are taken from healthy individuals not suspected of having a neurogenerative disease. In some embodiments, the plurality of samples from the reference subjects are taken from individuals at an early disease stage of a suspected neurogenerative disease. In some embodiments, the therapeutic agent is administered according to a body weight of the subject at a dosage of between about 0.1-100 mg/kg of the body weight. In some embodiments, the therapeutic agent is administered to the subject at a dosage of between about 200-1500 mg by intravenous administration. In some embodiments, the therapeutic agent is administered to the subject at a dosage of between about 700-1400 mg by intravenous administration. In some embodiments, the therapeutic agent is administered to the subject according to a treatment regimen comprising administering the therapeutic agent about once every 4 weeks (q4 week). In some embodiments, the first three doses administered to the subject comprise a dosage of about 700 mg of the therapeutic agent. In some embodiments, the doses administered to the subject after the first three doses administered comprise a dosage of about 1400 mg of the therapeutic agent. In some embodiments, systemic administration of the therapeutic agent. In some embodiments, systemic administration comprises i.v. administration of the therapeutic agent. In some embodiments, systemic administration comprises subcutaneous administration of the therapeutic agent. In some embodiments, systemic administration comprises oral administration of the therapeutic agent. In some embodiments, the anti-tau antibody comprises a HC comprising the sequence of SEQ ID NO: 55 and a LC comprising the sequence of SEQ ID NO: 56. In some embodiments, the anti-tau antibody comprises a HC comprising the sequence of SEQ ID NO: 57 and a LC comprising the sequence of SEQ ID NO: 58. In some embodiments, the anti-tau antibody comprises a HC comprising the sequence of SEQ ID NO: 57 and a LC comprising the sequence of SEQ ID NO: 59. In some embodiments, the anti-tau antibody comprises a HC comprising the sequence of SEQ ID NO: 60 and a LC comprising the sequence of SEQ ID NO: 61. In some embodiments, the anti-tau antibody comprises a HC comprising the sequence of SEQ ID NO: 62 and a LC comprising the sequence of SEQ ID NO: 63. In some embodiments, the anti-tau antibody comprises a HC comprising the sequence of SEQ ID NO: 64 and a LC comprising the sequence of SEQ ID NO: 65.

Described herein, in certain aspects, are methods of treating a neurodegenerative disease in a subject, the methods comprising: administering to the subject a therapeutically effective amount of an anti-sense therapy targeting huntingtin protein (HTT) listed in Table 10, wherein the subject is selected based on a detection of a level of pT217 tau in a sample from the subject above a standard value of pT217 tau level derived from a plurality of reference samples and wherein the level of pT217 tau predicts a positive therapeutic response in the subject to a treatment targeting HHT mRNA. In some embodiments, detection of the level of pT217 tau in the sample is performing using an anti-anti-tau antibody or the antigen-binding fragment thereof. In some embodiments, the anti-tau antibody or the antigen-binding fragment thereof comprises a variable heavy (VH) domain and a variable light (VL) domain. In some embodiments, the VH domain comprises at least 80%, at least 85%, at least 90%, at least 95% sequence identity to a sequence selected from SEQ ID NOs: 44-48. In some embodiments, the VL domain comprises at least 80%, at least 85%, at least 90%, at least 95% sequence identity to a sequence selected from SEQ ID NOs: 49-54. In some embodiments, the anti-tau antibody or antigen-binding fragment thereof comprises an IgG-scFv, nanobody, BiTE, diabody, DART, TandAb, scDiabody, scDiabody-CH3, triple body, mini-antibody, minibody, TriBi minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, tetravalent HCAb, scDiabody-Fc, diabody-Fc, tandem scFv-Fc, or intrabody. In some embodiments, the anti-tau antibody has an isotype selected from the group consisting of IgA1, IgA2, IgD, IgE, IgG1, IgG2, IgG3, IgG4, and IgM. In some embodiments, the anti-tau antibody or antigen-binding fragment thereof specifically binds an epitope in an N-terminal region of a tau polypeptide. In some embodiments, the anti-tau antibody or antigen-binding fragment thereof specifically binds to pT217-tau protein. In some embodiments, the anti-tau antibody or antigen-binding fragment thereof specifically binds to a cis-conformation of pT217-tau protein. In some embodiments, the anti-tau antibody or antigen-binding fragment thereof binds specifically to a tau polypeptide comprising phosphorylation at a site selected from a group consisting of pT181-tau, pT212-tau, phosphorylated-serine (pS)214-tau, pT217-tau, pT220-tau, and pT231-tau. In some embodiments, the anti-tau antibody or antigen-binding fragment thereof binds specifically to a tau polypeptide comprising phosphorylation at site pT217-tau. In some embodiments, the anti-tau antibody or antigen-binding fragment thereof binds specifically to a tau polypeptide comprising phosphorylation at site pT217-tau and pT231-tau. In some embodiments, the neurodegenerative disease comprises Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, Hallevorden-Spatz disease, mild cognitive impairment (MCI), Lewy Body's disease (LBD), Amyotrophic lateral sclerosis, spinal muscular atrophy, Friedreich's ataxia, Wernicke-Korsakoff syndrome, a prion disease, vascular dementia, Alzheimer's related dementia, Huntington's disease (HD), or a transgene-induced tauopathy. In some embodiments, the sample is selected from a cerebrospinal fluid (CSF) sample, a plasma sample, a blood sample, or a serum sample. In some embodiments, the neurodegenerative disease comprises Alzheimer's disease. In some embodiments, one or more symptoms of the neurodegenerative disease in the subject is ameliorated. In some embodiments, a progression of one or more symptoms of the neurodegenerative disease in the subject is slowed. In some embodiments, the therapeutic agent is administered intravenously, intramuscularly, intrathecally, intracerebrally, subcutaneously, orally, nasally, topically, buccally, or sublingually. In some embodiments, the therapeutic agent is administered directly to the CNS of the subject via intravenous delivery, intravascular delivery, intrathecal delivery, intracisternal delivery, intraspinal delivery, subpial delivery, or intracerebroventricular delivery. In some embodiments, the subject is at a preclinical stage of the neurogenerative disease. In some embodiments, the subject is at a prodromal stage of the neurogenerative disease. In some embodiments, the plurality of reference samples are taken from healthy individuals not suspected of having a neurogenerative disease. In some embodiments, the plurality of samples from the reference subjects are taken from individuals at an early disease stage of a suspected neurogenerative disease. In some embodiments, the therapeutic agent is administered according to a body weight of the subject at a dosage of between about 0.1-100 mg/kg of the body weight. In some embodiments, the therapeutic agent is administered to the subject at a dosage of between about 200-1500 mg by intravenous administration. In some embodiments, the therapeutic agent is administered to the subject at a dosage of between about 700-1400 mg by intravenous administration. In some embodiments, the therapeutic agent is administered to the subject according to a treatment regimen comprising administering the therapeutic agent about once every 4 weeks (q4 week). In some embodiments, the first three doses administered to the subject comprise a dosage of about 700 mg of the therapeutic agent. In some embodiments, the doses administered to the subject after the first three doses administered comprise a dosage of about 1400 mg of the therapeutic agent. In some embodiments, systemic administration of the therapeutic agent. In some embodiments, systemic administration comprises i.v. administration of the therapeutic agent. In some embodiments, systemic administration comprises subcutaneous administration of the therapeutic agent. In some embodiments, systemic administration comprises oral administration of the therapeutic agent. In some embodiments, the anti-tau antibody comprises a HC comprising the sequence of SEQ ID NO: 55 and a LC comprising the sequence of SEQ ID NO: 56. In some embodiments, the anti-tau antibody comprises a HC comprising the sequence of SEQ ID NO: 57 and a LC comprising the sequence of SEQ ID NO: 58. In some embodiments, the anti-tau antibody comprises a HC comprising the sequence of SEQ ID NO: 57 and a LC comprising the sequence of SEQ ID NO: 59. In some embodiments, the anti-tau antibody comprises a HC comprising the sequence of SEQ ID NO: 60 and a LC comprising the sequence of SEQ ID NO: 61. In some embodiments, the anti-tau antibody comprises a HC comprising the sequence of SEQ ID NO: 62 and a LC comprising the sequence of SEQ ID NO: 63. In some embodiments, the anti-tau antibody comprises a HC comprising the sequence of SEQ ID NO: 64 and a LC comprising the sequence of SEQ ID NO: 65.

Described herein, in certain aspects, are methods of treating a neurodegenerative disease in a subject, the methods comprising: (a) determining whether the subject has an elevated risk of a neurodegenerative condition leading to pathological memory impairment and cognitive decline compared to a reference subject and is suitable for treatment with a small molecule tau inhibitor by detecting a level of pT217 tau in a sample from the subject and correlating the level of pT217 tau in the sample with a standard value of pT217 tau level derived from a plurality of reference samples, wherein the detecting comprises performing an immunoassay on the sample using an anti-tau antibody or antigen-binding fragment thereof comprising i) a heavy chain comprising a variable heavy chain (VH) domain and ii) a light chain comprising a variable light chain (VL) domain, wherein the VH domain comprises HCDR1 sequence selected from SEQ ID NOs: 1-9, HCDR2 sequence selected from SEQ ID NOs: 10-17, and HCDR3 sequence selected from SEQ ID NOs: 18-23, and the VL domain comprises LCDR1 sequence selected from SEQ ID NOs: 24-31, LCDR2 sequence selected from SEQ ID NOs: 32-36, and LCDR3 sequence selected from SEQ ID NOs: 37-43; and (b) treating the subject by administering a therapeutically effective amount of a small molecule tau inhibitor listed in Table 10. In some embodiments, the VH domain comprises at least 80%, at least 85%, at least 90%, at least 95% sequence identity to a sequence selected from SEQ ID NOs: 44-48. In some embodiments, the VL domain comprises at least 80%, at least 85%, at least 90%, at least 95% sequence identity to a sequence selected from SEQ ID NOs: 49-54. In some embodiments, the anti-tau antibody or antigen-binding fragment thereof comprises an IgG-scFv, nanobody, BiTE, diabody, DART, TandAb, scDiabody, scDiabody-CH3, triple body, mini-antibody, minibody, TriBi minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, tetravalent HCAb, scDiabody-Fc, diabody-Fc, tandem scFv-Fc, or intrabody. In some embodiments, the anti-tau antibody has an isotype selected from the group consisting of IgA1, IgA2, IgD, IgE, IgG1, IgG2, IgG3, IgG4, and IgM. In some embodiments, the anti-tau antibody or antigen-binding fragment thereof specifically binds an epitope in an N-terminal region of a tau polypeptide. In some embodiments, the anti-tau antibody or antigen-binding fragment thereof specifically binds to pT217-tau protein. In some embodiments, the anti-tau antibody or antigen-binding fragment thereof specifically binds to a cis-conformation of pT217-tau protein. In some embodiments, the anti-tau antibody or antigen-binding fragment thereof binds specifically to a tau polypeptide comprising phosphorylation at a site selected from a group consisting of pT181-tau, pT212-tau, phosphorylated-serine (pS)214-tau, pT217-tau, pT220-tau, and pT231-tau. In some embodiments, the anti-tau antibody or antigen-binding fragment thereof binds specifically to a tau polypeptide comprising phosphorylation at site pT217-tau. In some embodiments, the anti-tau antibody or antigen-binding fragment thereof binds specifically to a tau polypeptide comprising phosphorylation at site pT217-tau and pT231-tau. In some embodiments, the neurodegenerative disease comprises Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, Hallevorden-Spatz disease, mild cognitive impairment (MCI), Lewy Body's disease (LBD), Amyotrophic lateral sclerosis, spinal muscular atrophy, Friedreich's ataxia, Wemicke-Korsakoff syndrome, a prion disease, vascular dementia, Alzheimer's related dementia, Huntington's disease (HD), or a transgene-induced tauopathy. In some embodiments, the sample is selected from a cerebrospinal fluid (CSF) sample, a plasma sample, a blood sample, or a serum sample. In some embodiments, the neurodegenerative disease comprises Alzheimer's disease. In some embodiments, one or more symptoms of the neurodegenerative disease in the subject is ameliorated. In some embodiments, a progression of one or more symptoms of the neurodegenerative disease in the subject is slowed. In some embodiments, the therapeutic agent is administered intravenously, intramuscularly, intrathecally, intracerebrally, subcutaneously, orally, nasally, topically, buccally, or sublingually. In some embodiments, the therapeutic agent is administered directly to the CNS of the subject via intravenous delivery, intravascular delivery, intrathecal delivery, intracisternal delivery, intraspinal delivery, subpial delivery, or intracerebroventricular delivery. In some embodiments, the subject is at a preclinical stage of the neurogenerative disease. In some embodiments, the subject is at a prodromal stage of the neurogenerative disease. In some embodiments, the plurality of reference samples are taken from healthy individuals not suspected of having a neurogenerative disease. In some embodiments, the plurality of samples from the reference subjects are taken from individuals at an early disease stage of a suspected neurogenerative disease. In some embodiments, the therapeutic agent is administered according to a body weight of the subject at a dosage of between about 0.1-100 mg/kg of the body weight. In some embodiments, the therapeutic agent is administered to the subject at a dosage of between about 200-1500 mg by intravenous administration. In some embodiments, the therapeutic agent is administered to the subject at a dosage of between about 700-1400 mg by intravenous administration. In some embodiments, the therapeutic agent is administered to the subject according to a treatment regimen comprising administering the therapeutic agent about once every 4 weeks (q4 week). In some embodiments, the first three doses administered to the subject comprise a dosage of about 700 mg of the therapeutic agent. In some embodiments, the doses administered to the subject after the first three doses administered comprise a dosage of about 1400 mg of the therapeutic agent. In some embodiments, systemic administration of the therapeutic agent. In some embodiments, systemic administration comprises i.v. administration of the therapeutic agent. In some embodiments, systemic administration comprises subcutaneous administration of the therapeutic agent. In some embodiments, systemic administration comprises oral administration of the therapeutic agent. In some embodiments, the anti-tau antibody comprises a HC comprising the sequence of SEQ ID NO: 55 and a LC comprising the sequence of SEQ ID NO: 56. In some embodiments, the anti-tau antibody comprises a HC comprising the sequence of SEQ ID NO: 57 and a LC comprising the sequence of SEQ ID NO: 58. In some embodiments, the anti-tau antibody comprises a HC comprising the sequence of SEQ ID NO: 57 and a LC comprising the sequence of SEQ ID NO: 59. In some embodiments, the anti-tau antibody comprises a HC comprising the sequence of SEQ ID NO: 60 and a LC comprising the sequence of SEQ ID NO: 61. In some embodiments, the anti-tau antibody comprises a HC comprising the sequence of SEQ ID NO: 62 and a LC comprising the sequence of SEQ ID NO: 63. In some embodiments, the anti-tau antibody comprises a HC comprising the sequence of SEQ ID NO: 64 and a LC comprising the sequence of SEQ ID NO: 65.

Described herein, in certain aspects, are methods of treating a neurodegenerative disease in a subject, the methods comprising: (a) determining whether the subject has an elevated risk of a neurodegenerative condition leading to pathological memory impairment and cognitive decline compared to a reference subject and is suitable for treatment with an anti-tau antibody by detecting a level of pT217 tau in a sample from the subject and correlating the level of pT217 tau in the sample with a standard value of pT217 tau level derived from a plurality of reference samples, wherein the detecting comprises performing an immunoassay on the sample using an antibody or antigen-binding fragment thereof comprising i) a heavy chain comprising a variable heavy chain (VH) domain and ii) a light chain comprising a variable light chain (VL) domain, wherein the VH domain comprises HCDR1 sequence selected from SEQ ID NOs: 1-9, HCDR2 sequence selected from SEQ ID NOs: 10-17, and HCDR3 sequence selected from SEQ ID NOs: 18-23, and the VL domain comprises LCDR1 sequence selected from SEQ ID NOs: 24-31, LCDR2 sequence selected from SEQ ID NOs: 32-36, and LCDR3 sequence selected from SEQ ID NOs: 37-43; and (b) treating the subject by administering a therapeutically effective amount of an anti-tau antibody listed in Table 10. In some embodiments, the VH domain comprises at least 80%, at least 85%, at least 90%, at least 95% sequence identity to a sequence selected from SEQ ID NOs: 44-48. In some embodiments, the VL domain comprises at least 80%, at least 85%, at least 90%, at least 95% sequence identity to a sequence selected from SEQ ID NOs: 49-54. In some embodiments, the anti-tau antibody or antigen-binding fragment thereof comprises an IgG-scFv, nanobody, BiTE, diabody, DART, TandAb, scDiabody, scDiabody-CH3, triple body, mini-antibody, minibody, TrⁱBi minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, tetravalent HCAb, scDiabody-Fc, diabody-Fc, tandem scFv-Fc, or intrabody. In some embodiments, the anti-tau antibody has an isotype selected from the group consisting of IgA1, IgA2, IgD, IgE, IgG1, IgG2, IgG3, IgG4, and IgM. In some embodiments, the anti-tau antibody or antigen-binding fragment thereof specifically binds an epitope in an N-terminal region of a tau polypeptide. In some embodiments, the anti-tau antibody or antigen-binding fragment thereof specifically binds to pT217-tau protein. In some embodiments, the anti-tau antibody or antigen-binding fragment thereof specifically binds to a cis-conformation of pT217-tau protein. In some embodiments, the anti-tau antibody or antigen-binding fragment thereof binds specifically to a tau polypeptide comprising phosphorylation at a site selected from a group consisting of pT181-tau, pT212-tau, phosphorylated-serine (pS)214-tau, pT217-tau, pT220-tau, and pT231-tau. In some embodiments, the anti-tau antibody or antigen-binding fragment thereof binds specifically to a tau polypeptide comprising phosphorylation at site pT217-tau. In some embodiments, the anti-tau antibody or antigen-binding fragment thereof binds specifically to a tau polypeptide comprising phosphorylation at site pT217-tau and pT231-tau. In some embodiments, the neurodegenerative disease comprises Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, Hallevorden-Spatz disease, mild cognitive impairment (MCI), Lewy Body's disease (LBD), Amyotrophic lateral sclerosis, spinal muscular atrophy, Friedreich's ataxia, Wemicke-Korsakoff syndrome, a prion disease, vascular dementia, Alzheimer's related dementia, Huntington's disease (HD), or a transgene-induced tauopathy. In some embodiments, the sample is selected from a cerebrospinal fluid (CSF) sample, a plasma sample, a blood sample, or a serum sample. In some embodiments, the neurodegenerative disease comprises Alzheimer's disease. In some embodiments, one or more symptoms of the neurodegenerative disease in the subject is ameliorated. In some embodiments, a progression of one or more symptoms of the neurodegenerative disease in the subject is slowed. In some embodiments, the therapeutic agent is administered intravenously, intramuscularly, intrathecally, intracerebrally, subcutaneously, orally, nasally, topically, buccally, or sublingually. In some embodiments, the therapeutic agent is administered directly to the CNS of the subject via intravenous delivery, intravascular delivery, intrathecal delivery, intracisternal delivery, intraspinal delivery, subpial delivery, or intracerebroventricular delivery. In some embodiments, the subject is at a preclinical stage of the neurogenerative disease. In some embodiments, the subject is at a prodromal stage of the neurogenerative disease. In some embodiments, the plurality of reference samples are taken from healthy individuals not suspected of having a neurogenerative disease. In some embodiments, the plurality of samples from the reference subjects are taken from individuals at an early disease stage of a suspected neurogenerative disease. In some embodiments, the therapeutic agent is administered according to a body weight of the subject at a dosage of between about 0.1-100 mg/kg of the body weight. In some embodiments, the therapeutic agent is administered to the subject at a dosage of between about 200-1500 mg by intravenous administration. In some embodiments, the therapeutic agent is administered to the subject at a dosage of between about 700-1400 mg by intravenous administration. In some embodiments, the therapeutic agent is administered to the subject according to a treatment regimen comprising administering the therapeutic agent about once every 4 weeks (q4 week). In some embodiments, the first three doses administered to the subject comprise a dosage of about 700 mg of the therapeutic agent. In some embodiments, the doses administered to the subject after the first three doses administered comprise a dosage of about 1400 mg of the therapeutic agent. In some embodiments, systemic administration of the therapeutic agent. In some embodiments, systemic administration comprises i.v. administration of the therapeutic agent. In some embodiments, systemic administration comprises subcutaneous administration of the therapeutic agent. In some embodiments, systemic administration comprises oral administration of the therapeutic agent. In some embodiments, the anti-tau antibody comprises a HC comprising the sequence of SEQ ID NO: 55 and a LC comprising the sequence of SEQ ID NO: 56. In some embodiments, the anti-tau antibody comprises a HC comprising the sequence of SEQ ID NO: 57 and a LC comprising the sequence of SEQ ID NO: 58. In some embodiments, the anti-tau antibody comprises a HC comprising the sequence of SEQ ID NO: 57 and a LC comprising the sequence of SEQ ID NO: 59. In some embodiments, the anti-tau antibody comprises a HC comprising the sequence of SEQ ID NO: 60 and a LC comprising the sequence of SEQ ID NO: 61. In some embodiments, the anti-tau antibody comprises a HC comprising the sequence of SEQ ID NO: 62 and a LC comprising the sequence of SEQ ID NO: 63. In some embodiments, the anti-tau antibody comprises a HC comprising the sequence of SEQ ID NO: 64 and a LC comprising the sequence of SEQ ID NO: 65.

Described herein, in certain aspects, are methods of treating a neurodegenerative disease in a subject, the method comprising: (a) determining whether the subject has an elevated risk of a neurodegenerative condition leading to pathological memory impairment and cognitive decline compared to a reference subject and is suitable for treatment with an anti-AD antibody by detecting a level of pT217 tau in a sample from the subject and correlating the level of pT217 tau in the sample with a standard value of pT217 tau level derived from a plurality of reference samples, wherein the detecting comprises performing an immunoassay on the sample using an antibody or antigen-binding fragment thereof comprising i) a heavy chain comprising a variable heavy chain (VH) domain and ii) a light chain comprising a variable light chain (VL) domain, wherein the VH domain comprises HCDR1 sequence selected from SEQ ID NOs: 1-9, HCDR2 sequence selected from SEQ ID NOs: 10-17, and HCDR3 sequence selected from SEQ ID NOs: 18-23, and the VL domain comprises LCDR1 sequence selected from SEQ ID NOs: 24-31, LCDR2 sequence selected from SEQ ID NOs: 32-36, and LCDR3 sequence selected from SEQ ID NOs: 37-43; and (b) treating the subject by administering a therapeutically effective amount of an anti-AD antibody listed in Table 10. In some embodiments, the VH domain comprises at least 80%, at least 85%, at least 90%, at least 95% sequence identity to a sequence selected from SEQ ID NOs: 44-48. In some embodiments, the VL domain comprises at least 80%, at least 85%, at least 90%, at least 95% sequence identity to a sequence selected from SEQ ID NOs: 49-54. In some embodiments, the anti-tau antibody or antigen-binding fragment thereof comprises an IgG-scFv, nanobody, BiTE, diabody, DART, TandAb, scDiabody, scDiabody-CH3, triple body, mini-antibody, minibody, TriBi minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, tetravalent HCAb, scDiabody-Fc, diabody-Fc, tandem scFv-Fc, or intrabody. In some embodiments, the anti-tau antibody has an isotype selected from the group consisting of IgA1, IgA2, IgD, IgE, IgG1, IgG2, IgG3, IgG4, and IgM. In some embodiments, the anti-tau antibody or antigen-binding fragment thereof specifically binds an epitope in an N-terminal region of a tau polypeptide. In some embodiments, the anti-tau antibody or antigen-binding fragment thereof specifically binds to pT217-tau protein. In some embodiments, the anti-tau antibody or antigen-binding fragment thereof specifically binds to a cis-conformation of pT217-tau protein. In some embodiments, the anti-tau antibody or antigen-binding fragment thereof binds specifically to a tau polypeptide comprising phosphorylation at a site selected from a group consisting of pT181-tau, pT212-tau, phosphorylated-serine (pS)214-tau, pT217-tau, pT220-tau, and pT231-tau. In some embodiments, the anti-tau antibody or antigen-binding fragment thereof binds specifically to a tau polypeptide comprising phosphorylation at site pT217-tau. In some embodiments, the anti-tau antibody or antigen-binding fragment thereof binds specifically to a tau polypeptide comprising phosphorylation at site pT217-tau and pT231-tau. In some embodiments, the neurodegenerative disease comprises Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, Hallevorden-Spatz disease, mild cognitive impairment (MCI), Lewy Body's disease (LBD), Amyotrophic lateral sclerosis, spinal muscular atrophy, Friedreich's ataxia, Wemicke-Korsakoff syndrome, a prion disease, vascular dementia, Alzheimer's related dementia, Huntington's disease (HD), or a transgene-induced tauopathy. In some embodiments, the sample is selected from a cerebrospinal fluid (CSF) sample, a plasma sample, a blood sample, or a serum sample. In some embodiments, the neurodegenerative disease comprises Alzheimer's disease. In some embodiments, one or more symptoms of the neurodegenerative disease in the subject is ameliorated. In some embodiments, a progression of one or more symptoms of the neurodegenerative disease in the subject is slowed. In some embodiments, the therapeutic agent is administered intravenously, intramuscularly, intrathecally, intracerebrally, subcutaneously, orally, nasally, topically, buccally, or sublingually. In some embodiments, the therapeutic agent is administered directly to the CNS of the subject via intravenous delivery, intravascular delivery, intrathecal delivery, intracisternal delivery, intraspinal delivery, subpial delivery, or intracerebroventricular delivery. In some embodiments, the subject is at a preclinical stage of the neurogenerative disease. In some embodiments, the subject is at a prodromal stage of the neurogenerative disease. In some embodiments, the plurality of reference samples are taken from healthy individuals not suspected of having a neurogenerative disease. In some embodiments, the plurality of samples from the reference subjects are taken from individuals at an early disease stage of a suspected neurogenerative disease. In some embodiments, the therapeutic agent is administered according to a body weight of the subject at a dosage of between about 0.1-100 mg/kg of the body weight. In some embodiments, the therapeutic agent is administered to the subject at a dosage of between about 200-1500 mg by intravenous administration. In some embodiments, the therapeutic agent is administered to the subject at a dosage of between about 700-1400 mg by intravenous administration. In some embodiments, the therapeutic agent is administered to the subject according to a treatment regimen comprising administering the therapeutic agent about once every 4 weeks (q4 week). In some embodiments, the first three doses administered to the subject comprise a dosage of about 700 mg of the therapeutic agent. In some embodiments, the doses administered to the subject after the first three doses administered comprise a dosage of about 1400 mg of the therapeutic agent. In some embodiments, systemic administration of the therapeutic agent. In some embodiments, systemic administration comprises i.v. administration of the therapeutic agent. In some embodiments, systemic administration comprises subcutaneous administration of the therapeutic agent. In some embodiments, systemic administration comprises oral administration of the therapeutic agent. In some embodiments, the anti-tau antibody comprises a HC comprising the sequence of SEQ ID NO: 55 and a LC comprising the sequence of SEQ ID NO: 56. In some embodiments, the anti-tau antibody comprises a HC comprising the sequence of SEQ ID NO: 57 and a LC comprising the sequence of SEQ ID NO: 58. In some embodiments, the anti-tau antibody comprises a HC comprising the sequence of SEQ ID NO: 57 and a LC comprising the sequence of SEQ ID NO: 59. In some embodiments, the anti-tau antibody comprises a HC comprising the sequence of SEQ ID NO: 60 and a LC comprising the sequence of SEQ ID NO: 61. In some embodiments, the anti-tau antibody comprises a HC comprising the sequence of SEQ ID NO: 62 and a LC comprising the sequence of SEQ ID NO: 63. In some embodiments, the anti-tau antibody comprises a HC comprising the sequence of SEQ ID NO: 64 and a LC comprising the sequence of SEQ ID NO: 65.

Described herein, in certain aspects, are methods of treating a neurodegenerative disease in a subject, the methods comprising: (a) determining whether the subject has an elevated risk of a neurodegenerative condition leading to pathological memory impairment and cognitive decline compared to a reference subject and is suitable for treatment with a small molecule amyloid-beta peptide inhibitor by detecting a level of pT217 tau in a sample from the subject and correlating the level of pT217 tau in the sample with a standard value of pT217 tau level derived from a plurality of reference samples, wherein the detecting comprises performing an immunoassay on the sample using an antibody or antigen-binding fragment thereof comprising i) a heavy chain comprising a variable heavy chain (VH) domain and ii) a light chain comprising a variable light chain (VL) domain, wherein the VH domain comprises HCDR1 sequence selected from SEQ ID NOs: 1-9, HCDR2 sequence selected from SEQ ID NOs: 10-17, and HCDR3 sequence selected from SEQ ID NOs: 18-23, and the VL domain comprises LCDR1 sequence selected from SEQ ID NOs: 24-31, LCDR2 sequence selected from SEQ ID NOs: 32-36, and LCDR3 sequence selected from SEQ ID NOs: 37-43; and (b) treating the subject by administering a therapeutically effective amount of a small molecule amyloid-beta peptide inhibitor listed in Table 10. In some embodiments, the VH domain comprises at least 80%, at least 85%, at least 90%, at least 95% sequence identity to a sequence selected from SEQ ID NOs: 44-48. In some embodiments, the VL domain comprises at least 80%, at least 85%, at least 90%, at least 95% sequence identity to a sequence selected from SEQ ID NOs: 49-54. In some embodiments, the anti-tau antibody or antigen-binding fragment thereof comprises an IgG-scFv, nanobody, BiTE, diabody, DART, TandAb, scDiabody, scDiabody-CH3, triple body, mini-antibody, minibody, TriBi minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, tetravalent HCAb, scDiabody-Fc, diabody-Fc, tandem scFv-Fc, or intrabody. In some embodiments, the anti-tau antibody has an isotype selected from the group consisting of IgA1, IgA2, IgD, IgE, IgG1, IgG2, IgG3, IgG4, and IgM. In some embodiments, the anti-tau antibody or antigen-binding fragment thereof specifically binds an epitope in an N-terminal region of a tau polypeptide. In some embodiments, the anti-tau antibody or antigen-binding fragment thereof specifically binds to pT217-tau protein. In some embodiments, the anti-tau antibody or antigen-binding fragment thereof specifically binds to a cis-conformation of pT217-tau protein. In some embodiments, the anti-tau antibody or antigen-binding fragment thereof binds specifically to a tau polypeptide comprising phosphorylation at a site selected from a group consisting of pT181-tau, pT212-tau, phosphorylated-serine (pS)214-tau, pT217-tau, pT220-tau, and pT231-tau. In some embodiments, the anti-tau antibody or antigen-binding fragment thereof binds specifically to a tau polypeptide comprising phosphorylation at site pT217-tau. In some embodiments, the anti-tau antibody or antigen-binding fragment thereof binds specifically to a tau polypeptide comprising phosphorylation at site pT217-tau and pT231-tau. In some embodiments, the neurodegenerative disease comprises Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, Hallevorden-Spatz disease, mild cognitive impairment (MCI), Lewy Body's disease (LBD), Amyotrophic lateral sclerosis, spinal muscular atrophy, Friedreich's ataxia, Wemicke-Korsakoff syndrome, a prion disease, vascular dementia, Alzheimer's related dementia, Huntington's disease (HD), or a transgene-induced tauopathy. In some embodiments, the sample is selected from a cerebrospinal fluid (CSF) sample, a plasma sample, a blood sample, or a serum sample. In some embodiments, the neurodegenerative disease comprises Alzheimer's disease. In some embodiments, one or more symptoms of the neurodegenerative disease in the subject is ameliorated. In some embodiments, a progression of one or more symptoms of the neurodegenerative disease in the subject is slowed. In some embodiments, the therapeutic agent is administered intravenously, intramuscularly, intrathecally, intracerebrally, subcutaneously, orally, nasally, topically, buccally, or sublingually. In some embodiments, the therapeutic agent is administered directly to the CNS of the subject via intravenous delivery, intravascular delivery, intrathecal delivery, intracisternal delivery, intraspinal delivery, subpial delivery, or intracerebroventricular delivery. In some embodiments, the subject is at a preclinical stage of the neurogenerative disease. In some embodiments, the subject is at a prodromal stage of the neurogenerative disease. In some embodiments, the plurality of reference samples are taken from healthy individuals not suspected of having a neurogenerative disease. In some embodiments, the plurality of samples from the reference subjects are taken from individuals at an early disease stage of a suspected neurogenerative disease. In some embodiments, the therapeutic agent is administered according to a body weight of the subject at a dosage of between about 0.1-100 mg/kg of the body weight. In some embodiments, the therapeutic agent is administered to the subject at a dosage of between about 200-1500 mg by intravenous administration. In some embodiments, the therapeutic agent is administered to the subject at a dosage of between about 700-1400 mg by intravenous administration. In some embodiments, the therapeutic agent is administered to the subject according to a treatment regimen comprising administering the therapeutic agent about once every 4 weeks (q4 week). In some embodiments, the first three doses administered to the subject comprise a dosage of about 700 mg of the therapeutic agent. In some embodiments, the doses administered to the subject after the first three doses administered comprise a dosage of about 1400 mg of the therapeutic agent. In some embodiments, systemic administration of the therapeutic agent. In some embodiments, systemic administration comprises i.v. administration of the therapeutic agent. In some embodiments, systemic administration comprises subcutaneous administration of the therapeutic agent. In some embodiments, systemic administration comprises oral administration of the therapeutic agent. In some embodiments, the anti-tau antibody comprises a HC comprising the sequence of SEQ ID NO: 55 and a LC comprising the sequence of SEQ ID NO: 56. In some embodiments, the anti-tau antibody comprises a HC comprising the sequence of SEQ ID NO: 57 and a LC comprising the sequence of SEQ ID NO: 58. In some embodiments, the anti-tau antibody comprises a HC comprising the sequence of SEQ ID NO: 57 and a LC comprising the sequence of SEQ ID NO: 59. In some embodiments, the anti-tau antibody comprises a HC comprising the sequence of SEQ ID NO: 60 and a LC comprising the sequence of SEQ ID NO: 61. In some embodiments, the anti-tau antibody comprises a HC comprising the sequence of SEQ ID NO: 62 and a LC comprising the sequence of SEQ ID NO: 63. In some embodiments, the anti-tau antibody comprises a HC comprising the sequence of SEQ ID NO: 64 and a LC comprising the sequence of SEQ ID NO: 65.

Described herein, in certain aspects, are methods of treating a neurodegenerative disease in a subject, the methods comprising: (a) receiving data obtained by detecting a level of pT217 tau in a sample from the subject and correlating the level of pT217 tau in the sample with a standard value of pT217 tau level derived from a plurality of reference samples, wherein the detecting comprises performing an immunoassay on the sample using an anti-tau antibody or antigen-binding fragment thereof comprising i) a heavy chain comprising a variable heavy chain (VH) domain and ii) a light chain comprising a variable light chain (VL) domain, wherein the VH domain comprises HCDR1 sequence selected from SEQ ID NOs: 1-9, HCDR2 sequence selected from SEQ ID NOs: 10-17, and HCDR3 sequence selected from SEQ ID NOs: 18-23, and the VL domain comprises LCDR1 sequence selected from SEQ ID NOs: 24-31, LCDR2 sequence selected from SEQ ID NOs: 32-36, and LCDR3 sequence selected from SEQ ID NOs: 37-43; (b) analyzing a likelihood of clinical diagnosis of the neurodegenerative disease in the subject based at least in part on the receiving; (c) providing an early prognosis of the neurodegenerative disease in the subject based at least in part on the analyzing, wherein the early prognosis predates supplying a diagnosis of a prodromal stage of the neurodegenerative disease in the subject; and (d) initiating a treatment comprising administration of a formulation comprising a therapeutic agent, wherein deciding the initiating is based at least in part on the providing, wherein the therapeutic agent is selected from the group consisting of: ABvac40LY3002813, AADvac-1, ACI-35, AAV-Htert, ACU193, ASN51, ACI-242, AD-35, Aducanumab, AGB101, AL002, Allopregnanolone, APH-1105, Atuzaginstat (COR388), AVP-786, AXS-05, Baricitinib, Bepranemab, BCG vaccine, BEIY2153, BPN14770, Bromocriptine, Bryostatin 1, BXCL-501, Blarcamesine (ANAVEX2-73), Bapineuzumab, BIIB092 (BMS-986168, IPN007, Gosuranemab), BIIB080/IONIS-MAPTRx, BIIB076, BPDO-1603, BMS-984923, BDPP (bioactive dietary polyphenol preparation), Brexpiprazole, Caffeine, CAD 1063, Canakinumab, Contraloid acetate, COR588, CORT108297, CNP520(AMG520), C2N-8E12 (ABBV-8E12), Crenezumab, CST-2032, Curcumin, CY6463, Dabigatran,DAOIB, Dapagliflozin, Daratumumab, Dasatinib, Deferiprone, Donanemab (LY3002813), Donepezil (Aricept), Dronabinol, Dutanumab, Edicotinib (JNJ-40346527), Efavirenz, Emtricitabine, Empagliflozin, Elenbecestat (E2609), E2814, Ednerpic (T-817 mA), Elayta (CT1812), ExPlas, Escitalopram, Fosgonimeton (ATH01017), Galantamine, Gantenerumab, Brain Shuttle Gantenerumab (R07126209), GB301, grapeseed extract, GV1001, Guanfacine, GV-971, Intranasal insulin, HT-ALZ, Huperzine A, Hydralazine, IGC AD1, Icosapent ethyl (IPE), JNJ-63733657, KHK 66401, Lu AF87908, Lu AF20513, Lamivudine (3TC), Lanabecestat, Lenalidomide, Levetiracetam, L-serine, Lupron, Remternetug (LY3372993), LY3372689, LMTX, LY3303560 (Zagotenemab), Lecanemab (BAN2401), Losartan, LX1001, MEDI 1814, Memantine, Metformin, MK-1942, MIB-626, Montelukast, MW150, Neflamapimod (VX-745), Nabilone, NE3107, nicotinamide, nicotine, NNI-362, Nilotinib BE, Octohydro-aminoacridine succinate, Obicetrapib, Omega-3 (DHA+EPA), perindopril, Pepinemab (VX15), Posiphen, Prazosin, Ponezumab, Protollin, PQ912, PNT001, PU-AD, Quercetin, rapamycin, REM0046127, RG7345 (R06926496), rivastigmine, RG6100, R07105705 (MTAU9937A, RG6100, Semorinemab), Salsalate, Sargramostim, Semorinemab, Senicapoc, Semaglutide, SHR-1707, Simufilam (PTI-125), Sodium oligomannate, Solanezumab, scyllo-inositol, Sovateltide (PMZ-1620), Suvorexant, T3D-959, TB006, Telmisartan, Trehalose, Tdap vaccine, THC-free CBD oil, Thiethylperazine (TEP), Troriluzole (BHV4157), TPI 2871, Tricaprilin (AC-1202), TRx0237, UB-311, UCB0107, valiltramiprosate (ALZ-801), Valacyclovir, varoglutamstat (PQ912), VGH-AD1, Vorinostat, VT301, XProl595, Xanamem, Yangxue Qingao pills, an antisense RNA directed to an isoform of human tau mRNA or an isoform of human amyloid beta, an siRNA directed to an isoform of human tau mRNA or an isoform of human amyloid beta, an antisense oligonucleotide directed to an isoform of human tau mRNA or an isoform of human amyloid beta, an LNA oligonucleotide directed to an isoform of human tau mRNA or an isoform of human amyloid beta, a CRISPRn-based therapeutic targeting the human MAPT or the human APP locus, a CRISPRi-based therapeutic targeting the human MAPT or the human APP locus, allogeneic human MSCs, SNKO1 (autologous natural killer cell), allogenic adipose MSC-exosomes, CA-AC-02 (placenta derived MSCs), HUVEC-derived MSCs (NEUROSTEM), and AstroStem (autologous adipose-derived MSCs). In some embodiments, the VH domain comprises at least 80%, at least 85%, at least 90%, at least 95% sequence identity to a sequence selected from SEQ ID NOs: 44-48. In some embodiments, the VL domain comprises at least 80%, at least 85%, at least 90%, at least 95% sequence identity to a sequence selected from SEQ ID NOs: 49-54. In some embodiments, the anti-tau antibody or antigen-binding fragment thereof comprises an IgG-scFv, nanobody, BiTE, diabody, DART, TandAb, scDiabody, scDiabody-CH3, triple body, mini-antibody, minibody, TriBi minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, tetravalent HCAb, scDiabody-Fc, diabody-Fc, tandem scFv-Fc, or intrabody. In some embodiments, the anti-tau antibody has an isotype selected from the group consisting of IgA1, IgA2, IgD, IgE, IgG1, IgG2, IgG3, IgG4, and IgM. In some embodiments, the anti-tau antibody or antigen-binding fragment thereof specifically binds an epitope in an N-terminal region of a tau polypeptide. In some embodiments, the anti-tau antibody or antigen-binding fragment thereof specifically binds to pT217-tau protein. In some embodiments, the anti-tau antibody or antigen-binding fragment thereof specifically binds to a cis-conformation of pT217-tau protein. In some embodiments, the anti-tau antibody or antigen-binding fragment thereof binds specifically to a tau polypeptide comprising phosphorylation at a site selected from a group consisting of pT181-tau, pT212-tau, phosphorylated-serine (pS)214-tau, pT217-tau, pT220-tau, and pT231-tau. In some embodiments, the anti-tau antibody or antigen-binding fragment thereof binds specifically to a tau polypeptide comprising phosphorylation at site pT217-tau. In some embodiments, the anti-tau antibody or antigen-binding fragment thereof binds specifically to a tau polypeptide comprising phosphorylation at site pT217-tau and pT231-tau. In some embodiments, the neurodegenerative disease comprises Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, Hallevorden-Spatz disease, mild cognitive impairment (MCI), Lewy Body's disease (LBD), Amyotrophic lateral sclerosis, spinal muscular atrophy, Friedreich's ataxia, Wemicke-Korsakoff syndrome, a prion disease, vascular dementia, Alzheimer's related dementia, Huntington's disease (HD), or a transgene-induced tauopathy. In some embodiments, the sample is selected from a cerebrospinal fluid (CSF) sample, a plasma sample, a blood sample, or a serum sample. In some embodiments, the neurodegenerative disease comprises Alzheimer's disease. In some embodiments, one or more symptoms of the neurodegenerative disease in the subject is ameliorated. In some embodiments, a progression of one or more symptoms of the neurodegenerative disease in the subject is slowed. In some embodiments, the therapeutic agent is administered intravenously, intramuscularly, intrathecally, intracerebrally, subcutaneously, orally, nasally, topically, buccally, or sublingually. In some embodiments, the therapeutic agent is administered directly to the CNS of the subject via intravenous delivery, intravascular delivery, intrathecal delivery, intracisternal delivery, intraspinal delivery, subpial delivery, or intracerebroventricular delivery. In some embodiments, the subject is at a preclinical stage of the neurogenerative disease. In some embodiments, the subject is at a prodromal stage of the neurogenerative disease. In some embodiments, the plurality of reference samples are taken from healthy individuals not suspected of having a neurogenerative disease. In some embodiments, the plurality of samples from the reference subjects are taken from individuals at an early disease stage of a suspected neurogenerative disease. In some embodiments, the therapeutic agent is administered according to a body weight of the subject at a dosage of between about 0.1-100 mg/kg of the body weight. In some embodiments, the therapeutic agent is administered to the subject at a dosage of between about 200-1500 mg by intravenous administration. In some embodiments, the therapeutic agent is administered to the subject at a dosage of between about 700-1400 mg by intravenous administration. In some embodiments, the therapeutic agent is administered to the subject according to a treatment regimen comprising administering the therapeutic agent about once every 4 weeks (q4 week). In some embodiments, the first three doses administered to the subject comprise a dosage of about 700 mg of the therapeutic agent. In some embodiments, the doses administered to the subject after the first three doses administered comprise a dosage of about 1400 mg of the therapeutic agent. In some embodiments, systemic administration of the therapeutic agent. In some embodiments, systemic administration comprises i.v. administration of the therapeutic agent. In some embodiments, systemic administration comprises subcutaneous administration of the therapeutic agent. In some embodiments, systemic administration comprises oral administration of the therapeutic agent. In some embodiments, the anti-tau antibody comprises a HC comprising the sequence of SEQ ID NO: 55 and a LC comprising the sequence of SEQ ID NO: 56. In some embodiments, the anti-tau antibody comprises a HC comprising the sequence of SEQ ID NO: 57 and a LC comprising the sequence of SEQ ID NO: 58. In some embodiments, the anti-tau antibody comprises a HC comprising the sequence of SEQ ID NO: 57 and a LC comprising the sequence of SEQ ID NO: 59. In some embodiments, the anti-tau antibody comprises a HC comprising the sequence of SEQ ID NO: 60 and a LC comprising the sequence of SEQ ID NO: 61. In some embodiments, the anti-tau antibody comprises a HC comprising the sequence of SEQ ID NO: 62 and a LC comprising the sequence of SEQ ID NO: 63. In some embodiments, the anti-tau antibody comprises a HC comprising the sequence of SEQ ID NO: 64 and a LC comprising the sequence of SEQ ID NO: 65.

Described herein, in certain aspects, are methods of treating a neurodegenerative disease in a subject, the methods comprising: (a) receiving data obtained by detecting a level of pT217 tau in a sample from the subject and correlating the level of pT217 tau in the sample with a standard value of pT217 tau level derived from a plurality of reference samples, wherein the detecting comprises performing an immunoassay on the sample using an anti-tau antibody or antigen-binding fragment thereof comprising i) a heavy chain comprising a variable heavy chain (VH) domain and ii) a light chain comprising a variable light chain (VL) domain, wherein the VH domain comprises HCDR1 sequence selected from SEQ ID NOs: 1-9, HCDR2 sequence selected from SEQ ID NOs: 10-17, and HCDR3 sequence selected from SEQ ID NOs: 18-23, and the VL domain comprises LCDR1 sequence selected from SEQ ID NOs: 24-31, LCDR2 sequence selected from SEQ ID NOs: 32-36, and LCDR3 sequence selected from SEQ ID NOs: 37-43; (b) analyzing a likelihood of clinical diagnosis of the neurodegenerative disease in the subject based at least in part on the receiving; (c) providing an early prognosis of the neurodegenerative disease in the subject based at least in part on the analyzing, wherein the early prognosis predates supplying a diagnosis of a prodromal stage of the neurodegenerative disease in the subject; and (d) initiating a treatment comprising administration of a formulation comprising a therapeutic agent, wherein deciding the initiating is based at least in part on the providing, wherein the therapeutic agent is selected from an inhibitor of tau listed in Table 10. In some embodiments, the VH domain comprises at least 80%, at least 85%, at least 90%, at least 95% sequence identity to a sequence selected from SEQ ID NOs: 44-48. In some embodiments, the VL domain comprises at least 80%, at least 85%, at least 90%, at least 95% sequence identity to a sequence selected from SEQ ID NOs: 49-54. In some embodiments, the anti-tau antibody or antigen-binding fragment thereof comprises an IgG-scFv, nanobody, BiTE, diabody, DART, TandAb, scDiabody, scDiabody-CH3, triple body, mini-antibody, minibody, TriBi minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, tetravalent HCAb, scDiabody-Fc, diabody-Fc, tandem scFv-Fc, or intrabody. In some embodiments, the anti-tau antibody has an isotype selected from the group consisting of IgA1, IgA2, IgD, IgE, IgG1, IgG2, IgG3, IgG4, and IgM. In some embodiments, the anti-tau antibody or antigen-binding fragment thereof specifically binds an epitope in an N-terminal region of a tau polypeptide. In some embodiments, the anti-tau antibody or antigen-binding fragment thereof specifically binds to pT217-tau protein. In some embodiments, the anti-tau antibody or antigen-binding fragment thereof specifically binds to a cis-conformation of pT217-tau protein. In some embodiments, the anti-tau antibody or antigen-binding fragment thereof binds specifically to a tau polypeptide comprising phosphorylation at a site selected from a group consisting of pT181-tau, pT212-tau, phosphorylated-serine (pS)214-tau, pT217-tau, pT220-tau, and pT231-tau. In some embodiments, the anti-tau antibody or antigen-binding fragment thereof binds specifically to a tau polypeptide comprising phosphorylation at site pT217-tau. In some embodiments, the anti-tau antibody or antigen-binding fragment thereof binds specifically to a tau polypeptide comprising phosphorylation at site pT217-tau and pT231-tau. In some embodiments, the neurodegenerative disease comprises Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, Hallevorden-Spatz disease, mild cognitive impairment (MCI), Lewy Body's disease (LBD), Amyotrophic lateral sclerosis, spinal muscular atrophy, Friedreich's ataxia, Wemicke-Korsakoff syndrome, a prion disease, vascular dementia, Alzheimer's related dementia, Huntington's disease (HD), or a transgene-induced tauopathy. In some embodiments, the sample is selected from a cerebrospinal fluid (CSF) sample, a plasma sample, a blood sample, or a serum sample. In some embodiments, the neurodegenerative disease comprises Alzheimer's disease. In some embodiments, one or more symptoms of the neurodegenerative disease in the subject is ameliorated. In some embodiments, a progression of one or more symptoms of the neurodegenerative disease in the subject is slowed. In some embodiments, the therapeutic agent is administered intravenously, intramuscularly, intrathecally, intracerebrally, subcutaneously, orally, nasally, topically, buccally, or sublingually. In some embodiments, the therapeutic agent is administered directly to the CNS of the subject via intravenous delivery, intravascular delivery, intrathecal delivery, intracisternal delivery, intraspinal delivery, subpial delivery, or intracerebroventricular delivery. In some embodiments, the subject is at a preclinical stage of the neurogenerative disease. In some embodiments, the subject is at a prodromal stage of the neurogenerative disease. In some embodiments, the plurality of reference samples are taken from healthy individuals not suspected of having a neurogenerative disease. In some embodiments, the plurality of samples from the reference subjects are taken from individuals at an early disease stage of a suspected neurogenerative disease. In some embodiments, the therapeutic agent is administered according to a body weight of the subject at a dosage of between about 0.1-100 mg/kg of the body weight. In some embodiments, the therapeutic agent is administered to the subject at a dosage of between about 200-1500 mg by intravenous administration. In some embodiments, the therapeutic agent is administered to the subject at a dosage of between about 700-1400 mg by intravenous administration. In some embodiments, the therapeutic agent is administered to the subject according to a treatment regimen comprising administering the therapeutic agent about once every 4 weeks (q4 week). In some embodiments, the first three doses administered to the subject comprise a dosage of about 700 mg of the therapeutic agent. In some embodiments, the doses administered to the subject after the first three doses administered comprise a dosage of about 1400 mg of the therapeutic agent. In some embodiments, systemic administration of the therapeutic agent. In some embodiments, systemic administration comprises i.v. administration of the therapeutic agent. In some embodiments, systemic administration comprises subcutaneous administration of the therapeutic agent. In some embodiments, systemic administration comprises oral administration of the therapeutic agent. In some embodiments, the anti-tau antibody comprises a HC comprising the sequence of SEQ ID NO: 55 and a LC comprising the sequence of SEQ ID NO: 56. In some embodiments, the anti-tau antibody comprises a HC comprising the sequence of SEQ ID NO: 57 and a LC comprising the sequence of SEQ ID NO: 58. In some embodiments, the anti-tau antibody comprises a HC comprising the sequence of SEQ ID NO: 57 and a LC comprising the sequence of SEQ ID NO: 59. In some embodiments, the anti-tau antibody comprises a HC comprising the sequence of SEQ ID NO: 60 and a LC comprising the sequence of SEQ ID NO: 61. In some embodiments, the anti-tau antibody comprises a HC comprising the sequence of SEQ ID NO: 62 and a LC comprising the sequence of SEQ ID NO: 63. In some embodiments, the anti-tau antibody comprises a HC comprising the sequence of SEQ ID NO: 64 and a LC comprising the sequence of SEQ ID NO: 65.

Described herein, in certain aspects, are methods of treating a neurodegenerative disease in a subject, the method comprising: (a) receiving data obtained by detecting a level of pT217 tau in sample from the subject and correlating the level of pT217 tau in the sample with a standard value of pT217 tau CSF level derived from a plurality of reference samples, wherein the detecting comprises performing an immunoassay on the sample using an anti-tau antibody or antigen-binding fragment thereof comprising i) a heavy chain comprising a variable heavy chain (VH) domain and ii) a light chain comprising a variable light chain (VL) domain, wherein the VH domain comprises HCDR1 sequence selected from SEQ ID NOs: 1-9, HCDR2 sequence selected from SEQ ID NOs: 10-17, and HCDR3 sequence selected from SEQ ID NOs: 18-23, and the VL domain comprises LCDR1 sequence selected from SEQ ID NOs: 24-31, LCDR2 sequence selected from SEQ ID NOs: 32-36, and LCDR3 sequence selected from SEQ ID NOs: 37-43; (b) analyzing a likelihood of clinical diagnosis of the neurodegenerative disease in the subject based at least in part on the receiving; (c) providing an early prognosis of the neurodegenerative disease in the subject based at least in part on the analyzing, wherein the early prognosis predates supplying a diagnosis of a prodromal stage of the neurodegenerative disease in the subject; and (d) initiating a treatment comprising administration of a formulation comprising a therapeutic agent, wherein deciding the initiating is based at least in part on the providing, wherein the therapeutic agent is selected from an inhibitor of amyloid-beta peptide listed in Table 10. In some embodiments, the VH domain comprises at least 80%, at least 85%, at least 90%, at least 95% sequence identity to a sequence selected from SEQ ID NOs: 44-48. In some embodiments, the VL domain comprises at least 80%, at least 85%, at least 90%, at least 95% sequence identity to a sequence selected from SEQ ID NOs: 49-54. In some embodiments, the anti-tau antibody or antigen-binding fragment thereof comprises an IgG-scFv, nanobody, BiTE, diabody, DART, TandAb, scDiabody, scDiabody-CH3, triple body, mini-antibody, minibody, TriBi minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, tetravalent HCAb, scDiabody-Fc, diabody-Fc, tandem scFv-Fc, or intrabody. In some embodiments, the anti-tau antibody has an isotype selected from the group consisting of IgA1, IgA2, IgD, IgE, IgG1, IgG2, IgG3, IgG4, and IgM. In some embodiments, the anti-tau antibody or antigen-binding fragment thereof specifically binds an epitope in an N-terminal region of a tau polypeptide. In some embodiments, the anti-tau antibody or antigen-binding fragment thereof specifically binds to pT217-tau protein. In some embodiments, the anti-tau antibody or antigen-binding fragment thereof specifically binds to a cis-conformation of pT217-tau protein. In some embodiments, the anti-tau antibody or antigen-binding fragment thereof binds specifically to a tau polypeptide comprising phosphorylation at a site selected from a group consisting of pT181-tau, pT212-tau, phosphorylated-serine (pS)214-tau, pT217-tau, pT220-tau, and pT231-tau. In some embodiments, the anti-tau antibody or antigen-binding fragment thereof binds specifically to a tau polypeptide comprising phosphorylation at site pT217-tau. In some embodiments, the anti-tau antibody or antigen-binding fragment thereof binds specifically to a tau polypeptide comprising phosphorylation at site pT217-tau and pT231-tau. In some embodiments, the neurodegenerative disease comprises Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, Hallevorden-Spatz disease, mild cognitive impairment (MCI), Lewy Body's disease (LBD), Amyotrophic lateral sclerosis, spinal muscular atrophy, Friedreich's ataxia, Wemicke-Korsakoff syndrome, a prion disease, vascular dementia, Alzheimer's related dementia, Huntington's disease (HD), or a transgene-induced tauopathy. In some embodiments, the sample is selected from a cerebrospinal fluid (CSF) sample, a plasma sample, a blood sample, or a serum sample. In some embodiments, the neurodegenerative disease comprises Alzheimer's disease. In some embodiments, one or more symptoms of the neurodegenerative disease in the subject is ameliorated. In some embodiments, a progression of one or more symptoms of the neurodegenerative disease in the subject is slowed. In some embodiments, the therapeutic agent is administered intravenously, intramuscularly, intrathecally, intracerebrally, subcutaneously, orally, nasally, topically, buccally, or sublingually. In some embodiments, the therapeutic agent is administered directly to the CNS of the subject via intravenous delivery, intravascular delivery, intrathecal delivery, intracisternal delivery, intraspinal delivery, subpial delivery, or intracerebroventricular delivery. In some embodiments, the subject is at a preclinical stage of the neurogenerative disease. In some embodiments, the subject is at a prodromal stage of the neurogenerative disease. In some embodiments, the plurality of reference samples are taken from healthy individuals not suspected of having a neurogenerative disease. In some embodiments, the plurality of samples from the reference subjects are taken from individuals at an early disease stage of a suspected neurogenerative disease. In some embodiments, the therapeutic agent is administered according to a body weight of the subject at a dosage of between about 0.1-100 mg/kg of the body weight. In some embodiments, the therapeutic agent is administered to the subject at a dosage of between about 200-1500 mg by intravenous administration. In some embodiments, the therapeutic agent is administered to the subject at a dosage of between about 700-1400 mg by intravenous administration. In some embodiments, the therapeutic agent is administered to the subject according to a treatment regimen comprising administering the therapeutic agent about once every 4 weeks (q4 week). In some embodiments, the first three doses administered to the subject comprise a dosage of about 700 mg of the therapeutic agent. In some embodiments, the doses administered to the subject after the first three doses administered comprise a dosage of about 1400 mg of the therapeutic agent. In some embodiments, systemic administration of the therapeutic agent. In some embodiments, systemic administration comprises i.v. administration of the therapeutic agent. In some embodiments, systemic administration comprises subcutaneous administration of the therapeutic agent. In some embodiments, systemic administration comprises oral administration of the therapeutic agent. In some embodiments, the anti-tau antibody comprises a HC comprising the sequence of SEQ ID NO: 55 and a LC comprising the sequence of SEQ ID NO: 56. In some embodiments, the anti-tau antibody comprises a HC comprising the sequence of SEQ ID NO: 57 and a LC comprising the sequence of SEQ ID NO: 58. In some embodiments, the anti-tau antibody comprises a HC comprising the sequence of SEQ ID NO: 57 and a LC comprising the sequence of SEQ ID NO: 59. In some embodiments, the anti-tau antibody comprises a HC comprising the sequence of SEQ ID NO: 60 and a LC comprising the sequence of SEQ ID NO: 61. In some embodiments, the anti-tau antibody comprises a HC comprising the sequence of SEQ ID NO: 62 and a LC comprising the sequence of SEQ ID NO: 63. In some embodiments, the anti-tau antibody comprises a HC comprising the sequence of SEQ ID NO: 64 and a LC comprising the sequence of SEQ ID NO: 65.

Described herein, in certain aspects, are methods of treating a neurodegenerative disease in a subject, the method comprising: (a) receiving data obtained by detecting a level of pT217 tau in a sample from the subject and correlating the level of pT217 tau in the sample with a standard value of pT217 tau level derived from a plurality of reference samples, wherein the detecting comprises performing an immunoassay on the sample using an anti-tau antibody or antigen-binding fragment thereof comprising i) a heavy chain comprising a variable heavy chain (VH) domain and ii) a light chain comprising a variable light chain (VL) domain, wherein the VH domain comprises HCDR1 sequence selected from SEQ ID NOs: 1-9, HCDR2 sequence selected from SEQ ID NOs: 10-17, and HCDR3 sequence selected from SEQ ID NOs: 18-23, and the VL domain comprises LCDR1 sequence selected from SEQ ID NOs: 24-31, LCDR2 sequence selected from SEQ ID NOs: 32-36, and LCDR3 sequence selected from SEQ ID NOs: 37-43; (b) analyzing a likelihood of clinical diagnosis of the neurodegenerative disease in the subject based at least in part on the receiving; (c) providing an early prognosis of the neurodegenerative disease in the subject based at least in part on the analyzing, wherein the early prognosis predates supplying a diagnosis of a prodromal stage of the neurodegenerative disease in the subject; and (d) initiating a treatment comprising administration of a formulation comprising a therapeutic agent, wherein deciding the initiating is based at least in part on the providing, wherein the therapeutic agent is selected from a cholinesterase inhibitor listed in Table 10. In some embodiments, the VH domain comprises at least 80%, at least 85%, at least 90%, at least 95% sequence identity to a sequence selected from SEQ ID NOs: 44-48. In some embodiments, the VL domain comprises at least 80%, at least 85%, at least 90%, at least 95% sequence identity to a sequence selected from SEQ ID NOs: 49-54. In some embodiments, the anti-tau antibody or antigen-binding fragment thereof comprises an IgG-scFv, nanobody, BiTE, diabody, DART, TandAb, scDiabody, scDiabody-CH3, triple body, mini-antibody, minibody, TriBi minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, tetravalent HCAb, scDiabody-Fc, diabody-Fc, tandem scFv-Fc, or intrabody. In some embodiments, the anti-tau antibody has an isotype selected from the group consisting of IgA1, IgA2, IgD, IgE, IgG1, IgG2, IgG3, IgG4, and IgM. In some embodiments, the anti-tau antibody or antigen-binding fragment thereof specifically binds an epitope in an N-terminal region of a tau polypeptide. In some embodiments, the anti-tau antibody or antigen-binding fragment thereof specifically binds to pT217-tau protein. In some embodiments, the anti-tau antibody or antigen-binding fragment thereof specifically binds to a cis-conformation of pT217-tau protein. In some embodiments, the anti-tau antibody or antigen-binding fragment thereof binds specifically to a tau polypeptide comprising phosphorylation at a site selected from a group consisting of pT181-tau, pT212-tau, phosphorylated-serine (pS)214-tau, pT217-tau, pT220-tau, and pT231-tau. In some embodiments, the anti-tau antibody or antigen-binding fragment thereof binds specifically to a tau polypeptide comprising phosphorylation at site pT217-tau. In some embodiments, the anti-tau antibody or antigen-binding fragment thereof binds specifically to a tau polypeptide comprising phosphorylation at site pT217-tau and pT231-tau. In some embodiments, the neurodegenerative disease comprises Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, Hallevorden-Spatz disease, mild cognitive impairment (MCI), Lewy Body's disease (LBD), Amyotrophic lateral sclerosis, spinal muscular atrophy, Friedreich's ataxia, Wemicke-Korsakoff syndrome, a prion disease, vascular dementia, Alzheimer's related dementia, Huntington's disease (HD), or a transgene-induced tauopathy. In some embodiments, the sample is selected from a cerebrospinal fluid (CSF) sample, a plasma sample, a blood sample, or a serum sample. In some embodiments, the neurodegenerative disease comprises Alzheimer's disease. In some embodiments, one or more symptoms of the neurodegenerative disease in the subject is ameliorated. In some embodiments, a progression of one or more symptoms of the neurodegenerative disease in the subject is slowed. In some embodiments, the therapeutic agent is administered intravenously, intramuscularly, intrathecally, intracerebrally, subcutaneously, orally, nasally, topically, buccally, or sublingually. In some embodiments, the therapeutic agent is administered directly to the CNS of the subject via intravenous delivery, intravascular delivery, intrathecal delivery, intracisternal delivery, intraspinal delivery, subpial delivery, or intracerebroventricular delivery. In some embodiments, the subject is at a preclinical stage of the neurogenerative disease. In some embodiments, the subject is at a prodromal stage of the neurogenerative disease. In some embodiments, the plurality of reference samples are taken from healthy individuals not suspected of having a neurogenerative disease. In some embodiments, the plurality of samples from the reference subjects are taken from individuals at an early disease stage of a suspected neurogenerative disease. In some embodiments, the therapeutic agent is administered according to a body weight of the subject at a dosage of between about 0.1-100 mg/kg of the body weight. In some embodiments, the therapeutic agent is administered to the subject at a dosage of between about 200-1500 mg by intravenous administration. In some embodiments, the therapeutic agent is administered to the subject at a dosage of between about 700-1400 mg by intravenous administration. In some embodiments, the therapeutic agent is administered to the subject according to a treatment regimen comprising administering the therapeutic agent about once every 4 weeks (q4 week). In some embodiments, the first three doses administered to the subject comprise a dosage of about 700 mg of the therapeutic agent. In some embodiments, the doses administered to the subject after the first three doses administered comprise a dosage of about 1400 mg of the therapeutic agent. In some embodiments, systemic administration of the therapeutic agent. In some embodiments, systemic administration comprises i.v. administration of the therapeutic agent. In some embodiments, systemic administration comprises subcutaneous administration of the therapeutic agent. In some embodiments, systemic administration comprises oral administration of the therapeutic agent. In some embodiments, the anti-tau antibody comprises a HC comprising the sequence of SEQ ID NO: 55 and a LC comprising the sequence of SEQ ID NO: 56. In some embodiments, the anti-tau antibody comprises a HC comprising the sequence of SEQ ID NO: 57 and a LC comprising the sequence of SEQ ID NO: 58. In some embodiments, the anti-tau antibody comprises a HC comprising the sequence of SEQ ID NO: 57 and a LC comprising the sequence of SEQ ID NO: 59. In some embodiments, the anti-tau antibody comprises a HC comprising the sequence of SEQ ID NO: 60 and a LC comprising the sequence of SEQ ID NO: 61. In some embodiments, the anti-tau antibody comprises a HC comprising the sequence of SEQ ID NO: 62 and a LC comprising the sequence of SEQ ID NO: 63. In some embodiments, the anti-tau antibody comprises a HC comprising the sequence of SEQ ID NO: 64 and a LC comprising the sequence of SEQ ID NO: 65.

Described herein, in certain aspects, are methods of treating a neurodegenerative disease in a subject, the method comprising: (a) receiving data obtained by detecting a level of pT217 tau in a sample from the subject and correlating the level of pT217 tau in the sample with a standard value of pT217 tau level derived from a plurality of reference samples, wherein the detecting comprises performing an immunoassay on the sample using an anti-tau antibody or antigen-binding fragment thereof comprising i) a heavy chain comprising a variable heavy chain (VH) domain and ii) a light chain comprising a variable light chain (VL) domain, wherein the VH domain comprises HCDR1 sequence selected from SEQ ID NOs: 1-9, HCDR2 sequence selected from SEQ ID NOs: 10-17, and HCDR3 sequence selected from SEQ ID NOs: 18-23, and the VL domain comprises LCDR1 sequence selected from SEQ ID NOs: 24-31, LCDR2 sequence selected from SEQ ID NOs: 32-36, and LCDR3 sequence selected from SEQ ID NOs: 37-43; (b) analyzing a likelihood of clinical diagnosis of the neurodegenerative disease in the subject based at least in part on the receiving; (c) providing an early prognosis of the neurodegenerative disease in the subject based at least in part on the analyzing, wherein the early prognosis predates supplying a diagnosis of a prodromal stage of the neurodegenerative disease in the subject; and (d) initiating a treatment comprising administration of a formulation comprising a therapeutic agent, wherein deciding the initiating is based at least in part on the providing, wherein the therapeutic agent is selected from an NMDA receptor antagonist listed in Table 10. In some embodiments, the VH domain comprises at least 80%, at least 85%, at least 90%, at least 95% sequence identity to a sequence selected from SEQ ID NOs: 44-48. In some embodiments, the VL domain comprises at least 80%, at least 85%, at least 90%, at least 95% sequence identity to a sequence selected from SEQ ID NOs: 49-54. In some embodiments, the anti-tau antibody or antigen-binding fragment thereof comprises an IgG-scFv, nanobody, BiTE, diabody, DART, TandAb, scDiabody, scDiabody-CH3, triple body, mini-antibody, minibody, TriBi minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, tetravalent HCAb, scDiabody-Fc, diabody-Fc, tandem scFv-Fc, or intrabody. In some embodiments, the anti-tau antibody has an isotype selected from the group consisting of IgA1, IgA2, IgD, IgE, IgG1, IgG2, IgG3, IgG4, and IgM. In some embodiments, the anti-tau antibody or antigen-binding fragment thereof specifically binds an epitope in an N-terminal region of a tau polypeptide. In some embodiments, the anti-tau antibody or antigen-binding fragment thereof specifically binds to pT217-tau protein. In some embodiments, the anti-tau antibody or antigen-binding fragment thereof specifically binds to a cis-conformation of pT217-tau protein. In some embodiments, the anti-tau antibody or antigen-binding fragment thereof binds specifically to a tau polypeptide comprising phosphorylation at a site selected from a group consisting of pT181-tau, pT212-tau, phosphorylated-serine (pS)214-tau, pT217-tau, pT220-tau, and pT231-tau. In some embodiments, the anti-tau antibody or antigen-binding fragment thereof binds specifically to a tau polypeptide comprising phosphorylation at site pT217-tau (p-Tau 217-tau protein). In some embodiments, the anti-tau antibody or antigen-binding fragment thereof binds specifically to a tau polypeptide comprising phosphorylation at site pT217-tau and pT231-tau. In some embodiments, the neurodegenerative disease comprises Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, Hallevorden-Spatz disease, mild cognitive impairment (MCI), Lewy Body's disease (LBD), Amyotrophic lateral sclerosis, spinal muscular atrophy, Friedreich's ataxia, Wernicke-Korsakoff syndrome, a prion disease, vascular dementia, Alzheimer's related dementia, Huntington's disease (HD), or a transgene-induced tauopathy. In some embodiments, the sample is selected from a cerebrospinal fluid (CSF) sample, a plasma sample, a blood sample, or a serum sample. In some embodiments, the neurodegenerative disease comprises Alzheimer's disease. In some embodiments, one or more symptoms of the neurodegenerative disease in the subject is ameliorated. In some embodiments, a progression of one or more symptoms of the neurodegenerative disease in the subject is slowed. In some embodiments, the therapeutic agent is administered intravenously, intramuscularly, intrathecally, intracerebrally, subcutaneously, orally, nasally, topically, buccally, or sublingually. In some embodiments, the therapeutic agent is administered directly to the CNS of the subject via intravenous delivery, intravascular delivery, intrathecal delivery, intracisternal delivery, intraspinal delivery, subpial delivery, or intracerebroventricular delivery. In some embodiments, the subject is at a preclinical stage of the neurogenerative disease. In some embodiments, the subject is at a prodromal stage of the neurogenerative disease. In some embodiments, the plurality of reference samples are taken from healthy individuals not suspected of having a neurogenerative disease. In some embodiments, the plurality of samples from the reference subjects are taken from individuals at an early disease stage of a suspected neurogenerative disease. In some embodiments, the therapeutic agent is administered according to a body weight of the subject at a dosage of between about 0.1-100 mg/kg of the body weight. In some embodiments, the therapeutic agent is administered to the subject at a dosage of between about 200-1500 mg by intravenous administration. In some embodiments, the therapeutic agent is administered to the subject at a dosage of between about 700-1400 mg by intravenous administration. In some embodiments, the therapeutic agent is administered to the subject according to a treatment regimen comprising administering the therapeutic agent about once every 4 weeks (q4 week). In some embodiments, the first three doses administered to the subject comprise a dosage of about 700 mg of the therapeutic agent. In some embodiments, the doses administered to the subject after the first three doses administered comprise a dosage of about 1400 mg of the therapeutic agent. In some embodiments, systemic administration of the therapeutic agent. In some embodiments, systemic administration comprises i.v. administration of the therapeutic agent. In some embodiments, systemic administration comprises subcutaneous administration of the therapeutic agent. In some embodiments, systemic administration comprises oral administration of the therapeutic agent. In some embodiments, the anti-tau antibody comprises a HC comprising the sequence of SEQ ID NO: 55 and a LC comprising the sequence of SEQ ID NO: 56. In some embodiments, the anti-tau antibody comprises a HC comprising the sequence of SEQ ID NO: 57 and a LC comprising the sequence of SEQ ID NO: 58. In some embodiments, the anti-tau antibody comprises a HC comprising the sequence of SEQ ID NO: 57 and a LC comprising the sequence of SEQ ID NO: 59. In some embodiments, the anti-tau antibody comprises a HC comprising the sequence of SEQ ID NO: 60 and a LC comprising the sequence of SEQ ID NO: 61. In some embodiments, the anti-tau antibody comprises a HC comprising the sequence of SEQ ID NO: 62 and a LC comprising the sequence of SEQ ID NO: 63. In some embodiments, the anti-tau antibody comprises a HC comprising the sequence of SEQ ID NO: 64 and a LC comprising the sequence of SEQ ID NO: 65.

Described herein, in certain aspects, are methods of treating a neurodegenerative disease in a subject, the method comprising: (a) receiving data obtained by detecting a level of pT217 tau in a sample from the subject and correlating the level of pT217 tau in the sample with a standard value of pT217 tau level derived from a plurality of reference samples, wherein the detecting comprises performing an immunoassay on the sample using an anti-tau antibody or antigen-binding fragment thereof comprising i) a heavy chain comprising a variable heavy chain (VH) domain and ii) a light chain comprising a variable light chain (VL) domain, wherein the VH domain comprises HCDR1 sequence selected from SEQ ID NOs: 1-9, HCDR2 sequence selected from SEQ ID NOs: 10-17, and HCDR3 sequence selected from SEQ ID NOs: 18-23, and the VL domain comprises LCDR1 sequence selected from SEQ ID NOs: 24-31, LCDR2 sequence selected from SEQ ID NOs: 32-36, and LCDR3 sequence selected from SEQ ID NOs: 37-43; (b) analyzing a likelihood of clinical diagnosis of the neurodegenerative disease in the subject based at least in part on the receiving; (c) providing an early prognosis of the neurodegenerative disease in the subject based at least in part on the analyzing, wherein the early prognosis predates supplying a diagnosis of a prodromal stage of the neurodegenerative disease in the subject; and (d) initiating a treatment comprising administration of a formulation comprising a therapeutic agent, wherein deciding the initiating is based at least in part on the providing, wherein the therapeutic agent is selected from an acetylcholinesterase and butyrylcholinesterase inhibitor listed in Table 10. In some embodiments, the VH domain comprises at least 80%, at least 85%, at least 90%, at least 95% sequence identity to a sequence selected from SEQ ID NOs: 44-48. In some embodiments, the VL domain comprises at least 80%, at least 85%, at least 90%, at least 95% sequence identity to a sequence selected from SEQ ID NOs: 49-54. In some embodiments, the anti-tau antibody or antigen-binding fragment thereof comprises an IgG-scFv, nanobody, BiTE, diabody, DART, TandAb, scDiabody, scDiabody-CH3, triple body, mini-antibody, minibody, TriBi minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fe, tetravalent HCAb, scDiabody-Fe, diabody-Fe, tandem scFv-Fc, or intrabody. In some embodiments, the anti-tau antibody has an isotype selected from the group consisting of IgA1, IgA2, IgD, IgE, IgG1, IgG2, IgG3, IgG4, and IgM. In some embodiments, the anti-tau antibody or antigen-binding fragment thereof specifically binds an epitope in an N-terminal region of a tau polypeptide. In some embodiments, the anti-tau antibody or antigen-binding fragment thereof specifically binds to pT217-tau protein. In some embodiments, the anti-tau antibody or antigen-binding fragment thereof specifically binds to a cis-conformation of pT217-tau protein. In some embodiments, the anti-tau antibody or antigen-binding fragment thereof binds specifically to a tau polypeptide comprising phosphorylation at a site selected from a group consisting of pT181-tau, pT212-tau, phosphorylated-serine (pS)214-tau, pT217-tau, pT220-tau, and pT231-tau. In some embodiments, the anti-tau antibody or antigen-binding fragment thereof binds specifically to a tau polypeptide comprising phosphorylation at site pT217-tau. In some embodiments, the anti-tau antibody or antigen-binding fragment thereof binds specifically to a tau polypeptide comprising phosphorylation at site pT217-tau and pT231-tau. In some embodiments, the neurodegenerative disease comprises Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, Hallevorden-Spatz disease, mild cognitive impairment (MCI), Lewy Body's disease (LBD), Amyotrophic lateral sclerosis, spinal muscular atrophy, Friedreich's ataxia, Wernicke-Korsakoff syndrome, a prion disease, vascular dementia, Alzheimer's related dementia, Huntington's disease (HD), or a transgene-induced tauopathy. In some embodiments, the sample is selected from a cerebrospinal fluid (CSF) sample, a plasma sample, a blood sample, or a serum sample. In some embodiments, the neurodegenerative disease comprises Alzheimer's disease. In some embodiments, one or more symptoms of the neurodegenerative disease in the subject is ameliorated. In some embodiments, a progression of one or more symptoms of the neurodegenerative disease in the subject is slowed. In some embodiments, the therapeutic agent is administered intravenously, intramuscularly, intrathecally, intracerebrally, subcutaneously, orally, nasally, topically, buccally, or sublingually. In some embodiments, the therapeutic agent is administered directly to the CNS of the subject via intravenous delivery, intravascular delivery, intrathecal delivery, intracisternal delivery, intraspinal delivery, subpial delivery, or intracerebroventricular delivery. In some embodiments, the subject is at a preclinical stage of the neurogenerative disease. In some embodiments, the subject is at a prodromal stage of the neurogenerative disease. In some embodiments, the plurality of reference samples are taken from healthy individuals not suspected of having a neurogenerative disease. In some embodiments, the plurality of samples from the reference subjects are taken from individuals at an early disease stage of a suspected neurogenerative disease. In some embodiments, the therapeutic agent is administered according to a body weight of the subject at a dosage of between about 0.1-100 mg/kg of the body weight. In some embodiments, the therapeutic agent is administered to the subject at a dosage of between about 200-1500 mg by intravenous administration. In some embodiments, the therapeutic agent is administered to the subject at a dosage of between about 700-1400 mg by intravenous administration. In some embodiments, the therapeutic agent is administered to the subject according to a treatment regimen comprising administering the therapeutic agent about once every 4 weeks (q4 week). In some embodiments, the first three doses administered to the subject comprise a dosage of about 700 mg of the therapeutic agent. In some embodiments, the doses administered to the subject after the first three doses administered comprise a dosage of about 1400 mg of the therapeutic agent. In some embodiments, systemic administration of the therapeutic agent. In some embodiments, systemic administration comprises i.v. administration of the therapeutic agent. In some embodiments, systemic administration comprises subcutaneous administration of the therapeutic agent. In some embodiments, systemic administration comprises oral administration of the therapeutic agent. In some embodiments, the anti-tau antibody comprises a HC comprising the sequence of SEQ ID NO: 55 and a LC comprising the sequence of SEQ ID NO: 56. In some embodiments, the anti-tau antibody comprises a HC comprising the sequence of SEQ ID NO: 57 and a LC comprising the sequence of SEQ ID NO: 58. In some embodiments, the anti-tau antibody comprises a HC comprising the sequence of SEQ ID NO: 57 and a LC comprising the sequence of SEQ ID NO: 59. In some embodiments, the anti-tau antibody comprises a HC comprising the sequence of SEQ ID NO: 60 and a LC comprising the sequence of SEQ ID NO: 61. In some embodiments, the anti-tau antibody comprises a HC comprising the sequence of SEQ ID NO: 62 and a LC comprising the sequence of SEQ ID NO: 63. In some embodiments, the anti-tau antibody comprises a HC comprising the sequence of SEQ ID NO: 64 and a LC comprising the sequence of SEQ ID NO: 65.

Described herein, in certain aspects, are methods of treating a neurodegenerative disease in a subject, the method comprising: (a) receiving data obtained by detecting a level of pT217 tau in a sample from the subject and correlating the level of pT217 tau in the sample with a standard value of pT217 tau level derived from a plurality of reference samples, wherein the detecting comprises performing an immunoassay on the sample using an anti-tau antibody or antigen-binding fragment thereof comprising i) a heavy chain comprising a variable heavy chain (VH) domain and ii) a light chain comprising a variable light chain (VL) domain, wherein the VH domain comprises HCDR1 sequence selected from SEQ ID NOs: 1-9, HCDR2 sequence selected from SEQ ID NOs: 10-17, and HCDR3 sequence selected from SEQ ID NOs: 18-23, and the VL domain comprises LCDR1 sequence selected from SEQ ID NOs: 24-31, LCDR2 sequence selected from SEQ ID NOs: 32-36, and LCDR3 sequence selected from SEQ ID NOs: 37-43; (b) analyzing a likelihood of clinical diagnosis of the neurodegenerative disease in the subject based at least in part on the receiving; (c) providing an early prognosis of the neurodegenerative disease in the subject based at least in part on the analyzing, wherein the early prognosis predates supplying a diagnosis of a prodromal stage of the neurodegenerative disease in the subject; and (d) initiating a treatment comprising administration of a formulation comprising two or more therapeutic agents, wherein deciding the initiating is based at least in part on the providing, wherein the two or more therapeutic agents selected from NMDA receptor antagonists and cholinesterase inhibitors listed in Table 10. In some embodiments, the VH domain comprises at least 80%, at least 85%, at least 90%, at least 95% sequence identity to a sequence selected from SEQ ID NOs: 44-48. In some embodiments, the VL domain comprises at least 80%, at least 85%, at least 90%, at least 95% sequence identity to a sequence selected from SEQ ID NOs: 49-54. In some embodiments, the anti-tau antibody or antigen-binding fragment thereof comprises an IgG-scFv, nanobody, BiTE, diabody, DART, TandAb, scDiabody, scDiabody-CH3, triple body, mini-antibody, minibody, TriBi minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, tetravalent HCAb, scDiabody-Fc, diabody-Fc, tandem scFv-Fc, or intrabody. In some embodiments, the anti-tau antibody has an isotype selected from the group consisting of IgA1, IgA2, IgD, IgE, IgG1, IgG2, IgG3, IgG4, and IgM. In some embodiments, the anti-tau antibody or antigen-binding fragment thereof specifically binds an epitope in an N-terminal region of a tau polypeptide. In some embodiments, the anti-tau antibody or antigen-binding fragment thereof specifically binds to pT217-tau protein. In some embodiments, the anti-tau antibody or antigen-binding fragment thereof specifically binds to a cis-conformation of pT217-tau protein. In some embodiments, the anti-tau antibody or antigen-binding fragment thereof binds specifically to a tau polypeptide comprising phosphorylation at a site selected from a group consisting of pT181-tau, pT212-tau, phosphorylated-serine (pS)214-tau, pT217-tau, pT220-tau, and pT231-tau. In some embodiments, the anti-tau antibody or antigen-binding fragment thereof binds specifically to a tau polypeptide comprising phosphorylation at site pT217-tau. In some embodiments, the anti-tau antibody or antigen-binding fragment thereof binds specifically to a tau polypeptide comprising phosphorylation at site pT217-tau and pT231-tau. In some embodiments, the neurodegenerative disease comprises Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, Hallevorden-Spatz disease, mild cognitive impairment (MCI), Lewy Body's disease (LBD), Amyotrophic lateral sclerosis, spinal muscular atrophy, Friedreich's ataxia, Wernicke-Korsakoff syndrome, a prion disease, vascular dementia, Alzheimer's related dementia, Huntington's disease (HD), or a transgene-induced tauopathy. In some embodiments, the sample is selected from a cerebrospinal fluid (CSF) sample, a plasma sample, a blood sample, or a serum sample. In some embodiments, the neurodegenerative disease comprises Alzheimer's disease. In some embodiments, one or more symptoms of the neurodegenerative disease in the subject is ameliorated. In some embodiments, a progression of one or more symptoms of the neurodegenerative disease in the subject is slowed. In some embodiments, the therapeutic agent is administered intravenously, intramuscularly, intrathecally, intracerebrally, subcutaneously, orally, nasally, topically, buccally, or sublingually. In some embodiments, the therapeutic agent is administered directly to the CNS of the subject via intravenous delivery, intravascular delivery, intrathecal delivery, intracisternal delivery, intraspinal delivery, subpial delivery, or intracerebroventricular delivery. In some embodiments, the subject is at a preclinical stage of the neurogenerative disease. In some embodiments, the subject is at a prodromal stage of the neurogenerative disease. In some embodiments, the plurality of reference samples are taken from healthy individuals not suspected of having a neurogenerative disease. In some embodiments, the plurality of samples from the reference subjects are taken from individuals at an early disease stage of a suspected neurogenerative disease. In some embodiments, the therapeutic agent is administered according to a body weight of the subject at a dosage of between about 0.1-100 mg/kg of the body weight. In some embodiments, the therapeutic agent is administered to the subject at a dosage of between about 200-1500 mg by intravenous administration. In some embodiments, the therapeutic agent is administered to the subject at a dosage of between about 700-1400 mg by intravenous administration. In some embodiments, the therapeutic agent is administered to the subject according to a treatment regimen comprising administering the therapeutic agent about once every 4 weeks (q4 week). In some embodiments, the first three doses administered to the subject comprise a dosage of about 700 mg of the therapeutic agent. In some embodiments, the doses administered to the subject after the first three doses administered comprise a dosage of about 1400 mg of the therapeutic agent. In some embodiments, systemic administration of the therapeutic agent. In some embodiments, systemic administration comprises i.v. administration of the therapeutic agent. In some embodiments, systemic administration comprises subcutaneous administration of the therapeutic agent. In some embodiments, systemic administration comprises oral administration of the therapeutic agent. In some embodiments, the anti-tau antibody comprises a HC comprising the sequence of SEQ ID NO: 55 and a LC comprising the sequence of SEQ ID NO: 56. In some embodiments, the anti-tau antibody comprises a HC comprising the sequence of SEQ ID NO: 57 and a LC comprising the sequence of SEQ ID NO: 58. In some embodiments, the anti-tau antibody comprises a HC comprising the sequence of SEQ ID NO: 57 and a LC comprising the sequence of SEQ ID NO: 59. In some embodiments, the anti-tau antibody comprises a HC comprising the sequence of SEQ ID NO: 60 and a LC comprising the sequence of SEQ ID NO: 61. In some embodiments, the anti-tau antibody comprises a HC comprising the sequence of SEQ ID NO: 62 and a LC comprising the sequence of SEQ ID NO: 63. In some embodiments, the anti-tau antibody comprises a HC comprising the sequence of SEQ ID NO: 64 and a LC comprising the sequence of SEQ ID NO: 65.

Described herein, in certain embodiments of the methods, the detection of the level of pT217 tau in the sample from the subject comprises performing an immunoassay on the sample using an anti-tau antibody or antigen-binding fragment thereof comprising i) a heavy chain comprising a variable heavy chain (VH) domain and ii) a light chain comprising a variable light chain (VL) domain, wherein the VH domain comprises HCDR1 sequence selected from SEQ ID NOs: 1-9, HCDR2 sequence selected from SEQ ID NOs: 10-17, and HCDR3 sequence selected from SEQ ID NOs: 18-23, and the VL domain comprises LCDR1 sequence selected from SEQ ID NOs: 24-31, LCDR2 sequence selected from SEQ ID NOs: 32-36, and LCDR3 sequence selected from SEQ ID NOs: 37-43. In some embodiments, the HCDR1 sequence comprises SEQ ID NO: 2, HCDR2 sequence comprises SEQ ID NO: 11, HCDR3 sequence comprises SEQ ID NO: 19, LCDR1 sequence comprises SEQ ID NO: 25, LCDR2 sequence comprises SEQ ID NO: 33, and LCDR3 sequence comprises SEQ ID NO: 38. In some embodiments, the HCDR1 sequence comprises SEQ ID NO: 1, HCDR2 sequence comprises SEQ ID NO: 10, HCDR3 sequence comprises SEQ ID NO: 18, LCDR1 sequence comprises SEQ ID NO: 24, LCDR2 sequence comprises SEQ ID NO: 32, and LCDR3 sequence comprises SEQ ID NO: 37. In some embodiments, the HCDR1 sequence comprises SEQ ID NO: 2, HCDR2 sequence comprises SEQ ID NO: 11, HCDR3 sequence comprises SEQ ID NO: 19, LCDR1 sequence comprises SEQ ID NO: 26, LCDR2 sequence comprises SEQ ID NO: 34, and LCDR3 sequence comprises SEQ ID NO: 39. In some embodiments, the SEQ ID NO: 3, HCDR2 sequence comprises SEQ ID NO: 12, HCDR3 sequence comprises SEQ ID NO: 18, LCDR1 sequence comprises SEQ ID NO: 27, LCDR2 sequence comprises SEQ ID NO: 32, and LCDR3 sequence comprises SEQ ID NO: 40. In some embodiments, the HCDR1 sequence comprises SEQ ID NO: 4, HCDR2 sequence comprises SEQ ID NO: 11, HCDR3 sequence comprises SEQ ID NO: 20, LCDR1 sequence comprises SEQ ID NO: 28, LCDR2 sequence comprises SEQ ID NO: 35, and LCDR3 sequence comprises SEQ ID NO: 41. In some embodiments, the SEQ ID NO: 5, HCDR2 sequence comprises SEQ ID NO: 13, HCDR3 sequence comprises SEQ ID NO: 21, LCDR1 sequence comprises SEQ ID NO: 29, LCDR2 sequence comprises SEQ ID NO: 33, and LCDR3 sequence comprises SEQ ID NO: 42. In some embodiments, the VH domain comprises at least 80%, at least 85%, at least 90%, at least 95% sequence identity to a sequence selected from SEQ ID NOs: 44-48. In some embodiments, the VL domain comprises at least 80%, at least 85%, at least 90%, at least 95% sequence identity to a sequence selected from SEQ ID NOs: 49-54. In some embodiments, the anti-tau antibody or antigen-binding fragment thereof comprises an IgG-scFv, nanobody, BiTE, diabody, DART, TandAb, scDiabody, scDiabody-CH3, triple body, mini-antibody, minibody, TriBi minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, tetravalent HCAb, scDiabody-Fc, diabody-Fc, tandem scFv-Fc, or intrabody. In some embodiments, the anti-tau antibody has an isotype selected from the group consisting of IgA1, IgA2, IgD, IgE, IgG1, IgG2, IgG3, IgG4, and IgM. In some embodiments, the anti-tau antibody or antigen-binding fragment thereof specifically binds an epitope in an N-terminal region of a tau polypeptide. In some embodiments, the anti-tau antibody or antigen-binding fragment thereof specifically binds to pT217-tau protein. In some embodiments, the anti-tau antibody or antigen-binding fragment thereof specifically binds to a cis-conformation of pT217-tau protein. In some embodiments, the anti-tau antibody or antigen-binding fragment thereof binds specifically to a tau polypeptide comprising phosphorylation at a site selected from a group consisting of pT181-tau, pT212-tau, phosphorylated-serine (pS)214-tau, pT217-tau, pT220-tau, and pT231-tau. In some embodiments, the anti-tau antibody or antigen-binding fragment thereof binds specifically to a tau polypeptide comprising phosphorylation at site pT217-tau. In some embodiments, the anti-tau antibody or antigen-binding fragment thereof binds specifically to a tau polypeptide comprising phosphorylation at site pT217-tau and pT231-tau. In some embodiments, the neurodegenerative disease comprises Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, Hallevorden-Spatz disease, mild cognitive impairment (MCI), Lewy Body's disease (LBD), Amyotrophic lateral sclerosis, spinal muscular atrophy, Friedreich's ataxia, Wemicke-Korsakoff syndrome, a prion disease, vascular dementia, Alzheimer's related dementia, Huntington's disease (HD), or a transgene-induced tauopathy. In some embodiments, the sample is selected from a cerebrospinal fluid (CSF) sample, a plasma sample, a blood sample, or a serum sample. In some embodiments, the neurodegenerative disease comprises Alzheimer's disease. In some embodiments, one or more symptoms of the neurodegenerative disease in the subject is ameliorated. In some embodiments, a progression of one or more symptoms of the neurodegenerative disease in the subject is slowed. In some embodiments, the therapeutic agent is administered intravenously, intramuscularly, intrathecally, intracerebrally, subcutaneously, orally, nasally, topically, buccally, or sublingually. In some embodiments, the therapeutic agent is administered directly to the CNS of the subject via intravenous delivery, intravascular delivery, intrathecal delivery, intracisternal delivery, intraspinal delivery, subpial delivery, or intracerebroventricular delivery. In some embodiments, the subject is at a preclinical stage of the neurogenerative disease. In some embodiments, the subject is at a prodromal stage of the neurogenerative disease. In some embodiments, the plurality of reference samples are taken from healthy individuals not suspected of having a neurogenerative disease. In some embodiments, the plurality of samples from the reference subjects are taken from individuals at an early disease stage of a suspected neurogenerative disease. In some embodiments, the therapeutic agent is administered according to a body weight of the subject at a dosage of between about 0.1-100 mg/kg of the body weight. In some embodiments, the therapeutic agent is administered to the subject at a dosage of between about 200-1500 mg by intravenous administration. In some embodiments, the therapeutic agent is administered to the subject at a dosage of between about 700-1400 mg by intravenous administration. In some embodiments, the therapeutic agent is administered to the subject according to a treatment regimen comprising administering the therapeutic agent about once every 4 weeks (q4 week). In some embodiments, the first three doses administered to the subject comprise a dosage of about 700 mg of the therapeutic agent. In some embodiments, the doses administered to the subject after the first three doses administered comprise a dosage of about 1400 mg of the therapeutic agent. In some embodiments, systemic administration of the therapeutic agent. In some embodiments, systemic administration comprises i.v. administration of the therapeutic agent. In some embodiments, systemic administration comprises subcutaneous administration of the therapeutic agent. In some embodiments, systemic administration comprises oral administration of the therapeutic agent. In some embodiments, the anti-tau antibody comprises a HC comprising the sequence of SEQ ID NO: 55 and a LC comprising the sequence of SEQ ID NO: 56. In some embodiments, the anti-tau antibody comprises a HC comprising the sequence of SEQ ID NO: 57 and a LC comprising the sequence of SEQ ID NO: 58. In some embodiments, the anti-tau antibody comprises a HC comprising the sequence of SEQ ID NO: 57 and a LC comprising the sequence of SEQ ID NO: 59. In some embodiments, the anti-tau antibody comprises a HC comprising the sequence of SEQ ID NO: 60 and a LC comprising the sequence of SEQ ID NO: 61. In some embodiments, the anti-tau antibody comprises a HC comprising the sequence of SEQ ID NO: 62 and a LC comprising the sequence of SEQ ID NO: 63. In some embodiments, the anti-tau antibody comprises a HC comprising the sequence of SEQ ID NO: 64 and a LC comprising the sequence of SEQ ID NO: 65.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative aspects, in which the principles of the disclosure are utilized, and the accompanying drawings below.

FIG. 1 depicts a schema of the single molecule array (Simoa®) method used herein for assaying tau antibodies described herein. After substrate is added to sample (sandwich ELISA on bead, 1.1), sample is added to Simoa® Disk (1.2). Beads are given time to settle into microarray wells on disk (one bead per well) (1.3). Then, sealing oil is used to remove excess beads to allow for imaging (1.4). Beads that have sandwich complex (positive beads) will fluoresce with the substrate and show up during imaging; beads without sandwich complex (negative) will still show up in imaging but will not fluoresce (1.5). The percentage of positive beads is converted to an AEB (average enzymes per bead) value.

FIGS. 2A-2D depict data for Antibody 1, Antibody 2, Antibody 3, Antibody 4, Antibody 5, and Antibody 6 in the Simoa® assay. FIG. 2A depicts data from an 2-step protocol assay in which all capture antibodies were tested against Tau-12 detector. FIG. 2B depicts data from an 2-step protocol assay in which all capture antibodies were tested against HT7-BT2 detectors (detects Tau in the mid-domain region). FIG. 2C depicts data from an 3-step protocol assay in which all capture antibodies were tested against Tau-12 detector. FIG. 2D depicts data from an 3-step protocol assay in which all capture antibodies were tested against HT7-BT2 detectors.

FIG. 3 depict ELISA data of the different monoclonal antibodies to varying peptide concentrations.

FIG. 4 depicts a diagram of an indirect ELISA assay and graphs of ELISA data assaying antibody binding to pTau-217 peptides.

FIG. 5 depicts a graph of signal/noise (S/N) analysis of ELISA assay for Antibody 2 binding to a pTau-217 peptide for 120 clinic samples derived from plasma and a graph of a coefficient of variation (CV %) for ELISA assay for Antibody 2 binding to a pTau-217 peptide for 120 clinic samples derived from plasma.

FIG. 6 depicts graphs of calibration curves (Cal curves) for a Simoa®-based pTau-217 assay using Antibody 2 on groups (plates) designated QTx of clinical samples derived from cerebrospinal fluid (68 CSF samples) and plasma (120 plasma samples) compared to the assay using ADx p204 antibody.

FIG. 7 depicts a graph of Simoa®-based pTau assay-217 results using Antibody 2 in matched samples from the sample individual derived from either plasma (Y-axis) or CSF (X-axis) and statistical analysis of correlated results in individual with a clinical diagnosis of either non-Alzheimer's disease, an uncertain diagnosis, or Alzheimer's disease.

FIG. 8 depicts a graph of Simoa®-based pTau assay-217 results using Antibody 2 per sample vs Simoa®-based pTau assay-181 results using Antibody 2 per sample and statistical analysis of correlated results.

FIG. 9 depicts a graph of Simoa®-based pTau assay-217 results using Antibody 2 per sample vs Simoa®-based Tau assay results using Innotest pTau 181 antibody per sample and statistical analysis of correlated results.

FIG. 10 depicts a graph of Simoa®-based pTau assay results using Antibody 2 as a capture antibody, antibody ADx p204 as a detector antibody and a peptide as calibrator and statistical analysis of correlated results.

FIG. 11 depicts a graph of Simoa®-based pTau assay-217 results using Antibody 2 grouping together samples from individuals with a clinical diagnosis of Alzheimer's disease and samples from control individuals derived from either CSF or plasma.

FIG. 12 depicts a graph of Simoa®-based pTau assay-217 results using Antibody 2 on various concentrations of EDTA plasma samples and a chart of listing coefficient of variation for each sample concentration to illustrate the precision of the assay.

FIG. 13 depicts graphs of Simoa®-based pTau assay-217 results using Antibody 2 graphed as coefficient of variation (CV %) vs measured concentration.

FIG. 14 depicts a graph of Simoa®-based pTau assay-217 results using Antibody 2 and statistical analysis of parallelism with determines whether actual samples containing high endogenous analyte concentrations provide a similar degree of detection in a standard curve after dilutions.

FIG. 15 depicts a graph of Simoa®-based pTau assay-217 results using Antibody 2 and statistical analysis of linearity with determines whether sample matrices spiked with detection analyte above an upper limit of detect can still provide reliable quantification after dilution within standard curve ranges for four samples plus a buffer spike.

FIG. 16 depicts a graph of Simoa®-based pTau assay-217 results using Antibody 2 and statistical analysis of linearity with determines whether sample matrices spiked with detection analyte above an upper limit of detect can still provide reliable quantification after dilution within standard curve ranges for three samples plus a calibration sample.

FIG. 17 depicts a graph of Simoa®-based pTau assay-217 results using Antibody 2 in a clinical validation of a memory clinic cohort and a graph of receiver-operating characteristic (ROC) analysis graphed against pTau-217 assay sensitivity.

FIG. 18 depicts a graph of Simoa®-based pTau assay-217 results using Antibody 2 on groups from Control and AD dementia individuals.

FIG. 19 depicts a graph of Simoa®-based pTau assay-181 results using an antibody from Quanterix® on groups from Control and AD dementia individuals and a chart of sample stratification.

FIG. 20 depicts graphs of Simoa®-based pTau assay-217 results using Antibody 2, and Simoa®-based pTau assay-181 results using an antibody from Quanterix® showing precision plots with calculated coefficient of variation.

FIG. 21 depicts a graph of clinical performance of various pTau Simoa®-based assays comparing sensitivity and specificity and a chart with a statistical analysis of results.

FIG. 22 depicts a schematic diagram of Tau indicating the relative location of various protein domains and the locations of threonine residues which can be assayed for phosphorylation status using methods disclosed herein.

FIG. 23 depicts graphs of reactivity to a Tau fragment with non-phosphorylated T217 (Bio-pt654) and full length Tau (Tau441) in indirect ELISA for various antibodies.

FIG. 24 depicts graphs of reactivity to Tau fragments with phosphorylated T181 (Bio-pt126) and phosphorylated T231 (Bio-pt146) in indirect ELISA for various antibodies.

FIG. 25 depicts a diagram of an assay utilizing a p-Tau 217 monoclonal antibody as a capture tool for various synthetic peptides and a graph of results for this assay using Antibody 2 as the capture tool.

FIG. 26 depicts Western blot analysis using various Tau antibodies on brain lysate samples from AD patients or control subjects.

### DETAILED DESCRIPTION

Neurodegenerative diseases, such as Alzheimer's disease, are complex diseases and effective treatment requires an accurate diagnosis and treatment selection. Described herein are improved compositions and methods for identifying a subject as having or being a risk for developing a neurodegenerative disease, such as Alzheimer's disease, that may be characterized by accumulation in the brain of amyloid-0 peptide or deposition of abnormal phosphorylated tau, or both. Also provided are compositions and methods for selecting a subject for treatment with, and/or treating, the neurodegenerative disease based, at least in part, on the results of a test measuring abnormal phosphorylated tau levels in a sample (e.g., blood, cerebral spinal fluid) obtained from the subject. As will be explained further below, the inventors of the present disclosure discovered that abnormal phosphorylated tau is a useful biomarker for diagnosis and disease progression in tauopathies, such as Alzheimer's disease, and developed antibody reagents to measure abnormal phosphorylated tau levels (e.g., p-Tau 217) in a sample (e.g., plasma or CSF sample) of a subject, as provided in International Application No. PCT/US2022/042963, which is hereby incorporated by reference in its entirety.

In a secondary analysis of early symptomatic Alzheimer's disease patients in the TRAILBLAZER-ALZ randomized clinical trial (NCT03367403) receiving Donanemab, the authors found that plasma biomarkers (including p-Tau 217) were significantly lower following Donanemab treatment compared with placebo as early as 12 weeks after the start of treatment and persisting through 76 weeks of the study. Pontecorvo M J, et al. Association of Donanemab Treatment With Exploratory Plasma Biomarkers in Early Symptomatic Alzheimer Disease: A Secondary Analysis of the TRAILBLAZER-ALZ Randomized Clinical Trial. JAMA Neurol. 2022 Dec. 1; 79(12):1250-1259. In this study, the authors report that plasma p-Tau 217 levels significantly correlate with percentage change in amyloid plaque level as measured by amyloid PET imaging, thus supporting that inhibitors of amyloid-O peptide (e.g., Donanemab, Lecanemab, Remternetug, Aducanumab, etc.) may be therapeutically effective to treat early symptomatic Alzheimer's disease.

Building on the discovery of that abnormal phosphorylated tau levels (e.g., p-Tau 217) may be used as a biomarker of diagnosis and disease progression in tauopathies and findings by Pontecorvo M J, et al. that p-Tau 217 may be used as a biomarker of positive early symptomatic Alzheimer's disease patient outcomes in patients treated with Donanemab, the present disclosure provides compositions and methods for selecting a subject for treatment with a therapeutic agent (e.g., provided in Table 10 and elsewhere herein) for treatment of the neurodegenerative disease (e.g., Donanemab, Lecanemab, Remternetug, Aducanumab, etc.) using antibody reagents that are specific to phosphorylated tau protein (e.g., p-Tau 217).

Tau is an important microtubule-associated protein, abundantly expressed in CNS neurons, and serves critical roles in normal cellular physiology. Tau has also been found to be dysregulated in Alzheimer's disease and other tauopathies. Six isoforms of tau protein are generated from the TAU gene by alternative splicing. The isoforms differ from each other by the presence or absence of two N-terminal inserts and a repeat termed R2. All six protein isoforms of tau are highly soluble under normal and healthy cellular conditions and are typically regulated by phosphorylation and dephosphorylation. Tau has been demonstrated to interact with microtubules and promote microtubule assembly. In neurons, tau promotes the formation of axonal microtubules and stabilizes them. Tau has additional roles in driving neurite outgrowth. Impaired interaction of tau with microtubules may be an important component in the pathology, development, and progression of tauopathies. Hyperphosphorylation of tau is a hallmark feature of Alzheimer's disease and other tauopathies and the extent of hyperphosphorylation is often correlated with disease progression. Hyperphosphorylation of tau protein can result in the self-assembly of insoluble tangles of paired helical filaments and straight filaments of tau. These insoluble aggregates of tangles, termed neurofibrillary tangles (NFTs), are comprised of hyperphosphorylated tau and are considered to be pathological markers of tauopathies.

Phosphorylated tau (pTau), total tau, and A042 each detected from the cerebrospinal fluid (CSF) and/or the blood are individual biomarkers for Alzheimer's disease and several other related tauopathies. CSF pTau is increased in individuals later confirmed to have Alzheimer's disease both at the prodromal stages and the dementia stages compared to age- and gender-matched controls. CSF pTau levels exhibit a strong degree of correlation to the extent of cognitive impairment in individuals with Alzheimer's disease. In fact, CSF pTau levels may be used with some degree of precision as a biomarker to predict progression from cognitively unimpaired, to mild cognitive impairment (MCI) and then to Alzheimer's disease dementia. In terms of utility as a biomarker to predict even relatively early stages of Alzheimer's disease progression, CSF pTau has been shown to be significantly increased in samples from individuals with preclinical Alzheimer's disease. Changes in the extent of pTau phosphorylation have been demonstrated in both preclinical sporadic cases of Alzheimer's disease and in early stages of autosomal-dominant Alzheimer's disease. Blood levels of pTau, total tau, and A042 are generally lower than CSF levels when assayed within the same individual and may be utilized as informative biomarkers for Alzheimer's disease and other related tauopathies if blood levels of these biomarkers can be assayed with sufficient specificity and precision.

Several sites of phosphorylation contributing to hyperphosphorylated tau which aggregates into NFTs have been identified. In the longest tau isoform, 79 potential serine or threonine phosphorylation sites are present and at least 30 of these sites have been identified as phosphorylated in NFT aggregates. A common site used to assay tau molecules for phosphorylation status is at threonine-181. CSF fluid contains an array of tau fragments at various abundances. Fragments of tau from the N-terminal region and from the middle region of tau polypeptides are considerably more abundant in CSF samples than C-terminal tau fragments. Plasma samples from individuals also contain tau polypeptides and tau polypeptide fragments, however they tend to be present at lower concentrations that in matched CSF samples. Being able to detect tau phosphorylation at particular amino acid residues relevant for disease pathology and progression is a critical component of diagnosis, disease staging, and as a metric to measure treatment efficacy for Alzheimer's disease and other tauopathies. Detection and measurement of pTau levels at particular disease-relevant residues from plasma samples would aid greatly to the development of more sensitive and finely-tuned diagnosis, prognosis, and disease analysis for individuals who may be at risk for developing or are at early stages of Alzheimer's disease or other tauopathies. Phosphorylation of tau at threonine 217 (pTau 217) is one such residue of particular interest in development new biomarkers and diagnostic assays. Alterations in pTau biomarker concentration in CSF and in plasma are thought to precede measurable behavioral or cognitive changes in Alzheimer's disease and in other tauopathies. A development of new assays to enable a continuum of specific points and extents of tau phosphorylation of certain residues would undoubtedly aid in the clinically relevant medical diagnosis and treatment decisions. A comparison of results from new assays to results from existing assays can also yield further medically informative determinations. Results from plasma-based tau biomarker assays can be compared against matched CSF samples (detecting CSF pTau or CSF soluble AD) and also against positron emission tomography (PET) scans detecting an extent and locations of Aβ aggregates as metrics for their utility, especially for analysis at preclinical or early disease stages.

There remains a need for preventing, for preventing the progression of, and for effectively treating neurodegenerative disorders, including various tauopathies. There are currently limited options, also having a limited chance to success, to attempt treatment of a patient with a tauopathy using an existing disease-modifying therapy. A current challenge for treatment of tauopathies includes a lack of ability to diagnose the condition at an early time point prior to significant neurodegeneration. Another major obstacle is a lack of effective therapies that are capable of altering, slowing, stopping, or repairing the progressive, causative pathology of a tauopathy.

### Tau Antibodies

Provided herein are antibodies that bind to tau. In some instances, the antibodies that bind to tau are monoclonal antibodies. In certain aspects, disclosed herein is an anti-tau antibody. In some instances, the anti-tau antibody specifically binds to mammalian tau. In some instances, the anti-tau antibody specifically binds to a human tau. In some instances, the anti-tau antibody specifically binds to an N-terminal portion of tau. In some instances, the anti-tau antibody specifically binds to an N-terminal portion of human tau. In some instances, the anti-tau antibody specifically binds to an portion of tau comprising protein domain P2. In some instances, the anti-tau antibody specifically binds to an portion of human tau comprising protein domain P2. In some instances, the anti-tau antibody specifically binds to an portion of tau comprising protein domain P1. In some instances, the anti-tau antibody specifically binds to an portion of human tau comprising protein domain P1. In some instances, the anti-tau antibody specifically binds to an portion of tau comprising protein domains P1 and P2. In some instances, the anti-tau antibody specifically binds to an portion of human tau comprising protein domains P1 and P2.

In some embodiments, the anti-tau antibody comprises i) a heavy chain comprising a variable heavy chain (VH) domain and ii) a light chain comprising a variable light chain (VL) domain. In some embodiments, VH domain comprises heavy chain CDR1 (HCDR1) sequence comprising a sequence selected from SEQ ID NOs: 1-9, heavy chain CDR2 (HCDR2) sequence comprising a sequence selected from SEQ ID NOs: 10-17, and heavy chain CDR3 (HCDR3) sequence comprising a sequence selected from SEQ ID NOs: 18-23. In some embodiments, VL domain comprises light chain CDR1 (LCDR1) sequence comprising a sequence selected from SEQ ID NOs: 24-31, light chain CDR2 (LCDR2) sequence comprising a sequence selected from SEQ ID NOs: 32-36, and light chain CDR3 (LCDR3) sequence comprising a sequence selected from SEQ ID NOs: 37-43.

In some embodiments, the VH region of the anti-tau antibody comprises HCDR1, HCDR2, and HCDR3 sequences selected from Table 1.

TABLE 1
HCDR Amino Acid Sequences

| SEQ ID NO: | HCDR1 Sequence |
|---|---|
| 1 | SQKVG |
| 2 | SYAMI |
| 3 | NYKVG |
| 4 | NYAMS |
| 5 | THAMT |
| 6 | GFSLSSYA |
| 7 | GFSLSSYAMI |
| 8 | GFSLSSY |
| 9 | SSYAMI |
| SEQ ID NO: | HCDR2 Sequence |
| 10 | IINNYGSTYYASWAKG |
| 11 | FISRSGITYYASWAKG |
| 12 | IINYYSQTYYASWAKG |
| 13 | VINPSGSAYYATWVNG |
| 14 | ISRSGIT |
| 15 | FISRSGITY |
| 16 | SRSGI |
| 17 | WIGFISRSGITY |
| SEQ ID NO: | HCDR3 Sequence |
| 18 | DPDGSIVFDI |
| 19 | EFGAVGSDYYRDAFNL |
| 20 | EFGAVGSDYYRDALRL |
| 21 | DYITAGDYYMDAFDP |
| 22 | AREFGAVGSDYYRDAFNL |
| 23 | AREFGAVGSDYYRDAFN |

In some embodiments, the VH region comprises HCDR1 sequence comprising SEQ ID NO: 1; HCDR2 sequence comprising SEQ ID NO: 10; and HCDR3 sequence comprising SEQ ID NO: 18. In some embodiments, the VH region comprises HCDR1 sequence comprising SEQ ID NO: 2; HCDR2 sequence comprising SEQ ID NO: 11; and HCDR3 sequence comprising SEQ ID NO: 19. In some embodiments, the VH region comprises HCDR1 sequence comprising SEQ ID NO: 3; HCDR2 sequence comprising SEQ ID NO: 12; and HCDR3 sequence comprising SEQ ID NO: 18. In some embodiments, the VH region comprises HCDR1 sequence comprising SEQ ID NO: 4; HCDR2 sequence comprising SEQ ID NO: 11; and HCDR3 sequence comprising SEQ ID NO: 20. In some embodiments, the VH region comprises HCDR1 sequence comprising SEQ ID NO: 5; HCDR2 sequence comprising SEQ ID NO: 13; and HCDR3 sequence comprising SEQ ID NO: 21. In some embodiments, the VH region comprises HCDR1 sequence comprising SEQ ID NO: 6; HCDR2 sequence comprising SEQ ID NO: 14; and HCDR3 sequence comprising SEQ ID NO: 22. In some embodiments, the VH region comprises HCDR1 sequence comprising SEQ ID NO: 7; HCDR2 sequence comprising SEQ ID NO: 15; and HCDR3 sequence comprising SEQ ID NO: 19. In some embodiments, the VH region comprises HCDR1 sequence comprising SEQ ID NO: 8; HCDR2 sequence comprising SEQ ID NO: 16; and HCDR3 sequence comprising SEQ ID NO: 19. In some embodiments, the VH region comprises HCDR1 sequence comprising SEQ ID NO: 9; HCDR2 sequence comprising SEQ ID NO: 17; and HCDR3 sequence comprising SEQ ID NO: 23.

In some embodiments, the VL region of the anti-tau antibody comprises LCDR1, LCDR2, and LCDR3 sequences selected from Table 2.

TABLE 2
LCDR Amino Acid Sequences

| SEQ ID NO: | LCDR1 Sequence |
|---|---|
| 24 | QSSQSWYNNRLS |
| 25 | QASESINSWLS |
| 26 | QASQNIYSNLA |
| 27 | QSSQSVYSNKRLA |
| 28 | QASQSIGSNLA |
| 29 | QASQSISNQLS |
| 30 | ESINSW |
| 31 | NSWLSWY |
| SEQ ID NO: | LCDR2 Sequence |
| 32 | GASTLAS |
| 33 | RASTLAS |
| 34 | GASNLAS |
| 35 | GASTLES |
| 36 | LLIYRASTLA |
| SEQ ID NO: | LCDR3 Sequence |
| 37 | LGSYDCSSGDCHA |
| 38 | QSYYEEDGIGYA |
| 39 | QGYDYSTAGAYP |
| 40 | AGGYDCSTGDCWT |
| 41 | QSYYEGSDIGYA |
| 42 | QQGYNRDNVDNL |
| 43 | QSYYEEDGIGY |

In some embodiments, the VL region comprises LCDR1 sequence comprising SEQ ID NO: 24; LCDR2 sequence comprising SEQ ID NO: 32; and LCDR3 sequence comprising SEQ ID NO: 37. In some embodiments, the VL region comprises LCDR1 sequence comprising SEQ ID NO: 25; LCDR2 sequence comprising SEQ ID NO: 33; and LCDR3 sequence comprising SEQ ID NO: 38. In some embodiments, the VL region comprises LCDR1 sequence comprising SEQ ID NO: 26; LCDR2 sequence comprising SEQ ID NO: 34; and LCDR3 sequence comprising SEQ ID NO: 39. In some embodiments, the VL region comprises LCDR1 sequence comprising SEQ ID NO: 27; LCDR2 sequence comprising SEQ ID NO: 32; and LCDR3 sequence comprising SEQ ID NO: 40. In some embodiments, the VL region comprises LCDR1 sequence comprising SEQ ID NO: 28; LCDR2 sequence comprising SEQ ID NO: 35; and LCDR3 sequence comprising SEQ ID NO: 41. In some embodiments, the VL region comprises LCDR1 sequence comprising SEQ ID NO: 29; LCDR2 sequence comprising SEQ ID NO: 33; and LCDR3 sequence comprising SEQ ID NO: 42 In some embodiments, the VL region comprises LCDR1 sequence comprising SEQ ID NO: 30; LCDR2 sequence comprising RAS; and LCDR3 sequence comprising SEQ ID NO: 38. In some embodiments, the VL region comprises LCDR1 sequence comprising SEQ ID NO: 31; LCDR2 sequence comprising SEQ ID NO: 36; and LCDR3 sequence comprising SEQ ID NO: 44.

In some embodiments, the anti-tau antibody is an antigen binding fragment thereof. In some embodiments, the anti-tau antibody is a chimeric antibody or antigen binding fragment thereof. In some embodiments, the anti-tau antibody comprises an IgG-scFv, nanobody, mini-antibody, minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, or intrabody. In some embodiments, the anti-tau antibody comprises a bispecific antibody. In some embodiments, the anti-tau antibody comprises a multispecific antibody. In some embodiments, the anti-tau antibody is an IgG1 antibody. In some embodiments, the anti-tau antibody is an IgG2 antibody. In some embodiments, the anti-tau antibody is an IgG4 antibody. In some embodiments, the anti-tau antibody comprises a light chain wherein the light chain is a kappa chain.

In some embodiments, the anti-tau antibody specifically binds to a tau protein. In some embodiments, the anti-tau antibody specifically binds to N-terminal region of a tau protein. In some embodiments, the anti-tau antibody specifically binds to cis-conformation of a tau protein. In some embodiments, the anti-tau antibody specifically binds to a tau protein at any one of positions 1-441. In some embodiments, the anti-tau antibody specifically binds to a tau protein at any one of positions 152-400. In some embodiments, the anti-tau antibody specifically binds to a tau protein at any one of positions 198-275. In some embodiments, the anti-tau antibody specifically binds to a tau protein at any one of positions 244-369. In some embodiments, the anti-tau antibody specifically binds to a tau protein at any one of positions 152-275. In some embodiments, the anti-tau antibody specifically binds to a tau protein at any one of positions 152-244. In some embodiments, the anti-tau antibody specifically binds to a tau protein at any one of positions 181, 217, 231. In some embodiments, the anti-tau antibody specifically binds to a tau protein at position 181. In some embodiments, the anti-tau antibody specifically binds to a tau protein at position 217. In some embodiments, the anti-tau antibody specifically binds to a tau protein at position 231.

In some embodiments, the anti-tau antibody specifically binds to a phosphorylated tau protein. In some embodiments, the anti-tau antibody specifically binds to a phosphorylated tau protein at any one of positions 1-441. In some embodiments, the anti-tau antibody specifically binds to a phosphorylated tau protein at any one of positions 1-441, wherein any combination of the following residues are phosphorylated Y18, S46, S68, T69, T71, S113, T123, T153, T175, T181, S184, S185, S191, Y197, S198, S199, S208, S210, T212, S214, T217, T220, T231, S235, S237, S238, S258, S262, S289, S356, Y394, S396, S400, T403, S404, S409, S412, S413, S416, S422, T427, S433, or S435. In some embodiments, the anti-tau antibody specifically binds to one or any combination of phosphorylated serine, threonine, or tyrosine residues within a tau protein, wherein the one or any combination of phosphorylated serine, threonine, or tyrosine residues is selected from Y18, S46, S68, T69, T71, S113, T123, T153, T175, T181, S184, S185, S191, Y197, S198, S199, S208, S210, T212, S214, T217, T220, T231, S235, S237, S238, S258, S262, S289, S356, Y394, S396, S400, T403, S404, S409, S412, S413, S416, S422, T427, S433, and S435. In some embodiments, the anti-tau antibody specifically binds to a phosphorylated tau protein at any one of positions 152-400. In some embodiments, the anti-tau antibody specifically binds to a phosphorylated tau protein at any one of positions 198-275. In some embodiments, the anti-tau antibody specifically binds to a phosphorylated tau protein at any one of positions 244-369. In some embodiments, the anti-tau antibody specifically binds to a phosphorylated tau protein at any one of positions 152-275. In some embodiments, the anti-tau antibody specifically binds to a phosphorylated tau protein at any one of positions 152-244. In some embodiments, the anti-tau antibody specifically binds to a phosphorylated tau protein at any one of positions 181, 217, 231. In some embodiments, the anti-tau antibody specifically binds to a phosphorylated tau protein when any combination of T181, T217, and T231 is phosphorylated. In some embodiments, the anti-tau antibody specifically binds to a phosphorylated tau protein at position 181. In some embodiments, the anti-tau antibody specifically binds to a phosphorylated tau protein when T181 is phosphorylated. In some embodiments, the anti-tau antibody specifically binds to phosphorylated residue T181 in a tau protein. In some embodiments, the anti-tau antibody specifically binds to a phosphorylated tau protein at position 217. In some embodiments, the anti-tau antibody specifically binds to phosphorylated residue T217 in a tau protein. In some embodiments, the anti-tau antibody specifically binds to a phosphorylated tau protein when T217 is phosphorylated. In some embodiments, the anti-tau antibody specifically binds to a phosphorylated tau protein at position 231. In some embodiments, the anti-tau antibody specifically binds to phosphorylated residue T231 in a tau protein. In some embodiments, the anti-tau antibody specifically binds to a phosphorylated tau protein when T231 is phosphorylated. In some embodiments, the phosphorylated tau protein is a human tau protein. The amino acid residue numbering system for tau residues refers to positions in a full-length 441 amino acid isoform of human microtubule associated protein tau (MAPT; NBCI gene ID: 4137; exemplary protein sequence —NBCI Entry: 7SP1 A).

In some embodiments, the anti-tau antibody has a binding affinity to human tau of about 100 pM to about 3 nM. In some embodiments, the anti-tau antibody has a binding affinity to human tau of about 100 pM to 300 pM. In some embodiments, the anti-tau antibody has a binding affinity to human tau of about 100 pM to 500 pM. In some embodiments, the anti-tau antibody has a binding affinity to human tau of about 100 pM to 800 pM. In some embodiments, the anti-tau antibody has a binding affinity to human tau of about 300 pM to 600 pM. In some embodiments, the anti-tau antibody has a binding affinity to human tau of about 300 pM to 900 pM. In some embodiments, the anti-tau antibody has a binding affinity to human tau of about 400 pM to 1 nM. In some embodiments, the anti-tau antibody has a binding affinity to human tau of about 500 pM to 1.5 nM. In some embodiments, the anti-tau antibody has a binding affinity to human tau of about 500 pM to 2 nM. In some embodiments, the anti-tau antibody has a binding affinity to human tau of about 600 pM to 3 nM. In some embodiments, the anti-tau antibody has a binding affinity to human tau of about 100 pM to about 3 nM.

In some embodiments, the anti-tau antibody has a binding affinity to phosphorylated human tau of about 100 pM to 300 pM. In some embodiments, the anti-tau antibody has a binding affinity to phosphorylated human tau of about 100 pM to 500 pM. In some embodiments, the anti-tau antibody has a binding affinity to phosphorylated human tau of about 100 pM to 800 pM. In some embodiments, the anti-tau antibody has a binding affinity to phosphorylated human tau of about 300 pM to 600 pM. In some embodiments, the anti-tau antibody has a binding affinity to phosphorylated human tau of about 300 pM to 900 pM. In some embodiments, the anti-tau antibody has a binding affinity to phosphorylated human tau of about 400 pM to 1 nM. In some embodiments, the anti-tau antibody has a binding affinity to phosphorylated human tau of about 500 pM to 1.5 nM. In some embodiments, the anti-tau antibody has a binding affinity to phosphorylated human tau of about 500 pM to 2 nM. In some embodiments, the anti-tau antibody has a binding affinity to phosphorylated human tau of about 600 pM to 3 nM.

In some embodiments, the anti-tau antibody has a binding affinity to threonine phosphorylated tau 181 (ptau 181) of about 100 pM to 300 pM. In some embodiments, the anti-tau antibody has a binding affinity to ptau 181 of about 100 pM to 500 pM. In some embodiments, the anti-tau antibody has a binding affinity to ptau 181 of about 100 pM to 800 pM. In some embodiments, the anti-tau antibody has a binding affinity to ptau 181 of about 300 pM to 600 pM. In some embodiments, the anti-tau antibody has a binding affinity to ptau 181 of about 300 pM to 900 pM. In some embodiments, the anti-tau antibody has a binding affinity to ptau 181 of about 400 pM to 1 nM. In some embodiments, the anti-tau antibody has a binding affinity to ptau 181 of about 500 pM to 1.5 nM. In some embodiments, the anti-tau antibody has a binding affinity to ptau 181 of about 500 pM to 2 nM. In some embodiments, the anti-tau antibody has a binding affinity to ptau 181 of about 600 pM to 3 nM.

In some embodiments, the anti-tau antibody has a binding affinity to threonine phosphorylated tau 212 (ptau 212) of about 100 pM to 300 pM. In some embodiments, the anti-tau antibody has a binding affinity to ptau 212 of about 100 pM to 500 pM. In some embodiments, the anti-tau antibody has a binding affinity to ptau 212 of about 100 pM to 800 pM. In some embodiments, the anti-tau antibody has a binding affinity to ptau 212 of about 300 pM to 600 pM. In some embodiments, the anti-tau antibody has a binding affinity to ptau 212 of about 300 pM to 900 pM. In some embodiments, the anti-tau antibody has a binding affinity to ptau 212 of about 400 pM to 1 nM. In some embodiments, the anti-tau antibody has a binding affinity to ptau 212 of about 500 pM to 1.5 nM. In some embodiments, the anti-tau antibody has a binding affinity to ptau 212 of about 500 pM to 2 nM. In some embodiments, the anti-tau antibody has a binding affinity to ptau 212 of about 600 pM to 3 nM.

In some embodiments, the anti-tau antibody has a binding affinity to serine phosphorylated tau 214 (ptau 214) of about 100 pM to 300 pM. In some embodiments, the anti-tau antibody has a binding affinity to ptau 214 of about 100 pM to 500 pM. In some embodiments, the anti-tau antibody has a binding affinity to ptau 214 of about 100 pM to 800 pM. In some embodiments, the anti-tau antibody has a binding affinity to ptau 214 of about 300 pM to 600 pM. In some embodiments, the anti-tau antibody has a binding affinity to ptau 214 of about 300 pM to 900 pM. In some embodiments, the anti-tau antibody has a binding affinity to ptau 214 of about 400 pM to 1 nM. In some embodiments, the anti-tau antibody has a binding affinity to ptau 214 of about 500 pM to 1.5 nM. In some embodiments, the anti-tau antibody has a binding affinity to ptau 214 of about 500 pM to 2 nM. In some embodiments, the anti-tau antibody has a binding affinity to ptau 214 of about 600 pM to 3 nM.

In some embodiments, the anti-tau antibody has a binding affinity to threonine phosphorylated tau 217 (ptau 217) of about 100 pM to 300 pM. In some embodiments, the anti-tau antibody has a binding affinity to ptau 217 of about 100 pM to 500 pM. In some embodiments, the anti-tau antibody has a binding affinity to ptau 217 of about 100 pM to 800 pM. In some embodiments, the anti-tau antibody has a binding affinity to ptau 217 of about 300 pM to 600 pM. In some embodiments, the anti-tau antibody has a binding affinity to ptau 217 of about 300 pM to 900 pM. In some embodiments, the anti-tau antibody has a binding affinity to ptau 217 of about 400 pM to 1 nM. In some embodiments, the anti-tau antibody has a binding affinity to ptau 217 of about 500 pM to 1.5 nM. In some embodiments, the anti-tau antibody has a binding affinity to ptau 217 of about 500 pM to 2 nM. In some embodiments, the anti-tau antibody has a binding affinity to ptau 217 of about 600 pM to 3 nM.

In some embodiments, the anti-tau antibody has a binding affinity to threonine phosphorylated tau 220 (ptau 220) of about 100 pM to 300 pM. In some embodiments, the anti-tau antibody has a binding affinity to ptau 220 of about 100 pM to 500 pM. In some embodiments, the anti-tau antibody has a binding affinity to ptau 220 of about 100 pM to 800 pM. In some embodiments, the anti-tau antibody has a binding affinity to ptau 220 of about 300 pM to 600 pM. In some embodiments, the anti-tau antibody has a binding affinity to ptau 220 of about 300 pM to 900 pM. In some embodiments, the anti-tau antibody has a binding affinity to ptau 220 of about 400 pM to 1 nM. In some embodiments, the anti-tau antibody has a binding affinity to ptau 220 of about 500 pM to 1.5 nM. In some embodiments, the anti-tau antibody has a binding affinity to ptau 220 of about 500 pM to 2 nM. In some embodiments, the anti-tau antibody has a binding affinity to ptau 220 of about 600 pM to 3 nM.

In some embodiments, the anti-tau antibody has a binding affinity to threonine phosphorylated tau 231 (ptau 231) of about 100 pM to 300 pM. In some embodiments, the anti-tau antibody has a binding affinity to ptau 231of about 100 pM to 500 pM. In some embodiments, the anti-tau antibody has a binding affinity to ptau 231of about 100 pM to 800 pM. In some embodiments, the anti-tau antibody has a binding affinity to ptau 231of about 300 pM to 600 pM. In some embodiments, the anti-tau antibody has a binding affinity to ptau 231of about 300 pM to 900 pM. In some embodiments, the anti-tau antibody has a binding affinity to ptau 231of about 400 pM to 1 nM. In some embodiments, the anti-tau antibody has a binding affinity to ptau 231of about 500 pM to 1.5 nM. In some embodiments, the anti-tau antibody has a binding affinity to ptau 231of about 500 pM to 2 nM. In some embodiments, the anti-tau antibody has a binding affinity to ptau 231 of about 600 pM to 3 nM.

Described herein are antibodies comprising a sequence of any sequence set forth in Table 3 or Table 4.

TABLE 3
Variable Domain, Heavy Chain

| | SEQ ID | |
|---|---|---|
| Name | NO: | Amino Acid Sequence |
| Antibody 1 | 44 | METGLRWLLLVAVLKGVQCQSLEESGGRLVTPGTPLTLTCTVSGFSLSSQK |
| Variable | | VGWVRQAPGKGLEWIGIINNYGSTYYASWAKGRFTISKTSTTVDLRITSLTA |
| Domain, | | EDTATYFCARDPDGSIVFDIWGPGTLVTVSL |
| Heavy Chain | | |
| Antibody 2 | 45 | METGLRWLLLVAVLKGVQCQSVEESGGRLVTPGTPLTLTCTVSGFSLSSYA |
| and Antibody | | MIWVRQAPGKGLEWIGFISRSGITYYASWAKGRFTISKTSTTVDLKMTSLTT |
| 3 Variable | | EDTATYFCAREFGAVGSDYYRDAFNLWGPGTLVTVSS |
| Domain, | | |
| Heavy Chain | | |
| Antibody 4 | 46 | METGLRWLLLVAVLKGVQCQSLEESGGRLVTPGTPLTLTCTVSGFSLNNYK |
| Variable | | VGWVRQAPGKGLEWIGIINYYSQTYYASWAKGRFTISKTSTTVDLKLTSPTT |
| Domain, | | EDTATYFCARDPDGSIVFDIWGPGTLVTVSL |
| Heavy Chain | | |
| Antibody 5 | 47 | METGLRWLLLVAVLKGVQCQSVEESGGGLVTPGGTLTLTCTVSGFSLSNY |
| Variable | | AMSWVRQAPGKGLEWIGFISRSGITYYASWAKGRFTISKTSTTVDLKITSPT |
| Domain, | | TEDTAAYFCAREFGAVGSDYYRDALRLWGPGTLVTVSS |
| Heavy Chain | | |
| Antibody 6 | 48 | METGLRWLLLVAVLKGVQCQSLEESGGRLVTPGTPLTLTCTVSGIDLSTHA |
| Variable | | MTWVRQAPGKGLEWIGVINPSGSAYYATWVNGRFTISKTSTTVDLKITSPT |
| Domain, | | TGDTAKYFCARDYITAGDYYMDAFDPWGPGTLVTVSS |
| Heavy Chain | | |

TABLE 4
Variable Domain, Light Chain

| | SEQ ID | |
|---|---|---|
| Name | NO: | Amino Acid Sequence |
| Antibody 1 | 49 | MDTRAPTQLLGLLLLWLPGATFAQVLTQTASPVSAAVGGTVTINCQSSQSV |
| Variable | | VYNNRLSWFQQKPGQPPKLLIYGASTLASGVPSRFKGSGSGTQFTLTISDV |
| Domain, | | QCDDAATYYCLGSYDCSSGDCHAFGGGTEVVVK |
| Light Chain | | |
| Antibody 2 | 50 | MDMRAPTQLLGLLLLWLPGARCADIVMTQTPASVEAAVGGTVTINCQASES |
| Variable | | INSWLSWYQQKPGQPPNLLIYRASTLASGVPSRFSGGGSGTEYTLTISDLE |
| Domain, | | CADAVTYYCQSYYEEDGIGYAFGGGTEVVVE |
| Light Chain | | |
| Antibody 3 | 51 | MDMRAPTQLLGLLLLWLPGARCADIVMTQTPSSVSAAVGGTVTINCQASQ |
| Variable | | NIYSNLAWYQQKPGQRPRLLIYGASNLASGVPSRFKGSRSGTEFTLTISDLE |
| Domain, | | CADAATYYCQGYDYSTAGAYPFGGGTAVVVK |
| Light Chain | | |
| Antibody 4 | 52 | MDTRAPTQLLGLLLLWLPGATFAQVLTQTASPVSAAVGSTVTINCQSSQSV |
| Variable | | YSNKRLAWFQLKPGQPPKLLIYGASTLASGVPSRFKGSGSGTQFTLTISDV |
| Domain, | | QCDDAATYYCAGGYDCSTGDCWTFGGGTEVVVT |
| Light Chain | | |
| Antibody 5 | 53 | MDMRAPTQLLGLLLLWLPGARCADIVMTQTPSSVSAAVGGTVTIKCQASQS |
| Variable | | IGSNLAWYQQKPGQPPKLLIYGASTLESGVPSRFKGSGSGTEYTLTISDLEC |
| Domain, | | ADAATYYCQSYYEGSDIGYAFGGGTEVVVE |
| Light Chain | | |
| Antibody 6 | 54 | MDTRAPTQLLGLLLLWLPGARCADIVMTQTPASVSAAVGGTVTIKCQASQS |
| Variable | | ISNQLSWYQQKSGQPPKLLIYRASTLASGVPSRFKGSGSGTEFTLTISDLEC |
| Domain, | | ADAATYYCQQGYNRDNVDNLFGGGTEVVVK |
| Light Chain | | |

In some embodiments, the variable domain, heavy chain region (VH) comprises an amino acid sequence that has at least 70% sequence identity to the amino acid sequence according to any one of SEQ ID NOs: 44-48. In some embodiments, the VH comprises an amino acid sequence that has at least 80% sequence identity to the amino acid sequence according to any one of SEQ ID NOs: 44-48. In some embodiments, the VH comprises an amino acid sequence that has at least 85% sequence identity to the amino acid sequence according to any one of SEQ ID NOs: 44-48. In some embodiments, the VH comprises an amino acid sequence that has at least 90% sequence identity to the amino acid sequence according to any one of SEQ ID NOs: 44-48. In some embodiments, the VH comprises an amino acid sequence that has at least 91% sequence identity to the amino acid sequence according to any one of SEQ ID NOs: 44-48. In some embodiments, the VH comprises an amino acid sequence that has at least 92% sequence identity to the amino acid sequence according to any one of SEQ ID NOs: 44-48. In some embodiments, the VH comprises an amino acid sequence that has at least 93% sequence identity to the amino acid sequence according to any one of SEQ ID NOs: 44-48. In some embodiments, the VH comprises an amino acid sequence that has at least 94% sequence identity to the amino acid sequence according to any one of SEQ ID NOs: 44-48. In some embodiments, the VH comprises an amino acid sequence that has at least 95% sequence identity to the amino acid sequence according to any one of SEQ ID NOs: 44-48. In some embodiments, the VH comprises an amino acid sequence that has at least 96% sequence identity to the amino acid sequence according to any one of SEQ ID NOs: 44-48. In some embodiments, the VH comprises an amino acid sequence that has at least 97% sequence identity to the amino acid sequence according to any one of SEQ ID NOs: 44-48. In some embodiments, the VH comprises an amino acid sequence that has at least 98% sequence identity to the amino acid sequence according to any one of SEQ ID NOs: 44-48. In some embodiments, the VH comprises an amino acid sequence that has at least 99% sequence identity to the amino acid sequence according to any one of SEQ ID NOs: 44-48. In some embodiments, the VH comprises an amino acid sequence according to any one of SEQ ID NOs: 44-48.

In some embodiments, the VH comprises an amino acid sequence of at least 50 consecutive amino acid residues of any one of SEQ ID NOs: 44-48. In some embodiments, the VH comprises an amino acid sequence of at least 60 consecutive amino acid residues of any one of SEQ ID NOs: 44-48. In some embodiments, the VH comprises an amino acid sequence of at least 70 consecutive amino acid residues of any one of SEQ ID NOs: 44-48. In some embodiments, the VH comprises an amino acid sequence of at least 80 consecutive amino acid residues of any one of SEQ ID NOs: 44-48. In some embodiments, the VH comprises an amino acid sequence of at least 90 consecutive amino acid residues of any one of SEQ ID NOs: 44-48. In some embodiments, the VH comprises an amino acid sequence of at least 100 consecutive amino acid residues of any one of SEQ ID NOs: 44-48. In some embodiments, the VH comprises an amino acid sequence of at least 105 consecutive amino acid residues of any one of SEQ ID NOs: 44-48. In some embodiments, the VH comprises an amino acid sequence of at least 110 consecutive amino acid residues of any one of SEQ ID NOs: 44-48. In some embodiments, the VH comprises an amino acid sequence of at least 115 consecutive amino acid residues of any one of SEQ ID NOs: 44-48. In some embodiments, the VH comprises an amino acid sequence of at least 120 consecutive amino acid residues of any one of SEQ ID NOs: 44-48.

In some embodiments, the VH comprises an amino acid sequence of at least 50 consecutive amino acid residues of any one of SEQ ID NOs: 44-48, and has at least 80% sequence identity to the at least 50 consecutive amino acid residues of any one of SEQ ID NOs: 44-48. In some embodiments, the VH comprises an amino acid sequence of at least 60 consecutive amino acid residues of any one of SEQ ID NOs: 44-48, and has at least 80% sequence identity to the at least 60 consecutive amino acid residues of any one of SEQ ID NOs: 44-48. In some embodiments, the VH comprises an amino acid sequence of at least 70 consecutive amino acid residues of any one of SEQ ID NOs: 44-48, and has at least 80% sequence identity to the at least 70 consecutive amino acid residues of any one of SEQ ID NOs: 44-48. In some embodiments, the VH comprises an amino acid sequence of at least 80 consecutive amino acid residues of any one of SEQ ID NOs: 44-48, and has at least 80% sequence identity to the at least 80 consecutive amino acid residues of any one of SEQ ID NOs: 44-48. In some embodiments, the VH comprises an amino acid sequence of at least 90 consecutive amino acid residues of any one of SEQ ID NOs: 44-48, and has at least 80% sequence identity to the at least 90 consecutive amino acid residues of any one of SEQ ID NOs: 44-48. In some embodiments, the VH comprises an amino acid sequence of at least 100 consecutive amino acid residues of any one of SEQ ID NOs: 44-48, and has at least 80% sequence identity to the at least 100 consecutive amino acid residues of any one of SEQ ID NOs: 44-48. In some embodiments, the VH comprises an amino acid sequence of at least 105 consecutive amino acid residues of any one of SEQ ID NOs: 44-48, and has at least 80% sequence identity to the at least 105 consecutive amino acid residues of any one of SEQ ID NOs: 44-48. In some embodiments, the VH comprises an amino acid sequence of at least 110 consecutive amino acid residues of any one of SEQ ID NOs: 44-48, and has at least 80% sequence identity to the at least 110 consecutive amino acid residues of any one of SEQ ID NOs: 44-48. In some embodiments, the VH comprises an amino acid sequence of at least 115 consecutive amino acid residues of any one of SEQ ID NOs: 44-48, and has at least 80% sequence identity to the at least 115 consecutive amino acid residues of any one of SEQ ID NOs: 44-48. In some embodiments, the VH comprises an amino acid sequence of at least 120 consecutive amino acid residues of any one of SEQ ID NOs: 44-48, and has at least 80% sequence identity to the at least 120 consecutive amino acid residues of any one of SEQ ID NOs: 44-48.

In some embodiments, the VH comprises an amino acid sequence of at least 50 consecutive amino acid residues of any one of SEQ ID NOs: 44-48, and has at least 90% sequence identity to the at least 50 consecutive amino acid residues of any one of SEQ ID NOs: 44-48. In some embodiments, the VH comprises an amino acid sequence of at least 60 consecutive amino acid residues of any one of SEQ ID NOs: 44-48, and has at least 90% sequence identity to the at least 60 consecutive amino acid residues of any one of SEQ ID NOs: 44-48. In some embodiments, the VH comprises an amino acid sequence of at least 70 consecutive amino acid residues of any one of SEQ ID NOs: 44-48, and has at least 90% sequence identity to the at least 70 consecutive amino acid residues of any one of SEQ ID NOs: 44-48. In some embodiments, the VH comprises an amino acid sequence of at least 80 consecutive amino acid residues of any one of SEQ ID NOs: 44-48, and has at least 90% sequence identity to the at least 80 consecutive amino acid residues of any one of SEQ ID NOs: 44-48. In some embodiments, the VH comprises an amino acid sequence of at least 90 consecutive amino acid residues of any one of SEQ ID NOs: 44-48, and has at least 90% sequence identity to the at least 90 consecutive amino acid residues of any one of SEQ ID NOs: 44-48. In some embodiments, the VH comprises an amino acid sequence of at least 100 consecutive amino acid residues of any one of SEQ ID NOs: 44-48, and has at least 90% sequence identity to the at least 100 consecutive amino acid residues of any one of SEQ ID NOs: 44-48. In some embodiments, the VH comprises an amino acid sequence of at least 105 consecutive amino acid residues of any one of SEQ ID NOs: 44-48, and has at least 90% sequence identity to the at least 105 consecutive amino acid residues of any one of SEQ ID NOs: 44-48. In some embodiments, the VH comprises an amino acid sequence of at least 110 consecutive amino acid residues of any one of SEQ ID NOs: 44-48, and has at least 90% sequence identity to the at least 110 consecutive amino acid residues of any one of SEQ ID NOs: 44-48. In some embodiments, the VH comprises an amino acid sequence of at least 115 consecutive amino acid residues of any one of SEQ ID NOs: 44-48, and has at least 90% sequence identity to the at least 115 consecutive amino acid residues of any one of SEQ ID NOs: 44-48. In some embodiments, the VH comprises an amino acid sequence of at least 120 consecutive amino acid residues of any one of SEQ ID NOs: 44-48, and has at least 90% sequence identity to the at least 120 consecutive amino acid residues of any one of SEQ ID NOs: 44-48.

In some embodiments, the VH comprises an amino acid sequence of at least 50 consecutive amino acid residues of any one of SEQ ID NOs: 44-48, and has at least 95% sequence identity to the at least 50 consecutive amino acid residues of any one of SEQ ID NOs: 44-48. In some embodiments, the VH comprises an amino acid sequence of at least 60 consecutive amino acid residues of any one of SEQ ID NOs: 44-48, and has at least 95% sequence identity to the at least 60 consecutive amino acid residues of any one of SEQ ID NOs: 44-48. In some embodiments, the VH comprises an amino acid sequence of at least 70 consecutive amino acid residues of any one of SEQ ID NOs: 44-48, and has at least 95% sequence identity to the at least 70 consecutive amino acid residues of any one of SEQ ID NOs: 44-48. In some embodiments, the VH comprises an amino acid sequence of at least 80 consecutive amino acid residues of any one of SEQ ID NOs: 44-48, and has at least 95% sequence identity to the at least 80 consecutive amino acid residues of any one of SEQ ID NOs: 44-48. In some embodiments, the VH comprises an amino acid sequence of at least 90 consecutive amino acid residues of any one of SEQ ID NOs: 44-48, and has at least 95% sequence identity to the at least 90 consecutive amino acid residues of any one of SEQ ID NOs: 44-48. In some embodiments, the VH comprises an amino acid sequence of at least 100 consecutive amino acid residues of any one of SEQ ID NOs: 44-48, and has at least 95% sequence identity to the at least 100 consecutive amino acid residues of any one of SEQ ID NOs: 44-48. In some embodiments, the VH comprises an amino acid sequence of at least 105 consecutive amino acid residues of any one of SEQ ID NOs: 44-48, and has at least 95% sequence identity to the at least 105 consecutive amino acid residues of any one of SEQ ID NOs: 44-48. In some embodiments, the VH comprises an amino acid sequence of at least 110 consecutive amino acid residues of any one of SEQ ID NOs: 44-48, and has at least 95% sequence identity to the at least 110 consecutive amino acid residues of any one of SEQ ID NOs: 44-48. In some embodiments, the VH comprises an amino acid sequence of at least 115 consecutive amino acid residues of any one of SEQ ID NOs: 44-48, and has at least 95% sequence identity to the at least 115 consecutive amino acid residues of any one of SEQ ID NOs: 44-48. In some embodiments, the VH comprises an amino acid sequence of at least 120 consecutive amino acid residues of any one of SEQ ID NOs: 44-48, and has at least 95% sequence identity to the at least 120 consecutive amino acid residues of any one of SEQ ID NOs: 44-48.

In some embodiments, the VH comprises an amino acid sequence of at least 100 consecutive amino acid residues of any one of SEQ ID NOs: 44-48, and has at least 99% sequence identity to the at least 100 consecutive amino acid residues of any one of SEQ ID NOs: 44-48. In some embodiments, the VH comprises an amino acid sequence of at least 105 consecutive amino acid residues of any one of SEQ ID NOs: 44-48, and has at least 99% sequence identity to the at least 105 consecutive amino acid residues of any one of SEQ ID NOs: 44-48. In some embodiments, the VH comprises an amino acid sequence of at least 110 consecutive amino acid residues of any one of SEQ ID NOs: 44-48, and has at least 99% sequence identity to the at least 110 consecutive amino acid residues of any one of SEQ ID NOs: 44-48. In some embodiments, the VH comprises an amino acid sequence of at least 115 consecutive amino acid residues of any one of SEQ ID NOs: 44-48, and has at least 99% sequence identity to the at least 115 consecutive amino acid residues of any one of SEQ ID NOs: 44-48. In some embodiments, the VH comprises an amino acid sequence of at least 120 consecutive amino acid residues of any one of SEQ ID NOs: 44-48, and has at least 99% sequence identity to the at least 120 consecutive amino acid residues of any one of SEQ ID NOs: 44-48.

In some embodiments, the variable domain, light chain region (VL) comprises an amino acid sequence that has at least 70% sequence identity to the amino acid sequence according to any one of SEQ ID NOs: 49-54. In some embodiments, the VL comprises an amino acid sequence that has at least 80% sequence identity to the amino acid sequence according to any one of SEQ ID NOs: 49-54. In some embodiments, the VL comprises an amino acid sequence that has at least 85% sequence identity to the amino acid sequence according to any one of SEQ ID NOs: 49-54. In some embodiments, the VL comprises an amino acid sequence that has at least 90% sequence identity to the amino acid sequence according to any one of SEQ ID NOs: 49-54. In some embodiments, the VL comprises an amino acid sequence that has at least 91% sequence identity to the amino acid sequence according to any one of SEQ ID NOs: 49-54. In some embodiments, the VL comprises an amino acid sequence that has at least 92% sequence identity to the amino acid sequence according to any one of SEQ ID NOs: 49-54. In some embodiments, the VL comprises an amino acid sequence that has at least 93% sequence identity to the amino acid sequence according to any one of SEQ ID NOs: 49-54. In some embodiments, the VL comprises an amino acid sequence that has at least 94% sequence identity to the amino acid sequence according to any one of SEQ ID NOs: 49-54. In some embodiments, the VL comprises an amino acid sequence that has at least 95% sequence identity to the amino acid sequence according to any one of SEQ ID NOs: 49-54. In some embodiments, the VL comprises an amino acid sequence that has at least 96% sequence identity to the amino acid sequence according to any one of SEQ ID NOs: 49-54. In some embodiments, the VL comprises an amino acid sequence that has at least 97% sequence identity to the amino acid sequence according to any one of SEQ ID NOs: 49-54. In some embodiments, the VL comprises an amino acid sequence that has at least 98% sequence identity to the amino acid sequence according to any one of SEQ ID NOs: 49-54. In some embodiments, the VL comprises an amino acid sequence that has at least 99% sequence identity to the amino acid sequence according to any one of SEQ ID NOs: 49-54. In some embodiments, the VL comprises an amino acid sequence according to any one of SEQ ID NOs: 49-54.

In some embodiments, the VL comprises an amino acid sequence of at least 50 consecutive amino acid residues of any one of SEQ ID NOs: 49-54. In some embodiments, the VL comprises an amino acid sequence of at least 60 consecutive amino acid residues of any one of SEQ ID NOs: 49-54. In some embodiments, the VL comprises an amino acid sequence of at least 70 consecutive amino acid residues of any one of SEQ ID NOs: 49-54. In some embodiments, the VL comprises an amino acid sequence of at least 80 consecutive amino acid residues of any one of SEQ ID NOs: 49-54. In some embodiments, the VL comprises an amino acid sequence of at least 90 consecutive amino acid residues of any one of SEQ ID NOs: 49-54. In some embodiments, the VL comprises an amino acid sequence of at least 100 consecutive amino acid residues of any one of SEQ ID NOs: 49-54. In some embodiments, the VL comprises an amino acid sequence of at least 105 consecutive amino acid residues of any one of SEQ ID NOs: 49-54.

In some embodiments, the VL comprises an amino acid sequence of at least 50 consecutive amino acid residues of any one of SEQ ID NOs: 49-54, and has at least 80% sequence identity to the at least 50 consecutive amino acid residues of any one of SEQ ID NOs: 49-54. In some embodiments, the VL comprises an amino acid sequence of at least 60 consecutive amino acid residues of any one of SEQ ID NOs: 49-54, and has at least 80% sequence identity to the at least 60 consecutive amino acid residues of any one of SEQ ID NOs: 49-54. In some embodiments, the VL comprises an amino acid sequence of at least 70 consecutive amino acid residues of any one of SEQ ID NOs: 49-54, and has at least 80% sequence identity to the at least 70 consecutive amino acid residues of any one of SEQ ID NOs: 49-54. In some embodiments, the VL comprises an amino acid sequence of at least 80 consecutive amino acid residues of any one of SEQ ID NOs: 49-54, and has at least 80% sequence identity to the at least 80 consecutive amino acid residues of any one of SEQ ID NOs: 49-54. In some embodiments, the VL comprises an amino acid sequence of at least 90 consecutive amino acid residues of any one of SEQ ID NOs: 49-54, and has at least 80% sequence identity to the at least 90 consecutive amino acid residues of any one of SEQ ID NOs: 49-54. In some embodiments, the VL comprises an amino acid sequence of at least 100 consecutive amino acid residues of any one of SEQ ID NOs: 49-54, and has at least 80% sequence identity to the at least 100 consecutive amino acid residues of any one of SEQ ID NOs: 49-54. In some embodiments, the VL comprises an amino acid sequence of at least 105 consecutive amino acid residues of any one of SEQ ID NOs: 49-54, and has at least 80% sequence identity to the at least 105 consecutive amino acid residues of any one of SEQ ID NOs: 49-54.

In some embodiments, the VL comprises an amino acid sequence of at least 50 consecutive amino acid residues of any one of SEQ ID NOs: 49-54, and has at least 90% sequence identity to the at least 50 consecutive amino acid residues of any one of SEQ ID NOs: 49-54. In some embodiments, the VL comprises an amino acid sequence of at least 60 consecutive amino acid residues of any one of SEQ ID NOs: 49-54, and has at least 90% sequence identity to the at least 60 consecutive amino acid residues of any one of SEQ ID NOs: 49-54. In some embodiments, the VL comprises an amino acid sequence of at least 70 consecutive amino acid residues of any one of SEQ ID NOs: 49-54, and has at least 90% sequence identity to the at least 70 consecutive amino acid residues of any one of SEQ ID NOs: 49-54. In some embodiments, the VL comprises an amino acid sequence of at least 80 consecutive amino acid residues of any one of SEQ ID NOs: 49-54, and has at least 90% sequence identity to the at least 80 consecutive amino acid residues of any one of SEQ ID NOs: 49-54. In some embodiments, the VL comprises an amino acid sequence of at least 90 consecutive amino acid residues of any one of SEQ ID NOs: 49-54, and has at least 90% sequence identity to the at least 90 consecutive amino acid residues of any one of SEQ ID NOs: 49-54. In some embodiments, the VL comprises an amino acid sequence of at least 100 consecutive amino acid residues of any one of SEQ ID NOs: 49-54, and has at least 90% sequence identity to the at least 100 consecutive amino acid residues of any one of SEQ ID NOs: 49-54. In some embodiments, the VL comprises an amino acid sequence of at least 105 consecutive amino acid residues of any one of SEQ ID NOs: 49-54, and has at least 90% sequence identity to the at least 105 consecutive amino acid residues of any one of SEQ ID NOs: 49-54.

In some embodiments, the VL comprises an amino acid sequence of at least 50 consecutive amino acid residues of any one of SEQ ID NOs: 49-54, and has at least 95% sequence identity to the at least 50 consecutive amino acid residues of any one of SEQ ID NOs: 49-54. In some embodiments, the VL comprises an amino acid sequence of at least 60 consecutive amino acid residues of any one of SEQ ID NOs: 49-54, and has at least 95% sequence identity to the at least 60 consecutive amino acid residues of any one of SEQ ID NOs: 49-54. In some embodiments, the VL comprises an amino acid sequence of at least 70 consecutive amino acid residues of any one of SEQ ID NOs: 49-54, and has at least 95% sequence identity to the at least 70 consecutive amino acid residues of any one of SEQ ID NOs: 49-54. In some embodiments, the VL comprises an amino acid sequence of at least 80 consecutive amino acid residues of any one of SEQ ID NOs: 49-54, and has at least 95% sequence identity to the at least 80 consecutive amino acid residues of any one of SEQ ID NOs: 49-54. In some embodiments, the VL comprises an amino acid sequence of at least 90 consecutive amino acid residues of any one of SEQ ID NOs: 49-54, and has at least 95% sequence identity to the at least 90 consecutive amino acid residues of any one of SEQ ID NOs: 49-54. In some embodiments, the VL comprises an amino acid sequence of at least 100 consecutive amino acid residues of any one of SEQ ID NOs: 49-54, and has at least 95% sequence identity to the at least 100 consecutive amino acid residues of any one of SEQ ID NOs: 49-54. In some embodiments, the VL comprises an amino acid sequence of at least 105 consecutive amino acid residues of any one of SEQ ID NOs: 49-54, and has at least 95% sequence identity to the at least 105 consecutive amino acid residues of any one of SEQ ID NOs: 49-54. In some embodiments, the VL comprises an amino acid sequence of at least 100 consecutive amino acid residues of any one of SEQ ID NOs: 49-54, and has at least 99% sequence identity to the at least 100 consecutive amino acid residues of any one of SEQ ID NOs: 49-54. In some embodiments, the VL comprises an amino acid sequence of at least 105 consecutive amino acid residues of any one of SEQ ID NOs: 49-54, and has at least 99% sequence identity to the at least 105 consecutive amino acid residues of any one of SEQ ID NOs: 49-54.

In some embodiments, the VH comprises an amino acid sequence that has at least 70% sequence identity to the amino acid sequence according to any one of SEQ ID NOs: 44-48; and the VL comprises an amino acid sequence that has at least 70% sequence identity to the amino acid sequence according to any one of SEQ ID NOs: 49-54. In some embodiments, the VH comprises an amino acid sequence that has at least 80% sequence identity to the amino acid sequence according to any one of SEQ ID NOs: 44-48; and the VL comprises an amino acid sequence that has at least 80% sequence identity to the amino acid sequence according to any one of SEQ ID NOs: 49-54. In some embodiments, the VH comprises an amino acid sequence that has at least 85% sequence identity to the amino acid sequence according to any one of SEQ ID NOs: 44-48; and the VL comprises an amino acid sequence that has at least 85% sequence identity to the amino acid sequence according to any one of SEQ ID NOs: 49-54. In some embodiments, the VH comprises an amino acid sequence that has at least 90% sequence identity to the amino acid sequence according to any one of SEQ ID NOs: 44-48; and the VL comprises an amino acid sequence that has at least 90% sequence identity to the amino acid sequence according to any one of SEQ ID NOs: 49-54. In some embodiments, the VH comprises an amino acid sequence that has at least 91% sequence identity to the amino acid sequence according to any one of SEQ ID NOs: 44-48; and the VL comprises an amino acid sequence that has at least 91% sequence identity to the amino acid sequence according to any one of SEQ ID NOs: 49-54. In some embodiments, the VH comprises an amino acid sequence that has at least 92% sequence identity to the amino acid sequence according to any one of SEQ ID NOs: 44-48; and the VL comprises an amino acid sequence that has at least 92% sequence identity to the amino acid sequence according to any one of SEQ ID NOs: 49-54. In some embodiments, the VH comprises an amino acid sequence that has at least 93% sequence identity to the amino acid sequence according to any one of SEQ ID NOs: 44-48; and the VL comprises an amino acid sequence that has at least 93% sequence identity to the amino acid sequence according to any one of SEQ ID NOs: 49-54. In some embodiments, the VH comprises an amino acid sequence that has at least 94% sequence identity to the amino acid sequence according to any one of SEQ ID NOs: 44-48; and the VL comprises an amino acid sequence that has at least 94% sequence identity to the amino acid sequence according to any one of SEQ ID NOs: 49-54. In some embodiments, the VH comprises an amino acid sequence that has at least 95% sequence identity to the amino acid sequence according to any one of SEQ ID NOs: 44-48; and the VL comprises an amino acid sequence that has at least 95% sequence identity to the amino acid sequence according to any one of SEQ ID NOs: 49-54. In some embodiments, the VH comprises an amino acid sequence that has at least 96% sequence identity to the amino acid sequence according to any one of SEQ ID NOs: 44-48; and the VL comprises an amino acid sequence that has at least 96% sequence identity to the amino acid sequence according to any one of SEQ ID NOs: 49-54. In some embodiments, the VH comprises an amino acid sequence that has at least 97% sequence identity to the amino acid sequence according to any one of SEQ ID NOs: 44-48; and the VL comprises an amino acid sequence that has at least 97% sequence identity to the amino acid sequence according to any one of SEQ ID NOs: 49-54. In some embodiments, the VH comprises an amino acid sequence that has at least 98% sequence identity to the amino acid sequence according to any one of SEQ ID NOs: 44-48; and the VL comprises an amino acid sequence that has at least 98% sequence identity to the amino acid sequence according to any one of SEQ ID NOs: 49-54. In some embodiments, the VH comprises an amino acid sequence that has at least 99% sequence identity to the amino acid sequence according to any one of SEQ ID NOs: 44-48; and the VL comprises an amino acid sequence that has at least 99% sequence identity to the amino acid sequence according to any one of SEQ ID NOs: 49-54.

In some embodiments, the VH comprises an amino acid sequence that has at least 70% sequence identity to the amino acid sequence according to SEQ ID NO: 44; and the VL comprises an amino acid sequence that has at least 70% sequence identity to the amino acid sequence according to SEQ ID NO: 35. In some embodiments, the VH comprises an amino acid sequence that has at least 80% sequence identity to the amino acid sequence according to SEQ ID NO: 44; and the VL comprises an amino acid sequence that has at least 80% sequence identity to the amino acid sequence according to SEQ ID NO: 49. In some embodiments, the VH comprises an amino acid sequence that has at least 85% sequence identity to the amino acid sequence according to SEQ ID NO: 44; and the VL comprises an amino acid sequence that has at least 85% sequence identity to the amino acid sequence according to SEQ ID NO: 49. In some embodiments, the VH comprises an amino acid sequence that has at least 90% sequence identity to the amino acid sequence according to SEQ ID NO: 44; and the VL comprises an amino acid sequence that has at least 90% sequence identity to the amino acid sequence according to SEQ ID NO: 49. In some embodiments, the VH comprises an amino acid sequence that has at least 91% sequence identity to the amino acid sequence according to SEQ ID NO: 44; and the VL comprises an amino acid sequence that has at least 91% sequence identity to the amino acid sequence according to SEQ ID NO: 49. In some embodiments, the VH comprises an amino acid sequence that has at least 92% sequence identity to the amino acid sequence according to SEQ ID NO: 44; and the VL comprises an amino acid sequence that has at least 92% sequence identity to the amino acid sequence according to SEQ ID NO: 49. In some embodiments, the VH comprises an amino acid sequence that has at least 93% sequence identity to the amino acid sequence according to SEQ ID NO: 44; and the VL comprises an amino acid sequence that has at least 93% sequence identity to the amino acid sequence according to SEQ ID NO: 49. In some embodiments, the VH comprises an amino acid sequence that has at least 94% sequence identity to the amino acid sequence according to SEQ ID NO: 44; and the VL comprises an amino acid sequence that has at least 94% sequence identity to the amino acid sequence according to SEQ ID NO: 49. In some embodiments, the VH comprises an amino acid sequence that has at least 95% sequence identity to the amino acid sequence according to SEQ ID NO: 44; and the VL comprises an amino acid sequence that has at least 95% sequence identity to the amino acid sequence according to SEQ ID NO: 49. In some embodiments, the VH comprises an amino acid sequence that has at least 96% sequence identity to the amino acid sequence according to SEQ ID NO: 44; and the VL comprises an amino acid sequence that has at least 96% sequence identity to the amino acid sequence according to SEQ ID NO: 49. In some embodiments, the VH comprises an amino acid sequence that has at least 97% sequence identity to the amino acid sequence according to SEQ ID NO: 44; and the VL comprises an amino acid sequence that has at least 97% sequence identity to the amino acid sequence according to SEQ ID NO: 49. In some embodiments, the VH comprises an amino acid sequence that has at least 98% sequence identity to the amino acid sequence according to SEQ ID NO: 44; and the VL comprises an amino acid sequence that has at least 98% sequence identity to the amino acid sequence according to SEQ ID NO: 49. In some embodiments, the VH comprises an amino acid sequence that has at least 99% sequence identity to the amino acid sequence according to SEQ ID NO: 44; and the VL comprises an amino acid sequence that has at least 99% sequence identity to the amino acid sequence according to SEQ ID NO: 49.

In some embodiments, the VH comprises an amino acid sequence that has at least 70% sequence identity to the amino acid sequence according to SEQ ID NO: 45; and the VL comprises an amino acid sequence that has at least 70% sequence identity to the amino acid sequence according to SEQ ID NO: 50. In some embodiments, the VH comprises an amino acid sequence that has at least 80% sequence identity to the amino acid sequence according to SEQ ID NO: 45; and the VL comprises an amino acid sequence that has at least 80% sequence identity to the amino acid sequence according to SEQ ID NO: 50. In some embodiments, the VH comprises an amino acid sequence that has at least 85% sequence identity to the amino acid sequence according to SEQ ID NO: 45; and the VL comprises an amino acid sequence that has at least 85% sequence identity to the amino acid sequence according to SEQ ID NO: 50. In some embodiments, the VH comprises an amino acid sequence that has at least 90% sequence identity to the amino acid sequence according to SEQ ID NO: 45; and the VL comprises an amino acid sequence that has at least 90% sequence identity to the amino acid sequence according to SEQ ID NO: 50. In some embodiments, the VH comprises an amino acid sequence that has at least 91% sequence identity to the amino acid sequence according to SEQ ID NO: 45; and the VL comprises an amino acid sequence that has at least 91% sequence identity to the amino acid sequence according to SEQ ID NO: 50. In some embodiments, the VH comprises an amino acid sequence that has at least 92% sequence identity to the amino acid sequence according to SEQ ID NO: 45; and the VL comprises an amino acid sequence that has at least 92% sequence identity to the amino acid sequence according to SEQ ID NO: 50. In some embodiments, the VH comprises an amino acid sequence that has at least 93% sequence identity to the amino acid sequence according to SEQ ID NO: 45; and the VL comprises an amino acid sequence that has at least 93% sequence identity to the amino acid sequence according to SEQ ID NO: 50. In some embodiments, the VH comprises an amino acid sequence that has at least 94% sequence identity to the amino acid sequence according to SEQ ID NO: 45; and the VL comprises an amino acid sequence that has at least 94% sequence identity to the amino acid sequence according to SEQ ID NO: 50. In some embodiments, the VH comprises an amino acid sequence that has at least 95% sequence identity to the amino acid sequence according to SEQ ID NO: 45; and the VL comprises an amino acid sequence that has at least 95% sequence identity to the amino acid sequence according to SEQ ID NO: 50. In some embodiments, the VH comprises an amino acid sequence that has at least 96% sequence identity to the amino acid sequence according to SEQ ID NO: 45; and the VL comprises an amino acid sequence that has at least 96% sequence identity to the amino acid sequence according to SEQ ID NO: 50. In some embodiments, the VH comprises an amino acid sequence that has at least 97% sequence identity to the amino acid sequence according to SEQ ID NO: 45; and the VL comprises an amino acid sequence that has at least 97% sequence identity to the amino acid sequence according to SEQ ID NO: 50. In some embodiments, the VH comprises an amino acid sequence that has at least 98% sequence identity to the amino acid sequence according to SEQ ID NO: 45; and the VL comprises an amino acid sequence that has at least 98% sequence identity to the amino acid sequence according to SEQ ID NO: 50. In some embodiments, the VH comprises an amino acid sequence that has at least 99% sequence identity to the amino acid sequence according to SEQ ID NO: 45; and the VL comprises an amino acid sequence that has at least 99% sequence identity to the amino acid sequence according to SEQ ID NO: 50.

In some embodiments, the VH comprises an amino acid sequence that has at least 70% sequence identity to the amino acid sequence according to SEQ ID NO: 45; and the VL comprises an amino acid sequence that has at least 70% sequence identity to the amino acid sequence according to SEQ ID NO: 51. In some embodiments, the VH comprises an amino acid sequence that has at least 80% sequence identity to the amino acid sequence according to SEQ ID NO: 45; and the VL comprises an amino acid sequence that has at least 80% sequence identity to the amino acid sequence according to SEQ ID NO: 51. In some embodiments, the VH comprises an amino acid sequence that has at least 85% sequence identity to the amino acid sequence according to SEQ ID NO: 45; and the VL comprises an amino acid sequence that has at least 85% sequence identity to the amino acid sequence according to SEQ ID NO: 51. In some embodiments, the VH comprises an amino acid sequence that has at least 90% sequence identity to the amino acid sequence according to SEQ ID NO: 45; and the VL comprises an amino acid sequence that has at least 90% sequence identity to the amino acid sequence according to SEQ ID NO: 51. In some embodiments, the VH comprises an amino acid sequence that has at least 91% sequence identity to the amino acid sequence according to SEQ ID NO: 45; and the VL comprises an amino acid sequence that has at least 91% sequence identity to the amino acid sequence according to SEQ ID NO: 51. In some embodiments, the VH comprises an amino acid sequence that has at least 92% sequence identity to the amino acid sequence according to SEQ ID NO: 45; and the VL comprises an amino acid sequence that has at least 92% sequence identity to the amino acid sequence according to SEQ ID NO: 51. In some embodiments, the VH comprises an amino acid sequence that has at least 93% sequence identity to the amino acid sequence according to SEQ ID NO: 45; and the VL comprises an amino acid sequence that has at least 93% sequence identity to the amino acid sequence according to SEQ ID NO: 51. In some embodiments, the VH comprises an amino acid sequence that has at least 94% sequence identity to the amino acid sequence according to SEQ ID NO: 45; and the VL comprises an amino acid sequence that has at least 94% sequence identity to the amino acid sequence according to SEQ ID NO: 51. In some embodiments, the VH comprises an amino acid sequence that has at least 95% sequence identity to the amino acid sequence according to SEQ ID NO: 45; and the VL comprises an amino acid sequence that has at least 95% sequence identity to the amino acid sequence according to SEQ ID NO: 51. In some embodiments, the VH comprises an amino acid sequence that has at least 96% sequence identity to the amino acid sequence according to SEQ ID NO: 45; and the VL comprises an amino acid sequence that has at least 96% sequence identity to the amino acid sequence according to SEQ ID NO: 51. In some embodiments, the VH comprises an amino acid sequence that has at least 97% sequence identity to the amino acid sequence according to SEQ ID NO: 45; and the VL comprises an amino acid sequence that has at least 97% sequence identity to the amino acid sequence according to SEQ ID NO: 51. In some embodiments, the VH comprises an amino acid sequence that has at least 98% sequence identity to the amino acid sequence according to SEQ ID NO: 45; and the VL comprises an amino acid sequence that has at least 98% sequence identity to the amino acid sequence according to SEQ ID NO: 51. In some embodiments, the VH comprises an amino acid sequence that has at least 99% sequence identity to the amino acid sequence according to SEQ ID NO: 45; and the VL comprises an amino acid sequence that has at least 99% sequence identity to the amino acid sequence according to SEQ ID NO: 51.

In some embodiments, the VH comprises an amino acid sequence that has at least 70% sequence identity to the amino acid sequence according to SEQ ID NO: 46; and the VL comprises an amino acid sequence that has at least 70% sequence identity to the amino acid sequence according to SEQ ID NO: 52. In some embodiments, the VH comprises an amino acid sequence that has at least 80% sequence identity to the amino acid sequence according to SEQ ID NO: 46; and the VL comprises an amino acid sequence that has at least 80% sequence identity to the amino acid sequence according to SEQ ID NO: 52. In some embodiments, the VH comprises an amino acid sequence that has at least 85% sequence identity to the amino acid sequence according to SEQ ID NO: 46; and the VL comprises an amino acid sequence that has at least 85% sequence identity to the amino acid sequence according to SEQ ID NO: 52. In some embodiments, the VH comprises an amino acid sequence that has at least 90% sequence identity to the amino acid sequence according to SEQ ID NO: 46; and the VL comprises an amino acid sequence that has at least 90% sequence identity to the amino acid sequence according to SEQ ID NO: 52. In some embodiments, the VH comprises an amino acid sequence that has at least 91% sequence identity to the amino acid sequence according to SEQ ID NO: 46; and the VL comprises an amino acid sequence that has at least 91% sequence identity to the amino acid sequence according to SEQ ID NO: 52. In some embodiments, the VH comprises an amino acid sequence that has at least 92% sequence identity to the amino acid sequence according to SEQ ID NO: 46; and the VL comprises an amino acid sequence that has at least 92% sequence identity to the amino acid sequence according to SEQ ID NO: 52. In some embodiments, the VH comprises an amino acid sequence that has at least 93% sequence identity to the amino acid sequence according to SEQ ID NO: 46; and the VL comprises an amino acid sequence that has at least 93% sequence identity to the amino acid sequence according to SEQ ID NO: 52. In some embodiments, the VH comprises an amino acid sequence that has at least 94% sequence identity to the amino acid sequence according to SEQ ID NO: 46; and the VL comprises an amino acid sequence that has at least 94% sequence identity to the amino acid sequence according to SEQ ID NO: 52. In some embodiments, the VH comprises an amino acid sequence that has at least 95% sequence identity to the amino acid sequence according to SEQ ID NO: 46; and the VL comprises an amino acid sequence that has at least 95% sequence identity to the amino acid sequence according to SEQ ID NO: 52. In some embodiments, the VH comprises an amino acid sequence that has at least 96% sequence identity to the amino acid sequence according to SEQ ID NO: 46; and the VL comprises an amino acid sequence that has at least 96% sequence identity to the amino acid sequence according to SEQ ID NO: 52. In some embodiments, the VH comprises an amino acid sequence that has at least 97% sequence identity to the amino acid sequence according to SEQ ID NO: 46; and the VL comprises an amino acid sequence that has at least 97% sequence identity to the amino acid sequence according to SEQ ID NO: 52. In some embodiments, the VH comprises an amino acid sequence that has at least 98% sequence identity to the amino acid sequence according to SEQ ID NO: 46; and the VL comprises an amino acid sequence that has at least 98% sequence identity to the amino acid sequence according to SEQ ID NO: 52. In some embodiments, the VH comprises an amino acid sequence that has at least 99% sequence identity to the amino acid sequence according to SEQ ID NO: 46; and the VL comprises an amino acid sequence that has at least 99% sequence identity to the amino acid sequence according to SEQ ID NO: 52.

In some embodiments, the VH comprises an amino acid sequence that has at least 70% sequence identity to the amino acid sequence according to SEQ ID NO: 47; and the VL comprises an amino acid sequence that has at least 70% sequence identity to the amino acid sequence according to SEQ ID NO: 53. In some embodiments, the VH comprises an amino acid sequence that has at least 80% sequence identity to the amino acid sequence according to SEQ ID NO: 47; and the VL comprises an amino acid sequence that has at least 80% sequence identity to the amino acid sequence according to SEQ ID NO: 53. In some embodiments, the VH comprises an amino acid sequence that has at least 85% sequence identity to the amino acid sequence according to SEQ ID NO: 47; and the VL comprises an amino acid sequence that has at least 85% sequence identity to the amino acid sequence according to SEQ ID NO: 53. In some embodiments, the VH comprises an amino acid sequence that has at least 90% sequence identity to the amino acid sequence according to SEQ ID NO: 47; and the VL comprises an amino acid sequence that has at least 90% sequence identity to the amino acid sequence according to SEQ ID NO: 53. In some embodiments, the VH comprises an amino acid sequence that has at least 91% sequence identity to the amino acid sequence according to SEQ ID NO: 47; and the VL comprises an amino acid sequence that has at least 91% sequence identity to the amino acid sequence according to SEQ ID NO: 53. In some embodiments, the VH comprises an amino acid sequence that has at least 92% sequence identity to the amino acid sequence according to SEQ ID NO: 47; and the VL comprises an amino acid sequence that has at least 92% sequence identity to the amino acid sequence according to SEQ ID NO: 53. In some embodiments, the VH comprises an amino acid sequence that has at least 93% sequence identity to the amino acid sequence according to SEQ ID NO: 47; and the VL comprises an amino acid sequence that has at least 93% sequence identity to the amino acid sequence according to SEQ ID NO: 53. In some embodiments, the VH comprises an amino acid sequence that has at least 94% sequence identity to the amino acid sequence according to SEQ ID NO: 47; and the VL comprises an amino acid sequence that has at least 94% sequence identity to the amino acid sequence according to SEQ ID NO: 53. In some embodiments, the VH comprises an amino acid sequence that has at least 95% sequence identity to the amino acid sequence according to SEQ ID NO: 47; and the VL comprises an amino acid sequence that has at least 95% sequence identity to the amino acid sequence according to SEQ ID NO: 53. In some embodiments, the VH comprises an amino acid sequence that has at least 96% sequence identity to the amino acid sequence according to SEQ ID NO: 47; and the VL comprises an amino acid sequence that has at least 96% sequence identity to the amino acid sequence according to SEQ ID NO: 53. In some embodiments, the VH comprises an amino acid sequence that has at least 97% sequence identity to the amino acid sequence according to SEQ ID NO: 47; and the VL comprises an amino acid sequence that has at least 97% sequence identity to the amino acid sequence according to SEQ ID NO: 53. In some embodiments, the VH comprises an amino acid sequence that has at least 98% sequence identity to the amino acid sequence according to SEQ ID NO: 47; and the VL comprises an amino acid sequence that has at least 98% sequence identity to the amino acid sequence according to SEQ ID NO: 53. In some embodiments, the VH comprises an amino acid sequence that has at least 99% sequence identity to the amino acid sequence according to SEQ ID NO: 47; and the VL comprises an amino acid sequence that has at least 99% sequence identity to the amino acid sequence according to SEQ ID NO: 53.

In some embodiments, the VH comprises an amino acid sequence that has at least 70% sequence identity to the amino acid sequence according to SEQ ID NO: 48; and the VL comprises an amino acid sequence that has at least 70% sequence identity to the amino acid sequence according to SEQ ID NO: 54. In some embodiments, the VH comprises an amino acid sequence that has at least 80% sequence identity to the amino acid sequence according to SEQ ID NO: 48; and the VL comprises an amino acid sequence that has at least 80% sequence identity to the amino acid sequence according to SEQ ID NO: 54. In some embodiments, the VH comprises an amino acid sequence that has at least 85% sequence identity to the amino acid sequence according to SEQ ID NO: 48; and the VL comprises an amino acid sequence that has at least 85% sequence identity to the amino acid sequence according to SEQ ID NO: 54. In some embodiments, the VH comprises an amino acid sequence that has at least 90% sequence identity to the amino acid sequence according to SEQ ID NO: 48; and the VL comprises an amino acid sequence that has at least 90% sequence identity to the amino acid sequence according to SEQ ID NO: 54. In some embodiments, the VH comprises an amino acid sequence that has at least 91% sequence identity to the amino acid sequence according to SEQ ID NO: 48; and the VL comprises an amino acid sequence that has at least 91% sequence identity to the amino acid sequence according to SEQ ID NO: 54. In some embodiments, the VH comprises an amino acid sequence that has at least 92% sequence identity to the amino acid sequence according to SEQ ID NO: 48; and the VL comprises an amino acid sequence that has at least 92% sequence identity to the amino acid sequence according to SEQ ID NO: 54. In some embodiments, the VH comprises an amino acid sequence that has at least 93% sequence identity to the amino acid sequence according to SEQ ID NO: 48; and the VL comprises an amino acid sequence that has at least 93% sequence identity to the amino acid sequence according to SEQ ID NO: 54. In some embodiments, the VH comprises an amino acid sequence that has at least 94% sequence identity to the amino acid sequence according to SEQ ID NO: 48; and the VL comprises an amino acid sequence that has at least 94% sequence identity to the amino acid sequence according to SEQ ID NO: 54. In some embodiments, the VH comprises an amino acid sequence that has at least 95% sequence identity to the amino acid sequence according to SEQ ID NO: 48; and the VL comprises an amino acid sequence that has at least 95% sequence identity to the amino acid sequence according to SEQ ID NO: 54. In some embodiments, the VH comprises an amino acid sequence that has at least 96% sequence identity to the amino acid sequence according to SEQ ID NO: 48; and the VL comprises an amino acid sequence that has at least 96% sequence identity to the amino acid sequence according to SEQ ID NO: 54. In some embodiments, the VH comprises an amino acid sequence that has at least 97% sequence identity to the amino acid sequence according to SEQ ID NO: 48; and the VL comprises an amino acid sequence that has at least 97% sequence identity to the amino acid sequence according to SEQ ID NO: 54. In some embodiments, the VH comprises an amino acid sequence that has at least 98% sequence identity to the amino acid sequence according to SEQ ID NO: 48; and the VL comprises an amino acid sequence that has at least 98% sequence identity to the amino acid sequence according to SEQ ID NO: 54. In some embodiments, the VH comprises an amino acid sequence that has at least 99% sequence identity to the amino acid sequence according to SEQ ID NO: 48; and the VL comprises an amino acid sequence that has at least 99% sequence identity to the amino acid sequence according to SEQ ID NO: 54.

Described herein, in some embodiments, are antibodies or antibody fragments comprising a heavy chain sequence at least about 90% identical to a sequence as set forth in any one of SEQ ID NOs: 55, 57, 60, 62, and 64. In some instances, the antibodies or antibody fragments comprise a heavy chain sequence at least or about 70%, 80%, 85%, 90%, 91%, 92%, 93%, 94%, 95%, 96%, 97%, 98%, 99%, or 100% sequence identity to any one of SEQ ID NOs: 55, 57, 60, 62, and 64.

Described herein, in some embodiments, are antibodies or antibody fragments comprising a light chain sequence at least about 90% identical to a sequence as set forth in any one of SEQ ID NOs: 56, 58, 59, 61, 63, and 65. In some instances, the antibodies or antibody fragments comprise a light chain sequence at least or about 70%, 80%, 85%, 90%, 91%, 92%, 93%, 94%, 95%, 96%, 97%, 98%, 99%, or 100% sequence identity to any one of SEQ ID NOs: 56, 58, 59, 61, 63, and 65.

Described herein, in some embodiments, are antibodies or antibody fragments comprising a heavy chain sequence at least about 90% identical to a sequence as set forth in any one of SEQ ID NOs: 55, 57, 60, 62, and 64 and a light chain sequence at least about 90% identical to a sequence as set forth in any one of SEQ ID NOs: 56, 58, 59, 61, 63, and 65. In some instances, the antibodies or antibody fragments comprise a heavy chain sequence at least or about 70%, 80%, 85%, 90%, 91%, 92%, 93%, 94%, 95%, 96%, 97%, 98%, 99%, or 100% sequence identity to any one of SEQ ID NOs: 55, 57, 60, 62, and 64 and a light chain sequence at least or about 70%, 80%, 85%, 90%, 91%, 92%, 93%, 94%, 95%, 96%, 97%, 98%, 99%, or 100% sequence identity to any one of SEQ ID NOs: 56, 58, 59, 61, 63, and 65. Table 5A lists heavy chain and light chain sequences for antibodies described herein. Table 5B lists the antibody name and the pairing of a heavy chain with a light chain to form an antibody described herein.

Table 5A. Heavy Chain and Light Chain Sequences

| | SEQ ID | |
|---|---|---|
| Name | NO: | Amino Acid Sequence |
| Antibody 1 | 55 | METGLRWLLLVAVLKGVQCQSLEESGGRLVTPGTPLTLTCTVSGFSLSSQKV |
| Heavy | | GWVRQAPGKGLEWIGIINNYGSTYYASWAKGRFTISKTSTTVDLRITSLTAED |
| Chain | | TATYFCARDPDGSIVFDIWGPGTLVTVSLGQPKAPSVFPLAPCCGDTPSSTV |
| | | TLGCLVKGYLPEPVTVTWNSGTLTNGVRTFPSVRQSSGLYSLSSVVSVTSSS |
| | | QPVTCNVAHPATNTKVDKTVAPSTCSKPTCPPPELLGRSSVFIFPPKPKDTL |
| | | MISRTPEVTCVVVDVSQDDPEVQFTWYINNEQVRTARPPLREQQFNSTIRWV |
| | | STLPIAHQDWLRGKEFKCKVHNKALPAPIEKTISKARGQPLEPKVYTMGPPR |
| | | EELSSRSVSLTCMINGFYPSDISVEWEKNGKAEDNYKTTPAVLDSDGSYFLY |
| | | SKLSVPTSEWQRGDVFTCSVMHEALHNHYTQKSISRSPGK |
| Antibody 1 | 56 | MDTRAPTQLLGLLLLWLPGATFAQVLTQTASPVSAAVGGTVTINCQSSQSVV |
| Light Chain | | YNNRLSWFQQKPGQPPKLLIYGASTLASGVPSRFKGSGSGTQFTLTISDVQC |
| | | DDAATYYCLGSYDCSSGDCHAFGGGTEVVVKGDPVAPTVLIFPPAADQVAT |
| | | GTVTIVCVANKYFPDVTVTWEVDGTTQTTGIENSKTPQNSADCTYNLSSTLTL |
| | | TSTQYNSHKEYTCKVTQGTTSWQSFNRGDC |
| Antibody 2 | 57 | METGLRWLLLVAVLKGVQCQSVEESGGRLVTPGTPLTLTCTVSGFSLSSYA |
| and | | MIWVRQAPGKGLEWIGFISRSGITYYASWAKGRFTISKTSTTVDLKMTSLTTE |
| Antibody 3 | | DTATYFCAREFGAVGSDYYRDAFNLWGPGTLVTVSSGQPKAPSVFPLAPCC |
| Heavy | | GDTPSSTVTLGCLVKGYLPEPVTVTWNSGTLTNGVRTFPSVRQSSGLYSLSS |
| Chain | | VVSVTSSSQPVTCNVAHPATNTKVDKTVAPSTCSKPTCPPPELLGRSSVFIFP |
| | | PKPKDTLMISRTPEVTCVVVDVSQDDPEVQFTWYINNEQVRTARPPLREQQF |
| | | NSTIRVVSTLPIAHQDWLRGKEFKCKVHNKALPAPIEKTISKARGQPLEPKVY |
| | | TMGPPREELSSRSVSLTCMINGFYPSDISVEWEKNGKAEDNYKTTPAVLDSD |
| | | GSYFLYSKLSVPTSEWQRGDVFTCSVMHEALHNHYTQKSISRSPGK |
| Antibody 2 | 58 | MDMRAPTQLLGLLLLWLPGARCADIVMTQTPASVEAAVGGTVTINCQASESI |
| Light Chain | | NSWLSWYQQKPGQPPNLLIYRASTLASGVPSRFSGGGSGTEYTLTISDLECA |
| | | DAVTYYCQSYYEEDGIGYAFGGGTEVVVEGDPVAPTVLIFPPAADQVATGTV |
| | | TIVCVANKYFPDVTVTWEVDGTTQTTGIENSKTPQNSADCTYNLSSTLTLTST |
| | | QYNSHKEYTCKVTQGTTSVVQSFNRGDC |
| Antibody 3 | 59 | MDMRAPTQLLGLLLLWLPGARCADIVMTQTPSSVSAAVGGTVTINCQASQNI |
| Light Chain | | YSNLAWYQQKPGQRPRLLIYGASNLASGVPSRFKGSRSGTEFTLTISDLECA |
| | | DAATYYCQGYDYSTAGAYPFGGGTAVWVKGDPVAPTVLIFPPAADQVATGT |
| | | VTIVCVANKYFPDVTVTWEVDGTTQTTGIENSKTPQNSADCTYNLSSTLTLTS |
| | | TQYNSHKEYTCKVTQGTTSVVQSFNRGDC |
| Antibody 4 | 60 | METGLRWLLLVAVLKGVQCQSLEESGGRLVTPGTPLTLTCTVSGFSLNNYKV |
| Heavy | | GWVRQAPGKGLEWIGIINYYSQTYYASWAKGRFTISKTSTTVDLKLTSPTTED |
| Chain | | TATYFCARDPDGSIVFDIWGPGTLVTVSLGQPKAPSVFPLAPCCGDTPSSTV |
| | | TLGCLVKGYLPEPVTVTWNSGTLTNGVRTFPSVRQSSGLYSLSSVVSVTSSS |
| | | QPVTCNVAHPATNTKVDKTVVPSTCSKPTCPPPELLGRSSVFIFPPKPKDTL |
| | | MISRTPEVTCVVVDVSQDDPEVQFTWYINNEQVRTARPPLREQQFNSTIRW |
| | | STLPIAHQDWLRGKEFKCKVHNKALPAPIEKTISKARGQPLEPKVYTMGPPR |
| | | EELSSRSVSLTCMINGFYPSDISVEWEKNGKAEDNYKTTPAVLDSDGSYFLY |
| | | SKLSVPTSEWQRGDVFTCSVMHEALHNHYTQKSISRSPGK |
| Antibody 4 | 61 | MDTRAPTQLLGLLLLWLPGATFAQVLTQTASPVSAAVGSTVTINCQSSQSVY |
| Light Chain | | SNKRLAWFQLKPGQPPKLLIYGASTLASGVPSRFKGSGSGTQFTLTISDVQC |
| | | DDAATYYCAGGYDCSTGDCWTFGGGTEVVVTGDPVAPTVLIFPPAADQVAT |
| | | GTVTIVCVANKYFPDVTVTWEVDGTTQTTGIENSKTPQNSADCTYNLSSTLTL |
| | | TSTQYNSHKEYTCKVTQGTTSWQSFNRGDC |
| Antibody 5 | 62 | METGLRWLLLVAVLKGVQCQSVEESGGGLVTPGGTLTLTCTVSGFSLSNYA |
| Heavy | | MSWVRQAPGKGLEWIGFISRSGITYYASWAKGRFTISKTSTTVDLKITSPTTE |
| Chain | | DTAAYFCAREFGAVGSDYYRDALRLWGPGTLVTVSSGQPKAPSVFPLAPCC |
| | | GDTPSSTVTLGCLVKGYLPEPVTVTWNSGTLTNGVRTFPSVRQSSGLYSLSS |
| | | VVSVTSSSQPVTCNVAHPATNTKVDKTVAPSTCSKPTCPPPELLGRSSVFIFP |
| | | PKPKDTLMISRTPEVTCVVVDVSQDDPEVQFTWYINNEQVRTARPPLREQQF |
| | | NSTIRVVSTLPIAHQDWLRGKEFKCKVHNKALPAPIEKTISKARGQPLEPKVY |
| | | TMGPPREELSSRSVSLTCMINGFYPSDISVEWEKNGKAEDNYKTTPAVLDSD |
| | | GSYFLYSKLSVPTSEWQRGDVFTCSVMHEALHNHYTQKSISRSPGK |
| Antibody 5 | 63 | MDMRAPTQLLGLLLLWLPGARCADIVMTQTPSSVSAAVGGTVTIKCQASQSI |
| Light Chain | | GSNLAWYQQKPGQPPKLLIYGASTLESGVPSRFKGSGSGTEYTLTISDLECA |
| | | DAATYYCQSYYEGSDIGYAFGGGTEVVVEGDPVAPTVLIFPPAADQVATGTV |
| | | TIVCVANKYFPDVTVTWEVDGTTQTTGIENSKTPQNSADCTYNLSSTLTLTST |
| | | QYNSHKEYTCKVTQGTTSVVQSFNRGDC |
| Antibody 6 | 64 | METGLRWLLLVAVLKGVQCQSLEESGGRLVTPGTPLTLTCTVSGIDLSTHAM |
| Heavy | | TWVRQAPGKGLEWIGVINPSGSAYYATWVNGRFTISKTSTTVDLKITSPTTG |
| Chain | | DTAKYFCARDYITAGDYYMDAFDPWGPGTLVTVSSGQPKAPSVFPLAPCCG |
| | | DTPSSTVTLGCLVKGYLPEPVTVTWNSGTLTNGVRTFPSVRQSSGLYSLSSV |
| | | VSVTSSSQPVTCNVAHPATNTKVDKTVAPSTCSKPTCPPPELLGRSSVFIFPP |
| | | KPKDTLMISRTPEVTCVVVDVSQDDPEVQFTWYINNEQVRTARPPLREQQF |
| | | NSTIRVVSTLPIAHQDWLRGKEFKCKVHNKALPAPIEKTISKARGQPLEPKVY |
| | | TMGPPREELSSRSVSLTCMINGFYPSDISVEWEKNGKAEDNYKTTPAVLDSD |
| | | GSYFLYSKLSVPTSEWQRGDVFTCSVMHEALHNHYTQKSISRSPGK |
| Antibody 6 | 65 | MDTRAPTQLLGLLLLWLPGARCADIVMTQTPASVSAAVGGTVTIKCQASQSIS |
| Light Chain | | NQLSWYQQKSGQPPKLLIYRASTLASGVPSRFKGSGSGTEFTLTISDLECAD |
| | | AATYYCQQGYNRDNVDNLFGGGTEVWVKGDPVAPTVLIFPPAADQVATGTV |
| | | TIVCVANKYFPDVTVTWEVDGTTQTTGIENSKTPQNSADCTYNLSSTLTLTST |
| | | QYNSHKEYTCKVTQGTTSVVQSFNRGDC |

TABLE 5B
Antibody names and pairing of HC and LC

| Antibody Name | Paired HC and LC identified by SEQ ID NO: |
|---|---|
| Antibody 1 | SEQ ID NO: 55 and SEQ ID NO: 56 |
| Antibody 2 | SEQ ID NO: 57 and SEQ ID NO: 58 |
| Antibody 3 | SEQ ID NO: 57 and SEQ ID NO: 59 |
| Antibody 4 | SEQ ID NO: 60 and SEQ ID NO: 61 |
| Antibody 5 | SEQ ID NO: 62 and SEQ ID NO: 63 |
| Antibody 6 | SEQ ID NO: 64 and SEQ ID NO: 65 |

In some embodiments, the anti-tau antibody comprises a VH domain that is encoded by a nucleic acid comprising at least 80%, at least 85%, at least 90%, at least 95% sequence identity to a sequence selected from SEQ ID NOs: 66-70. In some embodiments, the anti-tau antibody comprises a VL domain that is encoded by a nucleic acid comprising at least 80%, at least 85%, at least 90%, at least 95% sequence identity to a sequence selected from SEQ ID NOs: 71-76. Nucleic acid sequences for VH domains for anti-tau-tau antibodies described here are listed in Table 6 and nucleic acid sequences for VL domains for anti-tau-tau antibodies described here are listed in Table 7. In some embodiments, the anti-tau-tau antibody comprises a VH domain that is encoded by a nucleic acid comprising at least 90% sequence identity to SEQ ID NO: 66. In some embodiments, the anti-tau-tau antibody comprises a VH domain that is encoded by a nucleic acid comprising at least 90% sequence identity to SEQ ID NO: 67. In some embodiments, the anti-tau-tau antibody comprises a VH domain that is encoded by a nucleic acid comprising at least 90% sequence identity to SEQ ID NO: 68. In some embodiments, the anti-tau-tau antibody comprises a VH domain that is encoded by a nucleic acid comprising at least 90% sequence identity to SEQ ID NO: 69. In some embodiments, the anti-tau-tau antibody comprises a VH domain that is encoded by a nucleic acid comprising at least 90% sequence identity to SEQ ID NO: 70. In some embodiments, the anti-tau-tau antibody comprises a VL domain that is encoded by a nucleic acid comprising at least 90% sequence identity to SEQ ID NO: 71. In some embodiments, the anti-tau-tau antibody comprises a VL domain that is encoded by a nucleic acid comprising at least 90% sequence identity to SEQ ID NO: 72. In some embodiments, the anti-tau antibody comprises a VL domain that is encoded by a nucleic acid comprising at least 90% sequence identity to SEQ ID NO: 73. In some embodiments, the anti-tau antibody comprises a VL domain that is encoded by a nucleic acid comprising at least 90% sequence identity to SEQ ID NO: 74. In some embodiments, the anti-tau antibody comprises a VL domain that is encoded by a nucleic acid comprising at least 90% sequence identity to SEQ ID NO: 75. In some embodiments, the anti-tau antibody comprises a VL domain that is encoded by a nucleic acid comprising at least 90% sequence identity to SEQ ID NO: 76. In some embodiments, the anti-tau antibody comprises a VH domain that is encoded by a nucleic acid comprising at least 90% sequence identity to SEQ ID NO: 66 and a VL domain that is encoded by a nucleic acid comprising at least 90% sequence identity to SEQ ID NO: 71. In some embodiments, the anti-tau antibody comprises a VH domain that is encoded by a nucleic acid comprising at least 90% sequence identity to SEQ ID NO: 67 and a VL domain that is encoded by a nucleic acid comprising at least 90% sequence identity to SEQ ID NO: 72. In some embodiments, the anti-tau antibody comprises a VH domain that is encoded by a nucleic acid comprising at least 90% sequence identity to SEQ ID NO: 67 and a VL domain that is encoded by a nucleic acid comprising at least 90% sequence identity to SEQ ID NO: 73. In some embodiments, the anti-tau antibody comprises a VH domain that is encoded by a nucleic acid comprising at least 90% sequence identity to SEQ ID NO: 68 and a VL domain that is encoded by a nucleic acid comprising at least 90% sequence identity to SEQ ID NO: 74. In some embodiments, the anti-tau antibody comprises a VH domain that is encoded by a nucleic acid comprising at least 90% sequence identity to SEQ ID NO: 69 and a VL domain that is encoded by a nucleic acid comprising at least 90% sequence identity to SEQ ID NO: 75. In some embodiments, the anti-tau antibody comprises a VH domain that is encoded by a nucleic acid comprising at least 90% sequence identity to SEQ ID NO: 70 and a VL domain that is encoded by a nucleic acid comprising at least 90% sequence identity to SEQ ID NO: 76. In some embodiments, the anti-tau antibody comprises a VH domain that is encoded by a nucleic acid comprising a sequence identical to SEQ ID NO: 66. In some embodiments, the anti-tau antibody comprises a VH domain that is encoded by a nucleic acid comprising a sequence identical to SEQ ID NO: 67. In some embodiments, the anti-tau antibody comprises a VH domain that is encoded by a nucleic acid comprising a sequence identical to SEQ ID NO: 68. In some embodiments, the anti-tau antibody comprises a VH domain that is encoded by a nucleic acid comprising a sequence identical to SEQ ID NO: 69. In some embodiments, the anti-tau antibody comprises a VH domain that is encoded by a nucleic acid comprising a sequence identical to SEQ ID NO: 70. In some embodiments, the anti-tau antibody comprises a VL domain that is encoded by a nucleic acid comprising a sequence identical to SEQ ID NO: 71. In some embodiments, the anti-tau antibody comprises a VL domain that is encoded by a nucleic acid comprising a sequence identical to SEQ ID NO: 72. In some embodiments, the anti-tau antibody comprises a VL domain that is encoded by a nucleic acid comprising a sequence identical to SEQ ID NO: 73. In some embodiments, the anti-tau antibody comprises a VL domain that is encoded by a nucleic acid comprising a sequence identical to SEQ ID NO: 74. In some embodiments, the anti-tau antibody comprises a VL domain that is encoded by a nucleic acid comprising a sequence identical to SEQ ID NO: 75. In some embodiments, the anti-tau antibody comprises a VL domain that is encoded by a nucleic acid comprising a sequence identical to SEQ ID NO: 76. In some embodiments, the anti-tau antibody comprises a VH domain that is encoded by a nucleic acid comprising a sequence identical to SEQ ID NO: 66 and a VL domain that is encoded by a nucleic acid comprising a sequence identical to SEQ ID NO: 71. In some embodiments, the anti-tau antibody comprises a VH domain that is encoded by a nucleic acid comprising a sequence identical to SEQ ID NO: 67 and a VL domain that is encoded by a nucleic acid comprising a sequence identical to SEQ ID NO: 72. In some embodiments, the anti-tau antibody comprises a VH domain that is encoded by a nucleic acid comprising a sequence identical to SEQ ID NO: 67 and a VL domain that is encoded by a nucleic acid comprising a sequence identical to SEQ ID NO: 73. In some embodiments, the anti-tau antibody comprises a VH domain that is encoded by a nucleic acid comprising a sequence identical to SEQ ID NO: 68 and a VL domain that is encoded by a nucleic acid comprising a sequence identical to SEQ ID NO: 74. In some embodiments, the anti-tau antibody comprises a VH domain that is encoded by a nucleic acid comprising a sequence identical to SEQ ID NO: 69 and a VL domain that is encoded by a nucleic acid comprising a sequence identical to SEQ ID NO: 75. In some embodiments, the anti-tau antibody comprises a VH domain that is encoded by a nucleic acid comprising a sequence identical to SEQ ID NO: 70 and a VL domain that is encoded by a nucleic acid comprising a sequence identical to SEQ ID NO: 76.

TABLE 6
Nucleic acid sequences encoding VH domains

| | SEQ ID | |
|---|---|---|
| Name | NO: | Nucleotide Sequence |
| Antibody | 66 | ATGGAGACTGGGCTGCGCTGGCTTCTCCTGGTCGCTGTGCTCAAAGGTGTCCA |
| 1 VH | | GTGTCAGTCGCTGGAGGAGTCCGGGGGTCGCCTGGTCACGCCTGGGACACCC |
| coding | | CTGACACTCACCTGCACAGTCTCTGGATTTTCCCTCAGTAGCCAGAAAGTGGGC |
| | | TGGGTCCGCCAGGCTCCAGGGAAGGGGCTGGAATGGATCGGAATCATTAATAA |
| | | TTATGGTAGCACATACTACGCGAGCTGGGCGAAAGGCCGATTCACCATCTCGAA |
| | | AACCTCGACCACAGTGGATCTGAGAATCACCAGTCTGACGGCCGAGGACACGG |
| | | CCACCTATTTCTGTGCCCGTGATCCTGATGGTAGTATTGTCTTTGACATCTGGG |
| | | GCCCAGGCACCCTTGTCACCGTCTCCTTG |
| Antibody | 67 | ATGGAGACTGGGCTGCGCTGGCTTCTCCTGGTCGCTGTGCTCAAAGGTGTCCA |
| 2 and 3 | | GTGTCAGTCGGTGGAGGAGTCCGGGGGTCGCCTGGTCACGCCTGGGACACCC |
| VH | | CTGACACTCACCTGCACCGTCTCTGGATTCTCCCTCAGTAGCTATGCAATGATC |
| coding | | TGGGTCCGCCAGGCTCCAGGGAAGGGGCTGGAATGGATCGGATTCATTAGTCG |
| | | TAGTGGTATCACATACTACGCGAGCTGGGCAAAAGGCCGATTCACCATCTCCAA |
| | | AACCTCGACCACGGTGGATCTGAAAATGACCAGTCTGACAACCGAGGACACGG |
| | | CCACCTATTTCTGTGCCAGAGAATTCGGTGCTGTTGGTAGTGATTATTATAGGGA |
| | | CGCCTTTAACTTGTGGGGCCCAGGCACCCTGGTCACCGTCTCCTCA |
| Antibody | 68 | ATGGAGACTGGGCTGCGCTGGCTTCTCCTGGTCGCTGTGCTCAAAGGTGTCCA |
| 4 VH | | GTGTCAGTCGCTGGAGGAGTCCGGGGGTCGCCTGGTCACGCCTGGGACACCC |
| coding | | CTGACACTCACCTGCACAGTCTCTGGATTTTCCCTAAATAACTACAAAGTGGGCT |
| | | GGGTCCGCCAGGCTCCAGGAAAGGGGCTGGAATGGATCGGAATCATTAACTAT |
| | | TATAGTCAGACATACTACGCGAGCTGGGCCAAAGGCCGATTCACCATCTCGAAA |
| | | ACCTCGACCACGGTG |
| | | GATCTGAAGCTCACCAGTCCGACAACCGAAGACACGGCCACCTATTTCTGTGCC |
| | | CGTGATCCTGATGGTAGTATTGTCTTTGACATCTGGGGCCCAGGCACCCTTGTC |
| | | ACCGTCTCCTTG |
| Antibody | 69 | ATGGAGACTGGGCTGCGCTGGCTTCTCCTGGTCGCTGTGCTCAAAGGTGTCCA |
| 5 VH | | GTGTCAGTCGGTGGAGGAGTCCGGAGGAGGCCTGGTAACGCCTGGAGGAACC |
| coding | | CTGACACTCACCTGCACCGTCTCTGGATTCTCCCTCAGTAACTATGCAATGAGC |
| | | TGGGTCCGCCAGGCTCCAGGGAAGGGGCTGGAATGGATCGGATTCATTAGTCG |
| | | TAGTGGTATTACATACTACGCGAGCTGGGCAAAAGGCCGATTCACCATCTCCAA |
| | | AACCTCGACCACGGTGGATCTGAAAATCACCAGTCCGACGACCGAGGACACGG |
| | | CCGCCTATTTCTGTGCCAGAGAATTCGGTGCTGTTGGTAGTGATTATTATAGGG |
| | | ACGCCTTGAGGTTGTGGGGCCCAGGCACCCTGGTCACCGTCTCCTCA |
| Antibody | 70 | ATGGAGACTGGGCTGCGCTGGCTTCTCCTGGTCGCTGTGCTCAAAGGTGTCCA |
| 6 VH | | GTGTCAGTCGCTGGAGGAGTCCGGGGGTCGCCTGGTAACGCCTGGGACACCC |
| coding | | CTGACACTCACCTGCACAGTCTCTGGAATCGACCTCAGTACCCATGCAATGACC |
| | | TGGGTCCGCCAGGCTCCAGGAAAGGGGCTGGAATGGATCGGAGTCATTAATCC |
| | | TAGTGGTAGCGCATACTACGCGACCTGGGTGAATGGCCGATTCACCATCTCCAA |
| | | AACCTCGACCACGGTGGATCTGAAAATCACCAGTCCGACAACCGGGGACACGG |
| | | CCAAGTATTTCTGTGCCAGAGATTATATTACTGCGGGTGATTATTATATGGATGC |
| | | TTTTGATCCCTGGGGCCCAGGCACCCTGGTCACCGTCTCCTCA |

TABLE 7
Nucleic acid sequences encoding VL domains

| | SEQ ID | |
|---|---|---|
| Name | NO: | Nucleotide Sequence |
| Antibody | 71 | ATGGACACGAGGGCCCCCACTCAGCTGCTGGGGCTCCTGCTGCTCTGGC |
| 1 VL | | TCCCAGGTGCCACATTTGCCCAAGTGCTGACCCAGACTGCATCCCCCGTG |
| coding | | TCTGCGGCTGTTGGAGGCACAGTCACCATCAATTGCCAGTCCAGTCAGAG |
| | | TGTTGTATATAACAACCGCTTATCCTGGTTTCAACAGAAACCAGGGCAGCC |
| | | TCCCAAGCTCCTGATCTATGGTGCATCCACTCTGGCATCTGGGGTCCCATC |
| | | GCGGTTCAAAGGCAGTGGATCTGGGACACAGTTCACTCTCACCATCAGCG |
| | | ACGTGCAGTGTGACGATGCTGCCACTTACTACTGTCTAGGCTCCTATGATT |
| | | GTAGTAGTGGTGATTGCCATGCTTTCGGCGGAGGGACCGAGGTGGTGGTC |
| | | AAA |
| Antibody | 72 | ATGGACATGAGGGCCCCCACTCAGCTGCTGGGGCTCCTACTGCTCTGGCT |
| 2 VL | | CCCAGGTGCCAGATGTGCTGACATTGTGATGACCCAGACTCCAGCCTCCG |
| coding | | TGGAGGCAGCTGTGGGAGGCACAGTCACCATCAATTGCCAAGCCAGTGAG |
| | | AGCATTAATAGTTGGTTGTCCTGGTATCAGCAGAAACCAGGGCAGCCTCCC |
| | | AACCTCCTGATCTACAGGGCATCCACTCTGGCATCTGGGGTCCCATCGCG |
| | | GTTCAGTGGCGGTGGATCTGGGACAGAGTACACTCTCACCATCAGCGACC |
| | | TGGAGTGTGCCGATGCTGTCACTTATTACTGTCAAAGCTATTATGAGGAGG |
| | | ATGGTATTGGTTATGCTTTCGGCGGAGGGACCGAGGTGGTGGTCGAA |
| Antibody | 73 | ATGGACATGAGGGCCCCCACTCAGCTGCTGGGGCTCCTGCTGCTCTGGCTCCC |
| 3 VL | | AGGTGCCAGATGTGCTGACATTGTGATGACCCAGACTCCATCCTCCGTGTCTGC |
| coding | | AGCTGTGGGAGGCACAGTCACCATCAATTGCCAGGCCAGTCAGAACATTTACAG |
| | | CAATTTAGCCTGGTATCAGCAGAAACCAGGGCAGCGTCCCAGGCTCCTGATCTA |
| | | TGGCGCATCCAATCTGGCATCTGGGGTCCCATCGCGGTTCAAAGGCAGTAGAT |
| | | CTGGGACAGAGTTCACTCTCACCATCAGCGACCTGGAGTGTGCCGATGCTGCC |
| | | ACTTACTACTGTCAAGGCTATGATTATAGTACTGCTGGTGCCTATCCTTTCGGCG |
| | | GAGGGACCGCGGTGGTGGTCAAA |
| Antibody | 74 | ATGGACACGAGGGCCCCCACTCAGCTGCTGGGGCTCCTGCTGCTCTGGCTCCC |
| 4 VL | | AGGTGCCACATTTGCCCAAGTGCTGACCCAGACTGCATCGCCCGTGTCTGCGG |
| coding | | CTGTGGGAAGCACAGTCACCATCAATTGCCAGTCCAGTCAGAGCGTTTATAGTA |
| | | ACAAGCGCTTAGCCTGGTTTCAGCTGAAACCAGGGCAGCCTCCCAAGCTCCTG |
| | | ATCTATGGTGCATCCACACTGGCATCTGGGGTCCCATCGCGATTCAAGGGCAGT |
| | | GGATCTGGGACACAGTTCACTCTCACCATCAGCGACGTGCAGTGTGACGATGC |
| | | TGCCACTTACTACTGTGCAGGCGGTTATGATTGTAGTACTGGTGATTGTTGGAC |
| | | TTTCGGCGGAGGGACCGAGGTGGTGGTCACA |
| Antibody | 75 | ATGGACATGAGGGCCCCCACTCAGCTGCTGGGGCTCCTGCTGCTCTGGCT |
| 5 VL | | CCCAGGTGCCAGATGTGCTGACATCGTGATGACCCAGACTCCATCCTCCGT |
| coding | | GTCTGCAGCTGTGGGAGGCACAGTCACCATCAAGTGCCAGGCCAGTCAGA |
| | | GCATTGGTAGTAATTTAGCCTGGTATCAGCAGAAACCAGGGCAGCCTCCCA |
| | | AGCTCCTGATCTATGGTGCATCCACTCTGGAATCTGGGGTCCCATCGCGGT |
| | | TTAAAGGCAGTGGATCTGGGACAGAGTACACTCTCACCATCAGCGACCTG |
| | | GAGTGTGCCGATGCTGCCACTTACTACTGTCAAAGCTATTATGAGGGTAGT |
| | | GATATTGGTTATGCTTTCGGCGGAGGGACCGAGGTGGTGGTCGAA |
| Antibody | 75 | ATGGACACGAGGGCCCCCACTCAGCTGCTGGGGCTCCTGCTGCTCTGGCT |
| 6 VL | | CCCAGGTGCCAGATGTGCTGACATCGTGATGACCCAGACTCCAGCCTCTGT |
| coding | | GTCTGCAGCTGTGGGAGGCACAGTCACCATCAAGTGCCAGGCCAGTCAGA |
| | | GCATTAGCAACCAACTATCCTGGTATCAGCAGAAATCAGGGCAGCCTCCCA |
| | | AGCTCCTGATCTACAGGGCATCTACTCTGGCATCTGGGGTCCCATCGCGGT |
| | | TCAAAGGCAGTGGATCTGGGACAGAGTTCACTCTCACCATCAGCGACCTGG |
| | | AGTGTGCCGATGCTGCCACTTACTACTGTCAACAGGGTTATAATAGAGATAA |
| | | TGTTGATAATCTTTTCGGCGGAGGGACCGAGGTGGTGGTCAAA |

In some embodiments, the anti-tau antibody comprises a heavy chain that is encoded by a nucleic acid comprising a sequence identical to a sequence selected from SEQ ID NOs: 77-81. In some embodiments, the anti-tau antibody comprises a light chain that is encoded by a nucleic acid comprising a sequence identical to a sequence selected from SEQ ID NOs: 82-87. Nucleic acid sequences for heavy chains for anti-tau antibodies described here are listed in Table 8 and nucleic acid sequences for light chains for anti-tau antibodies described here are listed in Table 9. Nucleic acid sequences listed in Table 8 and Table 9 may be used in the process of in vitro production of antibodies described herein. In some embodiments, the anti-tau antibody comprises a heavy chain that is encoded by a nucleic acid comprising a sequence identical to SEQ ID NO: 77. In some embodiments, the anti-tau antibody comprises a heavy chain that is encoded by a nucleic acid comprising a sequence identical to SEQ ID NO: 78. In some embodiments, the anti-tau antibody comprises a heavy chain that is encoded by a nucleic acid comprising a sequence identical to SEQ ID NO: 79. In some embodiments, the anti-tau antibody comprises a heavy chain that is encoded by a nucleic acid comprising a sequence identical to SEQ ID NO: 80. In some embodiments, the anti-tau antibody comprises a heavy chain that is encoded by a nucleic acid comprising a sequence identical to SEQ ID NO: 81. In some embodiments, the anti-tau antibody comprises a light chain that is encoded by a nucleic acid comprising a sequence identical to SEQ ID NO: 82. In some embodiments, the anti-tau antibody comprises a light chain that is encoded by a nucleic acid comprising a sequence identical to SEQ ID NO: 83. In some embodiments, the anti-tau antibody comprises a light chain that is encoded by a nucleic acid comprising a sequence identical to SEQ ID NO: 84. In some embodiments, the anti-tau antibody comprises a light chain that is encoded by a nucleic acid comprising a sequence identical to SEQ ID NO: 85. In some embodiments, the anti-tau antibody comprises a light chain that is encoded by a nucleic acid comprising a sequence identical to SEQ ID NO: 86. In some embodiments, the anti-tau antibody comprises a light chain that is encoded by a nucleic acid comprising a sequence identical to SEQ ID NO: 87. In some embodiments, the anti-tau antibody comprises a heavy chain that is encoded by a nucleic acid comprising a sequence identical to SEQ ID NO: 77 and a light chain that is encoded by a nucleic acid comprising a sequence identical to SEQ ID NO: 82. In some embodiments, the anti-tau antibody comprises a heavy chain that is encoded by a nucleic acid comprising a sequence identical to SEQ ID NO: 78 and a light chain that is encoded by a nucleic acid comprising a sequence identical to SEQ ID NO: 83. In some embodiments, the anti-tau antibody comprises a heavy chain that is encoded by a nucleic acid comprising a sequence identical to SEQ ID NO: 78 and a light chain that is encoded by a nucleic acid comprising a sequence identical to SEQ ID NO: 84. In some embodiments, the anti-tau antibody comprises a heavy chain that is encoded by a nucleic acid comprising a sequence identical to SEQ ID NO: 79 and a light chain that is encoded by a nucleic acid comprising a sequence identical to SEQ ID NO: 85. In some embodiments, the anti-tau antibody comprises a heavy chain that is encoded by a nucleic acid comprising a sequence identical to SEQ ID NO: 80 and a light chain that is encoded by a nucleic acid comprising a sequence identical to SEQ ID NO: 86. In some embodiments, the anti-tau antibody comprises a heavy chain that is encoded by a nucleic acid comprising a sequence identical to SEQ ID NO: 81 and a light chain that is encoded by a nucleic acid comprising a sequence identical to SEQ ID NO: 87.

TABLE 8
Nucleic acid sequences encoding heavy chains

| | SEQ ID | |
|---|---|---|
| Name | NO: | Nucleotide Sequence |
| Antibody | 77 | ATGGAGACTGGGCTGCGCTGGCTTCTCCTGGTCGCTGTGCTCAAAGGTGTCCA |
| 1 HC | | GTGTCAGTCGCTGGAGGAGTCCGGGGGTCGCCTGGTCACGCCTGGGACACCC |
| coding | | CTGACACTCACCTGCACAGTCTCTGGATTTTCCCTCAGTAGCCAGAAAGTGGGC |
| Antibody | | TGGGTCCGCCAGGCTCCAGGGAAGGGGCTGGAATGGATCGGAATCATTAATAA |
| | | TTATGGTAGCACATACTACGCGAGCTGGGCGAAAGGCCGATTCACCATCTCGAA |
| | | AACCTCGACCACAGTGGATCTGAGAATCACCAGTCTGACGGCCGAGGACACGG |
| | | CCACCTATTTCTGTGCCCGTGATCCTGATGGTAGTATTGTCTTTGACATCTGGG |
| | | GCCCAGGCACCCTTGTCACCGTCTCCTTGGGGCAACCTAAGGCTCCATCAGTC |
| | | TTCCCACTGGCCCCCTGCTGCGGGGACACACCCAGCTCCACGGTGACCCTGG |
| | | GCTGCCTGGTCAAAGGCTACCTCCCGGAGCCAGTGACCGTGACCTGGAACTCG |
| | | GGCACCCTCACCAATGGGGTACGCACCTTCCCGTCCGTCCGGCAGTCCTCAGG |
| | | CCTCTACTCGCTGAGCAGCGTGGTGAGCGTGACCTCAAGCAGCCAGCCCGTCA |
| | | CCTGCAACGTGGCCCACCCAGCCACCAACACCAAAGTGGACAAGACCGTTGCG |
| | | CCCTCGACATGCAGCAAGCCCACGTGCCCACCCCCTGAACTCCTGGGGCGATC |
| | | CTCTGTCTTCATCTTCCCCCCAAAACCCAAGGACACCCTCATGATCTCACGCAC |
| | | CCCCGAGGTCACATGCGTGGTGGTGGACGTGAGCCAGGATGACCCCGAGGTG |
| | | CAGTTCACATGGTACATAAACAACGAGCAGGTGCGCACCGCCCGGCCGCCGCT |
| | | ACGGGAGCAGCAGTTCAACAGCACGATCCGCGTGGTCAGCACCCTCCCCATCG |
| | | CGCACCAGGACTGGCTGAGGGGCAAGGAGTTCAAGTGCAAAGTCCACAACAAG |
| | | GCACTCCCGGCCCCCATCGAGAAAACCATCTCCAAAGCCAGAGGGCAGCCCCT |
| | | GGAGCCGAAGGTCTACACCATGGGCCCTCCCCGGGAGGAGCTGAGCAGCAGG |
| | | TCGGTCAGCCTGACCTGCATGATCAACGGCTTCTACCCTTCCGACATCTCGGTG |
| | | GAGTGGGAGAAGAACGGGAAGGCAGAGGACAACTACAAGACCACGCCGGCCG |
| | | TGCTGGACAGCGACGGCTCCTACTTCCTCTACAGCAAGCTCTCAGTGCCCACG |
| | | AGTGAGTGGCAGCGGGGCGACGTCTTCACCTGCTCCGTGATGCACGAGGCCTT |
| | | GCACAACCACTACACGCAGAAGTCCATCTCCCGCTCTCCGGGTAAATGA |
| 2 and 3 | 78 | ATGGAGACTGGGCTGCGCTGGCTTCTCCTGGTCGCTGTGCTCAAAGGTGTCCA |
| HC | | GTGTCAGTCGGTGGAGGAGTCCGGGGGTCGCCTGGTCACGCCTGGGACACCC |
| coding | | CTGACACTCACCTGCACCGTCTCTGGATTCTCCCTCAGTAGCTATGCAATGATC |
| | | TGGGTCCGCCAGGCTCCAGGGAAGGGGCTGGAATGGATCGGATTCATTAGTCG |
| | | TAGTGGTATCACATACTACGCGAGCTGGGCAAAAGGCCGATTCACCATCTCCAA |
| | | AACCTCGACCACGGTGGATCTGAAAATGACCAGTCTGACAACCGAGGACACGG |
| | | CCACCTATTTCTGTGCCAGAGAATTCGGTGCTGTTGGTAGTGATTATTATAGGGA |
| | | CGCCTTTAACTTGTGGGGCCCAGGCACCCTGGTCACCGTCTCCTCAGGGCAAC |
| | | CTAAGGCTCCATCAGTCTTCCCACTGGCCCCCTGCTGCGGGGACACACCCAGC |
| | | TCCACGGTGACCCTGGGCTGCCTGGTCAAAGGCTACCTCCCGGAGCCAGTGAC |
| | | CGTGACCTGGAACTCGGGCACCCTCACCAATGGGGTACGCACCTTCCCGTCCG |
| | | TCCGGCAGTCCTCAGGCCTCTACTCGCTGAGCAGCGTGGTGAGCGTGACCTCA |
| | | AGCAGCCAGCCCGTCACCTGCAACGTGGCCCACCCAGCCACCAACACCAAAGT |
| | | GGACAAGACCGTTGCGCCCTCGACATGCAGCAAGCCCACGTGCCCACCCCCTG |
| | | AACTCCTGGGGCGATCCTCTGTCTTCATCTTCCCCCCAAAACCCAAGGACACCC |
| | | TCATGATCTCACGCACCCCCGAGGTCACATGCGTGGTGGTGGACGTGAGCCAG |
| | | GATGACCCCGAGGTGCAGTTCACATGGTACATAAACAACGAGCAGGTGCGCAC |
| | | CGCCCGGCCGCCGCTACGGGAGCAGCAGTTCAACAGCACGATCCGCGTGGTC |
| | | AGCACCCTCCCCATCGCGCACCAGGACTGGCTGAGGGGCAAGGAGTTCAAGTG |
| | | CAAAGTCCACAACAAGGCACTCCCGGCCCCCATCGAGAAAACCATCTCCAAAG |
| | | CCAGAGGGCAGCCCCTGGAGCCGAAGGTCTACACCATGGGCCCTCCCCGGGA |
| | | GGAGCTGAGCAGCAGGTCGGTCAGCCTGACCTGCATGATCAACGGCTTCTACC |
| | | CTTCCGACATCTCGGTGGAGTGGGAGAAGAACGGGAAGGCAGAGGACAACTAC |
| | | AAGACCACGCCGGCCGTGCTGGACAGCGACGGCTCCTACTTCCTCTACAGCAA |
| | | GCTCTCAGTGCCCACGAGTGAGTGGCAGCGGGGCGACGTCTTCACCTGCTCCG |
| | | TGATGCACGAGGCCTTGCACAACCACTACACGCAGAAGTCCATCTCCCGCTCTC |
| | | CGGGTAAATGA |
| Antibody | 79 | ATGGAGACTGGGCTGCGCTGGCTTCTCCTGGTCGCTGTGCTCAAAGGTGTCCA |
| 4 HC | | GTGTCAGTCGCTGGAGGAGTCCGGGGGTCGCCTGGTCACGCCTGGGACACCC |
| coding | | CTGACACTCACCTGCACAGTCTCTGGATTTTCCCTAAATAACTACAAAGTGGGCT |
| | | GGGTCCGCCAGGCTCCAGGAAAGGGGCTGGAATGGATCGGAATCATTAACTAT |
| | | TATAGTCAGACATACTACGCGAGCTGGGCCAAAGGCCGATTCACCATCTCGAAA |
| | | ACCTCGACCACGGTGGATCTGAAGCTCACCAGTCCGACAACCGAAGACACGGC |
| | | CACCTATTTCTGTGCCCGTGATCCTGATGGTAGTATTGTCTTTGACATCTGGGG |
| | | CCCAGGCACCCTTGTCACCGTCTCCTTGGGGCAACCTAAGGCTCCATCAGTCTT |
| | | CCCACTGGCCCCCTGCTGCGGGGACACACCCAGCTCCACGGTGACCCTGGGC |
| | | TGCCTGGTCAAAGGCTACCTCCCGGAGCCAGTGACCGTGACCTGGAACTCGGG |
| | | CACCCTCACCAATGGGGTACGCACCTTCCCGTCCGTCCGGCAGTCCTCAGGCC |
| | | TCTACTCGCTGAGCAGCGTGGTGAGCGTGACCTCAAGCAGCCAGCCCGTCACC |
| | | TGCAACGTGGCCCACCCAGCCACCAACACCAAAGTGGACAAGACCGTTGTGCC |
| | | CTCGACATGCAGCAAGCCCACGTGCCCACCCCCTGAACTCCTGGGGCGATCCT |
| | | CTGTCTTCATCTTCCCCCCAAAACCCAAGGACACCCTCATGATCTCACGCACCC |
| | | CCGAGGTCACATGCGTGGTGGTGGACGTGAGCCAGGATGACCCCGAGGTGCA |
| | | GTTCACATGGTACATAAACAACGAGCAGGTGCGCACCGCCCGGCCGCCGCTAC |
| | | GGGAGCAGCAGTTCAACAGCACGATCCGCGTGGTCAGCACCCTCCCCATCGCG |
| | | CACCAGGACTGGCTGAGGGGCAAGGAGTTCAAGTGCAAAGTCCACAACAAGGC |
| | | ACTCCCGGCCCCCATCGAGAAAACCATCTCCAAAGCCAGAGGGCAGCCCCTGG |
| | | AGCCGAAGGTCTACACCATGGGCCCTCCCCGGGAGGAGCTGAGCAGCAGGTC |
| | | GGTCAGCCTGACCTGCATGATCAACGGCTTCTACCCTTCCGACATCTCGGTGGA |
| | | GTGGGAGAAGAACGGGAAGGCAGAGGACAACTACAAGACCACGCCGGCCGTG |
| | | CTGGACAGCGACGGCTCCTACTTCCTCTACAGCAAGCTCTCAGTGCCCACGAG |
| | | TGAGTGGCAGCGGGGCGACGTCTTCACCTGCTCCGTGATGCACGAGGCCTTGC |
| | | ACAACCACTACACGCAGAAGTCCATCTCCCGCTCTCCGGGTAAATGA |
| Antibody | 80 | ATGGAGACTGGGCTGCGCTGGCTTCTCCTGGTCGCTGTGCTCAAAGGTGTCCA |
| 5 HC | | GTGTCAGTCGGTGGAGGAGTCCGGAGGAGGCCTGGTAACGCCTGGAGGAACC |
| coding | | CTGACACTCACCTGCACCGTCTCTGGATTCTCCCTCAGTAACTATGCAATGAGC |
| | | TGGGTCCGCCAGGCTCCAGGGAAGGGGCTGGAATGGATCGGATTCATTAGTCG |
| | | TAGTGGTATTACATACTACGCGAGCTGGGCAAAAGGCCGATTCACCATCTCCAA |
| | | AACCTCGACCACGGTGGATCTGAAAATCACCAGTCCGACGACCGAGGACACGG |
| | | CCGCCTATTTCTGTGCCAGAGAATTCGGTGCTGTTGGTAGTGATTATTATAGGG |
| | | ACGCCTTGAGGTTGTGGGGCCCAGGCACCCTGGTCACCGTCTCCTCAGGGCAA |
| | | CCTAAGGCTCCATCAGTCTTCCCACTGGCCCCCTGCTGCGGGGACACACCCAG |
| | | CTCCACGGTGACCCTGGGCTGCCTGGTCAAAGGCTACCTCCCGGAGCCAGTGA |
| | | CCGTGACCTGGAACTCGGGCACCCTCACCAATGGGGTACGCACCTTCCCGTCC |
| | | GTCCGGCAGTCCTCAGGCCTCTACTCGCTGAGCAGCGTGGTGAGCGTGACCTC |
| | | AAGCAGCCAGCCCGTCACCTGCAACGTGGCCCACCCAGCCACCAACACCAAAG |
| | | TGGACAAGACCGTTGCGCCCTCGACATGCAGCAAGCCCACGTGCCCACCCCCT |
| | | GAACTCCTGGGGCGATCCTCTGTCTTCATCTTCCCCCCAAAACCCAAGGACACC |
| | | CTCATGATCTCACGCACCCCCGAGGTCACATGCGTGGTGGTGGACGTGAGCCA |
| | | GGATGACCCCGAGGTGCAGTTCACATGGTACATAAACAACGAGCAGGTGCGCA |
| | | CCGCCCGGCCGCCGCTACGGGAGCAGCAGTTCAACAGCACGATCCGCGTGGT |
| | | CAGCACCCTCCCCATCGCGCACCAGGACTGGCTGAGGGGCAAGGAGTTCAAGT |
| | | GCAAAGTCCACAACAAGGCACTCCCGGCCCCCATCGAGAAAACCATCTCCAAA |
| | | GCCAGAGGGCAGCCCCTGGAGCCGAAGGTCTACACCATGGGCCCTCCCCGGG |
| | | AGGAGCTGAGCAGCAGGTCGGTCAGCCTGACCTGCATGATCAACGGCTTCTAC |
| | | CCTTCCGACATCTCGGTGGAGTGGGAGAAGAACGGGAAGGCAGAGGACAACTA |
| | | CAAGACCACGCCGGCCGTGCTGGACAGCGACGGCTCCTACTTCCTCTACAGCA |
| | | AGCTCTCAGTGCCCACGAGTGAGTGGCAGCGGGGCGACGTCTTCACCTGCTCC |
| | | GTGATGCACGAGGCCTTGCACAACCACTACACGCAGAAGTCCATCTCCCGCTCT |
| | | CCGGGTAAATGA |
| Antibody | 81 | ATGGAGACTGGGCTGCGCTGGCTTCTCCTGGTCGCTGTGCTCAAAGGTGTCCA |
| 6 HC | | GTGTCAGTCGCTGGAGGAGTCCGGGGGTCGCCTGGTAACGCCTGGGACACCC |
| coding | | CTGACACTCACCTGCACAGTCTCTGGAATCGACCTCAGTACCCATGCAATGACC |
| | | TGGGTCCGCCAGGCTCCAGGAAAGGGGCTGGAATGGATCGGAGTCATTAATCC |
| | | TAGTGGTAGCGCATACTACGCGACCTGGGTGAATGGCCGATTCACCATCTCCAA |
| | | AACCTCGACCACGGTGGATCTGAAAATCACCAGTCCGACAACCGGGGACACGG |
| | | CCAAGTATTTCTGTGCCAGAGATTATATTACTGCGGGTGATTATTATATGGATGC |
| | | TTTTGATCCCTGGGGCCCAGGCACCCTGGTCACCGTCTCCTCAGGGCAACCTA |
| | | AGGCTCCATCAGTCTTCCCACTGGCCCCCTGCTGCGGGGACACACCCAGCTCC |
| | | ACGGTGACCCTGGGCTGCCTGGTCAAAGGCTACCTCCCGGAGCCAGTGACCGT |
| | | GACCTGGAACTCGGGCACCCTCACCAATGGGGTACGCACCTTCCCGTCCGTCC |
| | | GGCAGTCCTCAGGCCTCTACTCGCTGAGCAGCGTGGTGAGCGTGACCTCAAGC |
| | | AGCCAGCCCGTCACCTGCAACGTGGCCCACCCAGCCACCAACACCAAAGTGGA |
| | | CAAGACCGTTGCGCCCTCGACATGCAGCAAGCCCACGTGCCCACCCCCTGAAC |
| | | TCCTGGGGCGATCCTCTGTCTTCATCTTCCCCCCAAAACCCAAGGACACCCTCA |
| | | TGATCTCACGCACCCCCGAGGTCACATGCGTGGTGGTGGACGTGAGCCAGGAT |
| | | GACCCCGAGGTGCAGTTCACATGGTACATAAACAACGAGCAGGTGCGCACCGC |
| | | CCGGCCGCCGCTACGGGAGCAGCAGTTCAACAGCACGATCCGCGTGGTCAGC |
| | | ACCCTCCCCATCGCGCACCAGGACTGGCTGAGGGGCAAGGAGTTCAAGTGCAA |
| | | AGTCCACAACAAGGCACTCCCGGCCCCCATCGAGAAAACCATCTCCAAAGCCA |
| | | GAGGGCAGCCCCTGGAGCCGAAGGTCTACACCATGGGCCCTCCCCGGGAGGA |
| | | GCTGAGCAGCAGGTCGGTCAGCCTGACCTGCATGATCAACGGCTTCTACCCTT |
| | | CCGACATCTCGGTGGAGTGGGAGAAGAACGGGAAGGCAGAGGACAACTACAA |
| | | GACCACGCCGGCCGTGCTGGACAGCGACGGCTCCTACTTCCTCTACAGCAAGC |
| | | TCTCAGTGCCCACGAGTGAGTGGCAGCGGGGCGACGTCTTCACCTGCTCCGTG |
| | | ATGCACGAGGCCTTGCACAACCACTACACGCAGAAGTCCATCTCCCGCTCTCC |
| | | GGGTAAATGA |

TABLE 9
Nucleic acid sequences encoding light chains

| | SEQ ID | |
|---|---|---|
| Name | NO: | Nucleotide Sequence |
| Antibody | 82 | ATGGACACGAGGGCCCCCACTCAGCTGCTGGGGCTCCTGCTGCTCTGGCTCC |
| 1 LC | | CAGGTGCCACATTTGCCCAAGTGCTGACCCAGACTGCATCCCCCGTGTCTGC |
| coding | | GGCTGTTGGAGGCACAGTCACCATCAATTGCCAGTCCAGTCAGAGTGTTGTAT |
| | | ATAACAACCGCTTATCCTGGTTTCAACAGAAACCAGGGCAGCCTCCCAAGCTC |
| | | CTGATCTATGGTGCATCCACTCTGGCATCTGGGGTCCCATCGCGGTTCAAAGG |
| | | CAGTGGATCTGGGACACAGTTCACTCTCACCATCAGCGACGTGCAGTGTGAC |
| | | GATGCTGCCACTTACTACTGTCTAGGCTCCTATGATTGTAGTAGTGGTGATTG |
| | | CCATGCTTTCGGCGGAGGGACCGAGGTGGTGGTCAAAGGTGATCCAGTTGCA |
| | | CCTACTGTCCTCATCTTCCCACCAGCTGCTGATCAGGTGGCAACTGGAACAGT |
| | | CACCATCGTGTGTGTGGCGAATAAATACTTTCCCGATGTCACCGTCACCTGGG |
| | | AGGTGGATGGCACCACCCAAACAACTGGCATCGAGAACAGTAAAACACCGCA |
| | | GAATTCTGCAGATTGTACCTACAACCTCAGCAGCACTCTGACACTGACCAGCA |
| | | CACAGTACAACAGCCACAAAGAGTACACCTGCAAGGTGACCCAGGGCACGAC |
| | | CTCAGTCGTCCAGAGCTTCAATAGGGGTGACTGTTAG |
| Antibody | 83 | ATGGACATGAGGGCCCCCACTCAGCTGCTGGGGCTCCTACTGCTCTGGCTCC |
| 2 LC | | CAGGTGCCAGATGTGCTGACATTGTGATGACCCAGACTCCAGCCTCCGTGGA |
| coding | | GGCAGCTGTGGGAGGCACAGTCACCATCAATTGCCAAGCCAGTGAGAGCATT |
| | | AATAGTTGGTTGTCCTGGTATCAGCAGAAACCAGGGCAGCCTCCCAACCTCCT |
| | | GATCTACAGGGCATCCACTCTGGCATCTGGGGTCCCATCGCGGTTCAGTGGC |
| | | GGTGGATCTGGGACAGAGTACACTCTCACCATCAGCGACCTGGAGTGTGCCG |
| | | ATGCTGTCACTTATTACTGTCAAAGCTATTATGAGGAGGATGGTATTGGTTATG |
| | | CTTTCGGCGGAGGGACCGAGGTGGTGGTCGAAGGTGATCCAGTTGCACCTAC |
| | | TGTCCTCATCTTCCCACCAGCTGCTGATCAGGTGGCAACTGGAACAGTCACCA |
| | | TCGTGTGTGTGGCGAATAAATACTTTCCCGATGTCACCGTCACCTGGGAGGTG |
| | | GATGGCACCACCCAAACAACTGGCATCGAGAACAGTAAAACACCGCAGAATTC |
| | | TGCAGATTGTACCTACAACCTCAGCAGCACTCTGACACTGACCAGCACACAGT |
| | | ACAACAGCCACAAAGAGTACACCTGCAAGGTGACCCAGGGCACGACCTCAGT |
| | | CGTCCAGAGCTTCAATAGGGGTGACTGTTAG |
| Antibody | 84 | ATGGACATGAGGGCCCCCACTCAGCTGCTGGGGCTCCTGCTGCTCTGGCTCC |
| 3 LC | | CAGGTGCCAGATGTGCTGACATTGTGATGACCCAGACTCCATCCTCCGTGTCT |
| coding | | GCAGCTGTGGGAGGCACAGTCACCATCAATTGCCAGGCCAGTCAGAACATTT |
| | | ACAGCAATTTAGCCTGGTATCAGCAGAAACCAGGGCAGCGTCCCAGGCTCCT |
| | | GATCTATGGCGCATCCAATCTGGCATCTGGGGTCCCATCGCGGTTCAAAGGC |
| | | AGTAGATCTGGGACAGAGTTCACTCTCACCATCAGCGACCTGGAGTGTGCCG |
| | | ATGCTGCCACTTACTACTGTCAAGGCTATGATTATAGTACTGCTGGTGCCTATC |
| | | CTTTCGGCGGAGGGACCGCGGTGGTGGTCAAAGGTGATCCAGTTGCACCTAC |
| | | TGTCCTCATCTTCCCACCAGCTGCTGATCAGGTGGCAACTGGAACAGTCACCA |
| | | TCGTGTGTGTGGCGAATAAATACTTTCCCGATGTCACCGTCACCTGGGAGGTG |
| | | GATGGCACCACCCAAACAACTGGCATCGAGAACAGTAAAACACCGCAGAATTC |
| | | TGCAGATTGTACCTACAACCTCAGCAGCACTCTGACACTGACCAGCACACAGT |
| | | ACAACAGCCACAAAGAGTACACCTGCAAGGTGACCCAGGGCACGACCTCAGT |
| | | CGTCCAGAGCTTCAATAGGGGTGACTGTTAG |
| Antibody | 85 | ATGGACACGAGGGCCCCCACTCAGCTGCTGGGGCTCCTGCTGCTCTGGCTCC |
| 4 LC | | CAGGTGCCACATTTGCCCAAGTGCTGACCCAGACTGCATCGCCCGTGTCTGC |
| coding | | GGCTGTGGGAAGCACAGTCACCATCAATTGCCAGTCCAGTCAGAGCGTTTATA |
| | | GTAACAAGCGCTTAGCCTGGTTTCAGCTGAAACCAGGGCAGCCTCCCAAGCT |
| | | CCTGATCTATGGTGCATCCACACTGGCATCTGGGGTCCCATCGCGATTCAAGG |
| | | GCAGTGGATCTGGGACACAGTTCACTCTCACCATCAGCGACGTGCAGTGTGA |
| | | CGATGCTGCCACTTACTACTGTGCAGGCGGTTATGATTGTAGTACTGGTGATT |
| | | GTTGGACTTTCGGCGGAGGGACCGAGGTGGTGGTCACAGGTGATCCAGTTGC |
| | | ACCTACTGTCCTCATCTTCCCACCAGCTGCTGATCAGGTGGCAACTGGAACAG |
| | | TCACCATCGTGTGTGTGGCGAATAAATACTTTCCCGATGTCACCGTCACCTGG |
| | | GAGGTGGATGGCACCACCCAAACAACTGGCATCGAGAACAGTAAAACACCGC |
| | | AGAATTCTGCAGATTGTACCTACAACCTCAGCAGCACTCTGACACTGACCAGC |
| | | ACACAGTACAACAGCCACAAAGAGTACACCTGCAAGGTGACCCAGGGCACGA |
| | | CCTCAGTCGTCCAGAGCTTCAATAGGGGTGACTGTTAG |
| Antibody | 86 | ATGGACATGAGGGCCCCCACTCAGCTGCTGGGGCTCCTGCTGCTCTGGCTCC |
| 5 LC | | CAGGTGCCAGATGTGCTGACATCGTGATGACCCAGACTCCATCCTCCGTGTCT |
| coding | | GCAGCTGTGGGAGGCACAGTCACCATCAAGTGCCAGGCCAGTCAGAGCATTG |
| | | GTAGTAATTTAGCCTGGTATCAGCAGAAACCAGGGCAGCCTCCCAAGCTCCTG |
| | | ATCTATGGTGCATCCACTCTGGAATCTGGGGTCCCATCGCGGTTTAAAGGCAG |
| | | TGGATCTGGGACAGAGTACACTCTCACCATCAGCGACCTGGAGTGTGCCGAT |
| | | GCTGCCACTTACTACTGTCAAAGCTATTATGAGGGTAGTGATATTGGTTATGCT |
| | | TTCGGCGGAGGGACCGAGGTGGTGGTCGAAGGTGATCCAGTTGCACCTACTG |
| | | TCCTCATCTTCCCACCAGCTGCTGATCAGGTGGCAACTGGAACAGTCACCATC |
| | | GTGTGTGTGGCGAATAAATACTTTCCCGATGTCACCGTCACCTGGGAGGTGGA |
| | | TGGCACCACCCAAACAACTGGCATCGAGAACAGTAAAACACCGCAGAATTCTG |
| | | CAGATTGTACCTACAACCTCAGCAGCACTCTGACACTGACCAGCACACAGTAC |
| | | AACAGCCACAAAGAGTACACCTGCAAGGTGACCCAGGGCACGACCTCAGTCG |
| | | TCCAGAGCTTCAATAGGGGTGACTGTTAG |
| Antibody | 87 | ATGGACACGAGGGCCCCCACTCAGCTGCTGGGGCTCCTGCTGCTCTGGCTCC |
| 6 LC | | CAGGTGCCAGATGTGCTGACATCGTGATGACCCAGACTCCAGCCTCTGTGTCT |
| coding | | GCAGCTGTGGGAGGCACAGTCACCATCAAGTGCCAGGCCAGTCAGAGCATTA |
| | | GCAACCAACTATCCTGGTATCAGCAGAAATCAGGGCAGCCTCCCAAGCTCCTG |
| | | ATCTACAGGGCATCTACTCTGGCATCTGGGGTCCCATCGCGGTTCAAAGGCA |
| | | GTGGATCTGGGACAGAGTTCACTCTCACCATCAGCGACCTGGAGTGTGCCGA |
| | | TGCTGCCACTTACTACTGTCAACAGGGTTATAATAGAGATAATGTTGATAATCT |
| | | TTTCGGCGGAGGGACCGAGGTGGTGGTCAAAGGTGATCCAGTTGCACCTACT |
| | | GTCCTCATCTTCCCACCAGCTGCTGATCAGGTGGCAACTGGAACAGTCACCAT |
| | | CGTGTGTGTGGCGAATAAATACTTTCCCGATGTCACCGTCACCTGGGAGGTG |
| | | GATGGCACCACCCAAACAACTGGCATCGAGAACAGTAAAACACCGCAGAATTC |
| | | TGCAGATTGTACCTACAACCTCAGCAGCACTCTGACACTGACCAGCACACAGT |
| | | ACAACAGCCACAAAGAGTACACCTGCAAGGTGACCCAGGGCACGACCTCAGT |
| | | CGTCCAGAGCTTCAATAGGGGTGACTGTTAG |

### Methods of the Disclosure

Disclosed herein are methods for detecting phosphorylated tau in a sample from an individual using antibodies described herein. In some embodiments, the phosphorylated tau is selected from the group consisting of pTau-212, pTau-217, pTau-231, pTau-214, and pTau-220. In some embodiments, methods for detecting phosphorylated tau in a sample from an individual using antibodies described herein comprise improved specificity and sensitivity. Also provided are methods of treating or selecting for treatment of, monitoring a treatment of, and/or diagnosing a disease or a condition of the present disclosure based, at least in part, on the presence or the level of phosphorylated tau measured in the sample obtained from a subject. In some embodiments, the disease or condition is a neurodegenerative disease or disorder (e.g., Alzheimer's disease).

### Detection

Described herein are methods for detecting phosphorylated tau in a sample from an individual. In some embodiments, the methods comprise performing an assay on the sample using an antibody or antibody fragment that binds to phosphorylated tau. In some embodiments, the antibody or antibody fragment is used in an immunoassay.

Described herein are methods for detecting phosphorylated tau in a sample from an individual comprising: performing an immunoassay on the sample using an antibody or antibody fragment that binds to phosphorylated tau. In some embodiments, the phosphorylated tau is selected from the group consisting of pTau-212, pTau-217, pTau-231, pTau-214, pTau-220, and pTau-181. In some embodiments, the phosphorylated tau is selected from the group consisting of pTau-212, pTau-217, pTau-231, pTau-214, and pTau-220. In some embodiments, the phosphorylated tau is pTau-217. In some embodiments, the phosphorylated tau is pTau-231. In some embodiments, the phosphorylated tau is pTau-181. In some embodiments, the phosphorylated tau is pTau-212. In some embodiments, the phosphorylated tau is pTau-217. In some embodiments, the phosphorylated tau is pTau-214. In some embodiments, the phosphorylated tau is pTau-220. In some embodiments, the phosphorylated tau is pTau-181 and pTau-217. In some embodiments, the phosphorylated tau is pTau-181 and pTau-231. In some embodiments, the phosphorylated tau is pTau-217 and pTau-231. In some embodiments, the phosphorylated tau is pTau-181, pTau-217, and pTau-231.

Further described herein are methods for detecting phosphorylated tau in a sample from an individual comprising: performing an assay on the sample using an antibody or antibody fragment that binds to multiple phosphorylated tau proteins. In some embodiments, the methods detects pTau-217 and pTau-231. In some embodiments, the methods detects pTau-212 and pTau-217. In some embodiments, the methods detects pTau-212 and pTau-231. In some embodiments, the methods detects pTau-212, pTau-217 and pTau-231.

Described herein are methods for detecting phosphorylated tau in a sample from an individual, wherein the method detects pTau-217 and pTau-231 in a sample selected from the group consisting of a plasma sample and serum sample. In some embodiments, the methods detect pTau-212 and pTau-217 in a sample selected from the group consisting of a plasma sample and serum sample. In some embodiments, the methods detect pTau-212 and pTau-231 in a sample selected from the group consisting of a plasma sample and serum sample. In some embodiments, the methods detect pTau-212, pTau-217, and pTau-231 in a sample selected from the group consisting of a plasma sample and serum sample.

Methods as described herein can comprise performing an assay on a sample, wherein the sample is selected from the group consisting of a plasma sample and serum sample. In some instances, the sample is a blood sample. In some instances, the sample is a cerebrospinal fluid sample. The sample can be a blood sample obtained by a venous blood draw. The sample can be a blood sample obtained from a finger prick blood draw. The sample can be obtained by a health care provider or by the subject. The method can comprise obtaining a sample from a subject. In some cases, the sample is obtained from the subject during a visit to the clinic or the hospital.

Further described herein, in some embodiments, are methods to determine a level of a biomarker selected from the group consisting of A042, A040, A038, BACE1, hFABP, TREM2, YKL-40, IP-10, neurogranin, SNAP-25, synaptotagmin, alpha-synuclein, TDP-43, ferritin, VILIP-1, NfL, GFAP, and combinations thereof. In some instances, the biomarker is A042. In some instances, the biomarker is A040. In some instances, the biomarker is A042 and A040. In some instances, the biomarker is APOE. In some instances, the biomarker is selected from the group consisting of APOE2, APOE3, and APOE4. In some instances, the biomarker is APOE4.

In some embodiments, methods for detecting phosphorylated tau in a sample comprise an immunoassay or a ligand assay using the antibodies or antibody fragments described herein. In some cases, the assay is selected from the group consisting of enzyme-linked immunosorbent assay (ELISA), a colorimetric immunoassay, a homogeneous immunoassay, a non-optical immunoassay, a fluorescence immunoassay, a chemiluminescence immunoassay, an electro-chemiluminescence immunoassay, a fluorescence resonance energy transfer (FRET) immunoassay, a time resolved fluorescence immunoassay, a lateral flow immunoassay, a microspot immunoassay, a surface plasmon resonance assay, a ligand assay, a clotting assay, a chromatography assay, and immunocapture coupled with mass spectrometry. In some cases, the assay comprises an immunoassay. In some cases, the assay is selected from the group consisting of a Western blot, enzyme-linked immunosorbent assays (ELISA), and chromatography. In some cases, the immunoassays are single-plexed. In some cases, the immunoassays are multiplexed.

Methods as described herein can comprise a plurality of immunoassays using the antibodies or antibody fragments described herein. In some cases, the plurality of immunoassays are the same immunoassay (e.g., four or more ELISA assays). When the plurality of immunoassays are the same immunoassay, each of the plurality of immunoassays can detect a different phosphorylated tau. When the plurality of immunoassays are the same immunoassay, each of the plurality of immunoassays can be performed in the same reaction chamber or a different reaction chamber. A reaction chamber can be any suitable space for performing an immunoassay. Examples of reaction chambers include, but are not limited to, a well in a microplate, an Eppendorf tube, or a droplet.

In some cases, the plurality of immunoassays are different immunoassays. When the plurality of immunoassays are different immunoassays, each of the plurality of immunoassays can detect a different phosphorylated tau. When the plurality of immunoassays are different immunoassays, each of the plurality of immunoassays can be performed in the same reaction chamber or a different reaction chamber.

In some cases, the assay comprises a non-immunoassay. In some cases, the assay is selected from the group consisting of High Performance Liquid Chromatography (HPLC), High Performance Liquid Chromatography Mass spectrometry (HPLC-MS), Gas Chromatography Mass Spectrometry (GC-MS), Liquid Chromatography Mass spectrometry (LC-MS), Liquid Chromatography Tandem Mass spectrometry (LC-MS/MS), immunohistochemistry (IHC), polymerase chain reaction (PCR), quantitative PCR (qPCR), and combinations thereof.

Methods as described herein using the antibodies described herein may be capable of detecting phosphorylated tau in the sample at a low limit of detection. In some embodiments, the methods as described herein using the antibodies described herein are capable of detecting phosphorylated tau in the sample at a limit of detection of at least about 1.5 picogram per milliliter (pg/mL). In some embodiments, the methods as described herein using the antibodies described herein are capable of detecting phosphorylated tau in the sample at a limit of detection of at least about 5 picogram per milliliter (pg/mL). In some embodiments, the methods as described herein using the antibodies described herein are capable of detecting phosphorylated tau in the sample at a limit of detection in a range of about 0.5 pg/mL to about 10 μg/mL, about 1 μg/mL to about 8 μg/mL, about 1.5 pg/mL to about 7 μg/mL, about 2 μg/mL to about 6 μg/mL, or about 3 μg/mL to about 5 μg/mL.

### Diagnosis, Prognosis and Treatment

Neurodegenerative disorders describe a diverse group of conditions characterized by neuronal cell death and eventual subsequent atrophy of certain cellular systems, certain brain structures, certain brain regions, or affect the entire brain or CNS with atrophy not typica of the normal aging process. In many instances, neurodegenerative disorders are slowly progressing conditions, often with no improvement in the functional manifestations of the disease or condition in an affected subject. In many instances, there is also evidence of slowly progressing pathology associated with the disorder. Examples include the progressive deposition of amyloid beta plaques and neurofibrillary tangles in the CNS of patients affected by Alzheimer's disease. Other examples include pathological deposition of hyperphosphorylated tau protein in any number of tauopathies. Many neurodegenerative disorders have adult-onset or typical onset within an aged population (e.g., over age 60). In a variety of instances, adult-onset neurodegenerative disorders are characterized by misfolding of native proteins, native peptides, or modified proteins or peptides. Examples include amyloid-β (Aβ) peptide, tau, u-synuclein, transactive DNA-binding protein 43 (TDP43) and prion proteins, any of which may become misfolded in a disorder characterized by dysfunction of that protein. These misfolded proteins assemble into aggregates as the disorder progress and the aggregates may elongate with the addition of more subunits of misfolded, aggregated protein. These elongated aggregates can form filaments which have been shown to induce neuronal cell death and in some instances are capable of replicating and inducing the formation of further protein aggregates and filament formations. In some instances, these filaments form larger structures such as plaques or protein tangles visible by cytological examination. In some instances, these pathological depositions of proteins are visible via detections systems such as a PET scan. As an example, one of the diagnostic criteria for AD is visualization of abnormal amounts or deposited amyloid beta in the CNS detected by PET scan. Blood testing and genetic testing can also be used to diagnose certain neurodegenerative conditions. As an example of a genetic test that helps to inform a diagnosis of a neurodegenerative disorder, the apolipoprotein E (APOE) genotype is a genetic risk factor for known for dementia Alzheimer's disease. APOE genotypes include three alleles (82, E3, E4). APOE-E4 is the strongest genetic risk factor for Alzheimer's disease (AD), and is associated with an increase in the levels of amyloid deposition and an early age of onset. APOE-84/E4 homozygotes have the strongest genetic risk of developing AD compared to other APOE genotype combinations. Magnetic resonance imaging (MRI) may also be used to assess a subject for an abnormal CNS pathology (e.g., reduction in brain volume; or reduction in volume of a particular brain region or structure) indicative of a neurodegenerative disorder. Computer tomography (CT) scans may also be used in diagnosing neurodegenerative disorders. As neurodegenerative disorders involve progressive neuronal cell loss, early diagnosis and early treatment is critical to intervene in the typical course of the pathology and slow, mitigate, or halt further cellular loss. In some instances, further disease progression results in co-morbid pathological features such as local or system inflammation that can produce less favorable conditions for the chosen treatment. As such, early diagnosis or early prognosis by assaying one or more predictive biomarkers can provide crucial information for informing a decision to start a treatment for a neurodegenerative disorder. One predictive biomarker described herein is a measurement of p-Tau 217 present in the subject. An elevated level of p-Tau 217 present in the blood or in the cerebral spinal fluid compared to a reference level from healthy individuals can indicate an increased likelihood of the subject eventually developing a neurodegenerative disorder such as AD. An elevated level of p-Tau 217 present in the blood or in the cerebral spinal fluid compared to a reference level from healthy individuals can indicate that the subject already resides in a preclinical stage of a neurodegenerative disorder, and as such may be diagnosed with the disorder and have an opportunity to initiate treatment earlier than if not identified at this time. An elevated level of p-Tau 217 present in the blood or in the cerebral spinal fluid compared to a reference level from healthy individuals can indicate that the subject resides in a prodromal stage of a neurodegenerative disorder, and as such may be diagnosed with the disorder and have an opportunity to initiate treatment earlier than if not identified at this time. An elevated level of p-Tau 217 present in the blood or in the cerebral spinal fluid compared to a reference level from healthy individuals, or an increase in p-Tau 217 level in the subject than from an earlier assayed time point can indicate that a neurodegenerative disorder is progressing, or that a current treatment is not sufficiently effective to slow or halt the progression of the disorder. As such, a change in p-Tau 217 level in a subject can inform a decision to switch treatments or change dosage or administration frequency of a treatment. AD diagnosis may be confirmed by PET imaging for abnormal amyloid beta deposition in the brain of the subject. AD diagnosis may be confirmed by PET imaging for abnormal tau deposition in the brain of the subject. Diagnostic criteria for prodromal or a mild neurodegenerative disorder (e.g., MCI) may be made by determining the presence of a collection of functional deficits in the subject. A medical practitioner (e.g., a neurologist) may provide an AD diagnosis to a subject. Such functional deficits leading to a diagnosis of prodromal AD include evidence of modest cognitive decline from a previous level of performance in one or more domains listed below and a decline in neurocognitive performance, typically involving test performance in the range of one and two standard deviations below appropriate norms on formal testing or equivalent clinical evaluation. Additionally, the cognitive deficits are insufficient to interfere with independence. Additionally, The cognitive deficits do not occur exclusively in the context of a delirium. Additionally, the cognitive deficits are not primarily attributable to another mental disorder (e.g., major depressive disorder or schizophrenia). Diagnostic criteria for a major or advanced neurodegenerative disorder may be made by determining the presence of a collection of functional deficits in the subject. A major neurocognitive disorder is defined by the following:
Evidence of substantial cognitive decline from a previous level of performance in one or more of the domains listed below; and a decline in neurocognitive performance, typically involving test performance in the range of two or more standard deviations below appropriate norms (e.g., below the third percentile) on formal testing or equivalent clinical evaluation.
The cognitive deficits are sufficient to interfere with independence, and the cognitive deficits do not occur exclusively in the context of a delirium, and the cognitive deficits are not primarily attributable to another mental disorder.

Six cognitive domains may be affected in both Minor and Major neurodegenerative disorders affecting cognition: 1) Complex attention, 2) Executive ability, 3) Learning and memory, 4) Language, 5) Perceptual —Motor —Visual perception, praxis, and 6) Social cognition. Presence of deficiencies in any of these six cognitive domains may be used by a clinician or medical practitioner to provide a diagnosis of a neurodegenerative disorder to a subject. For neurodegenerative disorders characterized by pathological accumulation of beta amyloid in the brain (e.g., AD), a treatment may be selected to slow, mitigate, or halt the progression of the disorder. Such treatments include drugs designed to inhibit Aβ aggregation, inhibit AR plaque formation, decrease levels of Aβ systemically or within the brain, remove accumulated Aβ plaques within the CNS, or reduce the expression of MAPT protein. Exemplary treatment include administration of a monoclonal antibody that inhibits or targets Aβ. Exemplary monoclonal antibody therapies that inhibit or target Aβ include Donanemab, Lecanemab, Remternetug, or Aducanumab.

Methods as described herein using the antibodies described herein may be used for establishing Alzheimer's disease in the individual based on detection of phosphorylated tau. In some embodiments, Alzheimer's disease in the individual is established if pTau-212, pTau-217, pTau-231, pTau-214, pTau-220, or combinations thereof is detected in the sample from the individual. In some embodiments the phosphorylated tau is detected in the sample using one of the methods of detection of the present disclosure. In some embodiments, the method comprises performing an immunoassay with an antibody or antigen-fragment that binds phosphorylated tau of the present disclosures, such as those provided in Table 5B.

Methods as described herein using the antibodies described herein may be used for prognosis of the individual for developing Alzheimer's disease based on detection of phosphorylated tau. In some embodiments, prognosis of the individual for developing Alzheimer's disease is determined if pTau-212, pTau-217, pTau-231, pTau-214, pTau-220, or combinations thereof is detected in the sample from the individual.

Methods as described herein using the antibodies described herein may be used accurately and specifically establish Alzheimer's disease (AD) in an individual as compared to a disease or disorder or neurologically and cognitively unimpaired condition, selected from the group consisting of a non-Alzheimer's disease (AD) neurodegenerative disease, a A3-negative non-AD neurodegenerative disease, a AD-positive non-AD neurodegenerative diseases, behavioral variant of frontotemporal dementia (BvFTD), primary progressive aphasia (PPA), vascular dementia (VaD), Parkinson's disease (PD), PD with dementia (PDD), multiple system atrophy (MSA), progressive supranuclear palsy (PSP), corticobasal syndrome (CBS), AD-negative cognitively impaired or unimpaired controls and combinations thereof. In some embodiments, the methods as described herein using the antibodies described herein comprise an improved accuracy or specificity of at least or about 70%, 80%, 90%, 95%, 99%, or more at establishing AD as compared to a disease or disorder or neurologically and cognitively unimpaired condition.

Methods as described herein using the antibodies described herein may be used more accurately and with higher specificity to establish Alzheimer's disease (AD) in an individual as compared to a neuropathological examination or clinical diagnosis. In some embodiments, the methods as described herein using the antibodies described herein comprise an improved accuracy or specificity of at least or about 70%, 80%, 90%, 95%, 99%, or more at establishing AD as compared to a neuropathological examination or clinical diagnosis. In some embodiments, the neuropathological examination or clinical diagnosis comprises neurological tests, mental exams, or brain imaging (e.g. MRI, CT, or PET scans).

### Patient Selection

Disclosed herein are methods of selecting an individual for treatment of a neurodegenerative disease. In some instances, a subject is selected for treatment of a neurodegenerative disease following a clinical diagnosis of the particular disease. In some instances, a subject is selected for treatment of a neurodegenerative disease due to a collection of deficiencies corresponding to a prodromal stage of a neurodegenerative disease. In some instances, a subject is selected for treatment of a neurodegenerative disease due to positive biomarker results indicating that the subject resides in a preclinical state for a particular neurodegenerative disease. In some embodiments, the neurodegenerative disease is characterized by accumulation of amyloid-O (AD) peptide in a brain of the individual. In some embodiments, the neurodegenerative disease is Alzheimer's disease. The treatment may comprise administering a therapeutic agent to the individual. In some embodiments, the therapeutic agent comprises an inhibitor of the Aβ peptide or a modified form of the Aβ peptide. In some embodiments, the therapeutic agent comprises an inhibitor of a phosphorylated tau protein. In some embodiments, the methods of selection are based, at least in part, on a level of phosphorylated tau 217 (p-Tau 217) measured in a sample obtained from the individual using one or more of the methods of detection of the present disclosure. In some embodiments, the subject is selected for treatment if the level of P-Tau 217 is higher than a standard value of p-Tau 217 level derived from a plurality of reference samples from reference subjects that do not have the neurodegenerative disease. In some embodiments, the subject is not selected for treatment if the level of P-Tau 217 is lower or the same than a standard value of p-Tau 217 level. In some embodiments, the level of p-Tau 217 in the sample obtained from the individual is predictive that the subject has deposition of abnormal tau protein in the brain with a sensitivity, specificity, positive predictive value, negative predictive value, area under the curve (AUC) or accuracy that is higher than a neuropathological examination or clinical diagnosis comprises neurological tests, mental exams, or brain imaging (e.g. MRI, CT, or PET scans). In some embodiments, the level of p-Tau 217 in the sample obtained from the individual is predictive that the subject has deposition of abnormal tau protein in the brain with a sensitivity, specificity, positive predictive value, negative predictive value, or accuracy that is greater than or equal to about 60 μg/mL from a CSF sample. In some embodiments, the level of p-Tau 217 in the sample obtained from the individual is predictive that the subject has deposition of abnormal tau protein in the brain with a sensitivity, specificity, positive predictive value, negative predictive value, or accuracy that is greater than or equal to about 60 μg/mL from a CSF sample. In some instances, detected levels of A040 peptide in a plasma sample from a subject range from about 400-600 μg/mL, 450-580 pg/mL, 475-550 pg/mL, or 490-525 pg/mL. In sample instances, amyloid positive subjects (determine by PET) having normal cognition have detected levels of plasma A040 less than about 495 μg/mL. In sample instances, amyloid positive subjects (determine by PET) having normal cognition have detected levels of plasma A040 less than about 505 μg/mL. In sample instances, amyloid positive subjects (determine by PET) having MCI have detected levels of plasma A040 less than about 515 μg/mL. In sample instances, amyloid positive subjects (determine by PET) having MCI have detected levels of plasma A040 less than about 510 μg/mL. In sample instances, amyloid positive subjects (determine by PET) having mild AD have detected levels of plasma A040 less than about 505 μg/mL. In sample instances, amyloid positive subjects (determine by PET) having mild AD have detected levels of plasma A040 less than about 510 μg/mL. In some instances, detected levels of A042 peptide in a plasma sample from a subject range from about 40-60 pg/mL. In sample instances, amyloid positive subjects (determine by PET) having normal cognition have detected levels of plasma A042 less than about 50 μg/mL. In sample instances, amyloid positive subjects (determine by PET) having normal cognition have detected levels of plasma A042 less than about 46 μg/mL. In sample instances, amyloid positive subjects (determine by PET) having MCI have detected levels of plasma A042 less than about 55 μg/mL. In sample instances, amyloid positive subjects (determine by PET) having MCI have detected levels of plasma A042 less than about 46 μg/mL. In sample instances, amyloid positive subjects (determine by PET) having mild AD have detected levels of plasma A042 less than about 55 μg/mL. In sample instances, amyloid positive subjects (determine by PET) having mild AD have detected levels of plasma A042 less than about 50 μg/mL. In some instances the ratio for A042/A040 levels detected in plasma samples ranges from about 0.08-0.12. In some instances, detected levels of P-tau 181 in a plasma sample from a subject range from about 12-50 pg/mL. In sample instances, amyloid positive subjects (determine by PET) having normal cognition have detected levels of plasma P-tau 181 less than about 25 μg/mL. In sample instances, amyloid positive subjects (determine by PET) having normal cognition have detected levels of plasma P-tau 181 less than about 20 μg/mL. In sample instances, amyloid positive subjects (determine by PET) having MCI have detected levels of plasma P-tau 181 less than about 30 μg/mL. In sample instances, amyloid positive subjects (determine by PET) having MCI have detected levels of plasma P-tau 181 less than about 20 μg/mL. In sample instances, amyloid positive subjects (determine by PET) having mild AD have detected levels of plasma P-tau 181 less than about 35 μg/mL. In sample instances, amyloid positive subjects (determine by PET) having mild AD have detected levels of plasma P-tau 181 less than about 20 μg/mL. In some instances, detected levels of P-tau 217 in a plasma sample from a subject range from about 0.05-12 pg/mL. In sample instances, amyloid positive subjects (determine by PET) having normal cognition have detected levels of plasma P-tau 217 less than about 2 μg/mL. In sample instances, amyloid positive subjects (determine by PET) having normal cognition have detected levels of plasma P-tau 217 less than about 0.3 pg/mL. In sample instances, amyloid positive subjects (determine by PET) having MCI have detected levels of plasma P-tau 217 less than about 5 μg/mL. In sample instances, amyloid positive subjects (determine by PET) having MCI have detected levels of plasma P-tau 217 less than about 0.5 pg/mL. In sample instances, amyloid positive subjects (determine by PET) having mild AD have detected levels of plasma P-tau 217 less than about 1 μg/mL. In sample instances, amyloid positive subjects (determine by PET) having mild AD have detected levels of plasma P-tau 217 less than about 12 μg/mL.

In some embodiments, the level of p-Tau 217 in the sample obtained from the individual is predictive that the subject has deposition of abnormal tau protein in the brain with a sensitivity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The clinical sensitivity of identifying the deposition of abnormal tau protein in the brain using the biomarker test of the present disclosure may be calculated as the percentage of independent test samples associated with presence of the deposition of abnormal tau protein in the brain (e.g., subjects known to have the deposition of abnormal tau protein in the brain) that are correctly identified or classified as having the deposition of abnormal tau protein in the brain.

In some embodiments, the level of p-Tau 217 in the sample obtained from the individual is predictive that the subject has a high likelihood of a positive therapeutic response to a therapeutic agent with a sensitivity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The clinical sensitivity of identifying the a high likelihood of a positive therapeutic response to a therapeutic agent using the biomarker test of the present disclosure may be calculated as the percentage of independent test samples associated with presence of the a high likelihood of a positive therapeutic response to a therapeutic agent (e.g., subjects known to have the responded positively to the therapeutic agent) that are correctly identified or classified as having a high likelihood of a positive therapeutic response to a therapeutic agent.

In some embodiments, the level of p-Tau 217 in the sample obtained from the individual is predictive that the subject has a high likelihood of a positive therapeutic response to a therapeutic agent with a specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The clinical specificity of identifying the a high likelihood of a positive therapeutic response to a therapeutic agent using the biomarker test of the present disclosure may be calculated as the percentage of independent test samples associated with absence of the a high likelihood of a positive therapeutic response to a therapeutic agent (e.g., subjects that did not response or loss response to the therapeutic agent) that are correctly identified or classified as not having the high likelihood of a positive therapeutic response to the therapeutic agent.

In some embodiments, the level of p-Tau 217 in the sample obtained from the individual is predictive that the subject has a high likelihood of a positive therapeutic response to a therapeutic agent with a positive predictive value of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the abnormal tau protein in the brain using the biomarker test of the present disclosure may be calculated as the percentage of samples identified or classified as having the high likelihood of a positive therapeutic response to a therapeutic agent that correspond to subjects that truly exhibit a positive therapeutic response to the therapeutic agent.

In some embodiments, the level of p-Tau 217 in the sample obtained from the individual is predictive that the subject has a high likelihood of a positive therapeutic response to a therapeutic agent with a negative predictive value of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the high likelihood of a positive therapeutic response to a therapeutic agent using the biomarker test of the present disclosure may be calculated as the percentage of samples identified or classified as not having the high likelihood of a positive therapeutic response to the therapeutic agent that correspond to subjects that truly do not exhibit a positive therapeutic response to the therapeutic agent.

In some embodiments, the level of p-Tau 217 in the sample obtained from the individual is predictive that the subject has a high likelihood of a positive therapeutic response to a therapeutic agent with an area under the curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operator Characteristic (ROC) curve (e.g., the area under the ROC curve) in classifying samples as having or not having the high likelihood of a positive therapeutic response to a therapeutic agent. The AUC may range from a value of 0 to 1, where an AUC of 0.5 is indicative of a completely random classifier (e.g., a coin flip) and an AUC of 1 is indicative of a perfectly accurate classifier (with sensitivity of 100% and specificity of 100%).

In some embodiments, the level of p-Tau 217 in the sample obtained from the individual is predictive that the subject has deposition of abnormal tau protein in the brain with a specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The clinical specificity of identifying the deposition of abnormal tau protein in the brain using the biomarker test of the present disclosure may be calculated as the percentage of independent test samples associated with absence of the deposition of abnormal tau protein in the brain (e.g., subjects with negative clinical test results for the deposition of abnormal tau protein in the brain by CT scan, etc.) that are correctly identified or classified as not having the deposition of abnormal tau protein in the brain.

In some embodiments, the level of p-Tau 217 in the sample obtained from the individual is predictive that the subject has deposition of abnormal tau protein in the brain with a positive predictive value of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the abnormal tau protein in the brain using the biomarker test of the present disclosure may be calculated as the percentage of samples identified or classified as having the abnormal tau protein in the brain that correspond to subjects that truly have the abnormal tau protein in the brain.

In some embodiments, the level of p-Tau 217 in the sample obtained from the individual is predictive that the subject has deposition of abnormal tau protein in the brain with a negative predictive value of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the deposition of abnormal tau protein in the brain using the biomarker test of the present disclosure may be calculated as the percentage of samples identified or classified as not having the deposition of abnormal tau protein in the brain that correspond to subjects that truly do not have the deposition of abnormal tau protein in the brain.

In some embodiments, the level of p-Tau 217 in the sample obtained from the individual is predictive that the subject has deposition of abnormal tau protein in the brain with an area under the curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operator Characteristic (ROC) curve (e.g., the area under the ROC curve) in classifying samples as having or not having the deposition of abnormal tau protein in the brain. The AUC may range from a value of 0 to 1, where an AUC of 0.5 is indicative of a completely random classifier (e.g., a coin flip) and an AUC of 1 is indicative of a perfectly accurate classifier (with sensitivity of 100% and specificity of 100%).

### Treating Neurodegenerative Disease

Disclosed herein are methods of treating a neurodegenerative disease in an individual. In some embodiments, the neurodegenerative disease is characterized by accumulation of amyloid-P (AD) peptide in a brain of the individual. In some embodiments, the neurodegenerative disease is Alzheimer's disease. The treatment may comprise administering a therapeutic agent to the individual. In some embodiments, the therapeutic agent comprises an inhibitor of the Aβ peptide or a modified form of the Aβ peptide. In some embodiments, the therapeutic agent comprises an inhibitor of a phosphorylated tau protein. In some embodiments, the methods of selection are based, at least in part, on a level of phosphorylated tau 217 (p-Tau 217) measured in a sample obtained from the individual using one or more of the methods of detection of the present disclosure. In some embodiments, the subject is selected for treatment if the level of P-Tau 217 is higher than a standard value of p-Tau 217 level derived from a plurality of reference samples from reference subjects that do not have the neurodegenerative disease. In some embodiments, the subject is not selected for treatment if the level of P-Tau 217 is lower or the same than a standard value of p-Tau 217 level. In some embodiments, the level of p-Tau 217 in the sample obtained from the individual is predictive that the subject has deposition of abnormal tau protein in the brain with a sensitivity, specificity, positive predictive value, negative predictive value, area under the curve (AUC) or accuracy that is higher than a neuropathological examination or clinical diagnosis comprises neurological tests, mental exams, or brain imaging (e.g. MRI, CT, or PET scans). In some embodiments, the level of p-Tau 217 in the sample obtained from the individual is predictive that the subject has deposition of abnormal tau protein in the brain with a sensitivity, specificity, positive predictive value, negative predictive value, or accuracy that is greater than or equal to about 60 μg/mL from a CSF sample. In some embodiments, the level of p-Tau 217 in the sample obtained from the individual is predictive that the subject has deposition of abnormal tau protein in the brain with a sensitivity, specificity, positive predictive value, negative predictive value, or accuracy that is greater than or equal to about 60 μg/mL from a CSF sample. In some instances, detected levels of A040 peptide in a plasma sample from a subject range from about 400-600 μg/mL, 450-580 pg/mL, 475-550 pg/mL, or 490-525 pg/mL. In sample instances, amyloid positive subjects (determine by PET) having normal cognition have detected levels of plasma A040 less than about 495 μg/mL. In sample instances, amyloid positive subjects (determine by PET) having normal cognition have detected levels of plasma A040 less than about 505 μg/mL. In sample instances, amyloid positive subjects (determine by PET) having MCI have detected levels of plasma A040 less than about 515 μg/mL. In sample instances, amyloid positive subjects (determine by PET) having MCI have detected levels of plasma A040 less than about 510 μg/mL. In sample instances, amyloid positive subjects (determine by PET) having mild AD have detected levels of plasma A040 less than about 505 μg/mL. In sample instances, amyloid positive subjects (determine by PET) having mild AD have detected levels of plasma A040 less than about 510 μg/mL. In some instances, detected levels of A042 peptide in a plasma sample from a subject range from about 40-60 pg/mL. In sample instances, amyloid positive subjects (determine by PET) having normal cognition have detected levels of plasma A042 less than about 50 μg/mL. In sample instances, amyloid positive subjects (determine by PET) having normal cognition have detected levels of plasma A042 less than about 46 μg/mL. In sample instances, amyloid positive subjects (determine by PET) having MCI have detected levels of plasma A042 less than about 55 μg/mL. In sample instances, amyloid positive subjects (determine by PET) having MCI have detected levels of plasma A042 less than about 46 μg/mL. In sample instances, amyloid positive subjects (determine by PET) having mild AD have detected levels of plasma A042 less than about 55 μg/mL. In sample instances, amyloid positive subjects (determine by PET) having mild AD have detected levels of plasma A042 less than about 50 μg/mL. In some instances the ratio for A042/A040 levels detected in plasma samples ranges from about 0.08-0.12. In some instances, detected levels of P-tau 181 in a plasma sample from a subject range from about 12-50 pg/mL. In sample instances, amyloid positive subjects (determine by PET) having normal cognition have detected levels of plasma P-tau 181 less than about 25 μg/mL. In sample instances, amyloid positive subjects (determine by PET) having normal cognition have detected levels of plasma P-tau 181 less than about 20 μg/mL. In sample instances, amyloid positive subjects (determine by PET) having MCI have detected levels of plasma P-tau 181 less than about 30 μg/mL. In sample instances, amyloid positive subjects (determine by PET) having MCI have detected levels of plasma P-tau 181 less than about 20 μg/mL. In sample instances, amyloid positive subjects (determine by PET) having mild AD have detected levels of plasma P-tau 181 less than about 35 μg/mL. In sample instances, amyloid positive subjects (determine by PET) having mild AD have detected levels of plasma P-tau 181 less than about 20 μg/mL. In some instances, detected levels of P-tau 217 in a plasma sample from a subject range from about 0.05-12 pg/mL. In sample instances, amyloid positive subjects (determine by PET) having normal cognition have detected levels of plasma P-tau 217 less than about 2 μg/mL. In sample instances, amyloid positive subjects (determine by PET) having normal cognition have detected levels of plasma P-tau 217 less than about 0.3 pg/mL. In sample instances, amyloid positive subjects (determine by PET) having MCI have detected levels of plasma P-tau 217 less than about 5 μg/mL. In sample instances, amyloid positive subjects (determine by PET) having MCI have detected levels of plasma P-tau 217 less than about 0.5 pg/mL. In sample instances, amyloid positive subjects (determine by PET) having mild AD have detected levels of plasma P-tau 217 less than about 1 μg/mL. In sample instances, amyloid positive subjects (determine by PET) having mild AD have detected levels of plasma P-tau 217 less than about 12 μg/mL.

In some embodiments, the level of p-Tau 217 (also referred to here as “pT217 tau”) is detected in a sample from the individual using the methods of detection of the present disclosure, and compared to the level of pT217 tau in the sample with a standard value of pT217 tau level derived from a plurality of reference samples. In some embodiments, the reference samples are obtained from reference subjects that do not have the neurodegenerative disease. In some embodiments, the reference samples are obtained from reference subjects that are at an early stage of the neurodegenerative disease. In some embodiments, measuring the level of p-Tau 217 comprises performing an immunoassay on the sample using an anti-tau antibody or antigen-binding fragment thereof. In some embodiments, the anti-tau antibody or antigen-binding fragment thereof comprises i) a heavy chain comprising a variable heavy chain (VH) domain and ii) a light chain comprising a variable light chain (VL) domain, wherein the VH domain comprises HCDR1 sequence selected from SEQ ID NOs: 1-9, HCDR2 sequence selected from SEQ ID NOs: 10-17, and HCDR3 sequence selected from SEQ ID NOs: 18-23, and the VL domain comprises LCDR1 sequence selected from SEQ ID NOs: 24-31, LCDR2 sequence selected from SEQ ID NOs: 32-36, and LCDR3 sequence selected from SEQ ID NOs: 37-43. In some embodiments, the methods further comprise determining a likelihood of clinical diagnosis of the neurodegenerative disease in the subject based at least in part on the measuring. In some embodiments, the methods further comprise establishing an early prognosis of the neurodegenerative disease in the subject based at least in part on the determining, wherein the early prognosis predates providing a diagnosis of a prodromal stage of the neurodegenerative disease in the subject. In some embodiments, the methods further comprise initiating a treatment comprising administration of a formulation comprising a therapeutic agent, wherein deciding the initiating is based at least in part on the establishing.

### Neurodegenerative Disease or Condition

Disclosed herein, in some embodiments, are methods and compositions for the diagnosis, treatment, monitoring of a treatment, or selection for treatment, of a neurodegenerative disease or condition. Non-limiting of neurodegenerative disease and conditions include Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, Hallevorden-Spatz disease, mild cognitive impairment (MCI), Lewy Body's disease (LBD), Amyotrophic lateral sclerosis, spinal muscular atrophy, Friedreich's ataxia, Wemicke-Korsakoff syndrome, a prion disease, vascular dementia, Alzheimer's related dementia, Huntington's disease (HD), and a transgene-induced tauopathy. In some embodiments, the neurodegenerative disease or condition is a tauopathy. In some embodiments the tauopathy comprises Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, Hallevorden-Spatz disease, mild cognitive impairment (MCI), Lewy Body's disease (LBD), Amyotrophic lateral sclerosis, spinal muscular atrophy, Friedreich's ataxia, Wernicke-Korsakoff syndrome, a prion disease, vascular dementia, Alzheimer's related dementia, Huntington's disease (HD), or a transgene-induced tauopathy. In some embodiments the tauopathy comprises Pick's disease (PiD), Frontal temporal dementia (FTD), chronic traumatic encephalopathy (CTE), corticobasal degeneration (CBD), frontotemporal dementia with parkinsonism linked to chromosome 17 (FTDP-17), progressive supranuclear palsy (PSP), primary age-related tauopathy (PART), Argyrophilic grain disease (AGD), globular glial tauopathy (GGT), aging-related tau astrogliopathy (ARTAG), Richardson syndrome (RS), corticobasal syndrome (CBS), non-fluent agrammatic primary progressive aphasia (nfaPPA)/apraxia of speech, progressive akinesia with gait freezing (PAGF), and behavioral variant frontotemporal dementia (bvFTD), Parkinsonian syndrome, primary lateral sclerosis, amnestic syndrome of the hippocampal type, Gomo-Tempini Primary Progressive Aphasia (PPA), amyotrophic lateral sclerosis (ALS), apraxia of speech (AOS), Lewy body disease (LBD), primary progressive aphasia (nfaPPA), logopenic variant primary progressive aphasia (lvPPA), semantic variant primary progressive aphasia (svPPA), Late Onset Cerebellar Ataxia Syndrome (LOCA), TDP-43 encephalopathy (LATE), Down syndrome, Creutzfeldt-Jakob Disease, or any combination thereof.

In some embodiments, the neurodegenerative disease or condition is Alzheimer's disease (AD). In some embodiments, the prodromal stage of AD is characterized by mild cognitive impairment (MCI). In some embodiments, the prodromal stage of AD is characterized at least as Braak stage I by a determined a spatial extent of tau-PET. In some embodiments, the prodromal stage of AD is characterized at least as Braak stage II by a determined a spatial extent of tau-PET.

### Samples

Disclosed herein, in some embodiments, are methods of detecting the level of pT217 tau in a sample obtained from a subject. In some embodiments, the sample is whole blood, peripheral blood, plasma, serum, saliva, mucus, urine, semen, lymph, fecal extract, cheek swab, cells or other bodily fluid or tissue. In some embodiments, the sample is a cerebral spinal fluid (CSF) sample. Alternatively, a sample can be obtained through primary patient derived cell lines, or archived patient samples in the form of preserved samples, or fresh frozen samples.

In some embodiments, the methods comprise detecting a level of pT217 tau in a sample, wherein the level of pT217 tau higher than the standard value of pT217 tau level derived from the plurality of reference samples. In some embodiments, the plurality of reference samples is whole blood, peripheral blood, plasma, serum, saliva, mucus, urine, semen, lymph, fecal extract, cheek swab, cells or other bodily fluid, tissue, or CSF. In some embodiments, the plurality of reference samples are taken from healthy individuals not suspected of having, or having, the neurodegenerative disease (Alzheimer's disease).

### Subjects

Disclosed herein, in some embodiments, are subjects. In some embodiments, the subject is an animal, including, but not limited to, humans, non-human primates, rodents, and domestic and game animals. In some embodiments, the subject is to be the recipient of a particular treatment. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In various embodiments, a subject can be one who has been previously diagnosed with or identified as suffering from or having a condition in need of treatment. In certain embodiments, the subject is a human. In various other embodiments, the subject previously diagnosed with or identified as suffering from or having a condition may or may not have undergone treatment for a condition. In yet other embodiments, a subject can also be one who has not been previously diagnosed as having a condition (e.g., a subject who exhibits one or more risk factors for a condition). A “subject in need” of treatment for a particular condition can be a subject having that condition, diagnosed as having that condition, or at risk of developing that condition. In some embodiments, the subject is a female. In some embodiments, the subject is a male.

In some embodiments, the subject meets inclusion criteria for initiating the treatment, and wherein the inclusion criteria comprise detecting a level of pT217 tau in a CSF or plasma sample taken prior to initiating the treatment. In some embodiments, the subject is at least 60 years of age. In some embodiments, the subject is between 60-85 years of age. In some embodiments, the subject is at least 50, 51, 52, 53, 54, 55, 56, 57, 58, 59, 60, 61, 62, 63, 64, 65, 66, 67, 68, 69, 70, 71, 72, 73, 74, 75, 76,77, 78, 79, 80, 81, 82, 83, 84, 85, 86, 87, 88, 89, or 90 years of age. In some embodiments, the subject is the aforementioned age at the time of initiating the treatment. In some embodiments, the selected therapeutic agent is Donanemab. In some embodiments, the selected therapeutic agent is Lecanemab. In some embodiments, the selected therapeutic agent is Remtemetug. In some embodiments, the establishing the early prognosis comprises establishing a likelihood of the neurodegenerative disease (e.g., Alzheimer's disease) progressing to dementia (e.g., Alzheimer's disease dimension) in the subject based at least in part on the level of pT217 tau that is detected in the CSF or plasma sample.

Described herein are methods of treating a neurodegenerative disease in a subject. In some embodiments, the method comprises detecting a level of pT217 tau in a cerebrospinal fluid (CSF) sample from the subject and correlating the level of pT217 tau in the sample with a standard value of pT217 tau CSF level derived from a plurality of reference samples. In some embodiments, the method comprises receiving data obtained by detecting a level of pT217 tau in a cerebrospinal fluid (CSF) sample from the subject and correlating the level of pT217 tau in the sample with a standard value of pT217 tau CSF level derived from a plurality of reference samples, wherein the detecting comprises performing an immunoassay on the sample.

In some embodiments, the method comprises detecting a level of pT217 tau in a plasma sample from the subject and correlating the level of pT217 tau in the plasma sample with a standard value of pT217 tau plasma level derived from a plurality of reference samples. In some embodiments, the method comprises receiving data obtained by detecting a level of pT217 tau in a plasma sample from the subject and correlating the level of pT217 tau in the sample with a standard value of pT217 tau plasma level derived from a plurality of reference samples, wherein the detecting comprises performing an immunoassay on the sample. In some embodiments, the method comprises detecting a level of pT217 tau in a cerebrospinal fluid serum sample from the subject and correlating the level of pT217 tau in the sample with a standard value of pT217 tau serum level derived from a plurality of reference samples. In some embodiments, the method comprises receiving data obtained by detecting a level of pT217 tau in a serum sample from the subject and correlating the level of pT217 tau in the sample with a standard value of pT217 tau serum level derived from a plurality of reference samples, wherein the detecting comprises performing an immunoassay on the sample. In some embodiments, the method comprises detecting a level of pT217 tau in a peripheral blood sample from the subject and correlating the level of pT217 tau in the sample with a standard value of pT217 tau peripheral blood level derived from a plurality of reference samples. In some embodiments, the method comprises receiving data obtained by detecting a level of pT217 tau in a peripheral blood sample from the subject and correlating the level of pT217 tau in the sample with a standard value of pT217 tau peripheral level derived from a plurality of reference samples, wherein the detecting comprises performing an immunoassay on the sample. In some embodiments, the method comprises detecting a level of pT217 tau in a capillary blood sample from the subject and correlating the level of pT217 tau in the sample with a standard value of pT217 tau capillary blood level derived from a plurality of reference samples. In some embodiments, the method comprises receiving data obtained by detecting a level of pT217 tau in a capillary blood sample from the subject and correlating the level of pT217 tau in the sample with a standard value of pT217 tau capillary blood level derived from a plurality of reference samples, wherein the detecting comprises performing an immunoassay on the sample.

In some embodiments, the method comprises determining whether the subject has an elevated risk of a neurodegenerative condition leading to pathological memory impairment and cognitive decline compared to a reference subject and is suitable for treatment with a small molecule amyloid-beta peptide inhibitor by detecting a level of pT217 tau in a cerebrospinal fluid (CSF) sample or a plasma sample from the subject and correlating the level of pT217 tau in the sample with a standard value of pT217 tau CSF level or pT217 tau plasma level derived from a plurality of reference samples. In some embodiments, the method comprises analyzing a likelihood of clinical diagnosis of the neurodegenerative disease in the subject based at least in part on receiving data obtained by detecting a level of pT217 in a sample taken from the subject. In some embodiments, the method comprises providing an early prognosis of the neurodegenerative disease in the subject based at least in part on the analyzing, wherein the early prognosis predates supplying a diagnosis of a prodromal stage of the neurodegenerative disease in the subject. In some embodiments, the method comprises initiating a treatment comprising administration of a formulation comprising a therapeutic agent. In some embodiments, the method comprises detecting a level of pT217 tau in a cerebrospinal fluid (CSF) sample from the subject and correlating the level of pT217 tau in the sample with a standard value of pT217 tau CSF level derived from a plurality of reference samples, wherein the detecting comprises performing an immunoassay on the sample using an anti-tau antibody or antigen-binding fragment thereof comprising i) a heavy chain comprising a variable heavy chain (VH) domain and ii) a light chain comprising a variable light chain (VL) domain, wherein the VH domain comprises HCDR1 sequence selected from SEQ ID NOs: 1-9, HCDR2 sequence selected from SEQ ID NOs: 10-17, and HCDR3 sequence selected from SEQ ID NOs: 18-23, and the VL domain comprises LCDR1 sequence selected from SEQ ID NOs: 24-31, LCDR2 sequence selected from SEQ ID NOs: 32-36, and LCDR3 sequence selected from SEQ ID NOs: 37-43. In some embodiments, the method comprises determining a likelihood of clinical diagnosis of the neurodegenerative disease in the subject based at least in part on detecting a level of pT217 tau in a cerebrospinal fluid (CSF) sample from the subject and correlating the level of pT217 tau in the sample with a standard value of pT217 tau CSF level derived from a plurality of reference samples. In some embodiments, the method comprises establishing an early prognosis of the neurodegenerative disease in the subject based at least in part on the determining a likelihood of clinical diagnosis of the neurodegenerative disease in the subject, wherein the early prognosis predates providing a diagnosis of a prodromal stage of the neurodegenerative disease in the subject. In some embodiments, the method comprises initiating a treatment comprising administration of a formulation comprising a therapeutic agent, wherein deciding the initiating is based at least in part on the establishing an early prognosis of the neurodegenerative disease in the subject. In some embodiments, pT217 tau levels are determined in plasma or CSF samples taken from the subject following initiation of treatment. In some embodiments, pT217 tau levels are determined in a plasma sample taken from the subject following initiation of treatment. In some embodiments, pT217 tau levels are determined in a CSF sample taken from the subject following initiation of treatment. In some embodiments, a change in pT217 tau level is determined from baseline to a second level measured in a plasma or CSF sample taken from the subject during the period of treatment. In some embodiments, the second sample is a plasma sample. In some embodiments, the second sample is a CSF sample. In some embodiments, the second sample is taken about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 76 weeks after initiation of the treatment. In some embodiments, the pT217 tau level is correlated with a level of a different biomarker. In some embodiments, the different biomarker is assayed in a plasma or CSF sample. In some embodiments, the other biomarker is selected from p-Tau 181, p-Tau 231, pTau212, pTau-217+, BD-Tau, NfL, GFAP, A042 peptide, A040 peptide, A042/AD40 ratio, or any combination thereof. In some embodiments, the other biomarker is selected from GFAP, NfL, A042, A040, A042/A040 ratio, or any combination thereof. In some embodiments, the pT217 tau level is correlated with a level of GFAP prior to a start of treatment. In some embodiments, the pT217 tau level is correlated with a level of GFAP during treatment. In some embodiments, the pT217 tau level is correlated with a level of GFAP following a period after treatment conclusion. In some embodiments, the pT217 tau level is correlated with a level of NfL prior to a start of treatment. In some embodiments, the pT217 tau level is correlated with a level of NfL during treatment. In some embodiments, the pT217 tau level is correlated with a level of NfL following a period after treatment conclusion. In some embodiments, the pT217 tau level is correlated with a level of A042 prior to a start of treatment. In some embodiments, the pT217 tau level is correlated with a level of A042 during treatment. In some embodiments, the pT217 tau level is correlated with a level of A042 following a period after treatment conclusion. In some embodiments, the pT217 tau level is correlated with a level of A040 prior to a start of treatment. In some embodiments, the pT217 tau level is correlated with a level of A040 during treatment. In some embodiments, the pT217 tau level is correlated with a level of A040 following a period after treatment conclusion. In some embodiments, the pT217 tau level is correlated with a A042/AD40 ratio prior to a start of treatment. In some embodiments, the pT217 tau level is correlated with a A042/AD40 ratio during treatment. In some embodiments, the pT217 tau level is correlated with a A042/AD40 ratio following a period after treatment conclusion. In some embodiments, the pT217 tau level is correlated with a level of p-Tau 181 prior to a start of treatment. In some embodiments, the pT217 tau level is correlated with a level of p-Tau 181 during treatment. In some embodiments, the pT217 tau level is correlated with a level of p-Tau 181 following a period after treatment conclusion. In some embodiments, the pT217 tau level is correlated with a level of p-Tau 231 prior to a start of treatment. In some embodiments, the pT217 tau level is correlated with a level of p-Tau 231 during treatment. In some embodiments, the pT217 tau level is correlated with a level of p-Tau 231 following a period after treatment conclusion. In some embodiments, the pT217 tau level is correlated with a level of pTau212 prior to a start of treatment. In some embodiments, the pT217 tau level is correlated with a level of pTau212 during treatment. In some embodiments, the pT217 tau level is correlated with a level of pTau212 following a period after treatment conclusion. In some embodiments, the pT217 tau level is correlated with a level of pTau212+prior to a start of treatment. In some embodiments, the pT217 tau level is correlated with a level of pTau212+during treatment. In some embodiments, the pT217 tau level is correlated with a level of pTau212+following a period after treatment conclusion. In some embodiments, the pT217 tau level is correlated with a level of BD-Tau prior to a start of treatment. In some embodiments, the pT217 tau level is correlated with a level of BD-Tau during treatment. In some embodiments, the pT217 tau level is correlated with a level of BD-Tau following a period after treatment conclusion. In some embodiments, the pT217 tau level is correlated with MRI volumetric brain measurement prior to a start of treatment. In some embodiments, the pT217 tau level is correlated with MRI volumetric brain measurement during treatment. In some embodiments, the pT217 tau level is correlated with MRI volumetric brain measurement following a period after treatment conclusion. In some embodiments, the pT217 tau level is correlated with a value of brain tau deposition assessed via PET measurement prior to a start of treatment. In some embodiments, the pT217 tau level is correlated with a value of brain tau deposition assessed via PET measurement during treatment. In some embodiments, the pT217 tau level is correlated with a value of brain tau deposition assessed via PET measurement following a period after treatment conclusion. In some embodiments, the pT217 tau level is correlated with a value of brain amyloid deposition assessed via PET measurement prior to a start of treatment. In some embodiments, the pT217 tau level is correlated with a value of brain amyloid deposition assessed via PET measurement during treatment. In some embodiments, the pT217 tau level is correlated with a value of brain amyloid deposition assessed via PET measurement following a period after treatment conclusion. In some embodiments, additional biomarker measurements (e.g., p-Tau 181, p-Tau 231, pTau212, pTau-217+, BD-Tau, NfL, GFAP, A042 peptide, A040 peptide, A042/AD40 ratio, or any combination thereof) are also correlated with MRI volumetric brain measurements, values of brain tau deposition assessed via PET measurement, and/or values of brain amyloid deposition assessed via PET measurement in additional to the correlation with pT217 tau level detected. In some embodiments, one or more of the correlations are significant and inform treatment efficacy for neurodegeneration pathology, treatment efficacy for neurodegeneration functional deficits, treatment efficacy for a administered dosage of a therapeutic according to a treatment regimen, disease progression, or any combination thereof.

In some embodiments of methods described, a detected and quantitated level of pT217 tau in a sample compared to a standard value of pT217 tau in calculated from a plurality of reference samples. In some embodiments of methods described, a detected and quantitated level of pT217 tau in a sample is correlated to a value of one or more separate biomarkers. In some embodiments, an elevated level of pT217 tau in a sample compared to the standard value is used in determining a likelihood of clinical diagnosis of a neurodegenerative disease (e.g., AD). In some embodiments, the elevated level of pT217 tau in a sample compared to the standard value compared to a detected level of one or more separate biomarkers is used in determining a likelihood of clinical diagnosis of a neurodegenerative disease (e.g., AD). In some embodiments, the likelihood of clinical diagnosis of the neurodegenerative disease is increased with the detection and quantitation of the elevated level of pT217 tau in the sample from the subject. In some embodiments, the likelihood of clinical diagnosis informed by the elevated level of pT217 tau allows for an earlier determination presence and/or staging of the neurodegenerative disorder than considering factors that do not comprise detected pT217 tau levels. In some embodiments, the presence and/or stage of a neurodegenerative disorder in the subject is at least partly determined based on the detected and quantitated pT217 tau level.

### Monitoring a Treatment

Also provided herein are methods of monitoring a treatment, or assessing therapeutic efficacy of a particular treatment, of the neurodegenerative disease. In some embodiments, the correlation of pT217 tau level with the other biomarker reflects an effect of the treatment. In some embodiments, the correlation of pT217 tau level with the other biomarker reflects an effect on limiting a rate of exacerbation of a pathological feature or features of the neurodegenerative disorder following initiation of the treatment. In some embodiments, the correlation of pT217 tau level with the other biomarker reflects an effect on preventing further progression of a pathological feature or features of the neurodegenerative disorder following initiation of the treatment. In some embodiments, the correlation of pT217 tau level with the other biomarker reflects an effect on improving one or more pathological features of the neurodegenerative disorder following initiation of the treatment. In some embodiments, continued measurement of plasma pT217 tau levels or CSF pT217 tau level in the subject during treatment or following treatment informs treatment efficacy. In some embodiments, treatment efficacy is measured according to a pathological feature in the brain. In some embodiments, treatment efficacy is measured according to a function output of the patient (e.g., a behavioral component, memory performance, cognition, or any combination thereof.

In some embodiments of methods described herein, a sample collected from a subject is used for biomarker analysis. In some embodiments, the subject is at risk of developing memory impairment and/or cognitive impairment due to neurodegeneration. In some embodiments, the risk is at least partly due to an assessed apolipoprotein E (APOE) genotype in the subject. In some embodiments, the subject resides in a preclinical stage of a neurodegenerative disorder, characterized by normal cognitive ability. In some embodiments, the subject is suspected of having memory impairment and/or cognitive impairment. In some embodiments, the memory impairment and/or cognitive impairment may be due to the presence of a neurodegenerative disorder in the subject. In some embodiments, the subject resides in a prodromal stage of a neurodegenerative disorder, characterized by mild cognitive impairment (MCI). In some embodiments, the subject resides in a dementia stage of a neurodegenerative disorder, characterized by significant functional impairment in cognition, or memory, or an ability to perform activities of daily living (ADLs), or any combination thereof. In some embodiments, the subject is at a preclinical phase of AD. In some embodiments, the subject is at a prodromal phase of AD. In some embodiments, the subject is at a dementia phase of AD. In some embodiments, the sample used for biomarker analysis has been collected from the subject at previous time and stored prior to testing. In some embodiments, the previous time was during an assessment with a medical practitioner (e.g., a neurologist) in which the subject was also assessed for any cognitive and memory deficits. In some embodiments, the subject has, or is suspected of having, Alzheimer's disease (AD). In some embodiments, the AD causes, is associated with, or presents with a tauopathy in the subject. In some embodiments, the tauopathy comprises a secondary tauopathy comprising neurofibrillary tangle (NTF) pathology in the brain of the subject, wherein the tauopathy is secondary to amyloid-beta plaques in the brain of the subject. In some embodiments, the AD is categorized as sub-category of AD or a variant AD. In some embodiments, the AD comprises a variant AD selected from the group consisting of early-onset Alzheimer's disease, late-onset Alzheimer's disease, Familial Alzheimer's disease (FAD), a mixed dementia comprising Alzheimer's disease and vascular dementia, logopenic aphasia, posterior cortical atrophy, frontal variant Alzheimer's disease, and Alzheimer's disease combined with corticobasal syndrome (AD-CBS). In some embodiments, the subject is determined to have a risk for developing a tauopathy. In some embodiments, the subject has a preclinical tauopathy. In some embodiments, the subject has a prodromal tauopathy. In some embodiments, the subject has a tauopathy at a pre-degenerative stage. In some embodiments, the subject has an age-related tauopathy. In some embodiments, the subject has a tauopathy and demonstrated memory impairment and/or cognitive impairment. In some embodiments, the tauopathy comprises hyperphosphorylated tau. In some embodiments, the tauopathy comprises misfolded tau. In some embodiments, the tauopathy comprises oligomeric tau. In some embodiments, the tauopathy comprises aggregated paired helical filaments (PFHs) of tau. In some embodiments, the tauopathy comprises neurofibrillary tangles (NFTs), or any combination thereof.

In some embodiments, the sample used for biomarker analysis has been collected from the subject during a screening period of a clinical trial. In some embodiments, the sample used for biomarker analysis has been collected from the subject prior to initiation of a therapy. In some embodiments, the sample is selected from a cerebrospinal fluid (CSF) sample, a plasma sample, a blood sample, or a serum sample. In some embodiments, the sample is CSF sample or a plasma sample. In some embodiments, the sample is CSF sample. In some embodiments, the sample is a plasma sample. In some embodiments, one or more samples collected from a subject are used for biomarker analysis, wherein one or more samples have been collected at a time following collection of the first sample. In some embodiments, the one or more samples are collected after a treatment has commenced. In some embodiments, a change in a detected level of one or more biomarkers is analyzed to inform progression of pathology of a neurodegenerative condition. In some embodiments, a change in a detected level of one or more biomarkers is analyzed to inform progression of functional deficits (e.g., memory loss or cognitive impairment) of a neurodegenerative condition. In some embodiments, a first level of the biomarker tested from the sample collected from the subject is compared to a standard value derived from a plurality of reference samples, wherein the plurality of reference samples were collected from healthy individuals not suspected of having a neurodegenerative condition. In some embodiments, a second, third, fourth, fifth, sixth, or seventh level of the biomarker tested from the one or more samples collected from the subject is compared to a standard value derived from a plurality of reference samples, wherein the plurality of reference samples were collected from healthy individuals not suspected of having a neurodegenerative condition. In some embodiments, a second, third, fourth, fifth, sixth, or seventh level of the biomarker tested from the one or more samples collected from the subject is compared to a first level of the biomarker from a sample collected from the subject. In some embodiments, a change in a detected level of one or more biomarkers is analyzed to inform progression of pathology of a neurodegenerative condition in a subject undergoing a treatment. In some embodiments, a change in a detected level of one or more biomarkers is analyzed to inform progression of functional deficits (e.g., memory loss or cognitive impairment) of a neurodegenerative condition in a subject undergoing a treatment. In some embodiments, one or more samples collected from the subject is used for biomarker analysis, wherein the samples have been collected following completion of a treatment regimen. In some embodiments, progression of one or more biomarkers tested informs treatment efficacy. In some embodiments, progression of one or more biomarkers tested informs an early prognosis for the subject. In some embodiments, detection and quantitation of levels of one or more biomarkers informs an early clinical diagnosis of a neurodegenerative disorder (e.g., AD). In some embodiments, detection and quantitation of levels of one or more biomarkers informs selection of a patient population likely to respond favorably to a given treatment. In some embodiments, detection and quantitation of levels of one or more biomarkers informs that the subject from which the sample was collected is likely to progress from a preclinical stage of a neurodegenerative disorder to a prodromal stage (e.g., preclinical AD to prodromal AD). In some embodiments, detection and quantitation of levels of one or more biomarkers informs that the subject from which the sample was collected is likely to progress from a prodromal stage of a neurodegenerative disorder to a dementia stage (e.g., prodromal AD to AD dementia). In some embodiments, establishing an early prognosis of a neurodegenerative disease in the subject based at least in part on a detected and quantitated pT217 tau level in a sample from the subject informs initiating a therapeutic treatment comprising administering a therapeutic agent directed to the neurodegenerative disease indication. In some embodiments, the method comprises deciding on initiating the treatment based at least in part on establishing the early prognosis.

In some embodiments, one or more biomarkers is tested in a sample collected from the subject. In some embodiments, a single biomarker is used to inform diagnosis, prognosis, and/or progression of a neurodegenerative condition. In some embodiments, more than one biomarker is used to inform diagnosis, prognosis, and/or progression of a neurodegenerative condition. In some embodiments, detected and quantitated levels of two or more biomarkers are correlated to inform diagnosis, prognosis, and/or progression of a neurodegenerative condition. In some embodiments, the one or more biomarkers tested comprise phosphorylated Tau-217 (p-Tau 217), phosphorylated Tau-181 (p-Tau 181), phosphorylated Tau-231 (p-Tau 231), phosphorylated Tau-212 (pTau212), phosphorylated Tau-212+217 (pTau-217+), brain-derived tau (BD-Tau), neurofilament light chain (NfL), glial fibrillary acidic protein (GFAP), 42-amino acid R amyloid (AP42) peptide, 40-amino acid R amyloid (AP40) peptide, A042/AD40 ratio, or any combination thereof.

### Therapeutic Agents

Disclosed herein, in some embodiments, are methods of treating a neurodegenerative disease with a therapeutic agent, or monitoring the treatment of the neurodegenerative disease. In some embodiments, the therapeutic agent is provided in Table 10. In some embodiments, the methods of treatment comprise administering more than one therapeutic agent, such as at least two, three, four, five, six, seven, eight, nine or 10 therapeutic agents provided in Table 10. In some embodiments, one or more therapeutic agents is or comprise ABvac40LY3002813, AADvac-1, ACI-35, AAV-Htert, ACU193, ASN51, ACI-242, AD-35, Aducanumab, AGB101, AL002, Allopregnanolone, APH-1105, Atuzaginstat (COR388), AVP-786, AXS-05, Baricitinib, Bepranemab, BCG vaccine, BE1Y2153, BPN14770, Bromocriptine, Bryostatin 1, BXCL-501, Blarcamesine (ANAVEX2-73), Bapineuzumab, BIIB092 (BMS-986168, IPN007, Gosuranemab), BIIB080/IONIS-MAPTRx, BIIB076, BPDO-1603, BMS-984923, BDPP (bioactive dietary polyphenol preparation), Brexpiprazole, Caffeine, CAD 1063, Canakinumab, Contraloid acetate, COR588, CORT108297, CNP520(AMG520), C2N-8E12 (ABBV-8E12), Crenezumab, CST-2032, Curcumin, CY6463, Dabigatran,DAOIB, Dapagliflozin, Daratumumab, Dasatinib, Deferiprone, Donanemab (LY3002813), Donepezil (Aricept), Dronabinol, Dutanumab, Edicotinib (JNJ-40346527), Efavirenz, Emtricitabine, Empagliflozin, Elenbecestat (E2609), E2814, Ednerpic (T-817 mA), Elayta (CT1812), ExPlas, Escitalopram, Fosgonimeton (ATH01017), Galantamine, Gantenerumab, Brain Shuttle Gantenerumab (R07126209), GB301, grapeseed extract, GV1001, Guanfacine, GV-971, Intranasal insulin, HT-ALZ, Huperzine A, Hydralazine, IGC AD1, Icosapent ethyl (IPE), JNJ-63733657, KHK 66401, Lu AF87908, Lu AF20513, Lamivudine (3TC), Lanabecestat, Lenalidomide, Levetiracetam, L-serine, Lupron, Remternetug (LY3372993), LY3372689, LMTX, LY3303560 (Zagotenemab), Lecanemab (BAN2401), Losartan, LX1001, MEDI 1814, Memantine, Metformin, MK-1942, MIB-626, Montelukast, MW150, Neflamapimod (VX-745), Nabilone, NE3107, nicotinamide, nicotine, NNI-362, Nilotinib BE, Octohydro-aminoacridine succinate, Obicetrapib, Omega-3 (DHA+EPA), perindopril, Pepinemab (VX15), Posiphen, Prazosin, Ponezumab, Protollin, PQ912, PNT001, PU-AD, Quercetin, rapamycin, REM0046127, RG7345 (R06926496), rivastigmine, RG6100, R07105705 (MTAU9937A, RG6100, Semorinemab), Salsalate, Sargramostim, Semorinemab, Senicapoc, Semaglutide, SHR-1707, Simufilam (PTI-125), Sodium oligomannate, Solanezumab, scyllo-inositol, Sovateltide (PMZ-1620), Suvorexant, T3D-959, TB006, Telmisartan, Trehalose, Tdap vaccine, THC-free CBD oil, Thiethylperazine (TEP), Troriluzole (BHV4157), TPI 2871, Tricaprilin (AC-1202), TRx0237, UB-311, UCB0107, valiltramiprosate (ALZ-801), Valacyclovir, varoglutamstat (PQ912), VGH-AD1, Vorinostat, VT301, XProl595, Xanamem, Yangxue Qingao pills, an antisense RNA directed to an isoform of human tau mRNA or an isoform of human amyloid beta, an siRNA directed to an isoform of human tau mRNA or an isoform of human amyloid beta, an antisense oligonucleotide directed to an isoform of human tau mRNA or an isoform of human amyloid beta, an LNA oligonucleotide directed to an isoform of human tau mRNA or an isoform of human amyloid beta, a CRISPRn-based therapeutic targeting the human MAPT or the human APP locus, a CRISPRi-based therapeutic targeting the human MAPT or the human APP locus, allogeneic human MSCs, SNKO1 (autologous natural killer cell), allogenic adipose MSC-exosomes, CA-AC-02 (placenta derived MSCs), HUVEC-derived MSCs (NEUROSTEM), or AstroStem (autologous adipose-derived MSCs.

TABLE 10
Examples of therapeutic agent

| Agent | CADRO mechanism class | Mechanism of action | Therapeutic purpose |
|---|---|---|---|
| Aducanumab | Amyloid | Monoclonal antibody directed at | DMT |
| | | Aβ plaques and oligomers | |
| AGB101 (low-dose | Synaptic | SV2A modulator; to reduce Aβ- | DMT |
| levetiracetam) | Plasticity/Neuroprotection | induced neuronal hyperactivity | |
| Atuzaginstat | Synaptic | Bacterial protease inhibitor | DMT |
| (COR388) | Plasticity/Neuroprotection | targeting gingipain produced by | |
| | | P. gingivalis to reduce | |
| | | neuroinflammation and | |
| | | hippocampal degeneration | |
| AVP-786 | Neurotransmitter receptors | Sigma 1 receptor agonist; | Neuropsychiatric |
| | | NMDA receptor antagonist | symptoms agent |
| | | | (agitation) |
| AXS-05 | Neurotransmitter receptors | NMDA receptor antagonist; | Neuropsychiatric |
| | | combination of | symptoms agent |
| | | dextromethorphan and | (agitation) |
| | | bupropion | |
| Blarcamesine | Synaptic | Sigma-1 receptor agonist, M2 | DMT |
| (ANAVEX2-73) | plasticity/neuroprotection | autoreceptor antagonist; to | |
| | | ameliorate oxidative stress, | |
| | | protein misfolding, | |
| | | mitochondrial dysfunction, and | |
| | | inflammation | |
| BPDO-1603 | Undisclosed | Undisclosed | Cognitive enhancer |
| Brexpiprazole | Neurotransmitter | Atypical antipsychotic; D2 receptor | Neuropsychiatric symptoms |
| | receptors | partial agonist; serotonin-dopamine | agent (agitation) |
| | | modulator | |
| Caffeine | Neurotransmitter | Adenosine antagonist; non-specific | Cognitive enhancer |
| | receptors | phosphodiesterase inhibitor | |
| Donanemab | Amyloid | Monoclonal antibody specific for | DMT |
| | | pyroglutamate form of Aβ | |
| Donanemab & | Amyloid | Monoclonal antibody specific for | DMT |
| Aducanumab | | pyroglutamate form of Aβ | |
| | | (donanemab); monoclonal antibody | |
| | | directed at plaques and oligomers | |
| | | (aducanumab); given in separate arms | |
| | | of the trial | |
| Donepezil | Neurotransmitter | Acetylcholinesterase inhibitor | Cognitive enhancer |
| | receptors | | |
| Escitalopram | Neurotransmitter | Selective serotonin reuptake inhibitor | Neuropsychiatric symptoms |
| | receptors | | agent (agitation) |
| Gantenerumab | Amyloid | Monoclonal antibody directed at Aβ | DMT |
| | | plaques and oligomers | |
| Gantenerumab & | Amyloid | Monoclonal antibody directed at Aβ | DMT |
| Solanezumab | | plaques and oligomers | |
| | | (gantenerumab); Monoclonal | |
| | | antibody directed at Aβ monomers | |
| | | (solanezumab); given in separate | |
| | | arms of the trial | |
| Guanfacine | Neurotransmitter receptors | Alpha-2 adrenergic agonist | Cognitive enhancer |
| GV-971 | Gut-brain axis | Algae-derived acidic | DMT |
| | | oligosaccharides; changes | |
| | | microbiome to reduce peripheral | |
| | | and central inflammation | |
| Hydralazine | Oxidative stress | Free radical scavenger | DMT |
| Icosapent ethyl | Oxidative stress | Purified form of the omega-3 fatty | DMT |
| (IPE) | | acid EPA; to improve synaptic | |
| | | function and reduce inflammation | |
| Lecanemab | Amyloid | Monoclonal antibody directed at Aβ | DMT |
| (BAN2401) | | protofibrils | |
| Losartan & | Vasculature | Angiotensin II receptor blocker | DMT |
| Amlodipine & | | (losartan), calcium channel blocker | |
| Atorvastatin + | | (amlodipine), cholesterol agent | |
| exercise | | (atorvastatin) | |
| Metformin | Metabolism and bioenergetics | Insulin sensitizer to improve CNS | DMT |
| | | glucose metabolism | |
| Nabilone | Neurotransmitter receptors | Synthetic cannabinoid | Neuropsychiatric |
| | | | symptoms agent |
| | | | (agitation) |
| NE3107 | Inflammation | MAPK-1/3 inhibitor; reduces | DMT |
| | | proinflammatory NF?B activation | |
| Nilotinib BE | Proteostasis/Proteinopathies | Tyrosine kinase inhibitor; | DMT |
| | | autophagy enhancer; promotes | |
| | | clearance of Aβ and tau | |
| Octohydro- | Neurotransmitter receptors | Acetylcholinesterase inhibitor | Cognitive |
| aminoacridine | | | enhancer |
| Succinate | | | |
| Omega-3 | Oxidative stress | Antioxidant | DMT |
| (DHA + EPA) | | | |
| Remternetug | Amyloid | Anti-amyloid monoclonal antibody | DMT |
| | | targeting pyroglutamate amyloid | |
| Semaglutide | Metabolism and | GLP-1 agonist; reduces | DMT |
| | bioenergetics | neuroinflammation and improves | |
| | | insulin signaling in the brain | |
| Simufilam (PTI- | Synaptic | Filamin A protein inhibitor; stabilizes | DMT |
| 125) | Plasticity/Neuroprotection | amyloid-alpha-7 nicotinic receptor | |
| | | interaction | |
| Solanezumab | Amyloid | Monoclonal antibody directed at Aβ | DMT |
| | | monomers | |
| Tricaprilin | Metabolism and | Caprylic triglyceride; induces | DMT |
| | bioenergetics | ketosis and improves mitochondrial | |
| | | and neuronal function | |
| TRx0237 | Tau | Tau protein aggregation inhibitor | DMT |
| Valiltramiprosate | Amyloid | Prodrug of tramiprosate; inhibits Aβ | DMT |
| (ALZ-801) | | aggregation into toxic oligomers | |
| ABvac40 | Amyloid | Active immunotherapy to remove Aβ | DMT |
| ACI-35 | Tau | Active immunotherapy targeting tau | DMT |
| AD-35 | Neurotransmitter receptors | Acetylcholinesterase inhibitor | Cognitive |
| | | | enhancer |
| AL002 | Inflammation | Monoclonal antibody targeting | DMT |
| | | TREM2 receptors to promote | |
| | | microglial clearance of Aβ | |
| Allopregnanolone | Neurogenesis | Allosteric modulator of GABA-A | DMT |
| | | receptors | |
| APH-1105 | Amyloid | Alpha-secretase modulator to reduce | DMT |
| | | Aβ production | |
| Baricitinib | Inflammation | Janus kinase inhibitor; reduces | DMT |
| | | neuroinflammation | |
| Bepranemab | Tau | Anti-tau monoclonal antibody | DMT |
| BCG vaccine | Inflammation/Immunity | Immunomodulator | DMT |
| BPN14770 | Synaptic | PDE-4 inhibitor; prolongs cAMP | DMT |
| | plasticity/neuroprotection | activity and improves neuronal | |
| | | plasticity | |
| Bromocriptine | Neurotransmitter receptors | Dopamine agonist with anti-Aβ | DMT |
| | | effects | |
| Bryostatin 1 | Synaptic | Protein Kinase C inhibitor; facilitates | DMT |
| | plasticity/neuroprotection | synaptogenesis | |
| BXCL-501 | Neurotransmitter receptors | Sublingual dexmedetomidine; selective | Neuropsychiatric |
| | | a2-adrenergic receptor agonist | symptoms |
| | | | agent (agitation) |
| Canakinumab | Inflammation | Anti-IL-1β monoclonal antibody | DMT |
| CORT108297 | Hormones | Selective glucocorticoid receptor | Cognitive |
| | | antagonist; reduce neuroendocrine | enhancer |
| | | stress responses | |
| Crenezumab | Amyloid | Monoclonal antibody targeting soluble | DMT |
| | | Aβ oligomers | |
| CST-2032 | Neurotransmitter receptors | Noradrenergic agonist | Cognitive |
| | | | enhancer |
| Curcumin + | Inflammation | Herb with antioxidant and anti- | DMT |
| aerobic yoga | | inflammatory properties | |
| CY6463 | Synaptic | Guanylate cyclase positive allosteric | DMT |
| | Plasticity/Neuroprotection | modulator | |
| DAOIB | Neurotransmitter receptors | NMDA receptor antagonist to augment | Cognitive |
| | | the effect of tDCS | enhancer |
| Dapagliflozin | Metabolism and | SGLT2 inhibitor; to improve insulin | DMT |
| | bioenergetics | sensitivity and CNS glucose | |
| | | metabolism | |
| Daratumumab | Inflammation/immunity | Monoclonal antibody targeting CD38; | DMT |
| | | regulates microglial activity | |
| Dasatinib + | Inflammation/immunity | Tyrosine kinase inhibitor (dasatinib) and | DMT |
| Quercetin | | flavonoid (quercetin); senolytic therapy | |
| | | approach to reduce senescent cells and | |
| | | tau aggregation | |
| Deferiprone | Cell death | Iron chelating agent; reduce damaging | DMT |
| | | reactive oxygen species | |
| DHA | Oxidative stress | Omega 3 fatty acid; improve synaptic | DMT |
| | | function; antioxidant | |
| Donanemab | Amyloid | Monoclonal antibody specific for | DMT |
| (LY3002813) | | pyroglutamate Aβ | |
| Donanemab | Amyloid | Monoclonal antibody specific for | DMT |
| (LY3002813) | | pyroglutamate Aβ | |
| Dronabinol | Neurotransmitter | CB1 and CB2 endocannabinoid | Neuropsychiatric |
| | receptors | receptor partial agonist | symptoms agent |
| | | | (agitation) |
| E2814 | Tau | Anti-tau monoclonal antibody | DMT |
| Edonerpic (T- | Synaptic | Neurotrophic agent; activates sigma | DMT |
| 817MA) | plasticity/neuroprotection | receptors to preserve synaptic | |
| | | plasticity; protect against Aβ toxicity | |
| Elayta (CT1812) | Synaptic | Sigma-2 receptor antagonist; competes | DMT |
| | plasticity/neuroprotection | with oligomeric Aβ binding; protect | |
| | | against Aβ-induced synaptic toxicity | |
| ExPlas (exercised | Synaptic | Plasma transfusion from exercise- | DMT |
| plasma) | plasticity/neuroprotection | trained donors | |
| Fosgonimeton | Synaptic | Activates signaling via the hepatocyte | DMT |
| (ATH-1017) | plasticity/neuroprotection | growth factor system to regenerate | |
| | | neurons and enhance synaptic | |
| | | plasticity | |
| Gantenerumab | Amyloid | Monoclonal antibody directed at Aβ | DMT |
| | | plaques and oligomers | |
| Brain Shuttle | Amyloid | Anti-Aβ monoclonal antibody | DMT |
| Gantenerumab | | (gantenerumab) with enhanced blood- | |
| (RO7126209) | | brain barrier penetration | |
| GB301 | Inflammation/immunity | Regulatory T cells; reduce | DMT |
| | | neuroinflammation | |
| Grapeseed | Proteostasis/proteinopathies | Polyphenolic compound; antioxidant; | DMT |
| extract | | prevent aggregation of Aβ and tau | |
| GV1001 | Epigenetic | hTERT peptide vaccine; mimics extra- | DMT |
| | | telomeric functions to inhibit | |
| | | neurotoxicity, apoptosis, and reactive | |
| | | oxygen species | |
| Intranasal insulin | Metabolism and | Decrease glucose resistance and | DMT |
| | bioenergetics | increase insulin signaling in the brain | |
| Intranasal insulin + | Metabolism and | SGLT2 inhibitor (empagliflozin) and | DMT |
| Empagliflozin | bioenergetics | insulin combination therapy; decrease | |
| | | glucose resistance and increase insulin | |
| | | signaling in the brain | |
| IONIS MAPTRx | Tau | Antisense oligonucleotide targeting tau | DMT |
| (BIIB080) | | expression; MAPT RNA inhibitor | |
| JNJ-63733657 | Tau | Monoclonal antibody targeting soluble | DMT |
| | | tau | |
| Lamivudine (3TC) | Epigenetic | Nucleoside reverse transcriptase | DMT |
| | | inhibitor; reduces genetic | |
| | | rearrangements | |
| Lecanemab | Amyloid | Monoclonal antibody directed at | DMT |
| (BAN2401) | | protofibrils | |
| Lenalidomide | Inflammation/immunity | Reduce inflammatory cytokines; | DMT |
| | | modulate innate and adaptive immune | |
| | | responses | |
| Levetiracetam | Synaptic | SV2A modulator; improve synaptic | DMT |
| | plasticity/neuroprotection | function; reduce Aβ-induced neuronal | |
| | | hyperactivity | |
| L-Serine | Inflammation | Dietary amino acid; reduce brain | DMT |
| | | inflammation and preserve nerve cells | |
| Lupron (leuprolide | Growth factors and | GnRH receptor agonist; reduce effects | DMT |
| acetate depot) | hormones | of elevated GnRH and gonadotropins | |
| | | on the brain | |
| LY3372689 | Tau | O-GlycNAcase inhibitor; promote tau | DMT |
| | | glycosylation, prevent tau aggregation | |
| Memantine | Neurotransmitter | NMDA receptor antagonist | DMT |
| | receptors | | |
| Metabolic cofactor | Metabolism and | Mixture of N-acetylcysteine, L-carnitine | Cognitive |
| supplementation | bioenergetics | tartrate, nicotinamide roboside, and | enhancer |
| | | serine to increase mitochondrial activity | |
| MIB-626 | Amyloid | Sirtuin-nicotinamide adenine | DMT |
| | | dinucleotide stimulator to enhance | |
| | | alpha-secretase | |
| Montelukast | Inflammation | Cysteinyl leukotriene type 1 (cysLT-1) | DMT |
| | | receptor antagonist; effects on | |
| | | inflammatory processes, neuronal | |
| | | injury, blood-brain-barrier integrity, and | |
| | | Aβ protein accumulation | |
| MW150 | Synaptic | p38 MAPK-a inhibitor | DMT |
| | plasticity/neuroprotection | | |
| Neflamapimod | Synaptic | p38 MAPK-a inhibitor; enhance | DMT |
| (VX-745) | plasticity/neuroprotection | endolysosomal function to reduce | |
| | | synaptic dysfunction | |
| Nicotinamide | Tau | HDAC inhibitor; to reduce tau-induced | DMT |
| | | microtubule depolymerization and tau | |
| | | phosphorylation | |
| Nicotine | Neurotransmitter | Nicotinic acetylcholine receptor agonist | Cognitive |
| transdermal patch | receptors | | enhancer |
| Obicetrapib | Lipids and lipoprotein | Cholesteryl ester transfer protein | DMT |
| | receptors | (CETP) inhibitor | |
| Omega-3 PUFA | Vasculature | Polyunsaturated fatty acid; reduce | DMT |
| | | damage to small blood vessels | |
| Pepinemab | Inflammation | Monoclonal antibody directed at | DMT |
| (VX15) | | semaphorin 4D to reduce inflammation | |
| Posiphen | Proteostasis/proteinopathies | Inhibitor of APP and a-synuclein | DMT |
| Prazosin | Neurotransmitter | Alpha-1 adrenoreceptor antagonist | Neuropsychiatric |
| | receptors | | symptoms agent |
| | | | (agitation) |
| PU-AD | Tau | Heat shock protein 90 inhibitor; to | DMT |
| | | prevent aggregation and | |
| | | hyperphosphorylation of tau | |
| Rapamycin | Proteostasis/proteinopathies | mTOR inhibitor; ameliorate metabolic | DMT |
| (sirolimus) | | and vascular effects of aging | |
| Sargramostim | Inflammation/immunity | Granulocyte macrophage colony | DMT |
| | | stimulating factor | |
| Semorinemab | Tau | Monoclonal antibody to remove | DMT |
| (RO7105705) | | extracellular tau | |
| Senicapoc | Inflammation | Calcium-activated potassium channel | DMT |
| | | blocker | |
| Sovateltide (PMZ- | Neurogenesis | Endothelin B receptor agonist; | DMT |
| 1620) | | augments activity of neuronal | |
| | | progenitor cells | |
| Simufilam (PTI- | Synaptic | Filamin A protein inhibitor; stabilizes the | DMT |
| 125) | plasticity/neuroprotection | interaction of soluble Aβ and the alpha7 | |
| | | nicotinic acetylcholine receptor, | |
| | | reducing Aβ and synaptic dysfunction | |
| Suvorexant | Neurotransmitter | Dual Orexin receptor antagonist; | DMT |
| | receptors | improved sleep with effects on CSF Aβ | |
| T3D-959 | Metabolism and | Dual agonist of PPARd/?; reduce | DMT |
| | bioenergetics | glucose and lipid metabolism | |
| TB006 | Inflammation | Monoclonal antibody targeting galactin 3 | DMT |
| Telmisartan & | Vasculature | Angiotensin II receptor blocker | DMT |
| perindopril | | (telmisartan); angiotensin converting | |
| | | enzyme inhibitor (perindopril) | |
| Tdap vaccine | Inflammation and | Immune reaction to diphtheria, | DMT |
| | immunity | pertussis, tetanus vaccine | |
| THC-free CBD oil | Neurotransmitter | Cannabinoid with effects on | Neuropsychiatric |
| | receptors | cannabinoid receptors | symptoms agent |
| | | | (agitation) |
| Thiethylperazine | Amyloid | Activates transport protein ABCC1 to | DMT |
| (TEP) | | remove Aβ | |
| Troriluzole | Synaptic | Glutamate modulator; prodrug of | DMT |
| (BHV4157) | plasticity/neuroprotection | riluzole; improve synaptic function | |
| Valacyclovir | Infection/immunity | Antiviral against HSV-1 and -2 infection; | DMT |
| | | to prevent Aβ aggregation and plaque | |
| | | deposition | |
| Valiltramiprosate | Amyloid | Prodrug of tramiprosate; inhibits Aβ | DMT |
| (ALZ-801) | | aggregation into toxic oligomers | |
| Varoglutamstat | Amyloid | Glutaminyl cyclase (QC) enzyme | DMT |
| (PQ912) | | inhibitor to reduce pyroglutamate Aβ | |
| | | production | |
| VGH-AD1 | Undisclosed | Traditional Chinese herbal medicine | Cognitive |
| | | | enhancer |
| Xanamem | Growth factors and | 11-beta-hydroxysteroid dehydrogenase | DMT |
| | hormones | type 1 inhibitor | |
| Yangxue Qingnao | Vasculature | Cerebral blood flow enhancer | DMT |
| pills | | | |
| AAV-Htert | Epigenetic | Extending telomeres may benefit AD; | DMT |
| | | reduce Aβ-induced neurotoxicity; | |
| | | effects on multiple cellular pathways | |
| ACU193 | Amyloid | Monoclonal antibody targeting soluble | DMT |
| | | Aβ | |
| Allopregnanolone | Neurogenesis | GABA-A receptor modulator; promote | DMT |
| | | neurogenesis and reduce inflammation | |
| ASN51 | Tau | O-GlycNAcase Inhibitor | DMT |
| BEY2153 | Proteostasis/proteinopathies | Aβ and tau aggregation inhibitor; | DMT |
| | | inhibits neuronal death | |
| BMS-984923 | Synaptic | mGluR5 allosteric modulator | DMT |
| | plasticity/neuroprotection | | |
| BDPP (bioactive | Proteostasis/proteinopathies | Prevents Aβ and tau aggregation | DMT |
| dietary polyphenol | | | |
| preparation) | | | |
| Contraloid acetate | Proteostasis/proteinopathies | Aggregation inhibitor | DMT |
| COR588 | Synaptic | Lysine-gingipain inhibitor | DMT |
| | plasticity/neuroprotection | | |
| Dabigatran | Vasculature | Direct thrombin inhibitor; reduce | DMT |
| | | neurovascular damage | |
| Donepezil | Neurotransmitter | Cholinesterase inhibitor | Cognitive |
| | receptors | | enhancer |
| Edicotinib (JNJ- | Inflammation | CSF-1R antagonist; attenuates | DMT |
| 40346527) | | microglial proliferation and | |
| | | neurodegeneration | |
| Efavirenz | Epigenetics | NNRTI; promote cholesterol removal; | DMT |
| | | enhance amyloid reduction. | |
| Emtricitabine | Inflammation | NRTI; reduce neuroinflammation | DMT |
| IGC AD1 | Neurotransmitter | Tetrahydrocannabinol that binds to the | Neuropsychiatric |
| | receptors | CB1 receptor | symptoms agent |
| | | | (agitation) |
| Lu AF87908 | Tau | Monoclonal antibody to reduce tau | DMT |
| LX1001 | Epigenetic | 10hAPOE2, serotype rh. Ten AAV gene | DMT |
| | | transfer vector expressing the cDNA | |
| | | coding for human APOE e2, directly to | |
| | | the CNS/CSF of APOE e4 | |
| | | homozygotes with AD | |
| Remternetug | Amyloid | Monoclonal antibody to reduce Aβ | DMT |
| LY3372993 | | | |
| MK-1942 + | Neurotransmitter | Undisclosed (MK-1942) | Cognitive |
| donepezil | receptors | | enhancer |
| NNI-362 | Neurogenesis | Enhance neurogenesis; activates | DMT |
| | | progenitor cells | |
| REM0046127 | Synaptic | Regulates calcium dyshomeostasis; tau | DMT |
| | Plasticity/Neuroprotection | and Aβ reduction | |
| Salsalate | Inflammation | Non-steroidal anti-inflammatory to | DMT |
| | | reduce inflammation | |
| SHR-1707 | Amyloid | Anti-amyloid monoclonal antibody | DMT |

| Agent | Status (CT. gov ID) | Sponsor |
|---|---|---|
| Aducanumab | Active, not recruiting | Biogen |
| | (NCT04241068) | |
| AGB101 (low-dose | Active, not recruiting | AgeneBio, NIA |
| levetiracetam) | (NCT03486938) | |
| Atuzaginstat | Active, not recruiting | Cortexyme |
| (COR388) | (NCT03823404) | |
| AVP-786 | Recruiting | Avanin |
| | (NCT03393 520) | |
| | Recruiting, extension | Avanir |
| | study (NCT02446132) | |
| | Recruiting | Avanir |
| | (NCT04464564) | |
| | Recruiting, extension | Avanir |
| | study (NCT04408755) | |
| AXS-05 | Recruiting | Axsome therapeutics |
| | (NCT04797715) | |
| | Recruiting, extension | Axsome therapeutics |
| | study (NCT04947553) | |
| Blarcamesine | Active, not recruiting | Anavex life sciences |
| (ANAVEX2-73) | (NCT03790709) | |
| | recruiting | Anavex life sciences |
| | (NCT04314934) | |
| BPDO-1603 | Recruiting | Hyundai |
| | (NCT04229927) | pharmaceutical |
| Brexpiprazole | recruiting | Otsuka |
| | (NCT03620981) | |
| | Recruiting, | Otsuka |
| | extension study | |
| | (NCT03594123) | |
| | Recruiting | Otsuka |
| | (NCT03548584) | |
| Caffeine | Not yet recruiting | University Hospital, |
| | (NCT04570085) | Lille |
| Donanemab | Active, not | Eli Lilly |
| | recruiting | |
| | (NCT04437511) | |
| | Recruiting | Eli Lilly |
| | (NCT05026866) | |
| Donanemab & | Recruiting | Eli Lilly |
| Aducanumab | (NCT05108922) | |
| Donepezil | Not yet recruiting | Assistance Publique - |
| | (NCT04661280) | Hôpitaux de Paris |
| Escitalopram | Recruiting | Johns Hopkins |
| | (NCT03108846) | University, NIA |
| Gantenerumab | Recruiting | Roche |
| | (NCT03444870) | |
| | (NCT03443973) | Roche |
| | (NCT04339413) | Roche |
| | (NCT04374253) | Roche |
| Gantenerumab & | recruiting; | Washington |
| Solanezumab | (NCT01760005) | University, Eli Lilly, |
| | | Roche, NIA, |
| | | Alzheimer's |
| | | Association |
| Guanfacine | Recruiting; | Imperial College |
| | (NCT03116126) | London, UK NIHR |
| GV-971 | Recruiting; | Shanghai |
| | (NCT04520412) | Greenvalley |
| Hydralazine | Recruiting; | Shahid Sadoughi |
| | (NCT04842552) | University, Iran |
| Icosapent ethyl | Active, not | VA Office of |
| (IPE) | recruiting; | Research and |
| | (NCT02719327) | Development, |
| | | University of |
| | | Wisconsin, Madison |
| Lecanemab | Active, not | Eisai, Biogen |
| (BAN2401) | recruiting; | |
| | (NCT03887455) | |
| | Recruiting; | Eisai, Biogen, |
| | (NCT04468659) | ACTC, NIA |
| Losartan & | Active, not | University of Texas |
| Amlodipine & | recruiting; | Southwestern |
| Atorvastatin + | (NCT02913664) | |
| exercise | | |
| Metformin | recruiting; | Columbia |
| | (NCT04098666) | University, NIA |
| Nabilone | Recruiting | Sunnybrook Health |
| | (NCT04516057) | Sciences Center, |
| | | ADDF |
| NE3107 | Recruiting; | Neurmedix |
| | (NCT04669028) | |
| Nilotinib BE | Not yet recruiting; | KeifeRx |
| | (NCT05143528) | |
| Octohydro- | Recruiting; (NCT03283059) | Shanghai Mental |
| aminoacridine | | Health Center |
| Succinate | | |
| Omega-3 | Recruiting; (NCT03691519) | University Hospital, |
| (DHA + EPA) | | Toulouse |
| Remternetug | Recruiting (NCT05463731) | Eli Lilly |
| Semaglutide | Recruiting; (NCT04777396) | Novo Nordisk |
| | Recruiting; (NCT04777409) | Novo Nordisk |
| Simufilam (PTI- | Recruiting; (NCT04994483) | Cassava sciences |
| 125) | | |
| | Recruiting; (NCT05026177) | Cassava sciences |
| Solanezumab | Active, not recruiting; | Eli Lilly, ATRI |
| | (NCT02008357) | |
| Tricaprilin | Not yet recruiting; | Cerecin |
| | (NCT04187547) | |
| TRx0237 | Active, not recruiting; | TauRx Therapeutics |
| | (NCT03446001) | |
| Valiltramiprosate | Recruiting; (NCT04770220) | Alzheon, NIA |
| (ALZ-801) | | |
| ABvac40 | Active, not recruiting; | Araclon Biotech |
| | (NCT03461276) | |
| ACI-35 | recruiting; | AC Immune, Janssen |
| | (NCT04445831) | |
| AD-35 | Active, not recruiting; | Zhejiang Hisun |
| | (NCT03625401) | Pharmaceutical |
| AL002 | Recruiting; | Alector, AbbVie |
| | (NCT04592874) | |
| Allopregnanolone | Not yet recruiting; | University of Arizona, NIA |
| | (NCT04838301) | |
| APH-1105 | Not yet recruiting; | Aphios |
| | (NCT03806478) | |
| Baricitinib | Not yet recruiting; | Massachusetts General |
| | (NCT05189106) | Hospital |
| Bepranemab | Recruiting; | UCB Biopharma |
| | (NCT04867616) | |
| BCG vaccine | Not yet recruiting; | Massachusetts General |
| | (NCT05004688) | Hospital |
| BPN14770 | Active, not recruiting; | Tetra discovery partners |
| | (NCT03817684) | |
| Bromocriptine | Active, not recruiting; | Kyoto University |
| | (NCT04413344) | |
| Bryostatin 1 | Recruiting; | Neurotrope Bioscience, |
| | (NCT04538066) | NIH, NIA |
| BXCL-501 | Recruiting; | BioXcel therapeutics |
| | (NCT04251910) | |
| Canakinumab | Recruiting; | Novartis |
| | (NCT04795466) | |
| CORT108297 | Recruiting; | Johns Hopkins University |
| | (NCT04601038) | |
| Crenezumab | Active, not | Genentech, NIA Banner |
| | recruiting; | Alzheimer's Institute |
| | (NCT01998841) | |
| CST-2032 | Not yet recruiting; | CuraSen Therapeutics |
| | (NCT05104463) | |
| Curcumin + | Active, not | VA Office of Research |
| aerobic yoga | recruiting; | and Development |
| | (NCT01811381) | |
| CY6463 | Recruiting; | Cyclerion therapeutics |
| | (NCT04798989) | |
| DAOIB | NCT05006781 | Chang Gung Memorial |
| | | Hospital |
| Dapagliflozin | recruiting; | University of Kansas |
| | (NCT03801642) | |
| Daratumumab | Recruiting; | Northwell Health, Janssen |
| | (NCT04070378) | |
| Dasatinib + | recruiting; | The University of Texas at |
| Quercetin | (NCT04063124) | San Antonio, Mayo Clinic |
| | Recruiting; | Wake Forest |
| | (NCT04685590) | University, U. of |
| | | Texas at San Antonio |
| | recruiting; | Mayo Clinic |
| | (NCT04785300) | |
| Deferiprone | Active, not recruiting; | Neuroscience Trials |
| | (NCT03234686) | Australia |
| DHA | Recruiting; | University of Southern |
| | (NCT03613844) | California, NIA, ADDF |
| Donanemab | Active, not recruiting; | Eli Lilly |
| (LY3002813) | (NCT03367403) | |
| Donanemab | Recruiting; | Eli Lilly |
| (LY3002813) | (NCT04640077) | |
| Dronabinol | Recruiting; | Mclean Hospital, |
| | (NCT02792257) | Johns Hopkins |
| | | University |
| E2814 | recruiting; | Eisai |
| | (NCT04971733) | |
| Edonerpic (T- | Active, not recruiting; | Toyama Chemical |
| 817MA) | (NCT04191486) | |
| Elayta (CT1812) | Recruiting; | Cognition |
| | (NCT03507790) | therapeutics |
| | Recruiting; | Cognition |
| | (NCT04735536) | therapeutics |
| ExPlas (exercised | Recruiting; | Norwegian University, |
| plasma) | (NCT05068830) | St. Olavs Hospital |
| Fosgonimeton | Recruiting; | Athira Pharma |
| (ATH-1017) | (NCT04488419) | |
| | Active, not recruiting; | Athira Pharma |
| | (NCT04491006) | |
| | Recruiting, extension | Athira Pharma |
| | study; (NCT04886063) | |
| Gantenerumab | Active, not recruiting; | Roche |
| | (NCT04592341) | |
| Brain Shuttle | recruiting; | Roche |
| Gantenerumab | (NCT04639050) | |
| (RO7126209) | | |
| GB301 | Not yet recruiting; | GMP BIO, BHT |
| | (NCT03865017) | Lifescience Australia |
| Grapeseed | Active, not recruiting; | Mount Sinai School of |
| extract | (NCT02033941) | Medicine, NCCIH |
| GV1001 | Not yet recruiting; | GemVax & Kael |
| | (NCT03959553) | |
| | Not yet recruiting; | GemVax & Kael |
| | (NCT05189210) | |
| Intranasal insulin | Not yet recruiting; | Wake Forest University |
| | (NCT05006599) | |
| Intranasal insulin + | Not yet recruiting; | Wake Forest University |
| Empagliflozin | (NCT05081219) | |
| IONIS MAPTRx | Active, not recruiting; | Ionis Pharmaceuticals |
| (BIIB080) | (NCT03186989) | |
| JNJ-63733657 | Recruiting; | Janssen |
| | (NCT04619420) | |
| Lamivudine (3TC) | recruiting; | University of Texas |
| | (NCT04552795) | Health Science Center at |
| | | San Antonio |
| Lecanemab | Active, not recruiting; | Eisai |
| (BAN2401) | (NCT01767311) | |
| Lenalidomide | Recruiting; | Cleveland Clinic, NIA |
| | (NCT04032626) | |
| Levetiracetam | Active, not recruiting; | University of California, |
| | (NCT02002819) | San Francisco |
| | Active, not recruiting; | UCB Pharma, University |
| | (NCT03489044) | of Oxford, NHS |
| | | Foundation Trust |
| | Recruiting; | Beth Israel Deaconess |
| | (NCT03875638) | Medical Center |
| L-Serine | Active, not recruiting; | Dartmouth-Hitchcock |
| | (NCT03062449) | Medical Center |
| Lupron (leuprolide | Recruiting; | New York University |
| acetate depot) | (NCT03649724) | |
| LY3372689 | Recruiting; | Eli Lilly |
| | (NCT05063539) | |
| Memantine | Recruiting; | University of Virginia |
| | (NCT05063851) | |
| Metabolic cofactor | Recruiting; | Istanbul Medipol |
| supplementation | (NCT04044131) | University Hospital, |
| | | ScandiBio therapeutics |
| MIB-626 | Not yet recruiting; | Brigham and Women's |
| | (NCT05040321) | Hospital |
| Montelukast | Recruiting; | IntelGenx Corp. |
| | (NCT03402503) - | |
| | buccal film | |
| | Active, not recruiting; | Emory University |
| | (NCT03991988) - tablet | |
| MW150 | Not yet recruiting; | Neurokine Therapeutics, |
| | (NCT05194163) | Columbia University, NIA |
| Neflamapimod | Recruiting; | EIP Pharma |
| (VX-745) | (NCT03435861) | |
| Nicotinamide | Recruiting; | University of California, |
| | (NCT03061474) | Irvine |
| Nicotine | Recruiting; | University of Southern |
| transdermal patch | (NCT02720445) | California, NIA, ATRI, |
| | | Vanderbilt University |
| Obicetrapib | Not yet recruitin; | NewAmsterdam Pharma |
| | (NCT05161715) | |
| Omega-3 PUFA | Active, not recruiting; | Oregon Health and |
| | (NCT01953705) | Science University, NIA |
| Pepinemab | recruiting; | Vaccinex, ADDF, |
| (VX15) | (NCT04381468) | Alzheimer's Association |
| Posiphen | Active, not recruiting; | QR Pharma, ADCS |
| | (NCT02925650) | |
| | Active, not recruiting; | Annovis Bio, Parexel |
| | (NCT04524351) | |
| Prazosin | Active, not recruiting; | ADCS, NIA |
| | (NCT03710642) | |
| PU-AD | Active, not recruiting; | Samus therapeutics |
| | (NCT04311515) | |
| Rapamycin | Recruiting; | The University of Texas |
| (sirolimus) | (NCT04629495) | Health Science Center at |
| | | San Antonio |
| Sargramostim | Not yet recruiting; | University of Colorado, |
| | (NCT04902703) | NIA, Alz. Assn. |
| Semorinemab | Active, not recruiting; | Genentech |
| (RO7105705) | (NCT03828747) | |
| Senicapoc | Not yet recruiting; | University of California, |
| | (NCT04804241) | Davis |
| Sovateltide (PMZ- | Recruiting; | Pharmazz |
| 1620) | (NCT04052737) | |
| Simufilam (PTI- | Active, not recruiting; | Cassava Sciences, NIA |
| 125) | (NCT04388254) | |
| Suvorexant | Not yet recruiting; | Washington University |
| | (NCT04629547) | School of Medicine |
| T3D-959 | Recruiting; | T3D Therapeutics, |
| | (NCT04251182) | Alzheimer's Association, |
| | | NIA |
| TB006 | recruiting; | TrueBinding, Inc. |
| | (NCT05074498) | |
| Telmisartan & | Recruiting; | Sunnybrook Health |
| perindopril | (NCT02085265) | Sciences Centre, ADDF |
| Tdap vaccine | Not yet recruiting; | Mindful diagnostics and |
| | (NCT05183516) | therapeutics |
| THC-free CBD oil | Recruiting; | Eastern Virginia Medical |
| | (NCT04436081) | School, Ananda Hemp |
| Thiethylperazine | Active, not recruiting; | Immungenetics AG |
| (TEP) | (NCT03417986) | |
| Troriluzole | Active, not recruiting; | Biohaven Pharma, ADCS |
| (BHV4157) | (NCT03605667) | |
| Valacyclovir | Recruiting; | New York State |
| | (NCT03282916) | Psychiatric Institute, NIH, |
| | | NIA |
| Valiltramiprosate | Active, note recruiting; | Alzheon |
| (ALZ-801) | (NCT04693520) | |
| Varoglutamstat | Recruiting; | Vivoryon Therapeutics |
| (PQ912) | (NCT03919162) | AG, ADCS, NIA |
| | Recruiting; | Vivoryon Therapeutics |
| | (NCT04498650) | AG, ADCS, NIA |
| VGH-AD1 | Not yet recruiting; | Taipei Veterans General |
| | (NCT04249869) a | Hospital, Taiwan |
| Xanamem | Active, not | Actinogen Medical |
| | recruiting(NCT04983368) | |
| Yangxue Qingnao | Recruiting | Dongzhimen Hospital, |
| pills | (NCT04780399) | Beijing |
| AAV-Htert | Recruiting; | Libella Gene |
| | (NCT04133454) | Therapeutics |
| ACU193 | Recruiting; | Acumen |
| | (NCT04931459) | Pharmaceuticals, NIA |
| Allopregnanolone | Active, not recruiting; | University of Southern |
| | (NCT03748303) | California, University of |
| | | Arizona, Alzheimer's |
| | | Association |
| ASN51 | Recruiting; | Asceneuron |
| | (NCT04759365) | |
| BEY2153 | Recruiting; | BeyondBio |
| | (NCT04476303) | |
| BMS-984923 | Recruiting; | Yale University, NIA |
| | (NCT04805983) | |
| BDPP (bioactive | Recruiting; | Johns Hopkins |
| dietary polyphenol | (NCT02502253) | University, Mount Sinai |
| preparation) | | School of Medicine |
| Contraloid acetate | Recruiting; | Charite University, |
| | (NCT04711486) | Berlin, Germany |
| COR588 | Recruiting; | Cortexyme |
| | (NCT04920903) | |
| Dabigatran | Not yet recruiting; | University of Rhode |
| | (NCT03752294) | Island, ADDF, |
| | | Boehringer Ingelheim |
| Donepezil | Recruiting; | Merck |
| | (NCT04730635) | |
| Edicotinib (JNJ- | Not yet recruiting; | Janssen, University of |
| 40346527) | (NCT04121208) | Oxford |
| Efavirenz | Active, not recruiting; | Case Western Reserve |
| | (NCT03706885) | University, Cleveland |
| | | Medical Center, Mass. |
| | | General Hospital |
| Emtricitabine | Recruiting; | Butler Hospital, |
| | (NCT04500847) | Alzheimer's |
| | | Association, Brown |
| | | University |
| IGC AD1 | Recruiting; | IGC Pharma |
| | (NCT04749563) | |
| Lu AF87908 | Recruiting; | Lundbeck |
| | (NCT04149860) | |
| LX1001 | Recruiting; | Cornell University |
| | (NCT03634007) | |
| Remternetug | Recruiting; | Eli Lilly |
| LY3372993 | (NCT04451408) | |
| MK-1942 + | Recruiting; | Merck |
| donepezil | (NCT04308304) | |
| NNI-362 | Recruiting; | Neuronascent, NIA |
| | (NCT04074837) | |
| REM0046127 | Recruiting; | reMYND, NeuroScios |
| | (NCT04672135) | GmbH |
| Salsalate | Active, not recruiting; | University of California, |
| | (NCT03277573) | San Francisco |
| SHR-1707 | Recruiting; | Shanghai Hengrui |
| | (NCT04973189) | Pharmaceutical |
| | Recruiting; | Atridia Pty Ltd. |
| | (NCT04745104) | |

| | | | | | Amyloid evidence at |
|---|---|---|---|---|---|
| Agent | Phase | Status (CT. gov ID) | Sponsor | Subject characteristics | entry |
| Allogeneic human | 1 | Active, not recruiting; (NCT04040348) | University of Miami | Mild to moderate AD with | Amyloid PET or CSF |
| MSCs | | | | MMSE of 20 to 26 | |
| SNK01 (autologous | 1 | Recruiting; (NCT04678453) | NKMax America | MCI or AD | Not required |
| natural killer cell) | | | | | |
| Allogenic adipose | 1--2 | Recruiting; (NCT04388982) | Ruijin Hospital, Cellular | Mild to moderate AD with | Not required |
| MSC-Exosomes | | | Biomedicine Group | MMSE of 10 to 24 | |
| CB-AC-02 (placenta | 1--2 | Recruiting; (NCT02899091) | CHABiotech Co. | Mild to moderate AD with | Amyloid PET |
| derived MSCs) | | | | KMMSE of 10 to 26 | |
| Human umbilical cord | 1--2 | Recruiting, extension study; | Medipost | Probable AD with | Amyloid PET |
| blood-derived MSCs | | (NCT03172117) | | KMMSE of 18 to 26 | |
| (NEUROSTEM) | | | | | |
| Allogeneic human | 2 | Recruiting; (NCT02833792) | Stemedica | Mild to moderate AD with | Amyloid PET |
| MSCs | | | | MMSE of 12 to 24 | |
| AstroStem | 2 | Not yet recruiting; (NCT04482413) | Nature Cell Co. | Mild AD with MMSE of 20 | CSF amyloid |
| (autologous adipose- | | | | to 24 | |
| derived MSCs) | | | | | |

In some embodiments, the therapeutic agent is a cholinesterase inhibitor therapeutic agent listed in Table 10. In some embodiments, the therapeutic agent is an NMDA receptor antagonist therapeutic agent listed in Table 10. In some embodiments, the therapeutic agent is an acetylcholinesterase and butyrylcholinesterase inhibitor therapeutic agent listed in Table 10. In some embodiments, the therapeutic agent is an antibody therapeutic agent targeting amyloid-beta peptide listed in Table 10. In some embodiments, the therapeutic agent is an antibody therapeutic agent targeting tau listed in Table 10. In some embodiments, the therapeutic agent is an anti-sense therapy targeting huntingtin protein (HTT) listed in Table 10. In some embodiments, the therapeutic agent is Tominersen. In some embodiments, two or more therapeutic agents are administered. In some embodiments, the two or more therapeutic agents administered are listed in Table 10. In some embodiments, the two or more therapeutic agents administered are an NMDA receptor antagonist therapeutic agent listed in Table 10 and a cholinesterase inhibitor therapeutic agent listed in Table 10. Described herein, in certain aspects, are therapeutic agents for preventing, inhibiting the progress of, treating, or ameliorating one or more symptoms of a neurodegenerative disease in a subject. Described herein, in certain aspects, are therapeutic agents for preventing, inhibiting the progress of, treating, or ameliorating one or more symptoms of a tauopathy in a subject. Tauopathies are neurodegenerative diseases caused by abnormal aggregation of tau proteins in the form of neurofibrillary tangles (NFT) in the human brain. Examples of tauopathies include, but are not limited to, Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, Hallevorden-Spatz disease, or a transgene-induced tauopathy. In some embodiments, the therapeutic agents described herein are provided for preventing, inhibiting the progress of, treating, or ameliorating one or more symptoms of Alzheimer's disease in a subject. In some embodiments, the therapeutic agents described herein are provided for preventing, inhibiting the progress of, treating, or ameliorating one or more symptoms of Pick's disease in a subject. In some embodiments, the therapeutic agents described herein are provided for preventing, inhibiting the progress of, treating, or ameliorating one or more symptoms of frontotemporal dementia in a subject. In some embodiments, the therapeutic agents described herein are provided for preventing, inhibiting the progress of, treating, or ameliorating one or more symptoms of Parkinson's disease in a subject. In some embodiments, the therapeutic agents described herein prevent or inhibit the progress of Alzheimer's disease in a subject. Typically, Alzheimer's disease progress in three stages —early (mild), middle (moderate), and late (severe). In the early (mild) stage of Alzheimer's disease, patients may experience difficulty performing complex tasks, losing or misplacing belongings, or difficulty organizing or planning. In the middle (moderate) stage of Alzheimer's disease, patients may experience memory loss of events or personal history, depression, having trouble controlling their bladder and bowels, wandering and getting lost, or personality changes. In the late (severe) stage of Alzheimer's disease, patients may experience difficulty in communicating or inability to walk without assistance. In some embodiments, the therapeutic agents described herein prevent or inhibit the progress of early-stage Alzheimer's disease in a subject. In some embodiments, the therapeutic agents described herein prevent or inhibit the progress of middle-stage Alzheimer's disease in a subject. In some embodiments, the therapeutic agents described herein prevent or inhibit the progress of late-stage Alzheimer's disease in a subject. The degree ofNFT involvement AD is defined by Braak stages. When NFT involvement is mild and confined primarily to the transentorhinal region of the brain, this is termed Braak stage 1. In Braak stage 2, lesional density of NFTs increases and the pathology extends into the entorhinal area. Progression of AD to Braak stage 3 or Braak stage 4 includes NFT involvement in limbic regions of the CNS such as the hippocampus. In Braak stage 3, NFT pathology seen in the outer and inner entorhinal and transentorhinal cellular layers worsens compared with earlier stages and NFT lesions extend into adjoining neocortical association areas of the fusiform (occipito-temporal) gyrus. In Braak stage 4, lesional density increases in entorhinal areas and fusiform gyrus. NFT pathology extends up to the medial temporal gyrus. Progression of AD to Braak stage 5 or Braak stage 6 includes extensive NFT involvement throughout the neocortex. In Braak stage 5, NFT lesions extend throughout the occipital lobe and also appear in the peristriate area. In Braak stage 6, NFT lesions are seen in the parastriate and striate areas of the occipital neocortex. In some embodiments, progression through Braak stages is independent of progression of AD pathology characterized by amyloid plaques. In some embodiments, the therapeutic agents described herein prevent or inhibit the progress of Braak stage 1 Alzheimer's disease in a subject. In some embodiments, the therapeutic agents described herein prevent or inhibit the progress of Braak stage 2 Alzheimer's disease in a subject. In some embodiments, the therapeutic agents described herein prevent or inhibit the progress of Braak stage 3 Alzheimer's disease in a subject. In some embodiments, the therapeutic agents described herein prevent or inhibit the progress of Braak stage 4 Alzheimer's disease in a subject. In some embodiments, the therapeutic agents described herein prevent or inhibit the progress of Braak stage 5 Alzheimer's disease in a subject. In some embodiments, the therapeutic agents described herein prevent or inhibit the progress of Braak stage 6 Alzheimer's disease in a subject. In AD, the neuropathologic features of amyloid plaques and neurofibrillary, tau-based, tangles can begin about 15-20 years prior to the manifestation of obvious cognitive symptoms. Individuals with AD typically progress from their normal cognitive baseline abilities through subtle changes characteristic of the preclinical stages of AD to obvious symptoms of CNS dysfunction, termed prodromal AD, and finally, to AD dementia. The preclinical stage of AD is often termed as the stage occurring before a clinical diagnosis of a cognitive disorders is considered. In Preclinical stage 1 of AD: evidence of amyloidosis is found through PET imaging or CSF analysis. In Preclinical stage 2 of AD: evidence of amyloidosis and neurodegeneration is found through PET imaging and CSF analysis. In Preclinical stage 3 of AD: evidence of amyloidosis, neurodegeneration, and subtle cognitive changes are found. The prodromal stage of AD can be referred to as mild cognitive impairments (MCI) due to AD. At this stage, obvious symptoms of brain dysfunction manifest themselves and a subject at the prodromal stage of AD will meet the criteria for MCI and the primary underlying pathophysiology of judged by a clinician or medical professional will be considered to be due to AD. The dementia stage of AD occurs when there is significant impairments of a subject's social and occupational functioning, in some instances manifested by the loss of independence to perform activities of daily living due to cognitive impairments. Typically, AD dementia involves impairment of short term and semantic memory plus other cognitive domains (e.g., executive, language, and visuospatial functions). In some embodiments, the therapeutic agents described herein prevent or inhibit the progress of preclinical stages of Alzheimer's disease in a subject. In some embodiments, the therapeutic agents described herein prevent or inhibit the progress of Preclinical stage 1 of Alzheimer's disease in a subject. In some embodiments, the therapeutic agents described herein prevent or inhibit the progress of Preclinical stage 2 of Alzheimer's disease in a subject. In some embodiments, the therapeutic agents described herein prevent or inhibit the progress of Preclinical stage 3 of Alzheimer's disease in a subject. In some embodiments, the therapeutic agents described herein prevent or inhibit the progress of the prodromal stage of Alzheimer's disease in a subject. In some embodiments, the therapeutic agents described herein prevent or inhibit the progress of the dementia stage of Alzheimer's disease in a subject.

In some embodiments, the therapeutic agents described herein treat or ameliorate one or more symptoms of a tauopathy in a subject. In some embodiments, the therapeutic agents described herein treat or ameliorate one or more symptoms of Pick's disease in a subject. In some embodiments, the therapeutic agents described herein treat or ameliorate one or more symptoms ofNiemann-Pick disease type C in a subject. In some embodiments, the therapeutic agents described herein treat or ameliorate one or more symptoms of Frontal temporal dementia (FTD) in a subject. In some embodiments, the therapeutic agents described herein treat or ameliorate one or more symptoms of frontotemporal lobar degeneration in a subject. In some embodiments, the therapeutic agents described herein treat or ameliorate one or more symptoms of chronic traumatic encephalopathy (CTE) in a subject. In some embodiments, the therapeutic agents described herein treat or ameliorate one or more symptoms of progressive supranuclear palsy (PSP) in a subject. In some embodiments, the therapeutic agents described herein treat or ameliorate one or more symptoms of corticobasal degeneration (CBD) in a subject. In some embodiments, the therapeutic agents described herein treat or ameliorate one or more symptoms of Lytico-Bodig disease in a subject. In some embodiments, the therapeutic agents described herein treat or ameliorate one or more symptoms of tangle-predominant dementia in a subject. In some embodiments, the therapeutic agents described herein treat or ameliorate one or more symptoms of meningioaniomatosis in a subject. In some embodiments, the therapeutic agents described herein treat or ameliorate one or more symptoms of primary age-related tauopathy (PART) in a subject. In some embodiments, the therapeutic agents described herein treat or ameliorate one or more symptoms of argyrophilic grain disease (AGD) in a subject. In some embodiments, the therapeutic agents described herein treat or ameliorate one or more symptoms of globular glial tauopathy (GGT) in a subject. In some embodiments, the therapeutic agents described herein treat or ameliorate one or more symptoms of vacuolar tauopathy in a subject. In some embodiments, the therapeutic agents described herein treat or ameliorate one or more symptoms of tuberous sclerosis in a subject. In some embodiments, the therapeutic agents described herein treat or ameliorate one or more symptoms of postencephalitic parkinsonism in a subject. In some embodiments, the therapeutic agents described herein treat or ameliorate one or more symptoms of subacute sclerosing panencephalitis in a subject. In some embodiments, the therapeutic agents described herein treat or ameliorate one or more symptoms of amyotrophic lateral sclerosis in a subject. In some embodiments, the therapeutic agents described herein treat or ameliorate one or more symptoms of myotonic dystrophy in a subject. In some embodiments, the therapeutic agents described herein treat or ameliorate one or more symptoms of Pallido-ponto-nigral degeneration in a subject. In some embodiments, the therapeutic agents described herein treat or ameliorate one or more symptoms of Parkinson's disease in a subject. In some embodiments, the therapeutic agents described herein treat or ameliorate one or more symptoms of Creutzfeldt-Jacob disease in a subject. In some embodiments, the therapeutic agents described herein treat or ameliorate one or more symptoms of Dementia pugilistica in a subject. In some embodiments, the therapeutic agents described herein treat or ameliorate one or more symptoms of Down's syndrome in a subject. In some embodiments, the therapeutic agents described herein treat or ameliorate one or more symptoms of Gerstmann-Staussler-Scheinker disease in a subject. In some embodiments, the therapeutic agents described herein treat or ameliorate one or more symptoms of inclusion-body myositis in a subject. In some embodiments, the therapeutic agents described herein treat or ameliorate one or more symptoms of diffuse neurofibrillary tangles with calcification in a subject. In some embodiments, the therapeutic agents described herein treat or ameliorate one or more symptoms of Tangle-only dementia in a subject. In some embodiments, the therapeutic agents described herein treat or ameliorate one or more symptoms of Hallevorden-Spatz disease in a subject. In some embodiments, the therapeutic agents described herein treat or ameliorate one or more symptoms of a transgene-induced tauopathy in a subject.

In some embodiments, the therapeutic agents described herein treat or ameliorate one or more symptoms of a tauopathy in a subject. Examples of symptoms associated with a tauopathy include, but not limited to, mental decline, difficulty thinking and understanding, mental confusion, delusion, disorientation, forgetfulness, difficulty concentrating, inability to create new memories, inability to do simple math, inability to recognize common things, aggression, agitation, difficulty with self-care, irritability, meaningless repetition of own words, personality changes, lack of restraint, wandering and getting lost, anger, apathy, general discontent, loneliness, mood swings, depression, hallucination, paranoia, loss of appetite, restlessness, inability to combine muscle movements or jumbled speech.

In methods of treatment described herein, a subject is administered a therapeutic agent. In some embodiments, the therapeutic agent is formulated for delivery for a selected route of administration. In some embodiments, the therapeutic agent is formulated as a pharmaceutical composition. In some embodiments, the pharmaceutical composition administration can include injection or infusion, including intra-arterial, intracardiac, intradermal, intraduodenal, intramedullary, intramuscular, intraosseous, intraperitoneal, intravascular, intravenous, subcutaneous, inhalational, transdermal, transmucosal, sublingual, buccal and topical (including epicutaneous, dermal, enema, intranasal, vaginal) administration. In some exemplary embodiments, a route of administration can be via an injection such as an intramuscular, intravenous, subcutaneous, or intraperitoneal injection. In some embodiments, i.v. administration is a preferred route of administration. In some embodiments, the route of administration is by stereotactic injection. In some embodiments, stereotactic injection is used to target delivery of the therapeutic agent to the brain parenchyma or to one or more selected regions of the brain. In some embodiments, the therapeutic agent is administered intravenously, intramuscularly, intrathecally, intracerebrally, subcutaneously, orally, nasally, topically, buccally, or sublingually. In some embodiments, the therapeutic agent is administered directly to the CNS of the subject via intravenous delivery, intravascular delivery, intrathecal delivery, intracistemal delivery, intraspinal delivery, subpial delivery, or intracerebroventricular delivery. In some embodiments, the therapeutic agent is formulated as a liquid for delivery by the selected route of administration. A liquid formulation can include an intravenous formulation for i.v. administration. In some embodiments, the therapeutic agent is administered by oral administration. Solid dosage forms for oral administration can include capsules, tablets, caplets, pills, troches, lozenges, powders, and granules. A capsule can comprise a core material comprising a nutritive protein or composition and a shell wall that encapsulates a core material. In some embodiments a core material can comprise at least one of a solid, a liquid, and an emulsion. In some embodiments a shell wall material can comprise at least one of a soft gelatin, a hard gelatin, and a polymer. Suitable polymers can include but not limited to: cellulosic polymers such as hydroxypropyl cellulose, hydroxyethyl cellulose, hydroxypropyl methyl cellulose (HPMC), methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, such as those formed from acrylic acid, methacrylic acid, methyl acrylate, ammonio methylacrylate, ethyl acrylate, methyl methacrylate and/or ethyl methacrylate (e.g., those copolymers sold under the trade name “Eudragit”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac). In some embodiments at least one polymer can function as taste-masking agents. In some aspects, the pharmaceutical composition delivery is by systemic delivery. In some embodiments, the systemic delivery comprises oral, transdermal, subcutaneous, intravenous or local administration.

### Treatment Regimens

Described herein are methods of administration of a therapeutic agent described herein that comprise a treatment regimen. In some embodiments, the method comprises a treatment regimen delivering a pharmaceutical formulation of a therapeutic agent described herein according to the needs of a subject. In some embodiments, the treatment regimen comprises a single administration. In some embodiments, the treatment regimen comprises at least one administration. In some embodiments, the treatment regimen comprises 1, 2, 3, 4, 5, 6, 7, 8, 9, 10, 11, 12, 13, 14, 15, 16, 17, 18, 19, 20 or more administrations. In some embodiments, the effective amount contained within one administration is an effective amount for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 35, 42, 49, 56, 60, 61, 62, or 90 days. In some embodiments, one administration comprises an effective amount for at least 1 week, at least 2 weeks, at least 4 week, at least 2 months, or at least 6 months. In some embodiments, the method further comprises a treatment cycle. In some embodiments, the treatment cycle comprises 1, 2, 3, 4, 5, 6, 7, 8, 9, 10, 11, 12, 13, 14, 15, 16, 17, 18, 19, 20 or more administrations of the pharmaceutical composition. In some embodiments, the treatment regimen comprises a plurality of treatment cycles. In some embodiments, the plurality of treatment cycles is 1, 2, 3, 4, 5, 6, 7, 8, 9, or 10 more treatment cycles. In some embodiments, a treatment cycle comprises a length of time. In some embodiments, the length of time comprises 1, 2, 3, 4, 5, 6, 7, 8, 9, 10, 11, 12, 13, 14, 15, 16, 17, 18, 19, 20, 21, 22, 23, 24, 30, or 36 months. In some embodiments, a treatment is administered at a selected interval such as once every 1 day, 2 days, 3 days, 4 days, 5 days, 6 days, 7 days, 8 days, 9 days, 10 days 11 days, 12 days, 13 days, 14 days, 15 days, 16 days, 17 days, 18 days, 19 days, 20 days or 21 days. In some embodiments, a treatment is administered at a selected interval such as once every 1 week, 2 weeks, 3 weeks, 4 weeks, 5 weeks, 6 weeks, 7 weeks, 8 weeks, 9 weeks, 10 weeks, 11 weeks, or 12 weeks.

In some cases, the therapeutic agent described herein can be administered at a dose of from about 0.1 to about 1000 mg, from about 1 mg to about 1000 mg, from about 5 mg to about 1000 mg, from about 10 mg to about 1000 mg, from about 15 mg to about 1000 mg, from about 20 mg to about 1000 mg, from about 25 mg to about 1000 mg, from about 30 mg to about 1000 mg, from about 35 mg to about 1000 mg, from about 40 mg to about 1000 mg, from about 45 mg to about 1000 mg, from about 50 mg to about 1000 mg, from about 55 mg to about 1000 mg, from about 60 mg to about 1000 mg, from about 65 mg to about 1000 mg, from about 70 mg to about 1000 mg, from about 75 mg to about 1000 mg, from about 80 mg to about 1000 mg, from about 85 mg to about 1000 mg, from about 90 mg to about 1000 mg, from about 95 mg to about 1000 mg, from about 100 mg to about 1000 mg, from about 1 mg to about 100 mg, from about 2 mg to about 100 mg, from about 5 mg to about 100 mg, from about 10 mg to about 100 mg, from about 20 mg to about 100 mg, from about 40 mg to about 100 mg, from about 50 mg to about 100 mg, from about 60 mg to about 100 mg, from about 70 mg to about 100 mg, from about 80 mg to about 100 mg, or from about 90 mg to about 100 mg. In some cases, a therapeutic agent described herein can be administered at a dose of from about 0.1 to about 60 mg, from about 0.5 mg to about 60 mg, from about 1 mg to about 60 mg, from about 2 mg to about 60 mg, from about 3 mg to about 60 mg, from about 4 mg to about 60 mg, from about 5 mg to about 60 mg, from about 8 mg to about 60 mg, from about 10 mg to about 60 mg, from about 15 mg to about 60 mg, from about 20 mg to about 60 mg, from about 25 mg to about 60 mg, from about 30 mg to about 60 mg, or from about 40 mg to about 60 mg.

In some cases the therapeutic agent described herein is administered at a dosage of about from about 100 mg to about 2000 mg, from about 150 mg to about 2000 mg, from about 200 mg to about 2000 mg, from about 250 mg to about 2000 mg, from about 300 mg to about 2000 mg, from about 350 mg to about 2000 mg, from about 400 mg to about 2000 mg, from about 450 mg to about 2000 mg, from about 500 mg to about 2000 mg, from about 550 mg to about 2000 mg, from about 600 mg to about 2000 mg, from about 650 mg to about 2000 mg, from about 700 mg to about 2000 mg, from about 700 mg to about 1900 mg, from about 700 mg to about 1800 mg, from about 700 mg to about 1700 mg, from about 700 mg to about 1600 mg, from about 700 mg to about 1500 mg, from about 700 mg to about 1400 mg. In some cases the therapeutic agent described herein is administered at a dosage of 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, or 2000 mg. In some preferred embodiments, 700 mg is the dosage of the therapeutic agent. In some preferred embodiments, 1400 mg is the dosage of the therapeutic agent.

### Production of Tau Antibodies

In some embodiments, antibodies or antibody fragments described herein are produced using any method known in the art to be useful for the synthesis of antibodies or antibody fragments, in particular, by chemical synthesis or by recombinant expression, and are preferably produced by recombinant expression techniques.

In some instances, an antibody or its binding fragment thereof is expressed recombinantly, and the nucleic acid encoding the antibody or its binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or its binding is optionally generated by immunizing an animal, such as a mouse, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific embodiments, the expression of the antibody is regulated by a constitutive, an inducible or a tissue, specific promoter.

In some embodiments, a variety of host-expression vector systems is utilized to express an antibody, or its binding fragment described herein. Such host-expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long-term, high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to the herpes simplex virus thymidine kinase (Wigler et al., 1977, Cell 11:223), hypoxanthine-guanine phosphoribosyltransferase (Szybalska & Szybalski, 192, Proc. Natl. Acad. Sci. USA 48:202), and adenine phosphoribosyltransferase (Lowy et al., 1980, Cell 22:817) genes are employed in tk-, hgprt- or aprt-cells, respectively. Also, antimetabolite resistance are used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May 1993, TIB TECH 11(5):155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1).

In some instances, the expression levels of an antibody are increased by vector amplification (for a review, see Bebbington and Hentschel, the use of vectors based on gene amplification for the expression of cloned genes in mammalian cells in DNA cloning, Vol. 3. (Academic Press, New York, 1987)). When a marker in the vector system expressing an antibody is amplifiable, an increase in the level of inhibitor present in culture of host cell will increase the number of copies of the marker gene. Since the amplified region is associated with the nucleotide sequence of the antibody, production of the antibody will also increase (Crouse et al., 1983, Mol. Cell Biol. 3:257).

In some instances, any method known in the art for purification of an antibody is used, for example, by chromatography (e.g., ion exchange, affinity, particularly by affinity for the specific antigen after Protein A, and sizing column chromatography), centrifugation, differential solubility, or by any other standard technique for the purification of proteins.

### Expression Vectors

In some embodiments, vectors include any suitable vectors derived from either a eukaryotic or prokaryotic sources. In some cases, vectors are obtained from bacteria (e.g. E. coli), insects, yeast (e.g. Pichia pastoris), algae, or mammalian sources. Exemplary bacterial vectors include pACYC177, pASK75, pBAD vector series, pBADM vector series, pET vector series, pE™ vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Exemplary insect vectors include pFastBacl, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

In some cases, yeast vectors include Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichi pastoris vector, pGAPZA,B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Exemplary algae vectors include pChlamy-4 vector or MCS vector.

Examples of mammalian vectors include transient expression vectors or stable expression vectors. Mammalian transient expression vectors may include pRK5, p3xFLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3xFLAG-CMV 7.1, pFLAG-CMV 20, p3xFLAG-Myc-CMV 24, pCMV-FLAG-MAT1, pCMV-FLAG-MAT2, pBICEP-CMV 3, or pBICEP-CMV 4. Mammalian stable expression vector may include pFLAG-CMV 3, p3xFLAG-CMV 9, p3xFLAG-CMV 13, pFLAG-Myc-CMV 21, p3xFLAG-Myc-CMV 25, pFLAG-CMV 4, p3xFLAG-CMV 10, p3xFLAG-CMV 14, pFLAG-Myc-CMV 22, p3xFLAG-Myc-CMV 26, pBICEP-CMV 1, or pBICEP-CMV 2.

In some instances, a cell-free system is a mixture of cytoplasmic and/or nuclear components from a cell and is used for in vitro nucleic acid synthesis. In some cases, a cell-free system utilizes either prokaryotic cell components or eukaryotic cell components. Sometimes, a nucleic acid synthesis is obtained in a cell-free system based on for example Drosophila cell, Xenopus egg, or HeLa cells. Exemplary cell-free systems include, but are not limited to, E. coli S30 Extract system, E. coli T7 S30 system, or PURExpress®.

### Host Cells

In some embodiments, a host cell includes any suitable cell such as a naturally derived cell or a genetically modified cell. In some instances, a host cell is a production host cell. In some instances, a host cell is a eukaryotic cell. In other instances, a host cell is a prokaryotic cell. In some cases, a eukaryotic cell includes fungi (e.g., yeast cells), animal cell or plant cell. In some cases, a prokaryotic cell is a bacterial cell. Examples of bacterial cell include gram-positive bacteria or gram-negative bacteria. Sometimes the gram-negative bacteria is anaerobic, rod-shaped, or both.

In some instances, gram-positive bacteria include Actinobacteria, Firmicutes or Tenericutes. In some cases, gram-negative bacteria include Aquificae, Deinococcus-Thermus, Fibrobacteres-Chlorobi/Bacteroidetes (FCB group), Fusobacteria, Gemmatimonadetes, Nitrospirae, Planctomycetes-Verrucomicrobia/Chlamydiae (PVC group), Proteobacteria, Spirochaetes or Synergistetes. Other bacteria can be Acidobacteria, Chloroflexi, Chrysiogenetes, Cyanobacteria, Deferribacteres, Dictyoglomi, Thermodesulfobacteria or Thermotogae. A bacterial cell can be Escherichia coli, Clostridium botulinum, or Coli bacilli.

Exemplary prokaryotic host cells include, but are not limited to, BL21, Machl™, DH1OB™, TOP1O, DH5u, DH1OBac™, OmniMax™, MegaX™, DH12S™, INVI10, TOP1OF′, INVuF, TOP1O/P3, ccdB Survival, PIR1, PIR2, Stbl2™, Stbl3™, or Stbl4™.

In some instances, animal cells include a cell from a vertebrate or from an invertebrate. In some cases, an animal cell includes a cell from a marine invertebrate, fish, insects, amphibian, reptile, or mammal. In some cases, a fungus cell includes a yeast cell, such as brewer's yeast, baker's yeast, or wine yeast.

Fungi include ascomycetes such as yeast, mold, filamentous fungi, basidiomycetes, or zygomycetes. In some instances, yeast includes Ascomycota or Basidiomycota. In some cases, Ascomycota includes Saccharomycotina (true yeasts, e.g. Saccharomyces cerevisiae (baker's yeast)) or Taphrinomycotina (e.g. Schizosaccharomycetes (fission yeasts)). In some cases, Basidiomycota includes Agaricomycotina (e.g. Tremellomycetes) or Pucciniomycotina (e.g. Microbotryomycetes).

Exemplary yeast or filamentous fungi include, for example, the genus: Saccharomyces, Schizosaccharomyces, Candida, Pichia, Hansenula, Kluyveromyces, Zygosaccharomyces, Yarrowia, Trichosporon, Rhodosporidi, Aspergillus, Fusarium, or Trichoderma. Exemplary yeast or filamentous fungi include, for example, the species: Saccharomyces cerevisiae, Schizosaccharomyces pombe, Candida utilis, Candida boidini, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Pichia metanolica, Pichia angusta, Pichia pastoris, Pichia anomala, Hansenula polymorpha, Kluyveromyces lactis, Zygosaccharomyces rouxii, Yarrowia lipolytica, Trichosporon pullulans, Rhodosporidium toru-Aspergillus niger, Aspergillus nidulans, Aspergillus awamori, Aspergillus oryzae, Trichoderma reesei, Yarrowia lipolytica, Brettanomyces bruxellensis, Candida stellata, Schizosaccharomyces pombe, Torulaspora delbrueckii, Zygosaccharomyces bailii, Cryptococcus neoformans, Cryptococcus gattii, or Saccharomyces boulardii.

Exemplary yeast host cells include, but are not limited to, Pichia pastoris yeast strains such as GS115, KM71H, SMD1168, SMD1168H, and X-33; and Saccharomyces cerevisiae yeast strain such as INVScl.

In some instances, additional animal cells include cells obtained from a mollusk, arthropod, annelid or sponge. In some cases, an additional animal cell is a mammalian cell, e.g., from a primate, ape, equine, bovine, porcine, canine, feline or rodent. In some cases, a rodent includes mouse, rat, hamster, gerbil, hamster, chinchilla, fancy rat, or guinea pig.

Exemplary mammalian host cells include, but are not limited to, 293A cell line, 293FT cell line, 293F cells, 293 H cells, CHO DG44 cells, CHO—S cells, CHO-Kl cells, FUT8 KO CHOK1, Expi293F™ cells, Flp-In™ T-REx™ 293 cell line, Flp-In™-293 cell line, Flp-In™-3T3 cell line, Flp-In™—BHK cell line, Flp-In™—CHO cell line, Flp-In™—CV-1 cell line, Flp-In™-Jurkat cell line, FreeStyle™ 293-F cells, FreeStyle™ CHO—S cells, GripTite™ 293 MSR cell line, GS-CHO cell line, HepaRG™ cells, T-REx™ Jurkat cell line, Per.C6 cells, T-REx™-293 cell line, T-REx™—CHO cell line, and T-REx™—HeLa cell line.

In some instances, a mammalian host cell is a stable cell line, or a cell line that has incorporated a genetic material of interest into its own genome and has the capability to express the product of the genetic material after many generations of cell division. In some cases, a mammalian host cell is a transient cell line, or a cell line that has not incorporated a genetic material of interest into its own genome and does not have the capability to express the product of the genetic material after many generations of cell division.

Exemplary insect host cells include, but are not limited to, Drosophila S2 cells, S9 cells, Sf21 cells, High Five™ cells, and expresSF+® cells. In some instances, plant cells include a cell from algae. Exemplary insect cell lines include, but are not limited to, strains from Chlamydomonas reinhardtii 137c, or Synechococcus elongatus PPC 7942.

### Kits

Disclosed herein, in some embodiments, are kits comprising one or more compositions of the present disclosure and/or instructions comprising one or more methods of the present disclosure.

A kit-of-parts comprising a pharmaceutical composition together with instructions for use is further provided. For convenience, the kit-of-parts may comprise reagents in predetermined amounts with instructions for use. Provided herein, in some embodiments, are kits comprising the pharmaceutical composition described herein, the nucleic acid described herein, the host cells or vectors described herein, and instructions for use. In some embodiments, the kits comprise the pharmaceutical composition described herein and instructions for use. In some embodiments, the kits comprise the nucleic acid described herein and instructions for use. In some embodiments, the kits comprise the host cells described herein and instructions for use. In some embodiments, the kits comprise the vectors described herein and instructions for use.

In some embodiments, disclosed herein are kids comprising an anti-tau antibody or antigen-binding fragment thereof disclosed herein. In some embodiments, a kit can be a diagnostic kit. In some embodiments, a kit comprises an anti-tau antibody or antigen-binding fragment thereof disclosed herein and instructions for use. In some embodiments, a kit comprises means for measuring an anti-tau antibody or antigen-binding fragment thereof level in a sample and instructions for use. A kit may provide a unit or device for obtaining a sample from a subject (e.g., a device with a needle coupled to an aspirator). A kit may include a plurality of syringes, ampules, foil packets, or blister packs, each containing a single unit dose of a kit component described herein. Containers of a kit may be airtight, waterproof (e.g., impermeable to changes in moistures or evaporation), and/or light-tight. A kit may include a device suitable for administration of the components, e.g., a syringe, inhalant, pipette, forceps, measured spoon, dropper (e.g., eye dropper), swab (e.g., a cotton swab or wooden swab), or any such delivery device. In some embodiments, the device may be a medical implant device, e.g., packaged for surgical insertion. A kit disclosed herein may comprise one or more reagents or instruments which enable the method to be carried out. In some embodiments, reagents or instruments include one or more of the following: suitable buffer(s) (aqueous solutions), a support comprising wells on which quantitative reactions can be done. A kit may be a specific kit for a specific tissue sample. Further, a kit disclosed herein may comprise a control. In some embodiments, the kid may comprise any of the compositions (e.g., pharmaceutical compositions) described herein which include any of the nucleic acids, any of the polypeptides, or any of the vectors described herein. In some embodiments, a kit can include a solid composition (e.g., a lyophilized composition including any of the vectors, polypeptides, or nucleic acids described herein) and a liquid for solubilizing the lyophilized composition. In some embodiments, the kit includes a vial including any of the pharmaceutical compositions described herein (e.g., formulated as an aqueous pharmaceutical composition). In addition to the above components, instructions for use may be provided in a kit. These instructions may be presented in the kit in a variety of forms, such as printed information on a suitable medium or substate (e.g., a piece or pieces of paper on which the information is printed), in the packaging of the kit, in a package insert, etc. In some embodiments, instructions for use can be provided on a computer readable medium (e.g., jump/thumb drive, CD, etc.), or which the information has been recorded, or at a website address which may be used via the internet to access the information at a website.

Disclosed herein, in some embodiments, are kits for selecting a subject for treatment of a neurodegenerative disease. In some embodiments, the kit comprises an anti-tau antibody or the antigen-binding fragment thereof of the present disclosure. In some embodiments, the anti-tau antibody or the antigen-binding fragment thereof comprises a heavy chain comprising a variable heavy chain (VH) domain, wherein the VH domain comprises a HCDR1 sequence selected from SEQ ID NOs: 1-9, a HCDR2 sequence selected from SEQ ID NOs: 10-17, and a HCDR3 sequence selected from SEQ ID NOs: 18-23; and a light chain comprising a variable light chain (VL) domain, wherein the VL domain comprises a LCDR1 sequence selected from SEQ ID NOs: 24-31, a LCDR2 sequence selected from SEQ ID NOs: 32-36, and a LCDR3 sequence selected from SEQ ID NOs: 37-43. In some embodiments, the anti-tau antibody or the antigen-binding fragment thereof comprises a heavy chain (HC) sequence comprising any one of SEQ ID NOs: 57, 60, 62, 64, or 55; and a light chain (LC) sequence comprising any one of SEQ ID NOs: 58, 59, 63, 65, or 56. In some embodiments, the anti-tau antibody or the antigen-binding fragment thereof comprises a variable heavy (VH) domain comprising a sequence that has at least 80%, at least 85%, at least 90%, at least 95% sequence identity to a sequence selected from SEQ ID NOs: 44-48; and a variable light (VL) domain comprising a sequence that has at least 80%, at least 85%, at least 90%, at least 95% sequence identity to a sequence selected from SEQ ID NOs: 49-54. In some embodiments, the kits comprises instructions for analyzing a sample obtained from the subject using the anti-tau antibody or antigen-binding fragment thereof in an immunoassay. In some embodiments, the instructions for analyzing a sample further comprise a standard value of p-Tau 217 level derived from a plurality of reference samples from reference subjects that do not have the neurodegenerative disease. In some embodiments, the kits comprises instructions for treating the neurodegenerative disease in the subject with a therapeutic agent of the present disclosure. In some embodiments, the therapeutic agent comprises an inhibitor of the Aβ peptide or a modified form of the Aβ peptide. In some embodiments, the therapeutic agent comprises an inhibitor of a phosphorylated tau protein. In some embodiments, the kit comprises one or more immunoassay components or complete systems, wherein the immunoassay comprises Single Molecule Array (SIMOA), an enzyme-linked immunoassay (ELISA), a radioimmunoassay (RIA), a fluoroimmunoassay (FIA), a chemiluminescent immunoassay (CLIA), or a counting immunoassay (CIA).

In some embodiments, the kit comprises the therapeutic agent. In some embodiments, the kit comprises therapeutic agent comprises: ABvac40LY3002813, AADvac-1, ACI-35, AAV-Htert, ACU193, ASN51, ACI-242, AD-35, Aducanumab, AGB101, AL002, Allopregnanolone, APH-1105, Atuzaginstat (COR388), AVP-786, AXS-05, Baricitinib, Bepranemab, BCG vaccine, BEIY2153, BPN14770, Bromocriptine, Bryostatin 1, BXCL-501, Blarcamesine (ANAVEX2-73), Bapineuzumab, BIIB092 (BMS-986168, IPN007, Gosuranemab), BIIB080/IONIS-MAPTRx, B1II1B076, BPDO-1603, BMS-984923, BDPP (bioactive dietary polyphenol preparation), Brexpiprazole, Caffeine, CAD 1063, Canakinumab, Contraloid acetate, COR588, CORT108297, CNP520(AMG520), C2N-8E12 (ABBV-8E12), Crenezumab, CST-2032, Curcumin, CY6463, Dabigatran,DAOIB, Dapagliflozin, Daratumumab, Dasatinib, Deferiprone, Donanemab (LY3002813), Donepezil (Aricept), Dronabinol, Dutanumab, Edicotinib (JNJ-40346527), Efavirenz, Emtricitabine, Empagliflozin, Elenbecestat (E2609), E2814, Ednerpic (T-817 mA), Elayta (CT1812), ExPlas, Escitalopram, Fosgonimeton (ATH01017), Galantamine, Gantenerumab, Brain Shuttle Gantenerumab (R07126209), GB301, grapeseed extract, GV1001, Guanfacine, GV-971, Intranasal insulin, HT-ALZ, Huperzine A, Hydralazine, IGC AD1, Icosapent ethyl (IPE), JNJ-63733657, KHK 66401, Lu AF87908, Lu AF20513, Lamivudine (3TC), Lanabecestat, Lenalidomide, Levetiracetam, L-serine, Lupron, Remternetug (LY3372993), LY3372689, LMTX, LY3303560 (Zagotenemab), Lecanemab (BAN2401), Losartan, LX1001, MEDI 1814, Memantine, Metformin, MK-1942, MIB-626, Montelukast, MW150, Neflamapimod (VX-745), Nabilone, NE3107, nicotinamide, nicotine, NNI-362, Nilotinib BE, Octohydro-aminoacridine succinate, Obicetrapib, Omega-3 (DHA+EPA), perindopril, Pepinemab (VX15), Posiphen, Prazosin, Ponezumab, Protollin, PQ912, PNT001, PU-AD, Quercetin, rapamycin, REM0046127, RG7345 (R06926496), rivastigmine, RG6100, R07105705 (MTAU9937A, RG6100, Semorinemab), Salsalate, Sargramostim, Semorinemab, Senicapoc, Semaglutide, SHR-1707, Simufilam (PTI-125), Sodium oligomannate, Solanezumab, scyllo-inositol, Sovateltide (PMZ-1620), Suvorexant, T3D-959, TB006, Telmisartan, Trehalose, Tdap vaccine, THC-free CBD oil, Thiethylperazine (TEP), Troriluzole (BHV4157), TPI 2871, Tricaprilin (AC-1202), TRx0237, UB-311, UCB0107, valiltramiprosate (ALZ-801), Valacyclovir, varoglutamstat (PQ912), VGH-AD1, Vorinostat, VT301, XProl595, Xanamem, Yangxue Qingao pills, an antisense RNA directed to an isoform of human tau mRNA or an isoform of human amyloid beta, an siRNA directed to an isoform of human tau mRNA or an isoform of human amyloid beta, an antisense oligonucleotide directed to an isoform of human tau mRNA or an isoform of human amyloid beta, an LNA oligonucleotide directed to an isoform of human tau mRNA or an isoform of human amyloid beta, a CRISPRn-based therapeutic targeting the human MAPT or the human APP locus, a CRISPRi-based therapeutic targeting the human MAPT or the human APP locus, allogeneic human MSCs, SNKO1 (autologous natural killer cell), allogenic adipose MSC-exosomes, CA-AC-02 (placenta derived MSCs), HUVEC-derived MSCs (NEUROSTEM), or AstroStem (autologous adipose-derived MSCs).In some embodiments, the kit comprises Donanemab. In some embodiments, the kit comprises Lecanemab. In some embodiments, the kit comprises Remternetug. In some embodiments, the kit comprises Aducanumab. In some embodiments, the kit comprises a therapeutic agent that is an inhibitor of the phosphorylated tau protein. In some embodiments, the inhibitor of the phosphorylated tau protein is an inhibitor of p-Tau 217 or a modified form of p-Tau 217, p-Tau 181, p-Tau 212, p-Tau 220, p-Tau 231, or phosphorylated-serine (pS)214-tau. In some embodiments, the inhibitor of the phosphorylated tau protein comprises an inhibitor of p-Tau 217.

### Certain Terminologies

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers+/−10% thereof, or 10% below the lower listed limit and 10% above the higher listed limit for the values listed for a range.

The terms “individual,” or “subject” are used interchangeably. None of the terms require or are limited to a situation characterized by the supervision (e.g., constant or intermittent) of a health care worker (e.g., a doctor, a registered nurse, a nurse practitioner, a physician's assistant, an orderly, or a hospice worker). Further, these terms refer to human or animal subjects. In some cases, the individual or the subject is a “patient” in that they have been diagnosed with a disease or a condition herein.

The term “antibody” herein is used in the broadest sense and includes monoclonal antibodies, including intact antibodies and functional (antigen-binding) antibody fragments thereof, including fragment antigen binding (Fab) fragments, F(ab′)2 fragments, Fab′ fragments, Fv fragments, recombinant IgG (rIgG) fragments, single chain antibody fragments, including single chain variable fragments (sFv or scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, and heteroconjugate antibodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. The antibody can comprise a rabbit IgG1 constant region. The antibody can comprise a rabbit IgG4 constant region. An antibody includes, but is not limited to, full-length and native antibodies, as well as fragments and portion thereof retaining the binding specificities thereof, such as any specific binding portion thereof including those having any number of, immunoglobulin classes and/or isotypes (e.g., IgG1, IgG2, IgG3, IgG4, IgM, IgA, IgD, IgE and IgM); and biologically relevant (antigen-binding) fragments or specific binding portions thereof, including but not limited to Fab, F(ab′)2, Fv, and scFv (single chain or related entity). A monoclonal antibody is generally one within a composition of substantially homogeneous antibodies; thus, any individual antibodies comprised within the monoclonal antibody composition are identical except for possible naturally occurring mutations that may be present in minor amounts. A monoclonal antibody can comprise a rabbit IgG1 constant region or a rabbit IgG4 constant region.

The term “Fab” refers to a protein that contains the constant domain of the light chain and the first constant domain (CHI) of the heavy chain. Fab fragments differ from Fab′ fragments by the addition of a few residues at the carboxy terminus of the heavy chain CHI domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

A “single-chain variable fragment (scFv)” is a fusion protein of the variable regions of the heavy (VH) and light chains (VL) of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

As used herein, the term “percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR—H1, FR—H2, FR—H3, and FR—H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 Jan; 27(1):55-77 (“IMGT” numbering scheme); Honegger A and Plickthun A, “Yet another numbering scheme for immunoglobulin variable domains: an automatic modeling and analysis tool,” J Mol Biol, 2001 Jun 8; 309(3):657-70, (“Aho” numbering scheme); and Whitelegg NR and Rees AR, “WAM: an improved algorithm for modelling antibodies on the WEB,” Protein Eng. 2000 December; 13(12):819-24 (“AbM” numbering scheme. In certain embodiments the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

The terms, “Tauopathy” or “tauopathies” refer to neurodegenerative disorders characterized by the deposition of abnormal tau protein in the brain. In some cases, the abnormal tau proteins form “tangles” in the brain, referred to as “tau tangles.” In some cases, the abnormal tau protein is phosphorylated tau. In some cases, phosphorylated tau accumulation causes synaptic impairment, neuronal dysfunction and/or formation of tau tangles in the brain, which are key pathological features of Alzheimer's disease and other tauopathies. Non-limiting examples of tauopathies can be found in Olfati N, et al. Clinical Spectrum of Tauopathies. Front Neurol. 2022 Jul. 14; 13:944806, which is hereby incorporated by reference in its entirety.

Unless defined otherwise, all technical and scientific terms used herein have the same meaning as commonly understood by one of ordinary skill in the art to which the methods and compositions described herein belong. Although any methods and materials similar or equivalent to those described herein can also be used in the practice or testing of the methods and compositions described herein, representative illustrative methods and materials are now described.

### Examples

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Tau Antibody Screening

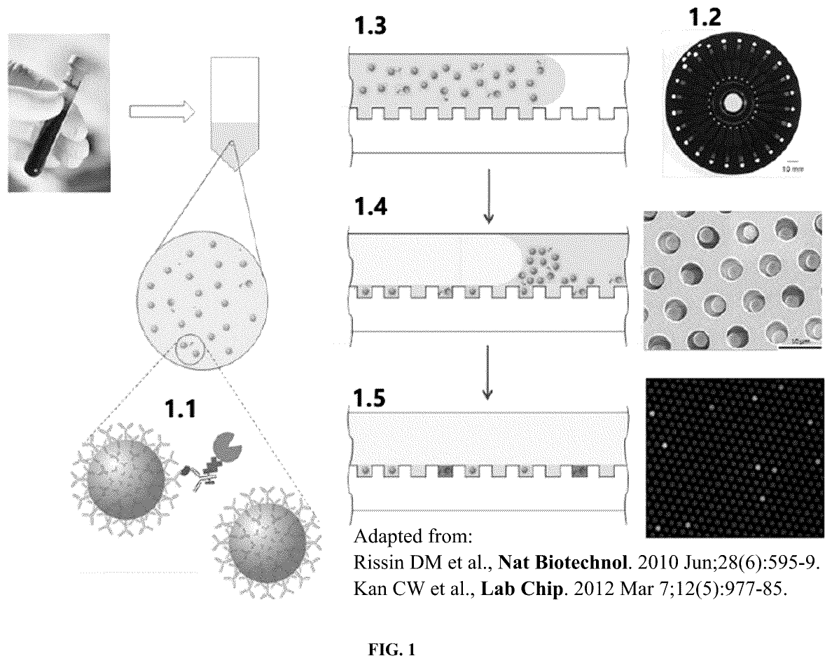

Tau antibodies that detect phosphorylated Tau were assayed in the Simoa® bead assay using a 2-step or 3-step protocol assay according to manufacturer's instructions. See FIG. 1.

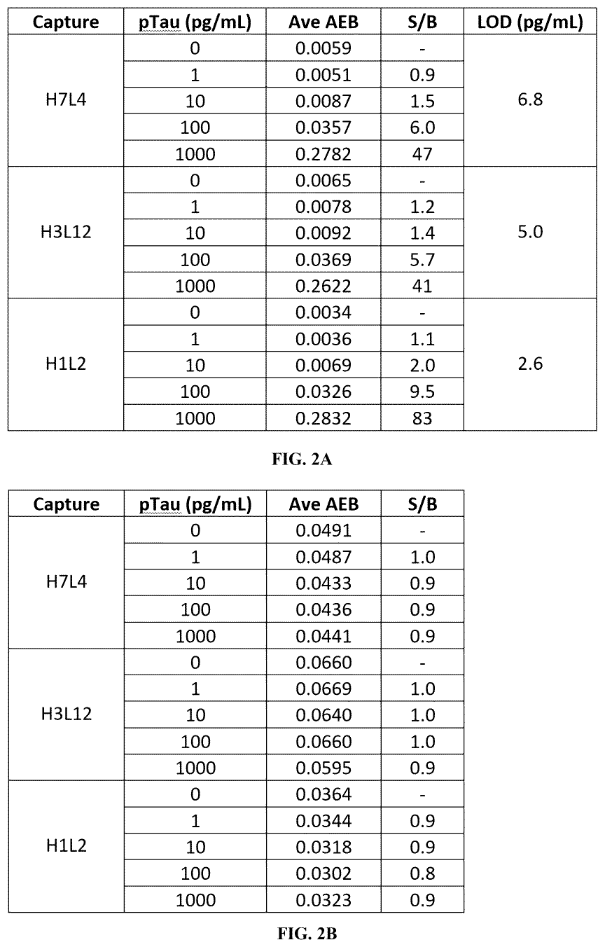

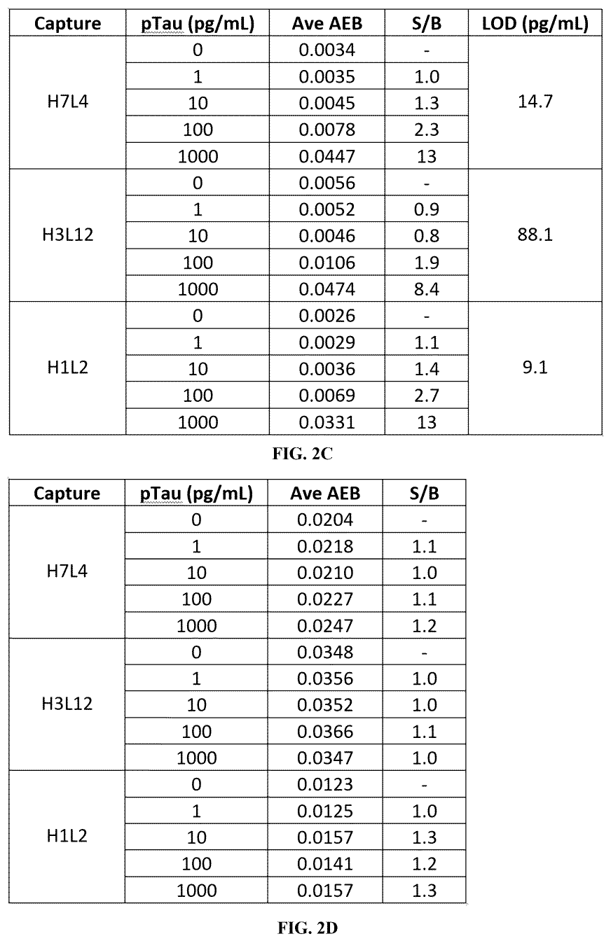

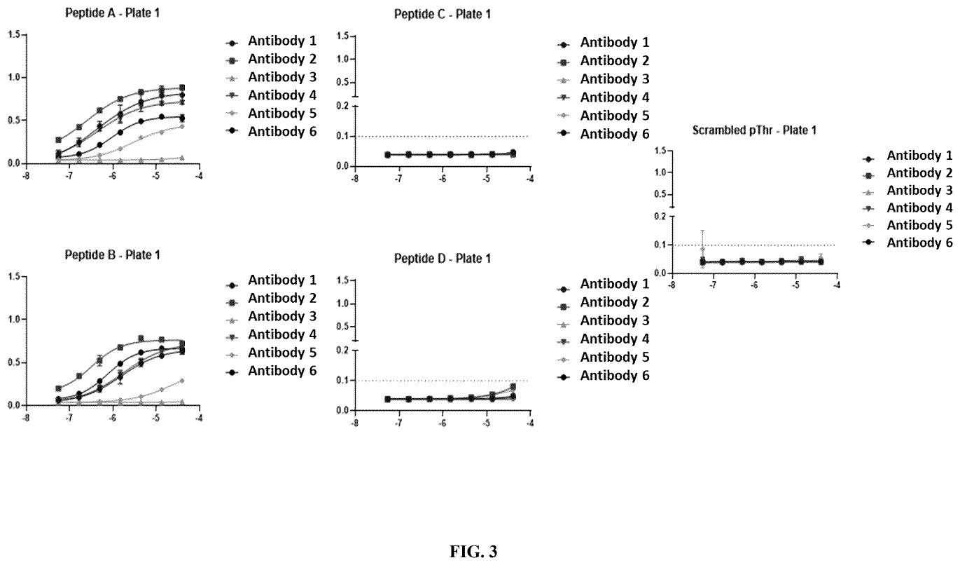

The antibodies tested were Antibody 1, Antibody 2, Antibody 3, Antibody 4, Antibody 5, and Antibody 6. The capture results can be seen in FIGS. 2A-2D. FIG. 2A is data from an 2-step protocol assay in which all capture antibodies were tested against Tau-12 detector (detects Tau at the N-terminus). The data demonstrates all captures had similar results with improved sensitivity seen with Antibody 6. FIG. 2B is data from an 2-step protocol assay in which all capture antibodies were tested against HT7-BT2 detectors (detects Tau in the mid-domain region). Both biotinylated antibodies were used. Data shows about a 10-fold increase in background compared to Tau-12 detector. No signal at 1000 μg/mL for any capture was detected. FIG. 2C is data from an 3-step protocol assay in which all capture antibodies were tested against Tau-12 detector. The data demonstrates reduced sensitivity compared to the 2-step protocol and improved sensitivity seen with Antibody 6. FIG. 2D is data from an 3-step protocol assay in which all capture antibodies were tested against HT7-BT2 detectors. Both biotinylated antibodies were used. Data shows about a 10-fold increase in background compared to Tau-12 detector. Based on the results, the 2-step protocol was then further optimized for sensitivity.

### Example 2. Pharmacokinetics of Tau Antibodies

Antibodies were tested for pharmacokinetic profile.

The antigen information for the antibodies is seen in Table 11.

TABLE 11

| | Antigen | SEQ ID | |
|---|---|---|---|
| Description | Name | NO: | Sequence |
| pT217a | WZN-1A | 88 | RSRTPSLP(pT)PPTREPKC |
| pT217b | WZN-1B | 89 | TPSLP(pT)PPTREPKKVAC |
| T217 | WZN-1C | 90 | RSRTPSLPTPPTREPKKVAC |
| pT212 | WZN-1D | 91 | RSR(pT)PSLPTPPTREPKC |
| pS214 | WZN-1E | 92 | RSRTP(pS)LPTPPTREPKC |
| pT220 | WZN-1F | 93 | RSRTPSLPTPP(pT)REPKKVAC |
| pT231 | WZN-1G | 94 | KVAVVR(pT)PPKSPSSAC |
| T231 | WZN-1H | 95 | KVAVVRTPPKSPSSAC |

Antibodies were generated and purified. The antibodies were assayed using a standard indirect ELISA protocol. Briefly, peptide antigens corresponding to SEQ ID NOs: 88-95 were diluted to 1 μg/ml in PBS and plated onto a Greiner Bio One Microlon 96 well plate. Peptide antigens were produced by the manufacturer Abcam. WZN-1A and WZN-1B served as targets. WZN-1C, WZN-1D, WZN-1E, WZN-1F, WZN-1G, and WZN-1H served as negative controls. In the peptide antigen sequences, a phosphorylated residue is indicated by (pT) for phosphorylated threonine or (pS) for phosphorylated serine. After blocking with 1% BSA in PBS pH 7.4, antibodies were serially diluted 1 to 4 with an initial concentration of 1 μg/ml. After incubation, unbound antibodies were washed off with 1X TBST and HRP labeled goat anti-rabbit secondary antibody was applied according to the manufacturer's instructions. Subsequently, unbound secondary antibody was washed off with 1X TBST and 3,3′5,5′-tetramethylbenzidine (TMB) was applied for 5 minutes at room temperature and plates were read at 650 nm. Data is seen in FIG. 3. FIG. 3 shows screening data of the different monoclonal antibodies to varying peptide concentrations.

### Example 3. Tau Antibodies for Immunohistochemistry

Tau antibodies described herein were tested in immunohistochemistry assays.

Briefly, all antibodies were optimised using a range of concentrations (0.01-3.00 μg/ml) and stained using a Leica Bond RX automated IHC platform: ER1 antigen retrieval (sodium citrate, pH 6) 20 mins at 100° C.; primary antibody 15 minutes at RT; IVD grade Leica Polymer Refine HRP detection 8 minutes at room temperature; DAβ chromogen 10 minutes at room temperature, and finally hematoxylin counterstain 5 minutes at room temperature. Antibodies that passed basic IHC staining went on to undergo IHC staining following alkaline phosphatase (AP) treatment (200 U/ml, 37° C. for 60 minutes). A vehicle-only control (buffer containing no AP) was also employed. Positive antigen control tissues were FFPE normal human cerebral cortex and cerebral cortex from an Alzheimer patient. Negative antigen control tissues were FFPE normal human liver, skeletal muscle, and heart muscle. All tissues were collated into a tissue micro array to streamline the IHC staining process. Negative reagent (detection system only) controls were employed and shown to be negative. Benchmark antibodies stained alongside the test antibodies were rabbit monoclonal [EPR22524-95]to Tau (ab254256, Abcam plc) and rabbit monoclonal [EPRi884(2)]to Tau (phospho S214) (ab170892, Abcam plc).

### Example 4. Detection of Tau Peptides Using Tau Antibodies

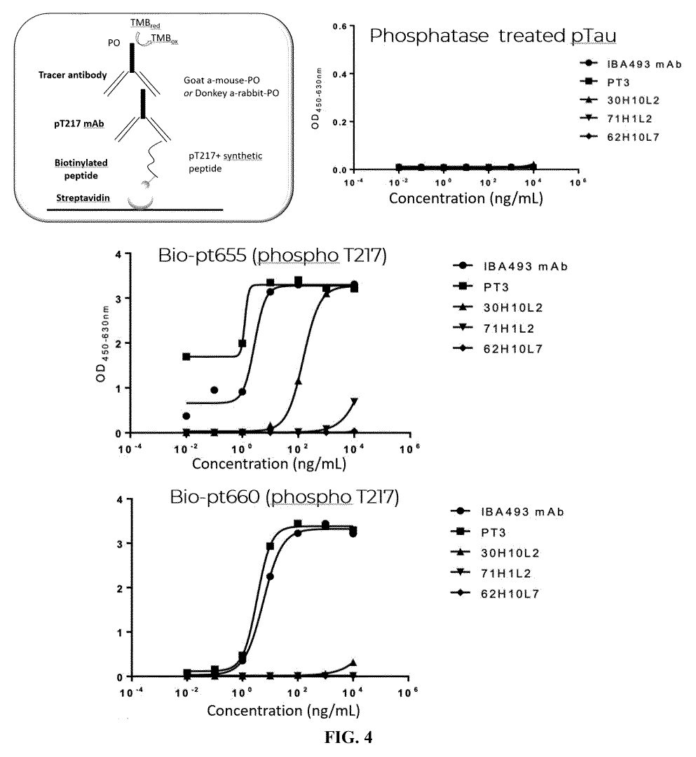

Tau antibodies described herein that detect phosphorylated Tau were tested in ELISA assays. Tau antibodies were first tested for pTau 217 reactivity by indirect ELISA. FIG. 4 displays a diagram depicting the indirect ELISA assay format utilized. Briefly, streptavidin beads were bound to a plate and biotinylated peptide was added to the plates under conditions allowing for biotin-streptavidin binding. The biotinylated peptide was a synthetic peptide comprising a portion of Tau and possessing a phosphorylated threonine residue at position 217 (pT217). This was the target peptide. After binding of the synthetic peptide to the plate by the formation of the biotin-streptavidin complex, Tau antibodies were added to the plates under conditions allowing for antibody-target peptide binding. After binding, plates were washed to remove any unbound antibody and plates then had a secondary antibody, or a tracer antibody, added (either goat anti-mouse antibody conjugated to peroxidase or donkey anti-rabbit antibody conjugated to peroxidase) directed to the species from which the Tau antibodies were derived. After binding, plates were washed to remove any unbound tracer antibody and plates next had TMB ELISA Peroxidase chromogenic substrate (3, 3′, 5, 5′-Tetramethylbenzidine) added to visualize antibody reactivity in indirect ELISA experiments. Antibody sample binding was quantitated using an ELISA microplate reader.

As shown in FIG. 4, five antibodies were tested for an ability to detect phosphorylated Tau using this indirect ELISA technique. IBA493 mAb corresponds to a rabbit anti-Tau antibody capable of binding to Tau phosphorylated at threonine residue 217 (pTau 217) (Eli Lilly and Company). PT3 corresponds to a mouse anti-phospho (T212/T217) Tau selective antibody (Janssen Biotech Inc.). 30H10L2 corresponds to Antibody 2 described herein. 71H1L2 corresponds to Antibody 6 described herein. 62H10L7 corresponds to Antibody 5 described herein. All five antibodies were assayed for reactivity with pTau 217 in two separate ELISA instruments at the following concentrations: 10-², 10-¹, 10-⁰, 10¹, 10², 103, and 104 ng/mL per plate. As can been seen in the Bio-pt655 (phospho T217) and Bio-pt660 (phospho T217) graphs, both IBA493 mAb and PT3 demonstrated a robust, concentration-dependent level reactivity to pTau 217. Antibody 2 demonstrated a more modest concentration-dependent level reactivity to pTau 217 revealed at 104 ng/mL per plate. Antibody 5 and Antibody 6 did not demonstrate reactivity to pTau 217 in these assays. A graph in FIG. 4 showing the results of this ELISA assay using the five test antibodies on phosphatase-treated pTau demonstrated the specificity of antibodies IBA493 mAb, PT3, and Antibody 2 in detecting phosphorylated Tau.

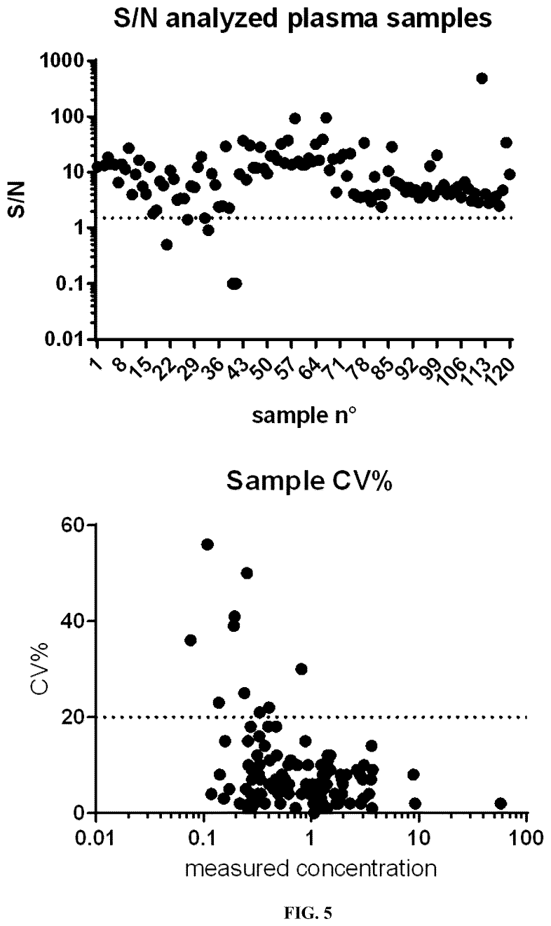

Tau antibodies described herein that detect phosphorylated Tau were tested in Simoa®-based assays. FIGS. 5-21 display results Simoa® assays designed to sensitive tests for Tau reactivity to antibodies described herein. In some aspects, Simoa®-based assays can be approximately 1000× more sensitivity at detecting a given analyte when compared to detection of the same analyte in an indirect ELISA assay. This elevated sensitivity of Simoa®-based assays allows for the development and use of biomarkers that previously could not have generated a detectable signal using a traditional assay such as indirect ELISA. The elevated sensitivity of Simoa®-based assays when compared to conventional immunoassays such as indirect ELISA is due to the fact that the Simoa® method is capable of detecting single target molecules whereas conventional immunoassays typically require large reaction volume and millions of fluorophores, or millions of antibody-conjugated enzymes reacted to a color-producing substrate, before an optical signal can be detected. For Simoa®-based assays, average enzyme per bead (AEB) denotes raw signal output.

In FIG. 5, Antibody 2, as described herein and is capable of detecting pTau 217, was used in a Simoa®-based assay to detect a level of an analyte per plasma sample derived from an individual. Signal-to-noise (S/N) ratio was determined by Simoa® for each sample and plotted in a graph. 120 plasma samples were taken from individuals and assayed. The graphed S/N ratio indicated that all tested samples apart from one yielded a signal within the expected concentration range. When plasma samples were diluted 1:3 and then assayed again, only 3 of the 120 samples yielded a result below measurement of a blank control and only 5 samples registered a measurement of S/N 1.5, which was determined to be the limit of detection (LOD). In FIG. 5, with each of the 120 plasma samples assayed, a calculation was made to determine the coefficient of variation (CV %) for each sample and the results were graphed against measured concentration. 10 of the 120 samples yielded a CV % greater than 20 and from this analysis, the estimated analytical lower limit of quantitation (LLOQ) was determined to be 0.08 pg/mL. This LLOQ value represents the lowest amount of an analyte (Tau phosphorylated at T217) that can be quantitatively determined with an acceptable level of precision. These results in FIG. 5 indicated the sensitivity of the Simoa® method to detect Tau phosphorylated at T217 using Antibody 2.

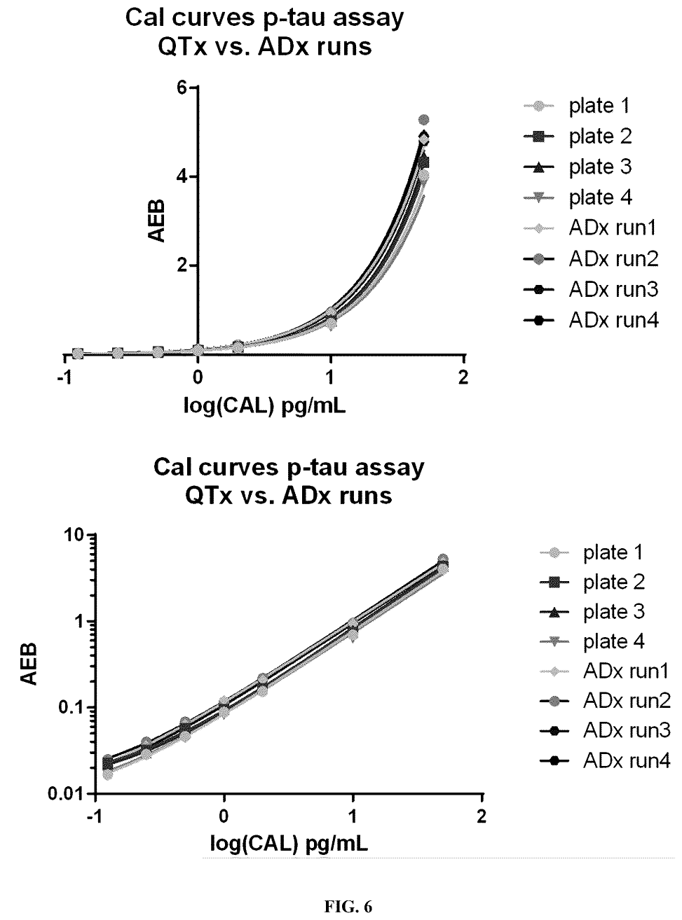

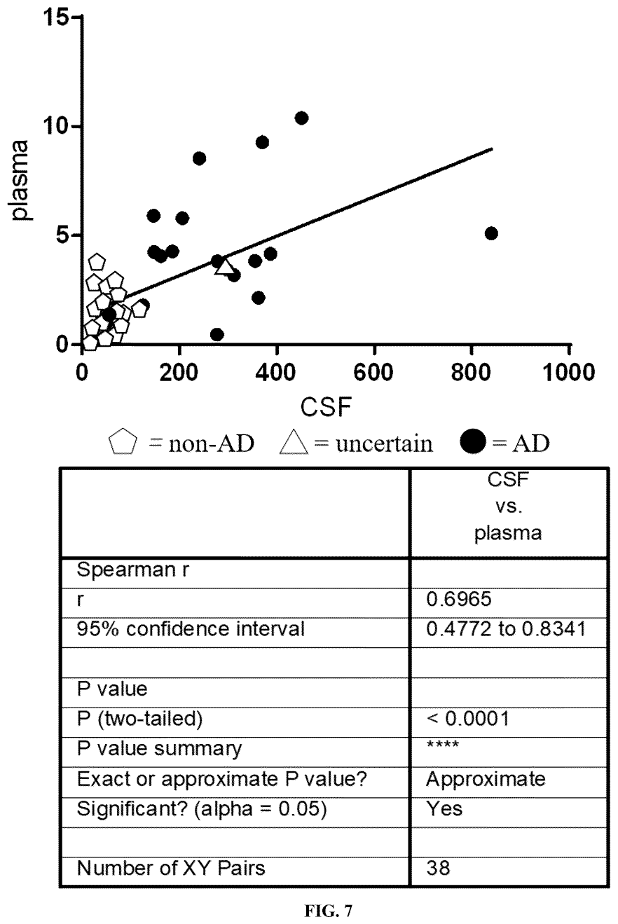

In FIG. 6 calibration curves were generated for the Simoa® pTau-217 assay and graphed [AEB vs log(CAL) pg/mL]using 68 CSF samples and 120 plasma samples separated into 4 plates using Antibody 2 and 4 plates using ADx Neuroscience antibody AD×204. In another graph from this assay performed on a separate instrument [AEB vs log(CAL) pg/mL]with AEB plotted on a log scale demonstrated the fit of the data can enable accurate analyte quantitation calculations when measuring samples.

In FIG. 7, 38 paired CSF and EDTA plasma samples were measured with the Simoa® pTau-217 assay using Antibody 2. Results were graphed and samples were indicated with their clinical diagnosis (non-AD, uncertain, or AD). These results, and the statistical analysis thereof, indicated a strong correlation between CSF and plasma pTau levels as measured with the Simoa® pTau-217 assay using Antibody 2 (R value˜0.7 and P value for two-tailed T test<0.0001 between non-AD and AD).

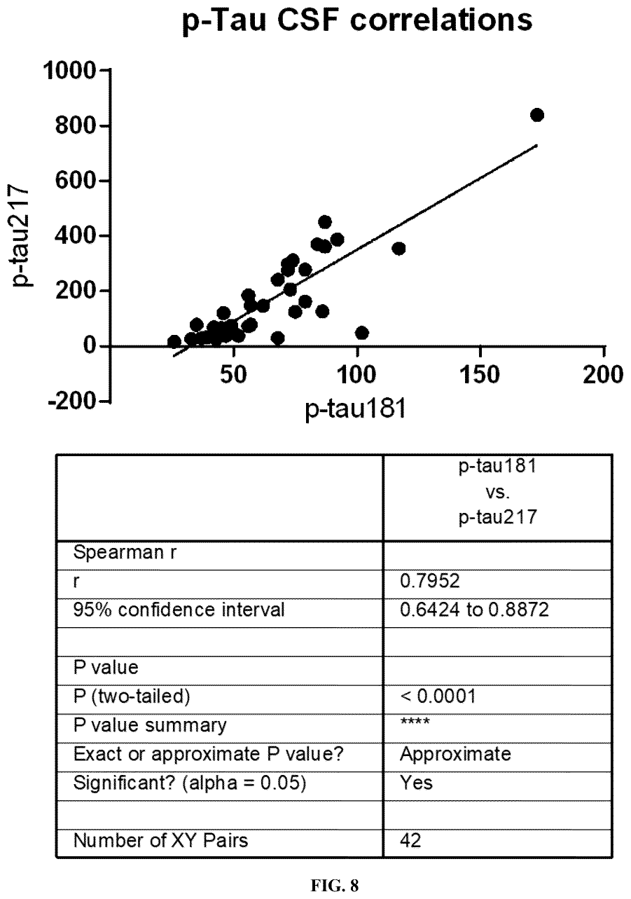

In FIG. 8, 42 CSF samples were measured with the Simoa® pTau-217 assay using Antibody 2 and a Simoa® pTau-181 assay using a pTau-181 antibody from Quanterix® (Quanterix® Corp., Item number 103714) and plotted against each other. This demonstrated that the Simoa® pTau-217 assay using Antibody 2 showed the expected relationship with an analyte implicated in AD detected in CSF (pTau-181). Statistical analysis indicated an R value˜0.8 and P value for two-tailed T test<0.0001.

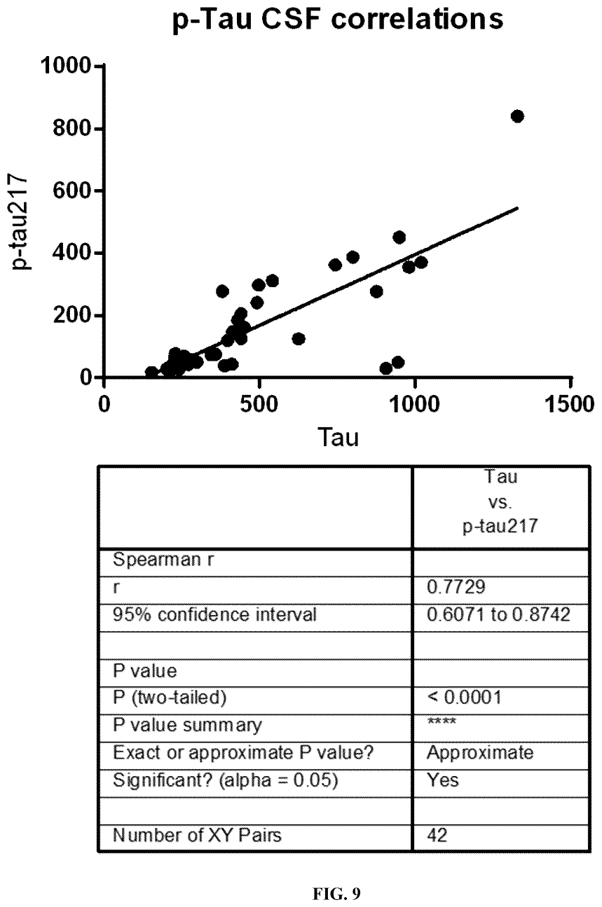

In FIG. 9, 42 CSF samples were measured with the Simoa® pTau-217 assay using Antibody 2 and a Simoa® pTau assay using Innotest pTau-181 antibody and plotted against each other. This demonstrated that the Simoa® pTau-217 assay using Antibody 2 showed the expected relationship with an analyte implicated in AD detected in CSF (pTau). Statistical analysis indicated an R value˜0.77 and P value for two-tailed T test<0.0001.

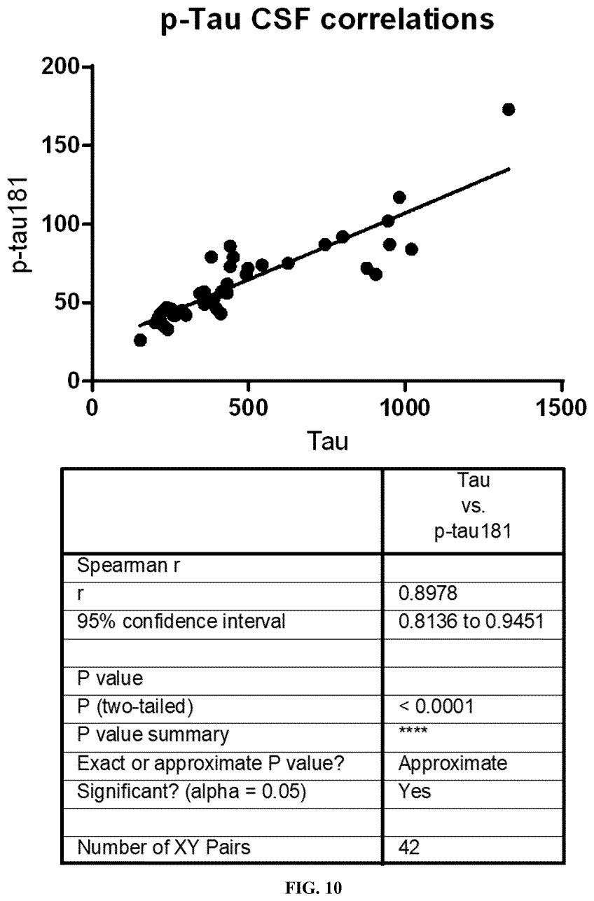

In FIG. 10, 42 CSF samples were measured with a Simoa® HD-X assay using Antibody 2 as a capture antibody, AD×204 antibody as a detector, and a peptide as calibrator. This demonstrated that the Simoa® assays using known AD biomarkers showed the expected relationship. Statistical analysis indicated an R value˜0.9 and P value for two-tailed T test<0.0001.

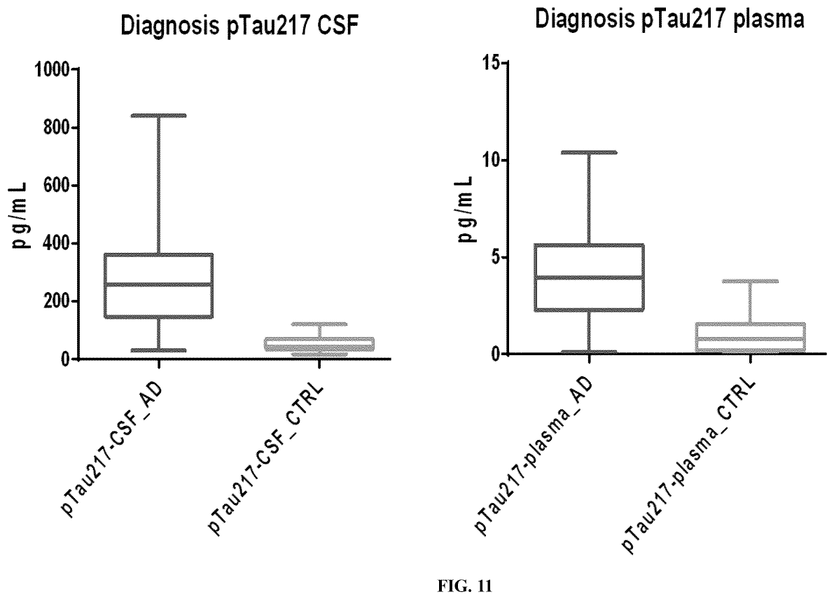

In FIG. 11, CSF samples and plasma samples were measured with the Simoa® pTau-217 assay using Antibody 2 and graphed in separate graphs. Clinical diagnosis of AD, or control with no AD diagnosis, was used as the classifier for each sample. Analysis of the graphed results indicated a significant difference between samples derived from individuals with a clinical AD diagnosis vs controls for both CSF samples and plasma samples. Area under the curve (AUC) calculation was 0.94 for CSF samples and 0.86 for plasma samples. These results indicated that the Simoa® pTau-217 assay using Antibody 2 was able to differentiate AD cases in CSF and plasma.

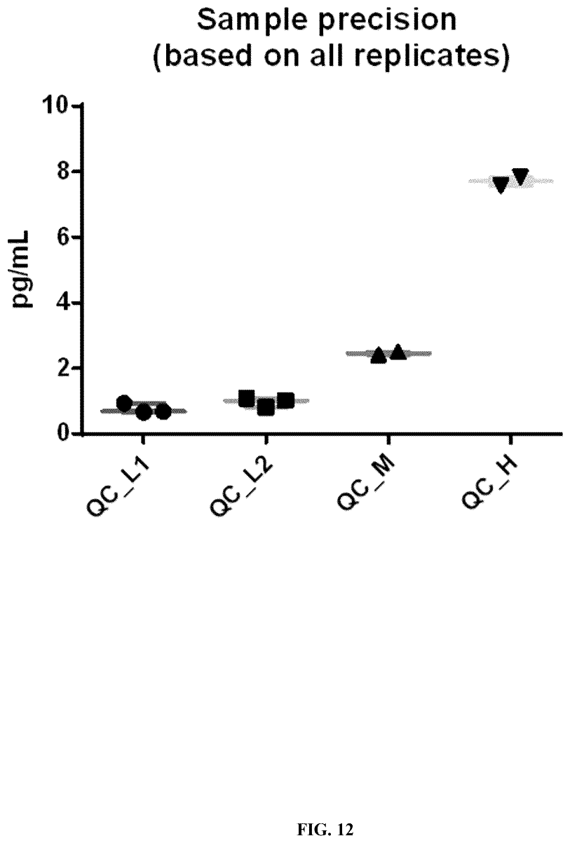

In FIG. 12, 4 EDTA plasma samples with high pTau levels serve as quality controls (labelled QC_L1, QC_L2, QC_M, and QC_H) were measured with the Simoa® pTau-217 assay using Antibody 2 in duplicate test and pTau levels were calculated. Plotting the results from repeated testing demonstrated the precision and reproducibility of the Simoa® pTau-217 assay using Antibody 2.

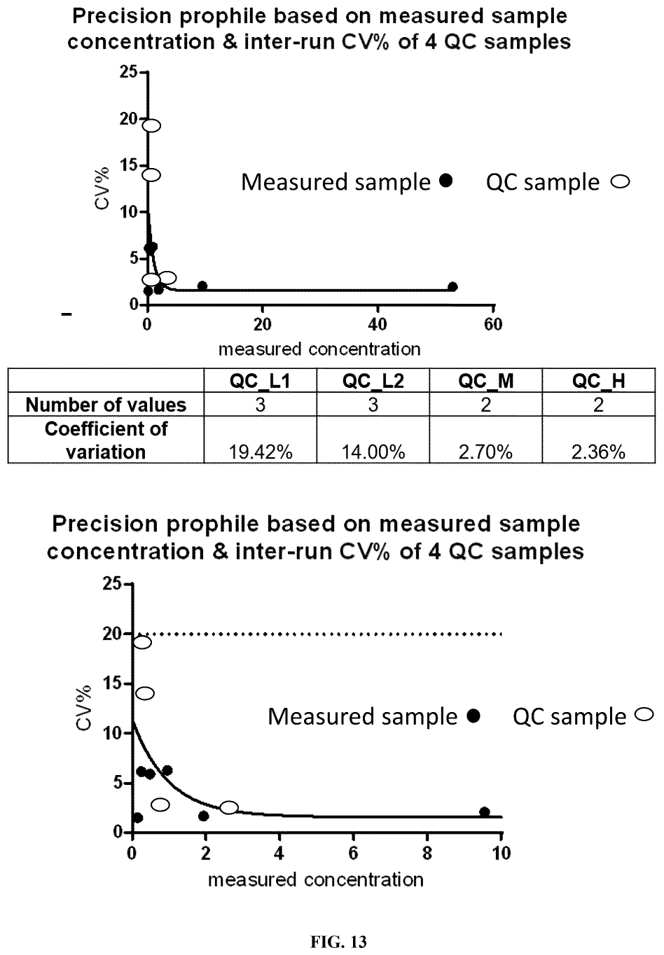

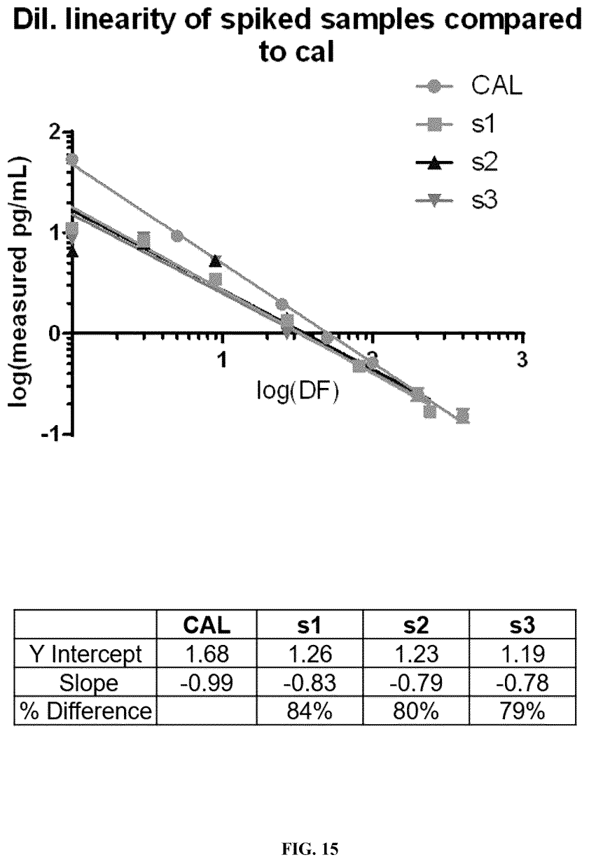

In FIG. 13, control samples from FIG. 15 and additional measured samples were measured with the Simoa® pTau-217 assay using Antibody 2 and plotted in two separate experiments to generate precision profiles. The precision profiles are based on measured sample concentration and inter-run CV %f the 4 QC samples. From this experiment, a functional LLOQ of pTau-217 in this assay was determined to be 0.26 pg/mL.

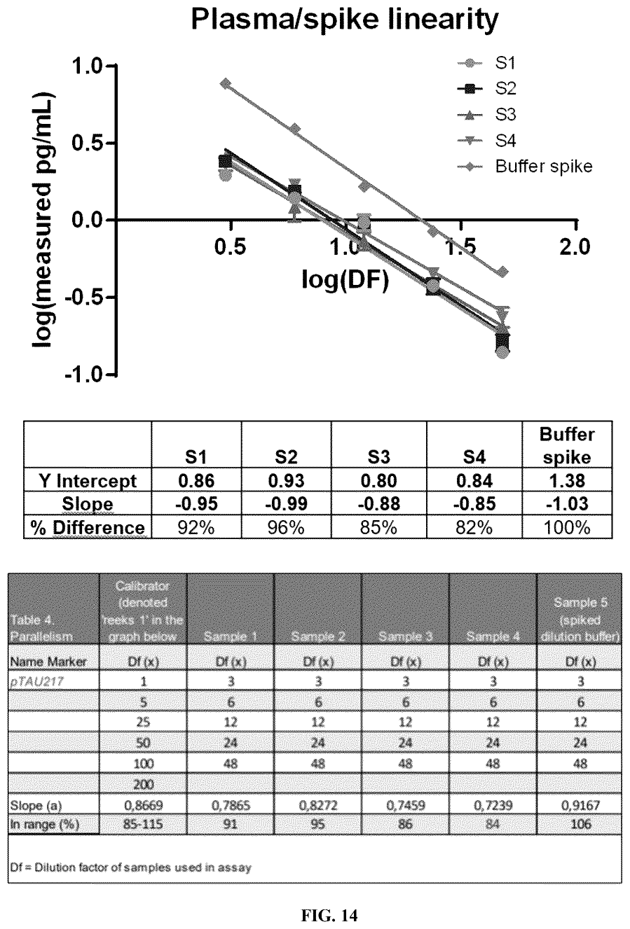

In FIG. 14, parallelism was assessed in the Simoa® pTau-217 assay using Antibody 2. A determination of parallelism is also important in that it shows if a signal is specific. Parallelism determines whether actual samples containing high endogenous analyte concentrations provide the same degree of detection in the assay in a standard curve after dilutions. This can represent differences in antibody binding affinity to endogenous analyte and a standard or calibration analyte. This can ensure that recombinant standards parallel native recognition of the endogenous analyte. In this experiment, 4 plasma samples, each from different donors, with relatively high concentration of detected pTau-217 and a spiked dilution buffer (sample 5) were diluted with a factor of 2 in 5 steps, starting at a dilution of 3X. The concentrations dropped below LLOD from dilution factor 12× onwards for all 4 plasma samples. In a graph of log (measured pg/mL) vs log [dilution factor (DF)]plasma measurements over spike measurements demonstrated linearity in detection along the various dilutions. 3 out of the 4 plasma samples were determined to fall within the accepted range of parallelism, with Sample 4 falling just outside of the accepted range. The accepted range of parallelism is <15%. These results demonstrated that the Simoa® pTau-217 assay using Antibody 2 on plasma samples yielded consistent and precise calculations of pTau-217 levels across various concentrations thus demonstrated its utility as a biomarker assay.

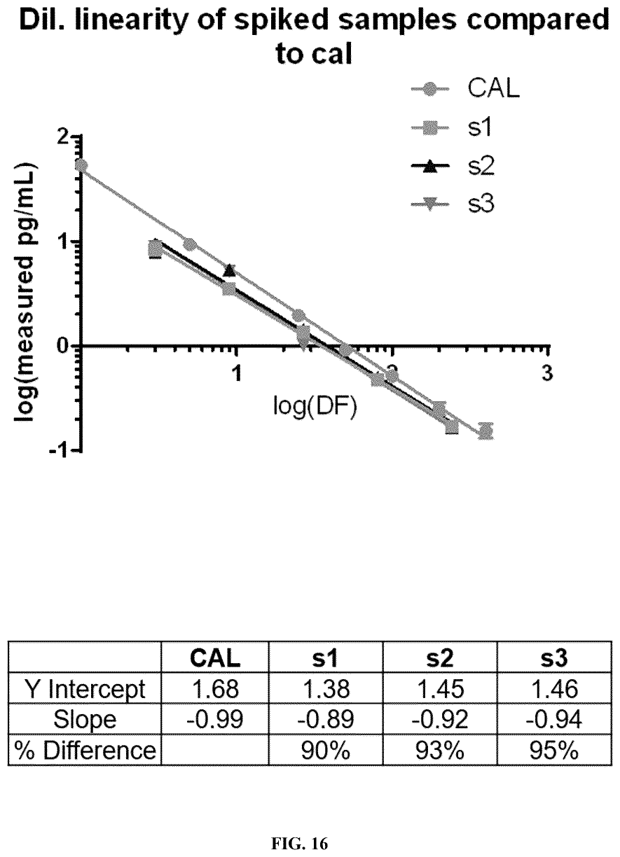

In FIGS. 15-16, dilution linearity using the Simoa® pTau-217 assay using Antibody 2 was performed to demonstrate that a sample with a spike concentration about the upper limit of quantification (ULOQ) can be diluted to a concentration within the working range while still yielding a reliable assay result. In FIG. 15, three spiked samples (sI, s2, and s3) and the Calibration sample were assayed and plotted as log(measured pg/mL) vs log (DF) to determine dilution linearity. In FIG. 16, three spiked samples (sI, s2, and s3) and the Calibration sample were assayed and plotted as log(measured pg/mL) vs log (DF) to determine dilution linearity with the highest spike point omitted from sI, s2, and s3 since it was out of the calibration range of 50 μg/mL.

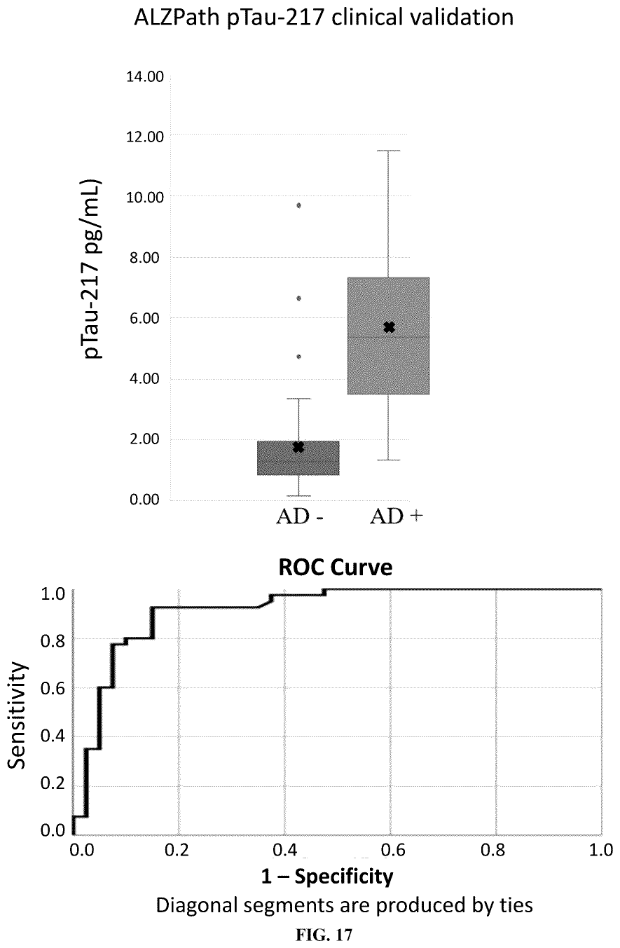

In FIG. 17, the Simoa® pTau-217 assay using Antibody 2 was used to assess samples taken from a memory clinic cohort. Plasma samples were measured and graphed for calculated p-Tau 217 concentrations. Clinical diagnosis of AD was used as the classifier. AUC was calculated at 0.916 indicating success in distinguishing AD+from AD- within this cohort with the Simoa® pTau-217 assay using Antibody 2. Also in FIG. 17, a receiver operating characteristic (ROC) curve was plotted to illustrate the diagnostic ability of this binary classifier (AD+ or AD- system as it is possible that the discrimination threshold between classifiers is varied.

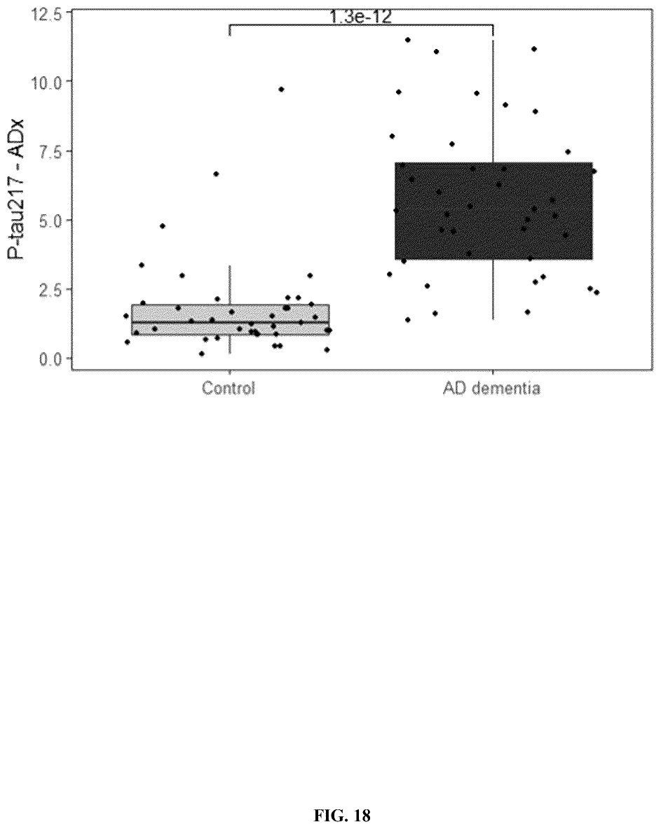

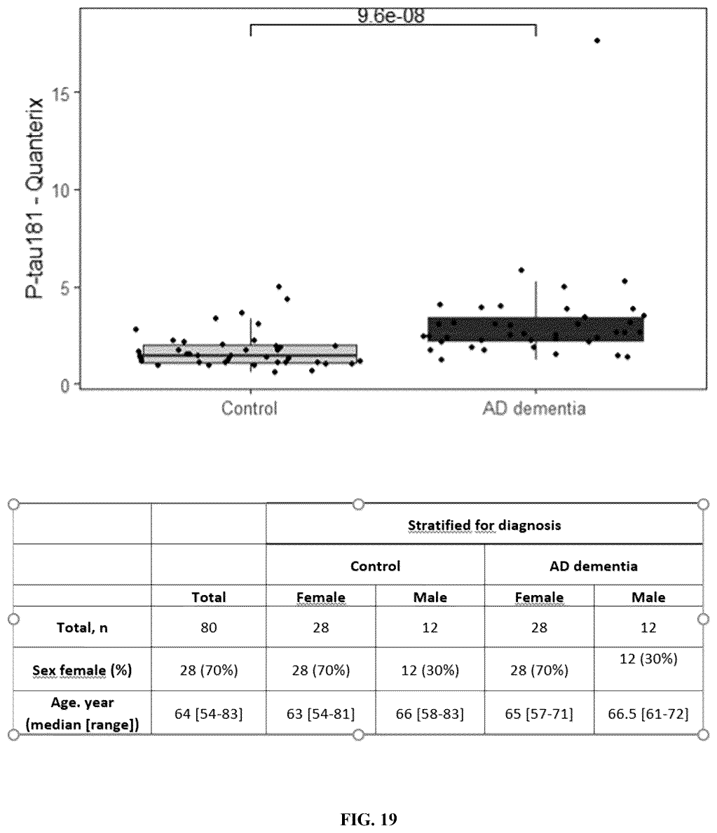

In FIG. 18-19, clinical performance of the Simoa® pTau-217 assay using Antibody 2 was compared to a Simoa® pTau-181 assays using antibody P-Tau 181-Quanterix®. In FIG. 18, Antibody 2 was able to distinguish assayed plasma samples from taken from AD dementia individuals vs Controls (P value 1.3e⁻¹² for Antibody 2). In FIG. 19, the commercially available P-Tau 181—Quanterix® Simoa® assay (Quanterix® Corp., Item number 103714) was also able to distinguish assayed plasma samples from taken from AD dementia individuals vs Controls (P value 9.6e-08). Data from the individuals from which the samples were derived for data from FIGS. 18-19 is listed in FIG. 19.

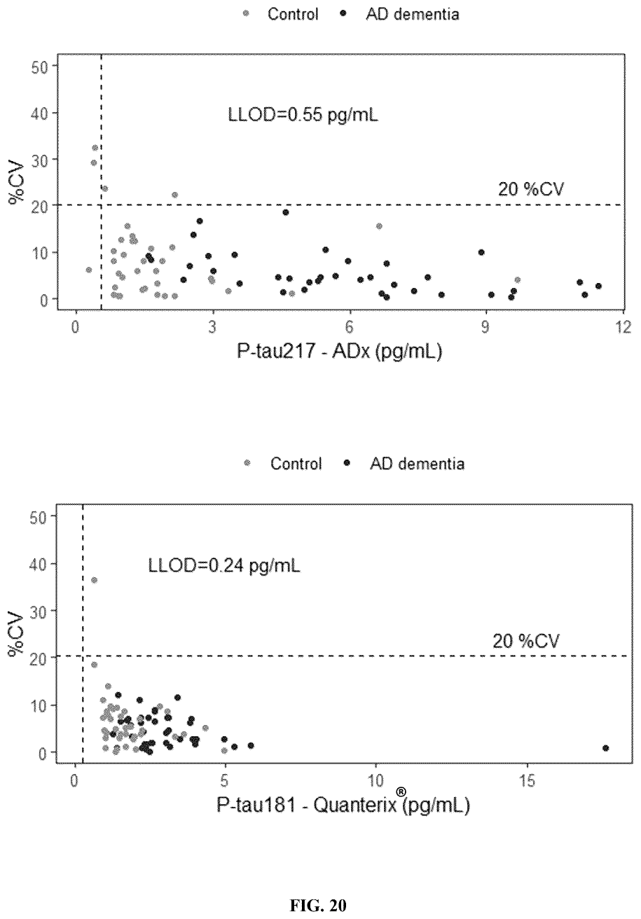

In FIG. 20 precision plots were generated for the Simoa® assay using P-Tau 217 Antibody 2 and P-Tau 181—Quanterix® antibody (Quanterix® Corp., Item number 103714). Calculated LLODs were 0.55 pg/mL and 0.24 pg/mL, respectively. Concentrations were not back calculated and LLOD values are accordingly not back calculated.

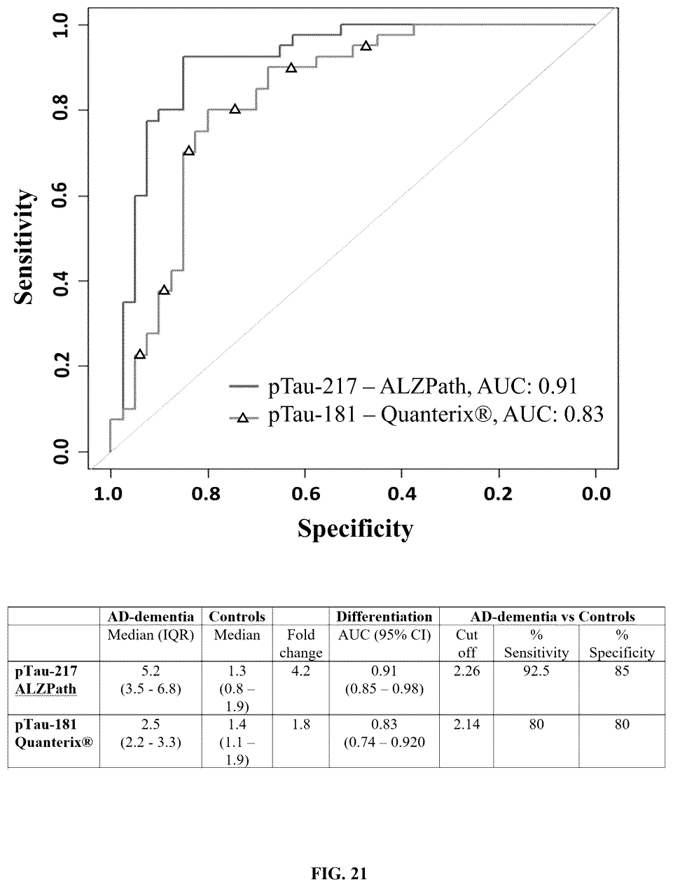

In FIG. 21 ROC curves were plotted for the P-Tau 181—Quanterix® antibody (Quanterix® Corp., Item number 103714) and P-Tau 217 Antibody 2. Analysis of the data indicated that P-Tau 217 Antibody 2 exhibited superior sensitivity and specificity when comparing to the Simoa® assay using the P-Tau 181—Quanterix® antibody in differentiating AD-dementia from controls. The diagnostic accuracy of Antibody 2 for AD dementia in this Simoa® method is 92.5% when tested on plasma samples. The diagnostic specificity of Antibody 2 for AD dementia in this Simoa® method is 85% when tested on plasma samples.

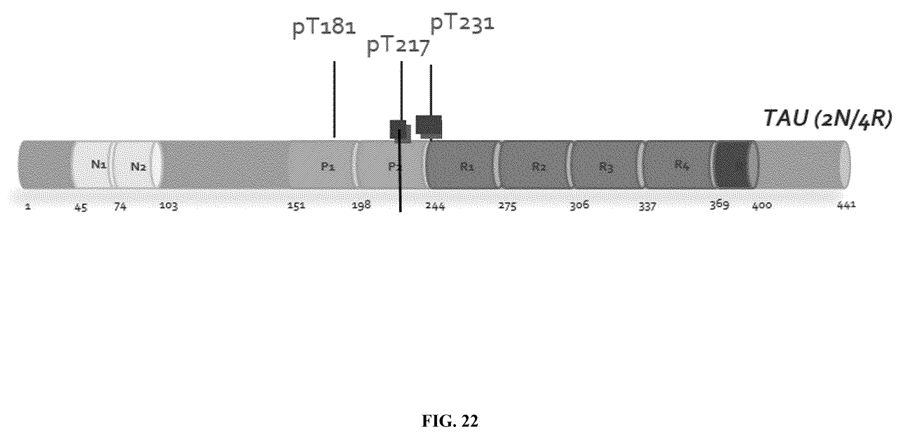

In FIG. 22 a schematic diagram of Tau polypeptide indicating the relative location of various protein domains and the locations of threonine residues which can be assayed for phosphorylation status using methods disclosed herein is depicted. The location of pT217 within the P2 domain of Tau is indicated. pT181 resides within the P1 domain and pT231 resides near the border between the P2 and R1 domains.

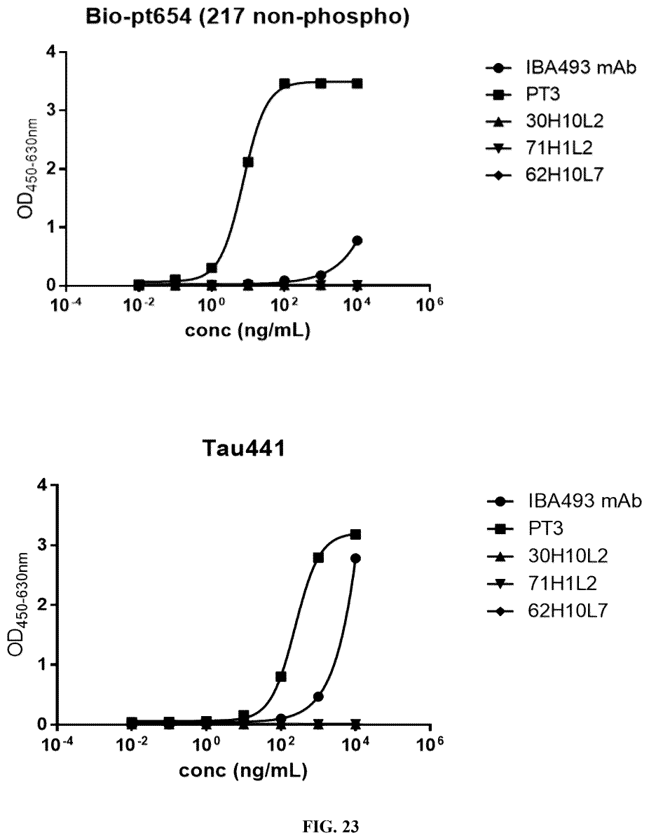

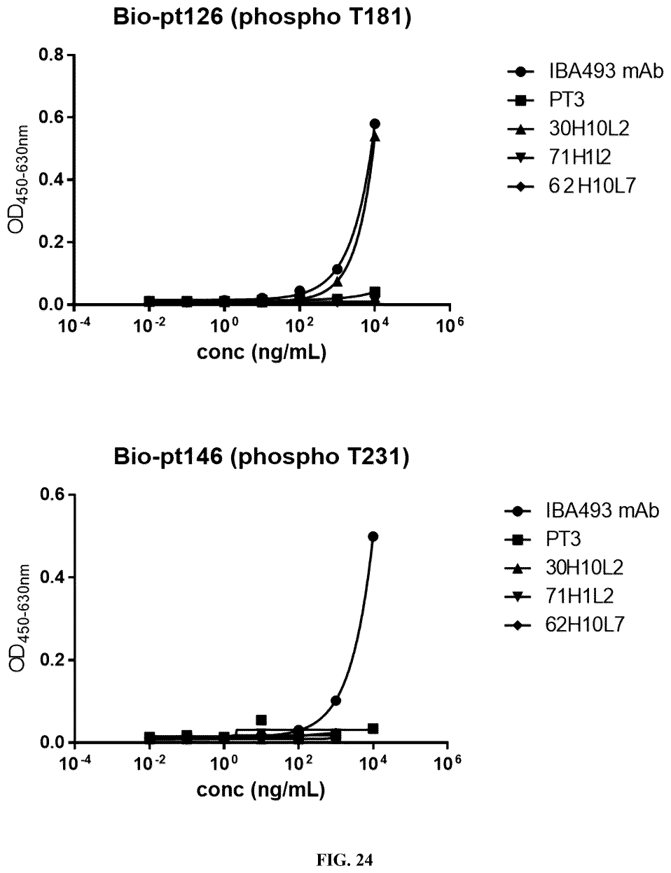

In FIG. 23 various Tau antibodies were assayed using indirect ELISA and extent of reactivity to a Tau fragment with non-phosphorylated T217 (Bio-pt654) and full length Tau (Tau441) were graphed. IBA493 mAβ and PT3 displayed concentration dependent reactivity to both Bio-pt654 and Tau441. Antibody 2, 5, and 6 described herein do not display any reactivity to either Bio-pt654 or Tau441 in this assaying demonstrating precision and specificity in pTau-217 detection for Antibodies 2, 5, and 6.

In FIG. 24 various Tau antibodies were assayed using indirect ELISA and extent of reactivity to a Tau fragment with phosphorylated T181 (Bio-pt126) and phosphorylated T231 (Bio-pt146) were graphed. IBA493 mAβ and Antibody 2 described herein displayed concentration dependent reactivity to Bio-pt126. IBA493 mAβ was the only antibody of those tested displaying concentration dependent reactivity to Bio-pt146. This demonstrates that IBA493 mAβ, PT3, and Antibody 2 described herein were each distinguishable based on which analytes each antibody interacted with via indirect ELISA. IBA493 mAβ interacts with pTau-217, non-phospho T217, full length Tau, pTau-181, and pTau-231. PT3 interacts with pTau-217, non-phospho T217, and full length Tau. Antibody 2 interacts with pTau-217 and pTau-181. IBA493 mAβ's interaction with non-phospho T217 was also shown to be considerably less than PT3's interaction with non-phospho T217.

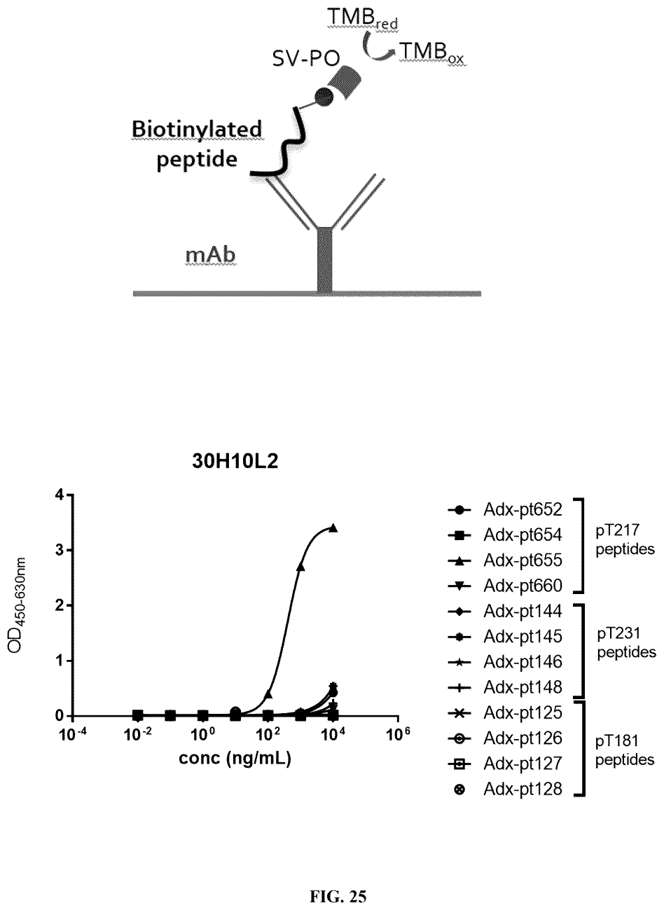

In FIG. 25, a diagram of an assay using Antibody 2 to detect capture of particular Tau peptides is depicted. In this assay, Antibody 2 is bound to a plate and sample wells from the plate are subjected to various biotinylated peptides under conditions conducive to forming specific antibody-ligand interactions. Samples are then washes to remove excess unbound biotinylated peptide. Streptavidin beads conjugated to peroxidase and then added to the samples to allow biotin-streptavidin complexes to form on peptide bound antibodies. TMB_{red} substrate is then added and samples are measured for colorimetric development using an ELISA plate reader. The results were graphed and out of various pTau-217, pTau-231, and pTau-181 peptides tested, Adx-pt655 yielded specific dose-dependent reactivity. These results illustrate the specificity of Antibody 2 to specific features of pTau (namely Tau phosphorylated at threonine 217). Antibody 5 and Antibody 6 tested by indirect ELISA under the same conditions using the same Tau peptides yielded no specific dose-dependent reactivity toward the Tau peptides tested.

Various pTau-217 antibodies corresponding to Antibody 1, Antibody 2, Antibody 3, Antibody 4, Antibody 5, and Antibody 5 described here were also evaluated as capture antibodies either directly coated onto or onto streptavidin-coated plates on the Mesoscale Discovery technology platform. This system uses non-radioactive, electrochemiluminescent labels, thereby conferring significant advantages over traditional ELISA assays. These advantages include lower background signal, improved sensitivity, and a dynamic range of detection.

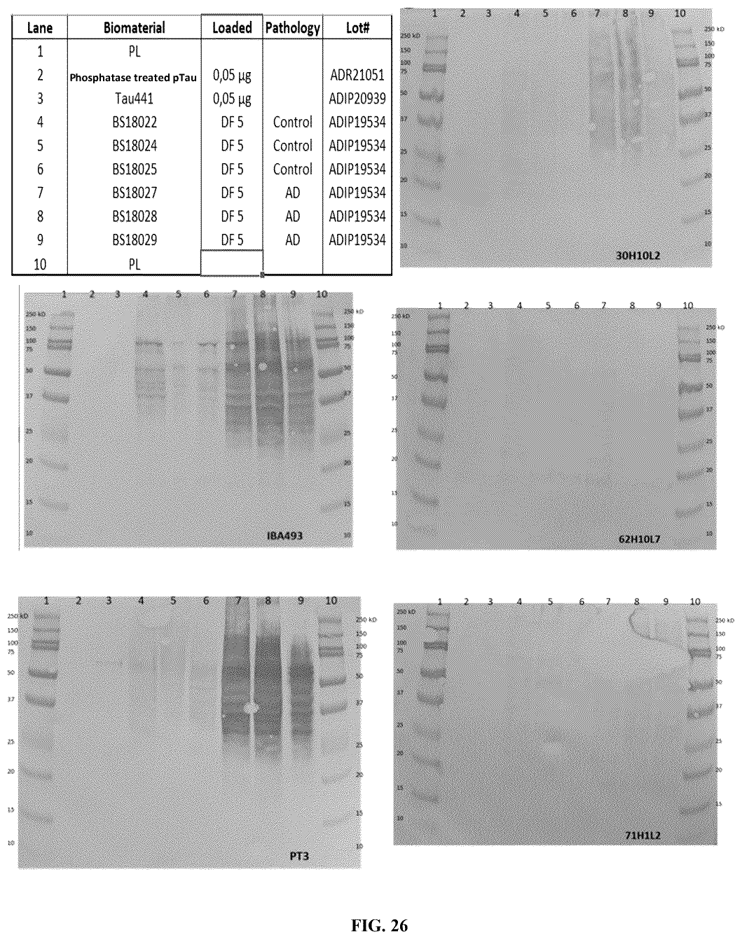

In FIG. 26, Western blots were used to assess binding of various antibodies to brain lysate samples from AD patients and Control subjects. In the five Western blots shown, samples are loaded according to the same sample key shown in FIG. 26. Protein ladders were run in lanes 1 and 10. Phosphatase treated pTau loaded in an amount of 0.05 ug was run in lane 2. Full-length Tau (Tau411) loaded in an amount of 0.05 ug was run in lane 3. Lanes 4-6 contain samples from different Control subjects with a dilution factor of 5. Lanes 7-9 contain samples from different AD subjects with a dilution factor of 5. The results indicated that IBA394 mAb and PT3 both bound to and immunoprecipitated different length isoforms of Tau in control samples and AD samples and immunoprecipitated significantly more Tau in AD samples while showing no interaction with synthetic full length Tau or phosphatase treated pTau. Antibody 2 (30H2L10) bound to and immunoprecipitated different length isoforms of Tau in AD samples but did not immunoprecipitate a significant amount to of Tau in samples from Control individuals. Antibody 5 and Antibody 6 did not yield detectable Western blot signals in this assay.

### Example 5: Early Diagnosis of Neurodegenerative Disease Using Anti-Tau Antibodies

This study shows that the anti-tau antibodies described herein are useful to diagnose early stage of neurodegenerative disease.

### Methods

Subjects are 1000 elderly people aged 60 years or older, who have one or more of the symptoms or conditions selected from cognitive impairment (mental decline, difficulty thinking and understanding, confusion in the evening hours, delusion, disorientation, forgetfulness, making things up, mental confusion, difficulty concentrating, inability to create new memories, inability to do simple math, or inability to recognize common things), behavioral changes (aggression, agitation, difficulty with self-care, irritability, meaningless repetition of own words, personality changes, lack of restraint, or wandering and getting lost), mood changes (anger, apathy, general discontent, loneliness, mood swings), psychological symptoms (depression, hallucination, or paranoia), or physical symptoms (inability to combine muscle movements orjumbled speech). As control group, 1000 elderly people aged 60 years or older with normal cognitive function are provided.

The information of clinical and neuropsychological assessment, individual traits and social environmental factors, and neuroimaging data is collected. The biological samples such as peripheral blood or cerebrospinal fluid are collected.

A level of pT217 tau in the cerebrospinal fluid (CSF) sample from each subject is detected by performing an immunoassay on the sample using anti-tau antibodies described herein. The level of pT217 tau in the CSF sample from the subject is correlated with a standard value of pT217 tau CSF level derived from control group. Based on the increased level of pT217 tau in the sample from the subject, early stage of neurodegenerative disease such as Alzheimer's disease and Huntington's disease is determined.

Alternatively, a level of pT217 tau in the plasma sample from peripheral blood from each subject is detected by performing an immunoassay on the sample using anti-tau antibodies described herein. The level of pT217 tau in the plasma sample from the subject is correlated with a standard value of pT217 tau plasma level derived from control group. Based on the increased level of pT217 tau in the sample from the subject, early stage of neurodegenerative disease such as Alzheimer's disease is determined.

For the next five years, the information of clinical and neuropsychological assessment, individual traits and social environmental factors, and neuroimaging data as well as biological samples are collected every year.

### Results

The subjects that show increased level of pT217 tau in the sample will develop neurodegenerative disease such as Alzheimer's disease and Huntington's disease. This study shows that the anti-tau antibodies described herein are useful in diagnosing early stages of neurodegenerative diseases such as Alzheimer's disease and Huntington's disease. This study also shows that increased level of pT217 tau in the sample at a pre-clinical stage prior to onset of neurodegeneration symptoms allow for an early prognosis of disease progression and for likelihood of eventual neurodegenerative disorder diagnosis in the subject.

### Example 6: Prevention of Neurodegenerative Disease

This study shows that the anti-tau antibodies described herein are useful to prevent neurodegenerative disease by detecting early stage of neurodegenerative disease.

### Methods

Subjects are 1000 elderly people aged 60 years or older, who has one or more of the symptoms or conditions selected from cognitive impairment (mental decline, difficulty thinking and understanding, confusion in the evening hours, delusion, disorientation, forgetfulness, making things up, mental confusion, difficulty concentrating, inability to create new memories, inability to do simple math, or inability to recognize common things), behavioral changes (aggression, agitation, difficulty with self-care, irritability, meaningless repetition of own words, personality changes, lack of restraint, or wandering and getting lost), mood changes (anger, apathy, general discontent, loneliness, mood swings), psychological symptoms (depression, hallucination, or paranoia), or physical symptoms (inability to combine muscle movements or jumbled speech). As control group, 1000 elderly people aged 60 years or older with normal cognitive function are provided.

The information of clinical and neuropsychological assessment, individual traits and social environmental factors, and neuroimaging data is collected. The biological samples such as peripheral blood or cerebrospinal fluid are collected.

A level of pT217 tau in the cerebrospinal fluid (CSF) sample from a subject in each cohort is detected by performing an immunoassay on the sample using anti-tau antibodies described herein. The level of pT217 tau in the CSF sample from the subject is correlated with a standard value of pT217 tau CSF level derived from control group. Based on the increased level of pT217 tau in the sample from the subject, early stage of neurodegenerative disease, such as Alzheimer's disease and Huntington disease, is determined.

Therapeutic agent (including commercially available product, clinical stage product, etc.) is administered to prevent or treat neurodegenerative disease. Donanemab, Lecanemab, or Remternetug is administered to prevent progression of AD or treat AD. Tominersen, is administered to prevent progression of or treat Huntington's disease.

For the next five years, the information of clinical and neuropsychological assessment, individual traits and social environmental factors, and neuroimaging data as well as biological samples are collected every year.

### Results

The results show that administering the therapeutic agent in early stage of neurodegenerative disease is critical to prevent progression of and treat neurodegenerative disease. This study shows that the anti-tau antibodies described herein are useful in preventing progression of or treating neurodegenerative disease by detecting early stage of neurodegenerative disease.

### Example 7: A Phase 2, Multi-enter, Double-Blind, Placebo Controlled Study to Evaluate Safety, Tolerability, and Efficacy of Donanemab in Early Symptomatic Alzheimer's Disease

To evaluate safety, tolerability, and efficacy of donanemab in treating early symptomatic Alzheimer's disease (AD), a phase 2 clinical trial was conducted. The detailed design of the clinical trial protocol is provided in the protocol synopsis of Table 12 below.

TABLE 12
PROTOCOL SYNOPOSIS

| | |
|---|---|
| TITLE: | Assessment of Safety, Tolerability, and Efficacy of LY3002813 in Early |
| | Symptomatic Alzheimer's Disease |
| PROJECT | Phase 2 |
| PHASE: | |
| PRIMARY | To test the hypothesis that LY3002813 (donanemab) administered for up to |
| OBJECTIVE: | 72 weeks decreases the cognitive and/or functional decline in patients with |
| | early symptomatic AD |
| SECONDARY | To assess the effect of donanemab vs. placebo on clinical progression in |
| OBJECTIVE: | patients with early symptomatic AD |
| | To test the effect of donanemab vs. placebo on deposition of amyloid (or |
| | Aβ plaques) in the brain, change in brain amyloid plaque deposition from |
| | baseline through 18 months was measured by florbetapir F18 PET scan |
| | To test the effect of donanemab vs. placebo on tau deposition in the brain |
| | of patients, change in brain tau deposition from baseline to 18 months |
| | was measured by flortaucipir F18 PET scan. |
| | To test the effect of donanemab vs. placebo on changes in brain volume, |
| | change in volumetric MRI measures from baseline to 18 months was |
| | conducted. |
| Safety | Standard safety assessments were conducted (e.g., spontaneously |
| Objective: to | reported adverse events (AEs), clinical laboratory tests performed on |
| evaluate safety | samples from patients, patient monitoring via body weight and vital sign |
| and tolerability | measurements, physical and neurological examination conducted during |
| of donanemab | treatment period, MRI (amyloid-related imaging abnormality (ARIA) |
| | and emergent radiologicals); Columbia Suicide Severity Rating Scale (C- |
| | SSRS) |
| Exploratory | Exploratory endpoint: the change in dependence level derived from |
| Objective: | ADCS-ADL scale scores |
| To assess effect | |
| of donanemab | |
| vs. placebo on | |
| clinical | |
| progression in | |
| patients with | |
| early | |
| symptomatic | |
| AD | |
| Study Design: | A multicenter, randomized, double-blind, placebo-controlled, Phase 2 |
| | study of donanemab in subjects with early symptomatic AD was |
| | conducted. |
| | Screening period up to 9 weeks. |
| | Treatment period up to 72 weeks with final evaluation of trial end |
| | points occurs at Week 76. |
| | Subjects meeting the entry criteria were randomized 1:1 into the |
| | following arms: |
| | 1) i.v. donanemab (700 mg Q4WK for the first 3 doses, then 1400 mg |
| | Q4WK) for up to 72 weeks; or |
| | 2) i.v. placebo Q4WK for up to 72 weeks. |
| Screening | Modified Hachinski Ischemic Scale (MHIS) completed at Visit 1 |
| Procedures: | to exclude patients likely to have dementia of vascular etiology. |
| | MMSE administered to patients at Visit 1 to determine if the |
| | patient meets entry criteria for cognitive impairment. The range |
| | for the total MMSE score is 0 to 30, with lower scores indicating |
| | great level of impairment. |
| | CogState Brief Battery (CBB) - a computer-based cognitive test |
| | battery designed to measure psychomotor function, attention, |
| | working memory, and memory, was administered. The CBB is |
| | administered at Visit 1 to better understand the utility of this test |
| | in assessment of cognitive impairment, but patient eligibility did |
| | not depend on the CBB results to determine if the patient meets |
| | the cognitive criteria for the study. |
| | C-SSRS administered to patients at Visit 1 to assess |
| | psychological health. |
| | PET and MRI screening: All other screening criteria should be |
| | met in order for the patient to proceed with flortaucipir F18 PET |
| | scan, followed by MRI, and a florbetapir F18 PET scan |
| | procedure. |
| | A screening flortaucipir F18 PET scan was performed as part of |
| | the study eligibility criteria to assess tau deposition in the brain |
| | prior to initiation of treatment. |
| | A local screening MRI was performed at Visit 1 as part of the |
| | study eligibility criteria to assess baseline brain volume prior to |
| | initiation of treatment. |
| | A screening florbetapir F18 PET scan was performed as part of |
| | the study eligibility criteria to assess amyloid deposition in the |
| | brain prior to initiation of treatment. |
| | The study includes a screening visit, which can last up to 63 days, |
| | at which patients were required to have PET flortaucipir F18 PET |
| | and florbetapir F18 PET imaging results consistent with tau and |
| | amyloid pathology in order to be randomized to the double-blind |
| | period. |
| Double- | Visit 1 was to determine patient eligibility and then to randomize |
| blinding | selected patients into a study arm. Treatments (either with |
| | donanemab IV administration or IV placebo) were done every |
| | four weeks for up to 72 weeks. For visits 2-21 of the treatment |
| | period, patients in the trial were double-blinded. The duration of |
| | the double-blind period of the study was 76 weeks and included |
| | up to 72 weeks of treatment. |
| Number of | Approximately 1955 participants were randomized, across 56 sites in US |
| participants | and Canada |
| Dosing | Donanemab (700 mg or 1400 mg) was administered every 4 weeks as an |
| | IV infusion of approximately 140 mL over a minimum of 30 minutes. |
| Study | Patients were eligible for enrollment in the study only if they meet all of |
| Population and | the following criteria: |
| Inclusion | Gradual and progressive change in memory function reported by |
| Criteria: | patients or informants for ≥6 months. |
| | An MMSE score of 20 to 28 (inclusive) at Visit 1 or an |
| | acceptable historical flortaucipir PET scan within 6 months prior |
| | to Visit 1 that meets the central read criteria. |
| | Meet flortaucipir F18 scan (central read) criteria (intermediate |
| | levels of tau). |
| | Meet florbetapir F18 scan (central read) criteria (elevated |
| | amyloid). |
| Patient/Subject | Men or women, 60 to 85 years of age, inclusive, at the time of informed |
| Characteristics: | consent. |
| | All subjects submit to use of contraception during trial |
| | Patients must have a study partner who will provide written |
| | informed consent to participate, is in frequent contact with the |
| | patient, and will accompany the patient to study visits or be |
| | available by telephone. A second study partner may serve as a |
| | backup to the primary study partner. Study partners must be able |
| | to communicate with site personnel and be willing to comply |
| | with protocol requirements. |
| | Have adequate premorbid literacy, vision, and hearing for |
| | neuropsychological testing in the opinion of the investigator. |
| | Have given written informed consent to participate in the trial |
| | Are reliable and willing to make themselves available for the |
| | duration of the study and are willing to follow study procedures |
| Exclusion | Have a MHIS score of greater than or equal to 4. |
| criteria: | Lack adequate premorbid literacy, adequate vision, or adequate |
| | hearing to complete the required psychometric tests. |
| | Significant neurological disease affecting the central nervous |
| | system (CNS), other than AD |
| | Current serious or unstable illnesses including cardiovascular, |
| | hepatic, renal, gastroenterological, respiratory, endocrinologic, |
| | neurologic (other than AD), psychiatric, immunologic, or |
| | hematologic disease |
| | History of cancer within the last 5 years |
| | Patients with any current primary psychiatric diagnosis other than |
| | AD |
| | Have a history of long QT syndrome |
| | Are clinically judged by the investigator to be at serious risk for |
| | suicide as assessed by medical history, examination, or the C- |
| | SSRS |
| | History of alcohol or drug use disorder (except tobacco use |
| | disorder) within 2 years before the screening visit |
| | Have a history of clinically significant multiple or severe drug |
| | allergies |
| | Known positive serologic findings for human immunodeficiency |
| | virus (HIV) antibodies. |
| | Have any clinically important abnormality at screening |
| | Screening MRI which shows evidence of significant abnormality |
| | that would suggest another potential etiology for progressive |
| | dementia |
| | Have any contraindications for MRI |
| | Have a centrally read MRI demonstrating presence of ARIA-E, >4 |
| | cerebral microhemorrhages, more than 1 area of superficial |
| | siderosis, any macrohemorrhage or severe white matter disease |
| | An average (ECG in triplicate) corrected QT (QTcF) interval |
| | measurement >450 msec (men) or >470 msec (women) at |
| | screening |
| | Positive hepatitis B antigens |
| | Positive hepatitis C RNA PCR. |
| | Calculated creatinine clearance <30 mL/min at initial screening. |
| | Alanine transaminase (ALT) ≥2X the upper limit of normal |
| | (ULN) of the performing laboratory, aspartate aminotransferase |
| | (AST) ≥2X ULN, total bilirubin level (TBL) ≥1.5X ULN, or |
| | alkaline phosphatase (ALP) ≥1.5X ULN at screening |
| Treatments | In the double-blind period, the treatment groups were given an IV |
| administered | infusion of donanemab or placebo for up to 72 weeks. |
| | Patients whose amyloid plaque reduction as measured by florbetapir F18 |
| | PET scans at Visit 8 (Week 24) and/or Visit 15 (Week 52) meet criteria |
| | had a double-blinded dose reduction of donanemab to 700 mg or IV |
| | placebo, based on the specific level of florbetapir F18 PET signal at that |
| | time, for the remaining duration of the double-blind treatment period. |
| | Visit 2 (Week 0) through Visit 21 (Week 76): |
| | Treatment regimen: donanemab administration (700 mg intravenous |
| | infusion every 4 weeks × 3 doses, then 1400 mg intravenous infusion |
| | every 4 weeks for up to 72 weeks) |
| | Placebo: Intravenously dosed placebo infusion every 4 weeks for up to |
| | 72 weeks |
| Primary | Integrated Alzheimer's Disease Rating Scale (iADRS). The iADRS is a |
| Efficacy | simple linear combination of scores from 2 well-established, |
| Assessments: | therapeutically sensitive, widely accepted measures in AD, the ADAS- |
| | Cog13 and the ADCS-iADL, measuring the core domains of AD. |
| | Change in iADRS from Week 1 to subsequent weeks tested in the study |
| | design indicate treatment efficacy of treatment group vs. placebo group. |
| | Change in cognition and function as measured by change in iADRS |
| | score from baseline to 18 months. Cognitive measurements takes at |
| | baseline and weeks 12, 24, 36, 52, 64, and 76. |
| Secondar | ADCS-ADL, clinical dementia rating scale (CDR-SB), MMSE, |
| Efficacy | biomarkers: florbetapir F18 PET signal, flortaucipir F18 PET signal, |
| Assessments: | volumetric MRI. |

Results: 272 participants (mean [SD]age, 75.2 [5.5]years; 145 [53.3%]female) were randomized.

A P-Tau 217 digital immunoassay for the quantitative determination of tau phosphorylated as threonine 217 in samples from human plasma using a Simoa HD-X analyzer was used. The p-Tau 217 antibody tested was from Quanterix. The analytical measurement range (AMR) for values of P-Tau 217 was determined to be 0.200 U/mL t 6.04 U/ni. The sensitivity (analytical) defined as the lower limit of quantitation of P-Tau 217 was determined to be 0.200 U/mL. No cross-reactivity was seen with spiked Tau protein in the P-Tau 217 assay. Table 13 lists baseline characteristics and biomarker levels analyzed.

TABLE 13
Baseline characteristics and biomarker levels

| | |
|---|---|
| Placebo | Donanemab |

Baseline Characteristics

| | | |
|---|---|---|
| Age (years) - N, mean (SD) | 120, 75.29 (5.438) | 125, 74.90 (5.504) |
| Amyloid (CL)- N, mean (SD) | 112, 103.09 (33.841) | 121, 107.18 (33.938) |
| Global Tau (SUVR)- N, mean (SD) | 113, 1.22 (0.132) | 122, 1.21 (0.142) |
| MMSE- N, mean (SD) | 115, 23.77 (2.878) | 121, 23.56 (3.033) |
| iADRS- N, mean (SD) | 120, 106.06 (13.050) | 125 106.30 (12.730) |
| P-Tau 217 (U/mL)- N, mean (SD) | 119, 0.42 (0.172) | 123, 0.41 (0.232) |
| GFAP (pg/mL)- N, mean (SD) | 103, 222.07 (103.406) | 104, 214.43 (88.891) |
| NfL (pg/mL)- N, mean (SD) | 119, 26.82 (24.847) | 121, 21.91 (8.491) |
| Aβ 42 (pg/mL)- N, mean (SD) | 103, 5.93 (1.382) | 104, 5.54 (1.395) |
| Aβ 40 (pg/mL)- N, mean (SD) | 103, 117.75 (29.559) | 104, 114.23 (28.395) |
| Aβ 42/40- N, mean (SD) | 103, 0.051 (0.0069) | 104, 0.049 (0.0089) |

Week 24

| | | |
|---|---|---|
| P-Tau 217 (U/mL)- N, mean (SD) | 110, 0.45 (0.257) | 111, 0.34 (0.120) |
| GFAP (pg/mL)- N, mean (SD) | 102, 234.40 (103.238) | 98, 203.12 (90.661) |
| NfL (pg/mL)- n, mean (SD) | 111, 28.12 (25.906) | 110, 25.56 (11.390) |
| Aβ 42 (pg/mL)- N, mean (SD) | 102, 6.33 (1.657) | 98, 6.04 (1.500) |
| Aβ 40 - N, mean (SD) | 102, 127.35 (36.264) | 98, 120.53 (31.935) |
| Aβ 42/40- N, mean (SD) | 102, 0.051 (0.0083) | 98, 0.051 (0.0096) |

Week 52

| | | |
|---|---|---|
| P-Tau 217 (U/mL)- N, mean (SD) | 90, 0.45 (0.225) | 90, 0.32 (0.178) |
| GFAP (pg/mL)- N, mean (SD) | 78, 228.14 (76.810) | 82, 188.10 (80.199) |
| NfL (pg/mL)- n, mean (SD) | 93, 29.47 (30.087) | 90, 23.43 (11.193) |
| Aβ 42 (pg/mL)- N, mean (SD) | 78, 6.19 (1.735) | 82, 6.17 (1.552) |
| Aβ 40 (pg/mL)- N, mean (SD) | 78, 121.42 (40.220) | 82, 120.68 (27.122) |
| Aβ 42/40- N, mean (SD) | 78, 0.052 (0.0075) | 82, 0.052 (0.0094) |

Week 76

| | | |
|---|---|---|
| P-Tau 217(U/mL)- N, mean (SD) | 87, 0.46 (0.245) | 86, 0.32 (0.167) |
| GFAP (pg/mL)- N, mean (SD) | 78, 242.25 (87.293) | 83, 189.99 (83.675) |
| NfL (pg/mL)- N, mean (SD) | 90, 26.05 (11.431) | 92, 23.86 (10.529) |
| Aβ 42 (pg/mL)- N, mean (SD) | 78, 6.65 (2.116) | 83, 6.31 (1.718) |
| Aβ 40 (pg/mL)- N, mean (SD) | 78, 132.30 (44.026) | 83, 123.68 (29.815) |
| Aβ 42/40- N, mean (SD) | 78, 0.051 (0.0090) | 83, 0.051 (0.0090) |

Table 14 lists changes from baseline in plasma biomarker levels

TABLE 14
Plasma biomarker change from baseline during study

| | |
|---|---|
| Placebo | Donanemab |

P-Tau 217 (U/mL) log10 transformed

| | | | |
|---|---|---|---|
| Week 12 | LS Mean Change (SE), N | 0.00 (0.010)11 | −0.04 (0.010), 119 |
| | LS Mean Diff (95% CI) vs. | | −0.04 (−0.07, −0.02) |
| | P-value vs. placebo | | 0.002 |
| Week 24 | LS Mean Change (SE), N | 0.01 (0.011)11 | −0.09 (0.011), 110 |
| | LS Mean Diff (95% CI) vs. | | −0.10 (−0.13, −0.07) |
| | P-value vs. placebo | | <0.001 |
| Week 36 | LS Mean Change (SE), N | −0.01 (0.014)10 | −0.09 (0.014), 100 |
| | LS Mean Diff (95% CI) vs. | | −0.08 (−0.12, −0.04) |
| | P-value vs. placebo | | <0.001 |
| Week 52 | LS Mean Change (SE), N | 0.02 (0.014)89 | −0.11 (0.014), 90 |
| | LS Mean Diff (95% CI) vs. | | −0.12 (−0.16, −0.09) |
| | P-value vs. placebo | | <0.001 |
| Week 64 | LS Mean Change (SE), N | 0.01 (0.011)85 | −0.13 (0.011), 84 |
| | LS Mean Diff (95% CI) vs. | | −0.14 (−0.17, −0.11) |
| | P-value vs. placebo | | <0.001 |
| Week 76 | LS Mean Change (SE), N | 0.03 (0.014)86 | −0.11 (0.014), 86 |
| | LS Mean Diff (95% CI) vs. | | −0.14 (−0.18, −0.10) |
| | P-value vs. placebo | | <0.001 |

GFAP (pg/mL) log10 transformed

| | | | |
|---|---|---|---|
| Week 12 | LS Mean Change (SE), N | 0.03 (0.011)87 | −0.01 (0.011), 93 |
| | LS Mean Diff (95% CI) vs. | | −0.04 (−0.07, −0.01) |
| | P-value vs. placebo | | 0.01 |
| Week 24 | LS Mean Change (SE), N | 0.03 (0.012)91 | −0.04 (0.013), 84 |
| | LS Mean Diff (95% CI) vs. | | −0.07 (−0.10, −0.03) |
| | P-value vs. placebo | | <0.001 |
| Week 36 | LS Mean Change (SE), N | 0.02 (0.012)85 | −0.07 (0.013), 79 |
| | LS Mean Diff (95% CI) vs. | | −0.09 (−0.12, −0.06) |
| | P-value vs. placebo | | <0.001 |
| Week 52 | LS Mean Change (SE), N | 0.05 (0.013)71 | −0.06 (0.013), 70 |
| | LS Mean Diff (95% CI) vs. | | −0.11 (−0.15, −0.08) |
| | P-value (vs. placebo) | | <0.001 |
| Week 64 | LS Mean Change (SE), N | 0.03 (0.015)69 | −0.06 (0.016), 63 |
| | LS Mean Diff (95% CI) vs. | | −0.09 (−0.14, −0.05) |
| | P-value (vs. placebo) | | <0.001 |
| Week 76 | LS Mean Change (SE), N | 0.06 (0.016)67 | −0.06 (0.016), 69 |
| | LS Mean Diff (95% CI) vs. | | −0.12 (−0.16, −0.07) |
| | P-value (vs. placebo) | | <0.001 |

NfL (pg/mL) log10 transformed

| | | | |
|---|---|---|---|
| Week 12 | LS Mean Change (SE), N | 0.03 (0.012) 87 | 0.03 (0.012), 93 |
| | LS Mean Diff (95% CI) vs. | | 0.00 (−0.03, |
| | P-value (vs. placebo) | | 0.89 |
| Week 24 | LS Mean Change (SE), N | 0.02 (0.011) 91 | 0.04 (0.012), 84 |
| | LS Mean Diff (95% CI) vs. | | 0.02 (−0.01, |
| | P-value (vs. placebo) | | 0.23 |
| Week 36 | LS Mean Change (SE), N | 0.02 (0.012) 85 | 0.02 (0.013), 79 |
| | LS Mean Diff (95% CI) vs. | | 0.00 (−0.03, |
| | P-value (vs. placebo) | | 0.97 |
| Week 52 | LS Mean Change (SE), N | 0.04 (0.015) 71 | 0.04 (0.015), 70 |
| | LS Mean Diff (95% CI) vs. | | −0.00 (−0.05, |
| | P-value (vs. placebo) | | 0.86 |
| Week 64 | LS Mean Change (SE), N | 0.05 (0.015) 69 | 0.03 (0.016), 63 |
| | LS Mean Diff (95% CI) vs. | | 0.02 (−0.06, |
| | P-value (vs. placebo) | | 0.44 |
| Week 76 | LS Mean Change (SE), N | 0.07 (0.015) 67 | 0.06 (0.015), 69 |
| | LS Mean Diff (95% CI) vs. | | −0.01 (−0.06, |
| | P-value (vs. placebo) | | 0.49 |

Aβ 42/40 log10 transformed

| | | | |
|---|---|---|---|
| Week 12 | LS Mean Change (SE), N | 0.00 (0.005) 87 | 0.00 (0.005), 93 |
| | LS Mean Diff (95% CI) vs. | | −0.00 (−0.02, |
| | P-value (vs. placebo) | | 0.85 |
| Week 24 | LS Mean Change (SE), N | 0.00 (0.005) 91 | 0.01 (0.006), 84 |
| | LS Mean Diff (95% CI) vs. | | 0.01 (−0.01, |
| | P-value (vs. placebo) | | 0.36 |
| Week 36 | LS Mean Change (SE), N | 0.01 (0.006) 85 | 0.02 (0.006), 79 |
| | LS Mean Diff (95% CI) vs. | | 0.02 (0.00, |
| | P-value (vs. placebo) | | 0.04 |
| Week 52 | LS Mean Change (SE), N | 0.01 (0.008) 71 | 0.02 (0.008), 70 |
| | LS Mean Diff (95% CI) vs. | | 0.00 (−0.02, |
| | P-value (vs. placebo) | | 0.68 |
| Week 64 | LS Mean Change (SE), N | 0.01 (0.007) 69 | 0.01 (0.008), 63 |
| | LS Mean Diff (95% CI) vs. | | 0.00 (−0.02, |
| | P-value (vs. placebo) | | 0.88 |
| Week 76 | LS Mean Change (SE), N | 0.01 (0.007) 67 | 0.02 (0.007), 69 |
| | LS Mean Diff (95% CI) vs. | | 0.01 (−0.01, |
| | P-value (vs. placebo) | | 0.26 |

Plasma levels of phosphorylated tau217 (p-Tau 217) and glial fibrillary acidic protein were measured and were found to be significantly lower with donanemab treatment compared with placebo as early as 12 weeks after the start of treatment (least square mean change difference vs placebo,−0.04 [95% Cl, −0.07 to −0.02]; P=0.002 and −0.04 [95% Cl, −0.07 to −0.01]; P=. 0.01, respectively.

Plasma levels of amyloid-B3.42/40 and neurofilament light chain were measured and no significant differences were observed.

Following treatment, changes in plasma p-Tau 217 and GFAP were significantly correlated with the Centiloid percent change in amyloid (Spearman rank correlation coefficient [R]=0.484 [95% Cl, 0.359-0.592]; P<0.001 and R=0.453 [95% Cl, 0.306-0.579]; P<0.001, respectively. The Centiloid scale has previously been developed to standardize results of beta-amyloid (AD3) PET. Additional correlations in patients prior to and during the study design were found to include plasma levels of p-Tau 217 and GFAP as significantly correlated at baseline and following treatment (R=0.399 [95% Cl, 0.278-0.508], P<0.001 and R=0.393 [95% Cl, 0.254-0.517]; P<0.001, respectively).

### Conclusions:

Plasma biomarkers p-Tau 217 and GFAP in donanemab-treated subjects compared to placebo-treated subjects were observed to have significant reductions in the levels as early as 12 weeks after treatment initiation in subjects meeting the criteria of early symptomatic AD. These changes in detectable and quantifiable plasma biomarker output could provide evidence of mitigation of AD pathology or the slowing down of AD pathology progression in response to an anti-amyloid therapy. These changes in detectable and quantifiable plasma p-Tau 217 and GFAP biomarker output could provide a significant correlation to memory and/or cognitive changes in response to an anti-amyloid therapy.

### Example 8: Phase 2A Clinical Trial: Lecanemab

A phase 2a clinical trial is performed to evaluate the efficacy of an antibody directed against amyloid beta (lecanemab or Legembi®) in patients with prodromal Alzheimer's disease (AD), or in patients residing in an early (mild) stage of AD progression, or in patients determined via PET-based imaging of tau to reside within Braak stage I or Braak stage II of AD pathology. The patients are positive for the presence of a level of p-Tau 217 detected in a plasma sample from the patient, wherein the p-Tau 217 level is above a threshold level of p-Tau 217 determined from assaying reference samples taken from healthy individuals not suspected of having a neurogenerative disease in accordance with various embodiments herein. The level of p-Tau 217 is determined using a Simoa®-based pTau assay-217 with an anti-p-Tau 217 antibody described herein (e.g., Antibody 2). Alternatively, or in addition, the patients are positive for the presence of a level of p-Tau 181 detected in a plasma sample from the patient, wherein the p-Tau 181 level is above a threshold level of p-Tau 181 determined from assaying reference samples taken from healthy individuals not suspected of having a neurogenerative disease. Optionally, the level of p-Tau 181 is determined using a Simoa®-based pTau assay-181 with an anti-p-Tau 181 antibody from Quanterix®. Lecanemab is a monoclonal antibody designed to preferentially target soluble aggregated amyloid beta (AD) in the form of Aβ oligomers, protofibrils, and insoluble fibrils; with administration of lecanemab resulting in lowering Aβ plaques in the brain.

Screening Procedures considered for use in this example: Modified Hachinski Ischemic Scale (MHIS), Mini-Mental State Examination (MMSE), CogState Brief Battery, Columbia Suicide Severity Rating Scale - Adult Version, Screening Positron Emission Tomography and Magnetic Resonance Imaging, Screening Flortaucipir F18 PET Scan, Screening MRI, Screening Florbetapir F18 PET Scan.

Scoring the MMSE: The MMSE is a brief instrument used to assess cognitive function in elderly patients as described in Folstein M F et al., which is hereby incorporated by reference regarding the screening protocols described therein (Folstein M F, et al. “Mini-mental state”. A practical method for grading the cognitive state of patients for the clinician. J Psychiatr Res. 1975 November; 12(3):189-98). The MMSE is administered to patients at Visit 1 to determine if the tested patient meets entry criteria for cognitive impairment. The MMSE is to be administered by the same scorer from visit to visit to reduce potential variability. The instrument is divided into 2 sections. The first section measures orientation, memory, and attention. The maximum score for the first section is 21. The second section tests the ability of the patient to name objects, follow verbal and written commands, write a sentence, and copy figures. The maximum score for the second section is 9. The range for the total MMSE score is 0 to 30, with lower scores indicating great level of impairment.

Screening Positron Emission Tomography (PET): If initial screening criteria are met, a patient may proceed with flortaucipir F18 PET scan, followed by MRI, and a florbetapir F18 PET scan procedure..

### Screening Flortaucipir F18 PET Scan:

A screening flortaucipir F18 PET scan is performed as part of the study eligibility criteria.

The flortaucipir F18 PET scan is submitted to a centralized PET imaging vendor selected for assessment of patient's eligibility. A historical flortaucipir F18 PET scan optionally may be submitted to be considered for eligibility if the PET scan was performed within about 6 months of Visit 1. If a historical flortaucipir PET scan is deemed acceptable for meeting entry criteria, then the MMSE, which is still to be administered at Visit 1, serves as the baseline MMSE for statistical analyses, but is not used for meeting entry criteria. The flortaucipir F18 PET screening criteria should be met (scan results consistent with trial sponsor-derived eligibility limits for flortaucipir F18 PET) before the patient may proceed to undergo the MRI and florbetapir F18 PET scan.

Flortaucipir F18 PET scan itself is performed according to instruction provided in the PET Imaging Manual.

### Screening MRI:

A local screening MRI is performed at Visit 1 as component of the study eligibility criteria. With the exception of the evidence of amyloid pathology by florbetapir F18 PET scan, a patient should meet all other Visit 1 eligibility criteria before having an MRI. The MRI scans are reviewed by the investigator or qualified designee for patient management. After the MRI scan is read locally, the scan is submitted to the centralized MRI vendor designated by the sponsor for final determination of MRI eligibility. Results of centrally read MRIs are used for data analysis and report-writing purposes and patient safety and eligibility is reported back to sites.

### Screening Florbetapir F18 PET Scan:

A screening florbetapir F18 PET scan is performed as part of the study eligibility criteria. A patient should meet all other Visit 1 eligibility criteria before having a screening florbetapir F18 PET scan. The florbetapir F18 PET scan is submitted to a centralized PET imaging vendor designated by the trial sponsor for assessment of patient's eligibility.

The florbetapir F18 PET screening criteria must be met (scan results consistent with sponsor-derived eligibility limits for amyloid plaque pathology) in order for the patient to proceed to Visit 2 and be randomized to treatment assignment. Specific instructions for administering the florbetapir F18 PET scan are provided in the PET Imaging Manual.

### Inclusion Criteria:

Patients are eligible for enrollment in the study if they meet all of the following criteria:

### Type of Patient and Disease Characteristics:

I) Gradual and progressive change in memory function (e.g., a metric of cognitive impairment) reported by patients or informants for a period of time>6 months prior to study enrollment.

II) An MMSE score of 20 to 28 (inclusive both scores at ends of range) at Visit 1 or an acceptable historical flortaucipir PET scan within 6 months prior to Visit 1 that meets the central read criteria.

III) Meet flortaucipir F18 scan (central read) criteria.

IV) Meet florbetapir F18 scan (central read) criteria.

### Patient Characteristics:

A) Men or women, 60 to 85 years of age, inclusive, at the time of informed consent given.

B) Have a study partner who will provide written informed consent to participate, is in frequent contact with the patient (defined as at least 10 hours per week), and will accompany the patient to study visits or be available by telephone. A second study partner may serve as backup.

C) Have adequate premorbid literacy, vision, and hearing for neuropsychological testing in the opinion of the investigator or designated scorer.

D) Have given written informed consent approved by the sponsor and the ethical review board (ERB) governing the site.

E) Are reliable and willing to make themselves easily available for the duration of the study and are also willing to follow study procedures.

Exclusion criteria:

F) Have a Modified Hachinski Ischemia Scale (MHIS; Hachinski et al. 1975) score of >4.

G) Lack adequate premorbid literacy, adequate vision, or adequate hearing to complete the required psychometric tests according to the investigator or designated scorer.

H) Significant neurological disease affecting the central nervous system (CNS), other than AD, that may affect cognition.

I) Current serious or unstable illnesses including cardiovascular, hepatic, renal, gastroenterological, respiratory, endocrinologic, neurologic (other than AD), psychiatric, immunologic, or hematologic disease and other conditions.

J) History of cancer within the last 5 years.

K) Patients with any current primary psychiatric diagnosis other than AD.

L) Have a history of long QT syndrome.

M) Are clinically judged by the investigator to be at a serious risk for suicide as assessed by medical history, in person examination, or the C—SSRS.

Arms: 40 patients per arm (antibody and placebo arms) are treated with antibody or placebo for a period of time up to 72 weeks. The treatment regimen comprises lecanemab or Legembi® administration by intravenous (i.v.) infusion every 4 weeks for up to 72 weeks. The placebo group receives intravenously dosed placebo infusion every 4 weeks for up to 72 weeks. Antibody and placebo arms are double-blinded from Visit 2 onward until the end of study. End of the study is the date of the last visit or last scheduled procedure. Dosages of i.v. infusions of lecanemab as monotherapy: 700 mg Q4WK for the first 3 doses, then 1400 mg Q4WK) for up to 72 weeks, in combination with daily oral placebo. Placebo group receives: intravenous placebo Q4WK for up to 72 weeks in combination with daily oral placebo.

An interim analysis is performed, for example, when all randomized subjects have had a chance to complete 52 weeks of treatment (Visit 15) and data are used to evaluated whether to stop the study. An interim analysis is performed in order to initiate the final population PK/PD model development processes for interim or final analyses. The interim analysis considers the level of p-Tau 217 detected in a plasma sample from the patient as a predicting factor in a later determination of positive diagnosis of AD or an evaluation of an extent of AD pathology progression. The level of p-Tau 217 detected in a plasma sample from the patient is considered as a biomarker for diagnosis in the patient. Optionally, a level of p-Tau 217 in the second plasma sample from the patient may be assessed during the study. This second level may be used as a proxy for treatment efficacy at the interim analysis period or at the end of the study. Maintenance or reduction in the level of p-Tau 217 in the subject following treatment is predicted to correlate with a favorable treatment outcome. A significant increase in the level of p-Tau 217 in the subject following treatment is predicted to correlate with a unfavorable treatment outcome.

### Primary Outcome Measures:

Electronic Clinical Outcome Assessment (eCOA) measures (e.g., a rating scale, including audio voice recordings of the rater's questions and the patient's and study partner's responses) are entered into an eCOA instrument (at the time that the information is obtained). The eCOA tablet has both keyboard entry and audio voice recording capabilities. Cognitive and functional testing are administered using an eCOA tablet. Cognitive and functional testing for each patient are to be performed at approximately the same time on each day that testing occurs to reduce potential variability. Note that the ADAS-Cog and MMSE are to be administered by a separate rater individual than the ADCS-ADL and CDR.

Integrated Alzheimer's Disease Rating Scale (iADRS) is used for the primary efficacy assessment according to Wessels A M, et al. which is hereby incorporated by reference for the procedures described therein (Wessels A M, et al. A Combined Measure of Cognition and Function for Clinical Trials: The Integrated Alzheimer's Disease Rating Scale (iADRS). J Prev Alzheimers Dis. 2015 Dec. 1; 2(4):227-241.).

Secondary Efficacy Assessments: Alzheimer's Disease Assessment Scale-Cognitive subscale (ADAS-Cog13), Alzheimer's Disease Cooperative Study-Activities of Daily Living Inventory (ADCS-ADL), Clinical Dementia Rating Scale (CDR-SB), Mini-Mental State Examination (MMSE), Biomarker Efficacy Measures (Double-Blind Period).

For the Biomarker Efficacy Measures, the following biomarkers are tested: Florbetapir F18 PET scan, Flortaucipir F18 PET scan, Volumetric MRI, p-Tau 217 plasma level, and optionally, p-Tau 181 plasma level.

Primary objective: To test the hypothesis that lecanemab or Legembi® alone administered for up to 72 weeks decreases cognitive and or functional decline in patients with early symptomatic AD and that initial testing for plasma levels of p-Tau 217 is an informative diagnostic biomarker for subjects that would otherwise go on to progress AD functional deficits and hence would benefit from early identification and thereby earlier therapeutic intervention.

Primary endpoint: Change in cognition and function as measured by the change in integrated Alzheimer's Disease Rating Scale (iADRS) score from baseline to 18 months. This change may be i) an improvement, ii) a similar score, or iii) a reduced score that less than a median reduction seen in the scores obtained from the placebo group. This indicates that treatment i) improves memory and/or cognition, ii) stabilizes a measurement of memory and/or cognition, or iii) significantly reduced worsening of memory and/or cognition.

Secondary objectives: To assess the effect of lecanemab or Legembi® alone administered for up to 72 weeks vs. placebo on clinical progression in patients with early symptomatic AD and that initial testing for plasma levels of p-Tau 217 is an informative diagnostic biomarker for subjects that would otherwise go on to progress AD clinical pathology and hence would benefit from early identification and thereby earlier therapeutic intervention. In this sense, a specific and sensitive assay for p-Tau 217 in plasma can lead to earlier diagnosis of AD than by other criteria.

Secondary endpoints: Change in cognition from baseline to 18 months as measured by: change in ADAS-Cog13 score, change in CDR-SB score, change in MMSE score, change in ADCS-iADL score.

Other secondary endpoints: change in brain amyloid plaque disposition from baseline through 18 months as measured via florbetapir F18 PET scan., change in brain tau disposition from baseline through 18 months as measured via flortaucipir F18 PET scan, change in volumetric MRI measures from baseline to 18 months, change in p-Tau 217 plasma levels as measured by Simoa®-based pTau assay-217.

These secondary endpoints are to assess the effect of lecanemab or Legembi® monotherapy administration vs. placebo in i) brain amyloid deposition, ii) brain tau deposition, iii) brain volume measurement changes, and iv) plasma p-Tau 217 levels.

### Example 9: Phase 2A Clinical Trial: Donanemab

A phase 2a clinical trial is performed to evaluate the efficacy of an antibody directed against amyloid beta (donanemab) in patients with prodromal Alzheimer's disease (AD), or in patients residing in an early (mild) stage of AD progression, or in patients determined via PET-based imaging of tau to reside within Braak stage I or Braak stage II of AD pathology. The patients are positive for the presence of a level of p-Tau 217 detected in a plasma sample from the patient, wherein the p-Tau 217 level is above a threshold level of p-Tau 217 determined from assaying reference samples taken from healthy individuals not suspected of having a neurogenerative disease in accordance with various embodiments herein. In this example, the level of p-Tau 217 is determined using a Simoa®-based pTau assay-217 with an anti-p-Tau 217 antibody described herein (e.g., Antibody 2). Alternatively, or in addition, patients are positive for the presence of a level of p-Tau 181 detected in a plasma sample from the patient, wherein the p-Tau 181 level is above a threshold level of p-Tau 181 determined from assaying reference samples taken from healthy individuals not suspected of having a neurogenerative disease. Optionally, the level of p-Tau 181 is determined using a Simoa®-based pTau assay-181 with an anti-p-Tau 181 antibody from Quanterix®. Donanemab is a humanized monoclonal antibody targeting N-truncated pyroglutamate amyloid-β peptide at position 3 (pGlu3-Ap, AppE3); with administration of donanemab clearing pGlu-Ap after formation and/or blocking aggregation, resulting in lowering Aβ plaques in the brain.

Screening Procedures considered for use in this example: Modified Hachinski Ischemic Scale (MHIS), Mini-Mental State Examination (MMSE), CogState Brief Battery, Columbia Suicide Severity Rating Scale - Adult Version, Screening Positron Emission Tomography and Magnetic Resonance Imaging, Screening Flortaucipir F18 PET Scan, Screening MRI, Screening Florbetapir F18 PET Scan.

Scoring the MMSE is handled in the same manner as in Example 8.

Screening Positron Emission Tomography (PET): If initial screening criteria are met, a patient may proceed with flortaucipir F18 PET scan, followed by MRI, and a florbetapir F18 PET scan procedure..

### Screening Flortaucipir F18 PET Scan:

A screening flortaucipir F18 PET scan is performed as part of the study eligibility criteria.

The flortaucipir F18 PET is performed and data is handled in the same manner as in Example 8.

### Screening MRI:

A local screening MRI is performed at Visit 1 as component of the study eligibility criteria. With the exception of the evidence of amyloid pathology by florbetapir F18 PET scan, a patient should meet all other Visit 1 eligibility criteria before having an MRI. The MRI scans are reviewed by the investigator or qualified designee for patient management. After the MRI scan is read locally, the scan is submitted to the centralized MRI vendor designated by the sponsor for final determination of MRI eligibility. Results of centrally read MRIs are used for data analysis and report-writing purposes and patient safety and eligibility is reported back to sites.

### Screening Florbetapir F18 PET Scan:

A screening florbetapir F18 PET scan is performed as part of the study eligibility criteria. A patient should meet all other Visit 1 eligibility criteria before having a screening florbetapir F18 PET scan. The florbetapir F18 PET scan is submitted to a centralized PET imaging vendor designated by the trial sponsor for assessment of patient's eligibility.

The florbetapir F18 PET screening is performed and data is handled in the same manner as in Example 8.

Inclusion criteria:

Patients are eligible for enrollment in the study if they meet all of the following criteria:

Type of patient and disease characteristics:

I) Gradual and progressive change in memory function (e.g., a metric of cognitive impairment) reported by patients or informants for a period of time>6 months prior to study enrollment.

II) An MMSE score of 20 to 28 (inclusive both scores at ends of range) at Visit 1 or an acceptable historical flortaucipir PET scan within 6 months prior to Visit 1 that meets the central read criteria.

III) Meet flortaucipir F18 scan (central read) criteria.

IV) Meet florbetapir F18 scan (central read) criteria.

### Patient Characteristics:

A) Men or women, 60 to 85 years of age, inclusive, at the time of informed consent given.

B) Have a study partner who will provide written informed consent to participate, is in frequent contact with the patient (defined as at least 10 hours per week), and will accompany the patient to study visits or be available by telephone. A second study partner may serve as backup.

C) Have adequate premorbid literacy, vision, and hearing for neuropsychological testing in the opinion of the investigator or designated scorer.

D) Have given written informed consent approved by the sponsor and the ethical review board (ERB) governing the site.

E) Are reliable and willing to make themselves easily available for the duration of the study and are also willing to follow study procedures.

### Exclusion Criteria:

F) Have a Modified Hachinski Ischemia Scale (MHIS; Hachinski et al. 1975) score of ≥4.

G) Lack adequate premorbid literacy, adequate vision, or adequate hearing to complete the required psychometric tests according to the investigator or designated scorer.

H) Significant neurological disease affecting the central nervous system (CNS), other than AD, that may affect cognition.

I) Current serious or unstable illnesses including cardiovascular, hepatic, renal, gastroenterological, respiratory, endocrinologic, neurologic (other than AD), psychiatric, immunologic, or hematologic disease and other conditions.

J) History of cancer within the last 5 years.

K) Patients with any current primary psychiatric diagnosis other than AD.

L) Have a history of long QT syndrome.

M) Are clinically judged by the investigator to be at a serious risk for suicide as assessed by medical history, in person examination, or the C—SSRS.

Arms: 40 patients per arm (antibody and placebo arms) are treated with antibody or placebo for a period of time up to 72 weeks. The treatment regimen comprises donanemab administration by intravenous (i.v.) infusion every 4 weeks for up to 72 weeks. The placebo group receives intravenously dosed placebo infusion every 4 weeks for up to 72 weeks. Antibody and placebo arms are double-blinded from Visit 2 onward until the end of study. End of the study is the date of the last visit or last scheduled procedure. Dosages of i.v. infusions of donanemab as monotherapy: 700 mg Q4WK for the first 3 doses, then 1400 mg Q4WK) for up to 72 weeks, in combination with daily oral placebo. Placebo group receives: intravenous placebo Q4WK for up to 72 weeks in combination with daily oral placebo.

An interim analysis is performed, for example, when all randomized subjects have had a chance to complete 52 weeks of treatment (Visit 15) and data are used to evaluated whether to stop the study. An interim analysis is performed in order to initiate the final population PK/PD model development processes for interim or final analyses. The interim analysis considers the level of p-Tau 217 detected in a plasma sample from the patient as a predicting factor in a later determination of positive diagnosis of AD or an evaluation of an extent of AD pathology progression. The level of p-Tau 217 detected in a plasma sample from the patient is considered as a biomarker for diagnosis in the patient. Optionally, a level of p-Tau 217 in the second plasma sample from the patient may be assessed during the study. This second level may be used as a proxy for treatment efficacy at the interim analysis period or at the end of the study. Maintenance or reduction in the level of p-Tau 217 in the subject following treatment is predicted to correlate with a favorable treatment outcome. A significant increase in the level of p-Tau 217 in the subject following treatment is predicted to correlate with a unfavorable treatment outcome.

### Primary Outcome Measures:

Electronic Clinical Outcome Assessment (eCOA) measures (e.g., a rating scale, including audio voice recordings of the rater's questions and the patient's and study partner's responses) are entered into an eCOA instrument (at the time that the information is obtained). The eCOA tablet has both keyboard entry and audio voice recording capabilities. Cognitive and functional testing are administered using an eCOA tablet. Cognitive and functional testing for each patient are to be performed at approximately the same time on each day that testing occurs to reduce potential variability. Note that the ADAS-Cog and MMSE are to be administered by a separate rater individual than the ADCS-ADL and CDR.

Integrated Alzheimer's Disease Rating Scale (iADRS) is used for the primary efficacy assessment according to Wessels A M, et al. in the same manner as in Example 8).Secondary Efficacy Assessments: Alzheimer's Disease Assessment Scale-Cognitive subscale (ADAS-Cog13), Alzheimer's Disease Cooperative Study-Activities of Daily Living Inventory (ADCS-ADL), Clinical Dementia Rating Scale (CDR-SB), Mini-Mental State Examination (MMSE), Biomarker Efficacy Measures (Double-Blind Period).For the Biomarker Efficacy Measures, the following biomarkers are tested: Florbetapir F18 PET scan, Flortaucipir F18 PET scan, Volumetric MRI, p-Tau 217 plasma level, and optionally, p-Tau 181 plasma level.

Primary objective: To test the hypothesis that donanemab alone administered for up to 72 weeks decreases cognitive and or functional decline in patients with early symptomatic AD and that initial testing for plasma levels of p-Tau 217 is an informative diagnostic biomarker for subjects that would otherwise go on to progress AD functional deficits and hence would benefit from early identification and thereby earlier therapeutic intervention.

Primary endpoint: Change in cognition and function as measured by the change in integrated Alzheimer's Disease Rating Scale (iADRS) score from baseline to 18 months. This change may be i) an improvement, ii) a similar score, or iii) a reduced score that less than a median reduction seen in the scores obtained from the placebo group. This indicates that treatment i) improves memory and/or cognition, ii) stabilizes a measurement of memory and/or cognition, or iii) significantly reduced worsening of memory and/or cognition.

Secondary objectives: To assess the effect of donanemab alone administered for up to 72 weeks vs. placebo on clinical progression in patients with early symptomatic AD and that initial testing for plasma levels of p-Tau 217 is an informative diagnostic biomarker for subjects that would otherwise go on to progress AD clinical pathology and hence would benefit from early identification and thereby earlier therapeutic intervention. In this sense, a specific and sensitive assay for p-Tau 217 in plasma can lead to earlier diagnosis of AD than by other criteria.

Secondary endpoints: Change in cognition from baseline to 18 months as measured by: change in ADAS-Cog13 score, change in CDR-SB score, change in MMSE score, change in ADCS-iADL score.

Other secondary endpoints: change in brain amyloid plaque disposition from baseline through 18 months as measured via florbetapir F18 PET scan., change in brain tau disposition from baseline through 18 months as measured via flortaucipir F18 PET scan, change in volumetric MRI measures from baseline to 18 months, change in p-Tau 217 plasma levels as measured by Simoa®-based pTau assay-217.

These secondary endpoints are to assess the effect of donanemab monotherapy administration vs. placebo in i) brain amyloid deposition, ii) brain tau deposition, iii) brain volume measurement changes, and iv) plasma p-Tau 217 levels.

### Example 10: Phase 2A Clinical Trial: Remternetug

A phase 2a clinical trial is performed to evaluate the efficacy of an antibody directed against amyloid beta (remtemetug) in patients with prodromal Alzheimer's disease (AD), or in patients residing in an early (mild) stage of AD progression, or in patients determined via PET-based imaging of tau to reside within Braak stage I or Braak stage II of AD pathology. Optionally, the patients are positive for the presence of a level of p-Tau 217 detected in a plasma sample from the patient, wherein the p-Tau 217 level is above a threshold level of p-Tau 217 determined from assaying reference samples taken from healthy individuals not suspected of having a neurogenerative disease. Optionally, the level of p-Tau 217 is determined using a Simoa®-based pTau assay-217 with an anti-p-Tau 217 antibody described herein (e.g., Antibody 2). Optionally, the patients are positive for the presence of a level of p-Tau 181 detected in a plasma sample from the patient, wherein the p-Tau 181 level is above a threshold level of p-Tau 181 determined from assaying reference samples taken from healthy individuals not suspected of having a neurogenerative disease. Optionally, the level of p-Tau 181 is determined using a Simoa®-based pTau assay-181 with an anti-p-Tau 181 antibody from Quanterix®. Remternetug is a humanized monoclonal IgG1-kappa antibody, (e.g., an anti-APP (amyloid beta A4 precursor protein) A042 N3pGlu peptide monoclonal antibody); with administration targeting pyroglutamate amyloid-β peptide that aggregates into amyloid plaques resulting in lowering Aβ plaques in the brain.

Screening Procedures considered for use in this example: Modified Hachinski Ischemic Scale (MHIS), Mini-Mental State Examination (MMSE), CogState Brief Battery, Columbia Suicide Severity Rating Scale - Adult Version, Screening Positron Emission Tomography and Magnetic Resonance Imaging, Screening Flortaucipir F18 PET Scan, Screening MRI, Screening Florbetapir F18 PET Scan.

Scoring the MMSE is handled in the same manner as in Example 8.

Screening Positron Emission Tomography (PET): If initial screening criteria are met, a patient may proceed with flortaucipir F18 PET scan, followed by MRI, and a florbetapir F18 PET scan procedure.

### Screening Flortaucipir F18 PET Scan:

A screening flortaucipir F18 PET scan is performed as part of the study eligibility criteria.

The flortaucipir F18 PET is performed and data is handled in the same manner as in Example 8.

### Screening MRI:

A local screening MRI is performed at Visit 1 as component of the study eligibility criteria. With the exception of the evidence of amyloid pathology by florbetapir F18 PET scan, a patient should meet all other Visit 1 eligibility criteria before having an MRI. The MRI scans are reviewed by the investigator or qualified designee for patient management. After the MRI scan is read locally, the scan is submitted to the centralized MRI vendor designated by the sponsor for final determination of MRI eligibility. Results of centrally read MRIs are used for data analysis and report-writing purposes and patient safety and eligibility is reported back to sites.

### Screening Florbetapir F18 PET Scan:

A screening florbetapir F18 PET scan is performed as part of the study eligibility criteria. A patient should meet all other Visit 1 eligibility criteria before having a screening florbetapir F18 PET scan. The florbetapir F18 PET scan is submitted to a centralized PET imaging vendor designated by the trial sponsor for assessment of patient's eligibility.

The florbetapir F18 PET screening is performed and data is handled in the same manner as in Example 8.

Inclusion criteria:

Patients are eligible for enrollment in the study if they meet all of the following criteria:

Type of patient and disease characteristics:

I) Gradual and progressive change in memory function (e.g., a metric of cognitive impairment) reported by patients or informants for a period of time>6 months prior to study enrollment.

II) An MMSE score of 20 to 28 (inclusive both scores at ends of range) at Visit 1 or an acceptable historical flortaucipir PET scan within 6 months prior to Visit 1 that meets the central read criteria.

III) Meet flortaucipir F18 scan (central read) criteria.

IV) Meet florbetapir F18 scan (central read) criteria.

### Patient Characteristics:

A) Men or women, 60 to 85 years of age, inclusive, at the time of informed consent given.

B) Have a study partner who will provide written informed consent to participate, is in frequent contact with the patient (defined as at least 10 hours per week), and will accompany the patient to study visits or be available by telephone. A second study partner may serve as backup.

C) Have adequate premorbid literacy, vision, and hearing for neuropsychological testing in the opinion of the investigator or designated scorer.

D) Have given written informed consent approved by the sponsor and the ethical review board (ERB) governing the site.

E) Are reliable and willing to make themselves easily available for the duration of the study and are also willing to follow study procedures.

### Exclusion Criteria:

F) Have a Modified Hachinski Ischemia Scale (MHIS; Hachinski et al. 1975) score of >4.

G) Lack adequate premorbid literacy, adequate vision, or adequate hearing to complete the required psychometric tests according to the investigator or designated scorer.

H) Significant neurological disease affecting the central nervous system (CNS), other than AD, that may affect cognition.

I) Current serious or unstable illnesses including cardiovascular, hepatic, renal, gastroenterological, respiratory, endocrinologic, neurologic (other than AD), psychiatric, immunologic, or hematologic disease and other conditions.

J) History of cancer within the last 5 years.

K) Patients with any current primary psychiatric diagnosis other than AD.

L) Have a history of long QT syndrome.

M) Are clinically judged by the investigator to be at a serious risk for suicide as assessed by medical history, in person examination, or the C—SSRS.

Arms: 40 patients per arm (antibody and placebo arms) are treated with antibody or placebo for a period of time up to 72 weeks. The treatment regimen comprises remtemetug administration by intravenous (i.v.) infusion every 4 weeks for up to 72 weeks. The placebo group receives intravenously dosed placebo infusion every 4 weeks for up to 72 weeks. Antibody and placebo arms are double-blinded from Visit 2 onward until the end of study. End of the study is the date of the last visit or last scheduled procedure. Dosages of i.v. infusions of remtemetug as monotherapy: 700 mg Q4WK for the first 3 doses, then 1400 mg Q4WK) for up to 72 weeks, in combination with daily oral placebo. Placebo group receives: intravenous placebo Q4WK for up to 72 weeks in combination with daily oral placebo.

An interim analysis is performed, for example, when all randomized subjects have had a chance to complete 52 weeks of treatment (Visit 15) and data are used to evaluated whether to stop the study. An interim analysis is performed in order to initiate the final population PK/PD model development processes for interim or final analyses. The interim analysis considers the level of p-Tau 217 detected in a plasma sample from the patient as a predicting factor in a later determination of positive diagnosis of AD or an evaluation of an extent of AD pathology progression. The level of p-Tau 217 detected in a plasma sample from the patient is considered as a biomarker for diagnosis in the patient. Optionally, a level of p-Tau 217 in the second plasma sample from the patient may be assessed during the study. This second level may be used as a proxy for treatment efficacy at the interim analysis period or at the end of the study. Maintenance or reduction in the level of p-Tau 217 in the subject following treatment is predicted to correlate with a favorable treatment outcome. A significant increase in the level of p-Tau 217 in the subject following treatment is predicted to correlate with a unfavorable treatment outcome.

Primary Outcome Measures:
Electronic Clinical Outcome Assessment (eCOA) measures (e.g., a rating scale, including audio voice recordings of the rater's questions and the patient's and study partner's responses) are entered into an eCOA instrument (at the time that the information is obtained). The eCOA tablet has both keyboard entry and audio voice recording capabilities. Cognitive and functional testing are administered using an eCOA tablet. Cognitive and functional testing for each patient are to be performed at approximately the same time on each day that testing occurs to reduce potential variability. Note that the ADAS-Cog and MMSE are to be administered by a separate rater individual than the ADCS-ADL and CDR.

Integrated Alzheimer's Disease Rating Scale (iADRS) is used for the primary efficacy assessment according to Wessels A M, et al. in the same manner as in Example 8.

Secondary Efficacy Assessments: Alzheimer's Disease Assessment Scale-Cognitive subscale (ADAS-Cog13), Alzheimer's Disease Cooperative Study-Activities of Daily Living Inventory (ADCS-ADL), Clinical Dementia Rating Scale (CDR-SB), Mini-Mental State Examination (MMSE), Biomarker Efficacy Measures (Double-Blind Period).

For the Biomarker Efficacy Measures, the following biomarkers are tested: Florbetapir F18 PET scan, Flortaucipir F18 PET scan, Volumetric MRI, p-Tau 217 plasma level, and optionally, p-Tau 181 plasma level.

Primary objective: To test the hypothesis that remternetug alone administered for up to 72 weeks decreases cognitive and or functional decline in patients with early symptomatic AD and that initial testing for plasma levels of p-Tau 217 is an informative diagnostic biomarker for subjects that would otherwise go on to progress AD functional deficits and hence would benefit from early identification and thereby earlier therapeutic intervention.

Primary endpoint: Change in cognition and function as measured by the change in integrated Alzheimer's Disease Rating Scale (iADRS) score from baseline to 18 months. This change may be i) an improvement, ii) a similar score, or iii) a reduced score that less than a median reduction seen in the scores obtained from the placebo group. This indicates that treatment i) improves memory and/or cognition, ii) stabilizes a measurement of memory and/or cognition, or iii) significantly reduced worsening of memory and/or cognition.

Secondary objectives: To assess the effect of remternetug alone administered for up to 72 weeks vs. placebo on clinical progression in patients with early symptomatic AD and that initial testing for plasma levels of p-Tau 217 is an informative diagnostic biomarker for subjects that would otherwise go on to progress AD clinical pathology and hence would benefit from early identification and thereby earlier therapeutic intervention. In this sense, a specific and sensitive assay for p-Tau 217 in plasma can lead to earlier diagnosis of AD than by other criteria.

Secondary endpoints: Change in cognition from baseline to 18 months as measured by: change in ADAS-Cog13 score, change in CDR-SB score, change in MMSE score, change in ADCS-iADL score.

Other secondary endpoints: change in brain amyloid plaque disposition from baseline through 18 months as measured via florbetapir F18 PET scan., change in brain tau disposition from baseline through 18 months as measured via flortaucipir F18 PET scan, change in volumetric MRI measures from baseline to 18 months, change in p-Tau 217 plasma levels as measured by Simoa®-based pTau assay-217.

These secondary endpoints are to assess the effect of remtemetug monotherapy administration vs. placebo in i) brain amyloid deposition, ii) brain tau deposition, iii) brain volume measurement changes, and iv) plasma p-Tau 217 levels.

### Example 11. Selecting a Subject for Treatment with Donanemab or Lecanemab

A patient presents with symptoms including cognitive decline, memory loss, difficulty completing routine tasks, and confusion with time and place. The patient's physician suspects that the patient has Alzheimer's disease. The physician obtains a blood sample (venous draw) from the patient and analyzes the patient's sera to measure p-Tau 217 levels in the sera compared with a standard value of p-Tau 217 level derived from a plurality of reference samples from reference subjects that do not have Alzheimer's disease or another type of tauopathy. The results come back positive for abnormally high levels of p-Tau 217, which is indicative of abnormal deposition of phosphorylated tau protein or tau tangles in the brain of the patient. The physician starts the patient on Donanemab or Lecanemab for the treatment of the Alzheimer's disease based on these results. While this example pertains to the treatment of Alzheimer's disease with Donanemab or Lecanemab, it may also be applied to other tauopathies of the present disclosures and therapeutic strategies including inhibiting phosphorylated tau protein (e.g., anti-pTau217 antibody or antigen binding fragment thereof).

### Example 12. Monitoring a Treatment for Alzheimer's Disease

The patient in Example 11 visits his physician for a checkup. To monitor the effectiveness of the Donanemab or Lecanemab in slowing or preventing disease progression of the Alzheimer's disease, the physician obtains a blood sample (venous draw) from the patient and analyzes the patient's sera to measure p-Tau 217 levels in the sera compared with a standard value of p-Tau 217 level derived from a plurality of reference samples from reference subjects that do not have Alzheimer's disease or another type of tauopathy; and compared with the level of p-Tau 217 measured in Example 11. The results come back positive for abnormally high levels of p-Tau 217; however, the level is lower than the level measured in Example 11. This evidences that the patient's disease progression is slowing but is still present. The physician instructs the patient to continue his treatment regimen with the Donanemab or Lecanemab treatment, based on these results. While this example pertains to the monitoring of the treatment of Alzheimer's disease with Donanemab or Lecanemab, it may also be applied to other tauopathies of the present disclosures and therapeutic strategies including inhibiting phosphorylated tau protein (e.g., anti-pTau217 antibody or antigen binding fragment thereof).

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A method of treating a neurodegenerative disease in a subject, wherein the neurodegenerative disease is characterized by accumulation of amyloid-O (AD) peptide in a brain of the subject, the method comprising: administering to the subject a therapeutic agent comprising (i) an inhibitor of the Aβ peptide or a modified form of the Aβ peptide or (ii) an inhibitor of a phosphorylated tau protein, for treatment of the neurodegenerative disease, wherein the subject is identified for the treatment based, at least in part, on a level of phosphorylated tau 217 (p-Tau 217) measured in a sample obtained from the subject that is above the level of the p-Tau 217 measured in the sample with a standard value of p-Tau 217 level derived from a plurality of reference samples from reference subjects that do not have the neurodegenerative disease, which is predictive that the subject has deposition of abnormal tau protein in the brain with a sensitivity that is greater than or equal to about 85%.

2. The method of claim 1, wherein the p-Tau 217 is measured in the sample by a method comprising performing an immunoassay on the sample using an anti-tau antibody or antigen-binding fragment thereof.

3. The method of claim 2, wherein the immunoassay is a digital immunoassay configured to measure the p-Tau 217 in a sample that is a fluid sample.

4. The method of claim 3, wherein the digital immunoassay is a Single Molecule Array (SIMOA).

5. The method of claim 2, wherein the immunoassay comprises an enzyme-linked immunoassay (ELISA), a radioimmunoassay (RIA), a fluoroimmunoassay (FIA), a chemiluminescent immunoassay (CLIA), or a counting immunoassay (CIA).

6. The method of claim 2, wherein the anti-tau antibody or the antigen-binding fragment thereof comprises:
a. a heavy chain comprising a variable heavy chain (VH) domain, wherein the VH domain comprises an HCDR1 sequence selected from SEQ ID NOs: 1-9, an HCDR2 sequence selected from SEQ ID NOs: 10-17, and an HCDR3 sequence selected from SEQ ID NOs: 18-23; and
b. a light chain comprising a variable light chain (VL) domain, wherein the VL domain comprises an LCDR1 sequence selected from SEQ ID NOs: 24-31, an LCDR2 sequence selected from SEQ ID NOs: 32-36, and an LCDR3 sequence selected from SEQ ID NOs: 37-43.

7. The method of claim 6, wherein:
a. the HCDR1 sequence comprises SEQ ID NO: 2, the HCDR2 sequence comprises SEQ ID NO: 11, and the HCDR3 sequence comprises SEQ ID NO: 19; and
b. the LCDR1 sequence comprises SEQ ID NO: 25, the LCDR2 sequence comprises SEQ ID NO: 33, and the LCDR3 sequence comprises SEQ ID NO: 38.

8. The method of claim 6, wherein:
a. the HCDR1 sequence comprises SEQ ID NO: 1, the HCDR2 sequence comprises SEQ ID NO: 10, and the HCDR3 sequence comprises SEQ ID NO: 18; and
b. the LCDR1 sequence comprises SEQ ID NO: 24, the LCDR2 sequence comprises SEQ ID NO: 32, and the LCDR3 sequence comprises SEQ ID NO: 37.

9. The method of claim 6, wherein:
a. the HCDR1 sequence comprises SEQ ID NO: 2, the HCDR2 sequence comprises SEQ ID NO: 11, and the HCDR3 sequence comprises SEQ ID NO: 19; and
b. the LCDR1 sequence comprises SEQ ID NO: 26, the LCDR2 sequence comprises SEQ ID NO: 34, and the LCDR3 sequence comprises SEQ ID NO: 39.

10. The method of claim 6, wherein:
a. the HCDR1 sequence comprises SEQ ID NO: 3, the HCDR2 sequence comprises SEQ ID NO: 12, and the HCDR3 sequence comprises SEQ ID NO: 18; and
b. the LCDR1 sequence comprises SEQ ID NO: 27, the LCDR2 sequence comprises SEQ ID NO: 32, and the LCDR3 sequence comprises SEQ ID NO: 40.

11. The method of claim 6, wherein:
a. the HCDR1 sequence comprises SEQ ID NO: 4, the HCDR2 sequence comprises SEQ ID NO: 11, and the HCDR3 sequence comprises SEQ ID NO: 20; and
b. the LCDR1 sequence comprises SEQ ID NO: 28, the LCDR2 sequence comprises SEQ ID NO: 35, and the LCDR3 sequence comprises SEQ ID NO: 41.

12. 7. The method of claim 6, wherein:
a. the HCDR1 sequence comprises SEQ ID NO: 5, the HCDR2 sequence comprises SEQ ID NO: 13, the HCDR3 sequence comprises SEQ ID NO: 21; and
b. the LCDR1 sequence comprises SEQ ID NO: 29, the LCDR2 sequence comprises SEQ ID NO: 33, and the LCDR3 sequence comprises SEQ ID NO: 42.

13. The method of claim 1, wherein the anti-tau antibody comprises a heavy chain (HC) sequence comprising SEQ ID NO: 57 and a light chain (LC) sequence comprising SEQ ID NO: 58.

14. The method of claim 1, wherein the anti-tau antibody comprises a HC sequence comprising SEQ ID NO: 57 and a LC sequence comprising SEQ ID NO: 59.

15. The method of claim 1, wherein the anti-tau antibody comprises a HC sequence comprising SEQ ID NO: 60 and a LC sequence comprising SEQ ID NO: 61.

16. The method of claim 1, wherein the anti-tau antibody comprises a HC sequence comprising SEQ ID NO: 62 and a LC sequence comprising SEQ ID NO: 63.

17. The method of claim 1, wherein the anti-tau antibody comprises a HC sequence comprising SEQ ID NO: 64 and a LC sequence comprising SEQ ID NO: 65.

18. The method of claim 1, wherein the anti-tau antibody comprises a HC sequence comprising SEQ ID NO: 55 and a LC sequence comprising SEQ ID NO: 56.

19. The method of claim 1, wherein the anti-tau antibody comprises a variable heavy (VH) domain comprising a sequence that has at least 80%, at least 85%, at least 90%, or at least 95% sequence identity to a sequence selected from SEQ ID NOs: 44-48.

20. The method of claim 1, wherein the anti-tau antibody comprises a variable light (VL) domain comprising a sequence that has at least 80%, at least 85%, at least 90%, or at least 95% sequence identity to a sequence selected from SEQ ID NOs: 49-54.

21. The method of claim 1, wherein the anti-tau antibody or antigen-binding fragment thereof comprises an IgG-scFv, nanobody, BiTE, diabody, DART, TandAb, scDiabody, scDiabody-CH3, triple body, mini-antibody, minibody, TriBi minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, tetravalent HCAb, scDiabody-Fc, diabody-Fc, tandem scFv-Fc, or intrabody.

22. The method of claim 1, wherein the anti-tau antibody has an isotype selected from the group consisting of IgA1, IgA2, IgD, IgE, IgG1, IgG2, IgG3, IgG4, and IgM.

23. The method of claim 1, wherein the anti-tau antibody or antigen-binding fragment thereof specifically binds an epitope in an N-terminal region of a tau polypeptide.

24. The method of claim 1, wherein the anti-tau antibody or antigen-binding fragment thereof specifically binds to p-Tau 217-tau protein.

25. The method of claim 1, wherein the anti-tau antibody or antigen-binding fragment thereof specifically binds to a cis-conformation of p-Tau 217-tau protein.

26. The method of claim 1, wherein the anti-tau antibody or antigen-binding fragment thereof binds specifically to a tau polypeptide comprising phosphorylation at a site selected from a group consisting of pT181-tau, pT212-tau, phosphorylated-serine (pS)214-tau, pT217-tau, pT220-tau, and pT231-tau.

27. The method of claim 1, wherein the anti-tau antibody or antigen-binding fragment thereof binds specifically to a tau polypeptide comprising phosphorylation at site pT217-tau.

28. The method of claim 1, wherein the anti-tau antibody or antigen-binding fragment thereof binds specifically to a tau polypeptide comprising phosphorylation at site pT217-tau and pT231-tau.

29. The method of claim 1, wherein the subject has, or is suspected of having, Alzheimer's disease (AD).

30. The method of claim 29, wherein the AD causes, is associated with, or presents with a tauopathy in the subject.

31. The method of claim 30, wherein the tauopathy comprises a secondary tauopathy comprising neurofibrillary tangle (NTF) pathology in the brain of the subject, wherein the tauopathy is secondary to amyloid-beta plaques in the brain of the subject.

32. The method of claim 30, wherein AD comprises a variant AD selected from the group consisting of early-onset Alzheimer's disease, late-onset Alzheimer's disease, Familial Alzheimer's disease (FAD), a mixed dementia comprising Alzheimer's disease and vascular dementia, logopenic aphasia, posterior cortical atrophy, frontal variant Alzheimer's disease, and Alzheimer's disease combined with corticobasal syndrome (AD-CBS).

33. The method of claim 30, wherein the tauopathy comprises hyperphosphorylated tau, misfolded tau, oligomeric tau, aggregated paired helical filaments (PFHs) of tau, neurofibrillary tangles (NFTs), or any combination thereof.

34. The method of claim 1, wherein the subject is a human is age 60 or older.

35. The method of claim 1, wherein the neurodegenerative disease comprises Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, Hallevorden-Spatz disease, mild cognitive impairment (MCI), Lewy Body's disease (LBD), Amyotrophic lateral sclerosis, spinal muscular atrophy, Friedreich's ataxia, Wernicke-Korsakoff syndrome, a prion disease, vascular dementia, Alzheimer's related dementia, Huntington's disease (HD), or a transgene-induced tauopathy.

36. The method of claim 1, wherein the neurodegenerative disease is Alzheimer's disease.

37. The method of claim 36, wherein a prodromal stage of the Alzheimer's disease is characterized by mild cognitive impairment (MCI).

38. The method of claim 36, wherein a prodromal stage of Alzheimer's disease is characterized at least as Braak stage I by a determined spatial extent of tau-PET.

39. The method of claim 36, wherein a prodromal stage of Alzheimer's disease is characterized at least as Braak stage II by a determined spatial extent of tau-PET.

40. The method of claim 1, wherein the neurodegenerative disease is a tauopathy comprising hyperphosphorylated tau, misfolded tau, oligomeric tau, aggregated paired helical filaments (PFHs) of tau, neurofibrillary tangles (NFTs), or any combination thereof.

41. The method of claim 1, wherein the inhibitor of the Aβ peptide or the modified form of the Aβ peptide comprises: ABvac40, ABBV-916, ACU193, AD-35, Aducanumab (Aduhelm®), APH-1105, BPN14770, Bapineuzumab, BMS-984923, Contraloid acetate, CNP520(AMG520), Crenezumab, Donanemab (LY3002813), Donepezil (Aricept), Elenbecestat (E2609), Gantenerumab, Brain Shuttle Gantenerumab (R07126209), GV-971, HT-ALZ, KHK 66401, Lanabecestat, Lecanemab (BAN2401) (Legembi®), Lu AF20513, MEDI 1814, Ponezumab, Remternetug (LY3372993), rivastigmine, SHR-1707, Simufilam (PTI-125), Sodium oligomannate, Solanezumab, scyllo-inositol, UB-311, valiltramiprosate (ALZ-801), varoglutamstat (PQ912), verubecestat (MK-8931), VGH-AD1, an antisense RNA directed to an isoform of human amyloid beta, an siRNA directed to an isoform of human amyloid beta, an antisense oligonucleotide directed to an isoform of human amyloid beta, an LNA oligonucleotide directed to an isoform of human amyloid beta, a CRISPRn-based therapeutic targeting the human APP locus, or a CRISPRi-based therapeutic targeting the human APP locus.

42. The method of claim 1, wherein the inhibitor of the Aβ peptide or the modified form of the Aβ peptide comprises Donanemab.

43. The method of claim 1, wherein the inhibitor of the Aβ peptide or the modified form of the Aβ peptide comprises Lecanemab.

44. The method of claim 1, wherein the inhibitor of the Aβ peptide or the modified form of the Aβ peptide comprises Remtemetug.

45. The method of claim 1, wherein the inhibitor of the Aβ peptide or the modified form of the Aβ peptide comprises Aducanumab.

46. The method of claim 1, wherein the inhibitor of the Aβ peptide or the modified form of the Aβ peptide is selected from Table 10.

47. The method of claim 1, wherein the inhibitor of the phosphorylated tau protein comprises an inhibitor of p-Tau 217 or a modified form of p-Tau 217, p-Tau 181, p-Tau 212, p-Tau 220, p-Tau 231, or phosphorylated-serine (pS)214-tau.

48. The method of claim 47, wherein the inhibitor of the phosphorylated tau protein comprises an inhibitor of p-Tau 217.

49. The method of claim 1, wherein the sensitivity is greater than or equal to about 90%.

50. The method of claim 1, wherein the sensitivity is greater than or equal to about 92%.

51. The method of claim 1, wherein the level of p-Tau 217 that is measured is predictive that the subject has the deposition of the abnormal tau protein in the brain with an area under the curve (AUC) that is greater than or equal to about 0.85.

52. The method of claim 51, wherein the AUC is greater than or equal to about 0.90.

53. The method of claim 1, wherein the level of p-Tau 217 that is measured is predictive that the subject has the deposition of the abnormal tau protein in the brain with a specificity that is greater than or equal to about 81%

54. The method of claim 53, wherein the specificity is greater than or equal to about 85%.

55. The method of claim 1, wherein the level of p-Tau 217 that is measured is predictive that the subject has the deposition of the abnormal tau protein in the brain with a positive predictive value (PPV) that is greater than or equal to about 51%.

56. The method of claim 55, wherein the PPV is greater than or equal to about 70%.

57. The method of claim 1, wherein the level of p-Tau 217 that is measured is predictive that the subject has the deposition of the abnormal tau protein in the brain with a negative predictive value (NPV) that is greater than or equal to about 51%.

58. The method of claim 57, wherein the NPV is greater than or equal to about 70%.

59. The method of claim 1, wherein the level of p-Tau 217 that is measured is predictive that the subject will exhibit a positive therapeutic response to the therapeutic agent with a specificity that is greater than or equal to about 70%.

60. The method of claim 1, wherein the level of p-Tau 217 that is measured is predictive that the subject will exhibit a positive therapeutic response to the therapeutic agent with a sensitivity that is greater than or equal to about 70%.

61. The method of claim 1, wherein the level of p-Tau 217 that is measured is predictive that the subject will exhibit a positive therapeutic response to the therapeutic agent with a PPV that is greater than or equal to about 70%.

62. The method of claim 1, wherein the level of p-Tau 217 that is measured is predictive that the subject will exhibit a positive therapeutic response to the therapeutic agent with a NPV that is greater than or equal to about 70%.

63. The method of claim 1, wherein the level of p-Tau 217 that is measured is predictive that the subject will exhibit a positive therapeutic response to the therapeutic agent with an AUC that is greater than or equal to about 0.70.

64. The method of claim 1, wherein the sample is a cerebrospinal fluid (CSF) sample, a plasma sample, a blood sample, or a serum sample.

65. The method of claim 1, wherein the sample is a plasma sample.

66. The method of claim 1, further comprising determining a likelihood of clinical diagnosis of the neurodegenerative disease in the subject based, at least in part, on the level of p-Tau 217 measured in the sample.

67. The method of claim 1, further comprising establishing an early prognosis of the neurodegenerative disease in the subject based, at least in part, on the level of p-Tau 217 measured in the sample, wherein the early prognosis predates providing a diagnosis of a prodromal stage of the neurodegenerative disease in the subject.

68. The method of claim 67, wherein the neurodegenerative disease is Alzheimer's disease, and wherein establishing the early prognosis of the Alzheimer's disease comprises establishing a likelihood of the Alzheimer's disease progressing to Alzheimer's disease dementia in the subject based, at least in part, on the level of p-Tau 217 measured in the sample.

69. The method of claim 1, further comprising identifying an elevated risk that the neurodegenerative disease will lead to pathological memory impairment and cognitive decline in the subject, based at least in part, on the level of p-Tau 217 measured in the sample.

70. The method of claim 1, further comprising ameliorating one or more symptoms of the neurodegenerative disease in the subject.

71. The method of claim 1, further comprising slowing a progression of one or more symptoms of the neurodegenerative disease in the subject.

72. The method of claim 1, wherein the therapeutic agent is administered intravenously, intramuscularly, intrathecally, intracerebrally, subcutaneously, orally, nasally, topically, buccally, or sublingually.

73. The method of claim 1, wherein the therapeutic agent is administered directly to the CNS of the subject via intravenous delivery, intravascular delivery, intrathecal delivery, intracisternal delivery, intraspinal delivery, subpial delivery, or intracerebroventricular delivery.

74. The method of claim 1, wherein the subject is at a preclinical stage of the neurogenerative disease.

75. The method of claim 1, wherein the subject is at a prodromal stage of the neurogenerative disease.

76. The method of claim 1, wherein the therapeutic agent is administered according to a body weight of the subject at a dosage of between about 0.1-100 mg/kg of the body weight.

77. The method of claim 1, wherein the therapeutic agent is administered to the subject at a dosage of between about 200-1500 mg by intravenous administration.

78. The method of claim 1, wherein the therapeutic agent is administered to the subject at a dosage of between about 700-1400 mg by intravenous administration.

79. The method of claim 1, wherein the therapeutic agent is administered according to a body weight of the subject at a dosage of about 10 mg/kg of the body weight.

80. The method of claim 1, wherein the therapeutic agent is administered to the subject according to a treatment regimen comprising administering the therapeutic agent about once every 2 weeks (q2 week).

81. The method of claim 1, wherein the therapeutic agent is administered to the subject according to a treatment regimen comprising administering the therapeutic agent about once every 4 weeks (q4 week).

82. The method of claim 1, wherein the therapeutic agent is administered to the subject is a plurality of doses, wherein a first three doses of the plurality of doses is administered to the subject at a dosage amount comprising about 700 mg per dose.

83. The method of claim 82, wherein the doses of the plurality of doses administered to the subject after the first three doses are at a dosage amount comprising about 1400 mg per dose.

84. The method of claim 1, wherein the administering is systemic administration.

85. The method of claim 84, wherein systemic administration comprises intravenous administration.

86. The method of claim 84, wherein systemic administration comprises subcutaneous administration.

87. The method of claim 84, wherein systemic administration comprises oral administration.

88. The method of claim 1, further comprising:
a. repeating the method following induction of the therapeutic agent, whereby a second level of p-Tau 217 is measured in a second sample obtained from the subject following induction of the therapeutic agent, and wherein the second level of the p-Tau 217 is lower than the level of p-Tau 217 measured prior to induction of the therapeutic agent; and
b. administering another dose of the therapeutic agent to the subject.

89. A kit for selecting a subject for treatment of a neurodegenerative disease, the kit comprising:
a. an anti-tau antibody or the antigen-binding fragment thereof, comprising:
i. a heavy chain comprising a variable heavy chain (VH) domain, wherein the VH domain comprises an HCDR1 sequence selected from SEQ ID NOs: 1-9, an HCDR2 sequence selected from SEQ ID NOs: 10-17, and an HCDR3 sequence selected from SEQ ID NOs: 18-23; and a light chain comprising a variable light chain (VL) domain, wherein the VL domain comprises an LCDR1 sequence selected from SEQ ID NOs: 24-31, an LCDR2 sequence selected from SEQ ID NOs: 32-36, and an LCDR3 sequence selected from SEQ ID NOs: 37-43;
ii. a heavy chain (HC) sequence comprising any one of SEQ ID NOs: 57, 60, 62, 64, or 55; and a light chain (LC) sequence comprising any one of SEQ ID NOs: 58, 59, 63, 65, or 56; or
iii. a variable heavy (VH) domain comprising a sequence that has at least 80%, at least 85%, at least 90%, at least 95% sequence identity to a sequence selected from SEQ ID NOs: 44-48; and a variable light (VL) domain comprising a sequence that has at least 80%, at least 85%, at least 90%, at least 95% sequence identity to a sequence selected from SEQ ID NOs: 49-54;
b. instructions for analyzing a sample obtained from the subject using the anti-tau antibody or antigen-binding fragment thereof in an immunoassay;
c. instructions for treating the neurodegenerative disease in the subject with a therapeutic agent comprising (i) an inhibitor of the Aβ peptide or a modified form of the Aβ peptide or (ii) an inhibitor of a phosphorylated tau protein; and
d. optionally, the therapeutic agent.

90. The kit of claim 89, wherein the kit comprises the therapeutic agent.

91. The kit of claim 89, further comprising one or more immunoassay components, wherein the immunoassay comprises Single Molecule Array (SIMOA), an enzyme-linked immunoassay (ELISA), a radioimmunoassay (RIA), a fluoroimmunoassay (FIA), a chemiluminescent immunoassay (CLIA), or a counting immunoassay (CIA).

92. The kit of claim 89, further comprising a system for performing the immunoassay, wherein the immunoassay comprises Single Molecule Array (SIMOA), an enzyme-linked immunoassay (ELISA), a radioimmunoassay (RIA), a fluoroimmunoassay (FIA), a chemiluminescent immunoassay (CLIA), or a counting immunoassay (CIA).

93. The kit of claim 89, wherein the anti-tau antibody or the antigen-binding fragment thereof comprises a heavy chain comprising a variable heavy chain (VH) domain, wherein the VH domain comprises a HCDR1 sequence selected from SEQ ID NOs: 1-9, a HCDR2 sequence selected from SEQ ID NOs: 10-17, and a HCDR3 sequence selected from SEQ ID NOs: 18-23; and a light chain comprising a variable light chain (VL) domain, wherein the VL domain comprises a LCDR1 sequence selected from SEQ ID NOs: 24-31, a LCDR2 sequence selected from SEQ ID NOs: 32-36, and a LCDR3 sequence selected from SEQ ID NOs: 37-43.

94. The kit of claim 89, wherein the anti-tau antibody or the antigen-binding fragment thereof comprises a heavy chain (HC) sequence comprising any one of SEQ ID NOs: 57, 60, 62, 64, or 55; and a light chain (LC) sequence comprising any one of SEQ ID NOs: 58, 59, 63, 65, or 56.

95. The kit of claim 89, wherein the anti-tau antibody or the antigen-binding fragment thereof comprises a variable heavy (VH) domain comprising a sequence that has at least 80%, at least 85%, at least 90%, at least 95% sequence identity to a sequence selected from SEQ ID NOs: 44-48; and a variable light (VL) domain comprising a sequence that has at least 80%, at least 85%, at least 90%, at least 95% sequence identity to a sequence selected from SEQ ID NOs: 49-54.

96. The kit of claim 90, wherein the inhibitor of the therapeutic agent comprises: ABvac40, ABBV-916, ACU193, AD-35, Aducanumab (Aduhelm®), APH-1105, BPN14770, Bapineuzumab, BMS-984923, Contraloid acetate, CNP520(AMG520), Crenezumab, Donanemab (LY3002813), Donepezil (Aricept), Elenbecestat (E2609), Gantenerumab, Brain Shuttle Gantenerumab (R07126209), GV-971, HT-ALZ, KHK 66401, Lanabecestat, Lecanemab (BAN2401) (Legembi®), Lu AF20513, MEDI 1814, Ponezumab, Remtemetug (LY3372993), rivastigmine, SHR-1707, Simufilam (PTI-125), Sodium oligomannate, Solanezumab, scyllo-inositol, UB-311, valiltramiprosate (ALZ-801), varoglutamstat (PQ912), verubecestat (MK-8931), VGH-AD1, an antisense RNA directed to an isoform of human amyloid beta, an siRNA directed to an isoform of human amyloid beta, an antisense oligonucleotide directed to an isoform of human amyloid beta, an LNA oligonucleotide directed to an isoform of human amyloid beta, a CRISPRn-based therapeutic targeting the human APP locus, or a CRISPRi-based therapeutic targeting the human APP locus.

97. The kit of claim 90, wherein the therapeutic agent comprises Donanemab.

98. The kit of claim 90, wherein the therapeutic agent comprises Lecanemab.

99. The kit of claim 90, wherein the therapeutic agent comprises Remtemetug.

100. The kit of claim 90, wherein the therapeutic agent comprises Aducanumab.

101. The kit of claim 90, wherein the therapeutic agent is selected from Table 10.

102. The kit of claim 90, wherein the therapeutic agent is the inhibitor of the phosphorylated tau protein.

103. The kit of claim 102, wherein the inhibitor of the phosphorylated tau protein is an inhibitor of p-Tau 217 or a modified form of p-Tau 217, p-Tau 181, p-Tau 212, p-Tau 220, p-Tau 231, or phosphorylated-serine (pS)214-tau.

104. The kit of claim 103, wherein the inhibitor of the phosphorylated tau protein comprises an inhibitor of p-Tau 217.

105. The kit of claim 89, wherein the instructions for analyzing a sample further comprise a standard value of p-Tau 217 level derived from a plurality of reference samples from reference subjects that do not have the neurodegenerative disease.


SEQUENCE LISTING (95 sequences)

SEQ ID NO: 1 - protein, 5 aa, synthetic construct
SQKVG

SEQ ID NO: 2 - protein, 5 aa, synthetic construct
SYAMI

SEQ ID NO: 3 - protein, 5 aa, synthetic construct
NYKVG

SEQ ID NO: 4 - protein, 5 aa, synthetic construct
NYAMS

SEQ ID NO: 5 - protein, 5 aa, synthetic construct
THAMT

SEQ ID NO: 6 - protein, 8 aa, synthetic construct
GFSLSSYA

SEQ ID NO: 7 - protein, 10 aa, synthetic construct
GFSLSSYAMI

SEQ ID NO: 8 - protein, 7 aa, synthetic construct
GFSLSSY

SEQ ID NO: 9 - protein, 6 aa, synthetic construct
SSYAMI

SEQ ID NO: 10 - protein, 16 aa, synthetic construct
IINNYGSTYYASWAKG

SEQ ID NO: 11 - protein, 16 aa, synthetic construct
FISRSGITYYASWAKG

SEQ ID NO: 12 - protein, 16 aa, synthetic construct
IINYYSQTYYASWAKG

SEQ ID NO: 13 - protein, 16 aa, synthetic construct
VINPSGSAYYATWVNG

SEQ ID NO: 14 - protein, 7 aa, synthetic construct
ISRSGIT

SEQ ID NO: 15 - protein, 9 aa, synthetic construct
FISRSGITY

SEQ ID NO: 16 - protein, 5 aa, synthetic construct
SRSGI

SEQ ID NO: 17 - protein, 12 aa, synthetic construct
WIGFISRSGITY

SEQ ID NO: 18 - protein, 10 aa, synthetic construct
DPDGSIVFDI

SEQ ID NO: 19 - protein, 16 aa, synthetic construct
EFGAVGSDYYRDAFNL

SEQ ID NO: 20 - protein, 16 aa, synthetic construct
EFGAVGSDYYRDALRL

SEQ ID NO: 21 - protein, 15 aa, synthetic construct
DYITAGDYYMDAFDP

SEQ ID NO: 22 - protein, 18 aa, synthetic construct
AREFGAVGSDYYRDAFNL

SEQ ID NO: 23 - protein, 17 aa, synthetic construct
AREFGAVGSDYYRDAFN

SEQ ID NO: 24 - protein, 13 aa, synthetic construct
QSSQSVVYNNRLS

SEQ ID NO: 25 - protein, 11 aa, synthetic construct
QASESINSWLS

SEQ ID NO: 26 - protein, 11 aa, synthetic construct
QASQNIYSNLA

SEQ ID NO: 27 - protein, 13 aa, synthetic construct
QSSQSVYSNKRLA

SEQ ID NO: 28 - protein, 11 aa, synthetic construct
QASQSIGSNLA

SEQ ID NO: 29 - protein, 11 aa, synthetic construct
QASQSISNQLS

SEQ ID NO: 30 - protein, 6 aa, synthetic construct
ESINSW

SEQ ID NO: 31 - protein, 7 aa, synthetic construct
NSWLSWY

SEQ ID NO: 32 - protein, 7 aa, synthetic construct
GASTLAS

SEQ ID NO: 33 - protein, 7 aa, synthetic construct
RASTLAS

SEQ ID NO: 34 - protein, 7 aa, synthetic construct
GASNLAS

SEQ ID NO: 35 - protein, 7 aa, synthetic construct
GASTLES

SEQ ID NO: 36 - protein, 10 aa, synthetic construct
LLIYRASTLA

SEQ ID NO: 37 - protein, 13 aa, synthetic construct
LGSYDCSSGDCHA

SEQ ID NO: 38 - protein, 12 aa, synthetic construct
QSYYEEDGIGYA

SEQ ID NO: 39 - protein, 12 aa, synthetic construct
QGYDYSTAGAYP

SEQ ID NO: 40 - protein, 13 aa, synthetic construct
AGGYDCSTGDCWT

SEQ ID NO: 41 - protein, 12 aa, synthetic construct
QSYYEGSDIGYA

SEQ ID NO: 42 - protein, 12 aa, synthetic construct
QQGYNRDNVDNL

SEQ ID NO: 43 - protein, 11 aa, synthetic construct
QSYYEEDGIGY

SEQ ID NO: 44 - protein, 134 aa, synthetic construct
METGLRWLLLVAVLKGVQCQSLEESGGRLVTPGTPLTLTCTVSGFSLSSQKVGWVRQAPGKGLEWIGIINNYGSTYYASWAKGRFTISKTSTTVDLRITSLTAEDTATYFCARDPDGSIVFDIWGPGTLVTVSL

SEQ ID NO: 45 - protein, 140 aa, synthetic construct
METGLRWLLLVAVLKGVQCQSVEESGGRLVTPGTPLTLTCTVSGFSLSSYAMIWVRQAPGKGLEWIGFISRSGITYYASWAKGRFTISKTSTTVDLKMTSLTTEDTATYFCAREFGAVGSDYYRDAFNLWGPGTLVTVSS

SEQ ID NO: 46 - protein, 134 aa, synthetic construct
METGLRWLLLVAVLKGVQCQSLEESGGRLVTPGTPLTLTCTVSGFSLNNYKVGWVRQAPGKGLEWIGIINYYSQTYYASWAKGRFTISKTSTTVDLKLTSPTTEDTATYFCARDPDGSIVFDIWGPGTLVTVSL

SEQ ID NO: 47 - protein, 140 aa, synthetic construct
METGLRWLLLVAVLKGVQCQSVEESGGGLVTPGGTLTLTCTVSGFSLSNYAMSWVRQAPGKGLEWIGFISRSGITYYASWAKGRFTISKTSTTVDLKITSPTTEDTAAYFCAREFGAVGSDYYRDALRLWGPGTLVTVSS

SEQ ID NO: 48 - protein, 139 aa, synthetic construct
METGLRWLLLVAVLKGVQCQSLEESGGRLVTPGTPLTLTCTVSGIDLSTHAMTWVRQAPGKGLEWIGVINPSGSAYYATWVNGRFTISKTSTTVDLKITSPTTGDTAKYFCARDYITAGDYYMDAFDPWGPGTLVTVSS

SEQ ID NO: 49 - protein, 135 aa, synthetic construct
MDTRAPTQLLGLLLLWLPGATFAQVLTQTASPVSAAVGGTVTINCQSSQSVVYNNRLSWFQQKPGQPPKLLIYGASTLASGVPSRFKGSGSGTQFTLTISDVQCDDAATYYCLGSYDCSSGDCHAFGGGTEVVVK

SEQ ID NO: 50 - protein, 133 aa, synthetic construct
MDMRAPTQLLGLLLLWLPGARCADIVMTQTPASVEAAVGGTVTINCQASESINSWLSWYQQKPGQPPNLLIYRASTLASGVPSRFSGGGSGTEYTLTISDLECADAVTYYCQSYYEEDGIGYAFGGGTEVVVE

SEQ ID NO: 51 - protein, 133 aa, synthetic construct
MDMRAPTQLLGLLLLWLPGARCADIVMTQTPSSVSAAVGGTVTINCQASQNIYSNLAWYQQKPGQRPRLLIYGASNLASGVPSRFKGSRSGTEFTLTISDLECADAATYYCQGYDYSTAGAYPFGGGTAVVVK

SEQ ID NO: 52 - protein, 135 aa, synthetic construct
MDTRAPTQLLGLLLLWLPGATFAQVLTQTASPVSAAVGSTVTINCQSSQSVYSNKRLAWFQLKPGQPPKLLIYGASTLASGVPSRFKGSGSGTQFTLTISDVQCDDAATYYCAGGYDCSTGDCWTFGGGTEVVVT

SEQ ID NO: 53 - protein, 133 aa, synthetic construct
MDMRAPTQLLGLLLLWLPGARCADIVMTQTPSSVSAAVGGTVTIKCQASQSIGSNLAWYQQKPGQPPKLLIYGASTLESGVPSRFKGSGSGTEYTLTISDLECADAATYYCQSYYEGSDIGYAFGGGTEVVVE

SEQ ID NO: 54 - protein, 133 aa, synthetic construct
MDTRAPTQLLGLLLLWLPGARCADIVMTQTPASVSAAVGGTVTIKCQASQSISNQLSWYQQKSGQPPKLLIYRASTLASGVPSRFKGSGSGTEFTLTISDLECADAATYYCQQGYNRDNVDNLFGGGTEVVVK

SEQ ID NO: 55 - protein, 457 aa, synthetic construct
METGLRWLLLVAVLKGVQCQSLEESGGRLVTPGTPLTLTCTVSGFSLSSQKVGWVRQAPGKGLEWIGIINNYGSTYYASWAKGRFTISKTSTTVDLRITSLTAEDTATYFCARDPDGSIVFDIWGPGTLVTVSLGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGRSSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 56 - protein, 239 aa, synthetic construct
MDTRAPTQLLGLLLLWLPGATFAQVLTQTASPVSAAVGGTVTINCQSSQSVVYNNRLSWFQQKPGQPPKLLIYGASTLASGVPSRFKGSGSGTQFTLTISDVQCDDAATYYCLGSYDCSSGDCHAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 57 - protein, 463 aa, synthetic construct
METGLRWLLLVAVLKGVQCQSVEESGGRLVTPGTPLTLTCTVSGFSLSSYAMIWVRQAPGKGLEWIGFISRSGITYYASWAKGRFTISKTSTTVDLKMTSLTTEDTATYFCAREFGAVGSDYYRDAFNLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGRSSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 58 - protein, 237 aa, synthetic construct
MDMRAPTQLLGLLLLWLPGARCADIVMTQTPASVEAAVGGTVTINCQASESINSWLSWYQQKPGQPPNLLIYRASTLASGVPSRFSGGGSGTEYTLTISDLECADAVTYYCQSYYEEDGIGYAFGGGTEVVVEGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 59 - protein, 237 aa, synthetic construct
MDMRAPTQLLGLLLLWLPGARCADIVMTQTPSSVSAAVGGTVTINCQASQNIYSNLAWYQQKPGQRPRLLIYGASNLASGVPSRFKGSRSGTEFTLTISDLECADAATYYCQGYDYSTAGAYPFGGGTAVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 60 - protein, 457 aa, synthetic construct
METGLRWLLLVAVLKGVQCQSLEESGGRLVTPGTPLTLTCTVSGFSLNNYKVGWVRQAPGKGLEWIGIINYYSQTYYASWAKGRFTISKTSTTVDLKLTSPTTEDTATYFCARDPDGSIVFDIWGPGTLVTVSLGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVVPSTCSKPTCPPPELLGRSSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 61 - protein, 239 aa, synthetic construct
MDTRAPTQLLGLLLLWLPGATFAQVLTQTASPVSAAVGSTVTINCQSSQSVYSNKRLAWFQLKPGQPPKLLIYGASTLASGVPSRFKGSGSGTQFTLTISDVQCDDAATYYCAGGYDCSTGDCWTFGGGTEVVVTGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 62 - protein, 463 aa, synthetic construct
METGLRWLLLVAVLKGVQCQSVEESGGGLVTPGGTLTLTCTVSGFSLSNYAMSWVRQAPGKGLEWIGFISRSGITYYASWAKGRFTISKTSTTVDLKITSPTTEDTAAYFCAREFGAVGSDYYRDALRLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGRSSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 63 - protein, 237 aa, synthetic construct
MDMRAPTQLLGLLLLWLPGARCADIVMTQTPSSVSAAVGGTVTIKCQASQSIGSNLAWYQQKPGQPPKLLIYGASTLESGVPSRFKGSGSGTEYTLTISDLECADAATYYCQSYYEGSDIGYAFGGGTEVVVEGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 64 - protein, 462 aa, synthetic construct
METGLRWLLLVAVLKGVQCQSLEESGGRLVTPGTPLTLTCTVSGIDLSTHAMTWVRQAPGKGLEWIGVINPSGSAYYATWVNGRFTISKTSTTVDLKITSPTTGDTAKYFCARDYITAGDYYMDAFDPWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGRSSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 65 - protein, 237 aa, synthetic construct
MDTRAPTQLLGLLLLWLPGARCADIVMTQTPASVSAAVGGTVTIKCQASQSISNQLSWYQQKSGQPPKLLIYRASTLASGVPSRFKGSGSGTEFTLTISDLECADAATYYCQQGYNRDNVDNLFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 66 - DNA, 402 bp, synthetic construct
  source 1..402: mol_type = other DNA
atggagactgggctgcgctggcttctcctggtcgctgtgctcaaaggtgtccagtgtcagtcgctggaggagtccgggggtcgcctggtcacgcctgggacacccctgacactcacctgcacagtctctggattttccctcagtagccagaaagtgggctgggtccgccaggctccagggaaggggctggaatggatcggaatcattaataattatggtagcacatactacgcgagctgggcgaaaggccgattcaccatctcgaaaacctcgaccacagtggatctgagaatcaccagtctgacggccgaggacacggccacctatttctgtgcccgtgatcctgatggtagtattgtctttgacatctggggcccaggcacccttgtcaccgtctccttg

SEQ ID NO: 67 - DNA, 420 bp, synthetic construct
  source 1..420: mol_type = other DNA
atggagactgggctgcgctggcttctcctggtcgctgtgctcaaaggtgtccagtgtcagtcggtggaggagtccgggggtcgcctggtcacgcctgggacacccctgacactcacctgcaccgtctctggattctccctcagtagctatgcaatgatctgggtccgccaggctccagggaaggggctggaatggatcggattcattagtcgtagtggtatcacatactacgcgagctgggcaaaaggccgattcaccatctccaaaacctcgaccacggtggatctgaaaatgaccagtctgacaaccgaggacacggccacctatttctgtgccagagaattcggtgctgttggtagtgattattatagggacgcctttaacttgtggggcccaggcaccctggtcaccgtctcctca

SEQ ID NO: 68 - DNA, 402 bp, synthetic construct
  source 1..402: mol_type = other DNA
atggagactgggctgcgctggcttctcctggtcgctgtgctcaaaggtgtccagtgtcagtcgctggaggagtccgggggtcgcctggtcacgcctgggacacccctgacactcacctgcacagtctctggattttccctaaataactacaaagtgggctgggtccgccaggctccaggaaaggggctggaatggatcggaatcattaactattatagtcagacatactacgcgagctgggccaaaggccgattcaccatctcgaaaacctcgaccacggtggatctgaagctcaccagtccgacaaccgaagacacggccacctatttctgtgcccgtgatcctgatggtagtattgtctttgacatctggggcccaggcacccttgtcaccgtctccttg

SEQ ID NO: 69 - DNA, 420 bp, synthetic construct
  source 1..420: mol_type = other DNA
atggagactgggctgcgctggcttctcctggtcgctgtgctcaaaggtgtccagtgtcagtcggtggaggagtccggaggaggcctggtaacgcctggaggaaccctgacactcacctgcaccgtctctggattctccctcagtaactatgcaatgagctgggtccgccaggctccagggaaggggctggaatggatcggattcattagtcgtagtggtattacatactacgcgagctgggcaaaaggccgattcaccatctccaaaacctcgaccacggtggatctgaaaatcaccagtccgacgaccgaggacacggccgcctatttctgtgccagagaattcggtgctgttggtagtgattattatagggacgccttgaggttgtggggcccaggcaccctggtcaccgtctcctca

SEQ ID NO: 70 - DNA, 417 bp, synthetic construct
  source 1..417: mol_type = other DNA
atggagactgggctgcgctggcttctcctggtcgctgtgctcaaaggtgtccagtgtcagtcgctggaggagtccgggggtcgcctggtaacgcctgggacacccctgacactcacctgcacagtctctggaatcgacctcagtacccatgcaatgacctgggtccgccaggctccaggaaaggggctggaatggatcggagtcattaatcctagtggtagcgcatactacgcgacctgggtgaatggccgattcaccatctccaaaacctcgaccacggtggatctgaaaatcaccagtccgacaaccggggacacggccaagtatttctgtgccagagattatattactgcgggtgattattatatggatgcttttgatccctggggcccaggcaccctggtcaccgtctcctca

SEQ ID NO: 71 - DNA, 405 bp, synthetic construct
  source 1..405: mol_type = other DNA
atggacacgagggcccccactcagctgctggggctcctgctgctctggctcccaggtgccacatttgcccaagtgctgacccagactgcatcccccgtgtctgcggctgttggaggcacagtcaccatcaattgccagtccagtcagagtgttgtatataacaaccgcttatcctggtttcaacagaaaccagggcagcctcccaagctcctgatctatggtgcatccactctggcatctggggtcccatcgcggttcaaaggcagtggatctgggacacagttcactctcaccatcagcgacgtgcagtgtgacgatgctgccacttactactgtctaggctcctatgattgtagtagtggtgattgccatgctttcggcggagggaccgaggtggtggtcaaa

SEQ ID NO: 72 - DNA, 399 bp, synthetic construct
  source 1..399: mol_type = other DNA
atggacatgagggcccccactcagctgctggggctcctactgctctggctcccaggtgccagatgtgctgacattgtgatgacccagactccagcctccgtggaggcagctgtgggaggcacagtcaccatcaattgccaagccagtgagagcattaatagttggttgtcctggtatcagcagaaaccagggcagcctcccaacctcctgatctacagggcatccactctggcatctggggtcccatcgcggttcagtggcggtggatctgggacagagtacactctcaccatcagcgacctggagtgtgccgatgctgtcacttattactgtcaaagctattatgaggaggatggtattggttatgctttcggcggagggaccgaggtggtggtcgaa

SEQ ID NO: 73 - DNA, 399 bp, synthetic construct
  source 1..399: mol_type = other DNA
atggacatgagggcccccactcagctgctggggctcctgctgctctggctcccaggtgccagatgtgctgacattgtgatgacccagactccatcctccgtgtctgcagctgtgggaggcacagtcaccatcaattgccaggccagtcagaacatttacagcaatttagcctggtatcagcagaaaccagggcagcgtcccaggctcctgatctatggcgcatccaatctggcatctggggtcccatcgcggttcaaaggcagtagatctgggacagagttcactctcaccatcagcgacctggagtgtgccgatgctgccacttactactgtcaaggctatgattatagtactgctggtgcctatcctttcggcggagggaccgcggtggtggtcaaa

SEQ ID NO: 74 - DNA, 405 bp, synthetic construct
  source 1..405: mol_type = other DNA
atggacacgagggcccccactcagctgctggggctcctgctgctctggctcccaggtgccacatttgcccaagtgctgacccagactgcatcgcccgtgtctgcggctgtgggaagcacagtcaccatcaattgccagtccagtcagagcgtttatagtaacaagcgcttagcctggtttcagctgaaaccagggcagcctcccaagctcctgatctatggtgcatccacactggcatctggggtcccatcgcgattcaagggcagtggatctgggacacagttcactctcaccatcagcgacgtgcagtgtgacgatgctgccacttactactgtgcaggcggttatgattgtagtactggtgattgttggactttcggcggagggaccgaggtggtggtcaca

SEQ ID NO: 75 - DNA, 399 bp, synthetic construct
  source 1..399: mol_type = other DNA
atggacatgagggcccccactcagctgctggggctcctgctgctctggctcccaggtgccagatgtgctgacatcgtgatgacccagactccatcctccgtgtctgcagctgtgggaggcacagtcaccatcaagtgccaggccagtcagagcattggtagtaatttagcctggtatcagcagaaaccagggcagcctcccaagctcctgatctatggtgcatccactctggaatctggggtcccatcgcggtttaaaggcagtggatctgggacagagtacactctcaccatcagcgacctggagtgtgccgatgctgccacttactactgtcaaagctattatgagggtagtgatattggttatgctttcggcggagggaccgaggtggtggtcgaa

SEQ ID NO: 76 - DNA, 399 bp, synthetic construct
  source 1..399: mol_type = other DNA
atggacacgagggcccccactcagctgctggggctcctgctgctctggctcccaggtgccagatgtgctgacatcgtgatgacccagactccagcctctgtgtctgcagctgtgggaggcacagtcaccatcaagtgccaggccagtcagagcattagcaaccaactatcctggtatcagcagaaatcagggcagcctcccaagctcctgatctacagggcatctactctggcatctggggtcccatcgcggttcaaaggcagtggatctgggacagagttcactctcaccatcagcgacctggagtgtgccgatgctgccacttactactgtcaacagggttataatagagataatgttgataatcttttcggcggagggaccgaggtggtggtcaaa

SEQ ID NO: 77 - DNA, 1374 bp, synthetic construct
  source 1..1374: mol_type = other DNA
atggagactgggctgcgctggcttctcctggtcgctgtgctcaaaggtgtccagtgtcagtcgctggaggagtccgggggtcgcctggtcacgcctgggacacccctgacactcacctgcacagtctctggattttccctcagtagccagaaagtgggctgggtccgccaggctccagggaaggggctggaatggatcggaatcattaataattatggtagcacatactacgcgagctgggcgaaaggccgattcaccatctcgaaaacctcgaccacagtggatctgagaatcaccagtctgacggccgaggacacggccacctatttctgtgcccgtgatcctgatggtagtattgtctttgacatctggggcccaggcacccttgtcaccgtctccttggggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggcgatcctctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 78 - DNA, 1392 bp, synthetic construct
  source 1..1392: mol_type = other DNA
atggagactgggctgcgctggcttctcctggtcgctgtgctcaaaggtgtccagtgtcagtcggtggaggagtccgggggtcgcctggtcacgcctgggacacccctgacactcacctgcaccgtctctggattctccctcagtagctatgcaatgatctgggtccgccaggctccagggaaggggctggaatggatcggattcattagtcgtagtggtatcacatactacgcgagctgggcaaaaggccgattcaccatctccaaaacctcgaccacggtggatctgaaaatgaccagtctgacaaccgaggacacggccacctatttctgtgccagagaattcggtgctgttggtagtgattattatagggacgcctttaacttgtggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggcgatcctctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 79 - DNA, 1374 bp, synthetic construct
  source 1..1374: mol_type = other DNA
atggagactgggctgcgctggcttctcctggtcgctgtgctcaaaggtgtccagtgtcagtcgctggaggagtccgggggtcgcctggtcacgcctgggacacccctgacactcacctgcacagtctctggattttccctaaataactacaaagtgggctgggtccgccaggctccaggaaaggggctggaatggatcggaatcattaactattatagtcagacatactacgcgagctgggccaaaggccgattcaccatctcgaaaacctcgaccacggtggatctgaagctcaccagtccgacaaccgaagacacggccacctatttctgtgcccgtgatcctgatggtagtattgtctttgacatctggggcccaggcacccttgtcaccgtctccttggggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgtgccctcgacatgcagcaagcccacgtgcccaccccctgaactcctggggcgatcctctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 80 - DNA, 1392 bp, synthetic construct
  source 1..1392: mol_type = other DNA
atggagactgggctgcgctggcttctcctggtcgctgtgctcaaaggtgtccagtgtcagtcggtggaggagtccggaggaggcctggtaacgcctggaggaaccctgacactcacctgcaccgtctctggattctccctcagtaactatgcaatgagctgggtccgccaggctccagggaaggggctggaatggatcggattcattagtcgtagtggtattacatactacgcgagctgggcaaaaggccgattcaccatctccaaaacctcgaccacggtggatctgaaaatcaccagtccgacgaccgaggacacggccgcctatttctgtgccagagaattcggtgctgttggtagtgattattatagggacgccttgaggttgtggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggcgatcctctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 81 - DNA, 1389 bp, synthetic construct
  source 1..1389: mol_type = other DNA
atggagactgggctgcgctggcttctcctggtcgctgtgctcaaaggtgtccagtgtcagtcgctggaggagtccgggggtcgcctggtaacgcctgggacacccctgacactcacctgcacagtctctggaatcgacctcagtacccatgcaatgacctgggtccgccaggctccaggaaaggggctggaatggatcggagtcattaatcctagtggtagcgcatactacgcgacctgggtgaatggccgattcaccatctccaaaacctcgaccacggtggatctgaaaatcaccagtccgacaaccggggacacggccaagtatttctgtgccagagattatattactgcgggtgattattatatggatgcttttgatccctggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggcgatcctctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 82 - DNA, 720 bp, synthetic construct
  source 1..720: mol_type = other DNA
atggacacgagggcccccactcagctgctggggctcctgctgctctggctcccaggtgccacatttgcccaagtgctgacccagactgcatcccccgtgtctgcggctgttggaggcacagtcaccatcaattgccagtccagtcagagtgttgtatataacaaccgcttatcctggtttcaacagaaaccagggcagcctcccaagctcctgatctatggtgcatccactctggcatctggggtcccatcgcggttcaaaggcagtggatctgggacacagttcactctcaccatcagcgacgtgcagtgtgacgatgctgccacttactactgtctaggctcctatgattgtagtagtggtgattgccatgctttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 83 - DNA, 714 bp, synthetic construct
  source 1..714: mol_type = other DNA
atggacatgagggcccccactcagctgctggggctcctactgctctggctcccaggtgccagatgtgctgacattgtgatgacccagactccagcctccgtggaggcagctgtgggaggcacagtcaccatcaattgccaagccagtgagagcattaatagttggttgtcctggtatcagcagaaaccagggcagcctcccaacctcctgatctacagggcatccactctggcatctggggtcccatcgcggttcagtggcggtggatctgggacagagtacactctcaccatcagcgacctggagtgtgccgatgctgtcacttattactgtcaaagctattatgaggaggatggtattggttatgctttcggcggagggaccgaggtggtggtcgaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 84 - DNA, 714 bp, synthetic construct
  source 1..714: mol_type = other DNA
atggacatgagggcccccactcagctgctggggctcctgctgctctggctcccaggtgccagatgtgctgacattgtgatgacccagactccatcctccgtgtctgcagctgtgggaggcacagtcaccatcaattgccaggccagtcagaacatttacagcaatttagcctggtatcagcagaaaccagggcagcgtcccaggctcctgatctatggcgcatccaatctggcatctggggtcccatcgcggttcaaaggcagtagatctgggacagagttcactctcaccatcagcgacctggagtgtgccgatgctgccacttactactgtcaaggctatgattatagtactgctggtgcctatcctttcggcggagggaccgcggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 85 - DNA, 720 bp, synthetic construct
  source 1..720: mol_type = other DNA
atggacacgagggcccccactcagctgctggggctcctgctgctctggctcccaggtgccacatttgcccaagtgctgacccagactgcatcgcccgtgtctgcggctgtgggaagcacagtcaccatcaattgccagtccagtcagagcgtttatagtaacaagcgcttagcctggtttcagctgaaaccagggcagcctcccaagctcctgatctatggtgcatccacactggcatctggggtcccatcgcgattcaagggcagtggatctgggacacagttcactctcaccatcagcgacgtgcagtgtgacgatgctgccacttactactgtgcaggcggttatgattgtagtactggtgattgttggactttcggcggagggaccgaggtggtggtcacaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 86 - DNA, 714 bp, synthetic construct
  source 1..714: mol_type = other DNA
atggacatgagggcccccactcagctgctggggctcctgctgctctggctcccaggtgccagatgtgctgacatcgtgatgacccagactccatcctccgtgtctgcagctgtgggaggcacagtcaccatcaagtgccaggccagtcagagcattggtagtaatttagcctggtatcagcagaaaccagggcagcctcccaagctcctgatctatggtgcatccactctggaatctggggtcccatcgcggtttaaaggcagtggatctgggacagagtacactctcaccatcagcgacctggagtgtgccgatgctgccacttactactgtcaaagctattatgagggtagtgatattggttatgctttcggcggagggaccgaggtggtggtcgaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 87 - DNA, 714 bp, synthetic construct
  source 1..714: mol_type = other DNA
atggacacgagggcccccactcagctgctggggctcctgctgctctggctcccaggtgccagatgtgctgacatcgtgatgacccagactccagcctctgtgtctgcagctgtgggaggcacagtcaccatcaagtgccaggccagtcagagcattagcaaccaactatcctggtatcagcagaaatcagggcagcctcccaagctcctgatctacagggcatctactctggcatctggggtcccatcgcggttcaaaggcagtggatctgggacagagttcactctcaccatcagcgacctggagtgtgccgatgctgccacttactactgtcaacagggttataatagagataatgttgataatcttttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 88 - protein, 17 aa, synthetic construct
  SITE 9: note = Phosphorylated threonine
RSRTPSLPTPPTREPKC

SEQ ID NO: 89 - protein, 17 aa, synthetic construct
  SITE 6: note = Phosphorylated threonine
TPSLPTPPTREPKKVAC

SEQ ID NO: 90 - protein, 20 aa, synthetic construct
RSRTPSLPTPPTREPKKVAC

SEQ ID NO: 91 - protein, 17 aa, synthetic construct
  SITE 4: note = Phosphorylated threonine
RSRTPSLPTPPTREPKC

SEQ ID NO: 92 - protein, 17 aa, synthetic construct
  SITE 6: note = Phosphorylated serine
RSRTPSLPTPPTREPKC

SEQ ID NO: 93 - protein, 20 aa, synthetic construct
  SITE 12: note = Phosphorylated threonine
RSRTPSLPTPPTREPKKVAC

SEQ ID NO: 94 - protein, 16 aa, synthetic construct
  SITE 7: note = Phosphorylated threonine
KVAVVRTPPKSPSSAC

SEQ ID NO: 95 - protein, 16 aa, synthetic construct
KVAVVRTPPKSPSSAC